(19) United States
(12) Patent Application Publication
(10) Pub. No.: US 2019/0008973 A1
(43) Pub. Date: Jan. 10, 2019

(54) INTRALIPID-IODINE FOR IMAGING

(71) Applicant: Joseph Eldor, Jerusalem (IL)

(72) Inventor: Joseph Eldor, Jerusalem (IL)

(21) Appl. No.: 16/028,804

(22) Filed: Jul. 6, 2018

Related U.S. Application Data
(60) Provisional application No. 62/529,073, filed on Jul. 6, 2017.

(51) Int. Cl. A61K 47/44; A61K 49/04; A61K 9/107; A61K 9/00

(52) U.S. Cl. CPC A61K 47/44; A61K 9/0019; A61K 9/107; A61K 49/0438

(57) ABSTRACT

The present invention relates to intralipid-iodine and intralipid-iodine compounds for use in imaging and therapy.

### FIELD OF THE INVENTION

The present invention relates to the field of imaging using intralipid-based compounds. Specifically, the present invention relates to intralipid-iodine compounds and compositions for use in imaging, such as hysterosalpingography in infertile women, X-ray imaging with contrast materials.

### SUMMARY OF THE INVENTION

The present invention provides a novel approach for imaging procedures that utilizes contrast materials, such as hysterosalpingography in infertile women, X-ray imaging, cardiac- and limbs-catheterization. Specifically, the present invention provides a multidisciplinary and multipotent intralipid-iodine compounds and compositions for use in imaging that utilizes contrast materials.

In certain embodiments, the present invention provides a multidisciplinary and multipotent intralipid-iodine for use in imaging.

In certain embodiments of the intralipid-iodine of the invention the imaging is hysterosalpingography in infertile women.

In certain embodiments of the intralipid-iodine of the invention the imaging is X-ray imaging or cardiac- and limbs-catheterization.

In certain embodiments of the intralipid-iodine according to the embodiments above, the intralipid-iodine is formulated for injection into the blood stream of a patient.

In certain embodiments of the intralipid-iodine according to the embodiments above, the intralipid-iodine is any lipid emulsion.

In certain embodiments of the intralipid-iodine according to the embodiments above, the intralipid-iodine absorbs/attaches obstructive or potentially-obstructive particles located within blood vessels and/or other organs in the body, and removes same to thereby open clogged blood vessels.

In certain embodiments, the present invention further provides a multidisciplinary and multipotent composition comprising intralipid-iodine for use in imaging.

In certain embodiments, the composition of the invention further comprises a pharmaceutically acceptable carrier adapted for injection.

In certain embodiments of the composition according to the embodiments above, the composition is formulated for administration into the blood stream.

In certain embodiments of the composition according to the embodiments above, the intralipid-iodine is any lipid emulsion.

In certain embodiments of the composition according to the embodiments above, the intralipid-iodine absorbs/attaches obstructive or potentially-obstructive particles located within blood vessels and/or other organs in the body, and removes same to thereby open clogged blood vessels.

In certain embodiments, the present invention further provides a method for imaging comprising administering to an individual in need thereof an effective amount of intralipid-iodine or a composition comprising same.

In certain embodiments, the method of the invention comprises a step of injecting the intralipid-iodine or the composition comprising same into the blood stream of the individual.

In certain embodiments of the method according to the embodiments above, the intralipid-iodine is any lipid emulsion.

In certain embodiments of the method according to the embodiments above, the intralipid-iodine absorbs/attaches obstructive or potentially-obstructive particles located within blood vessels and/or other organs in the body, and removes same to thereby open clogged blood vessels.

In certain embodiments, the present invention further provides an intravenous composition comprising intralipid-iodine for use in imaging.

In certain embodiments, the intravenous composition of the invention further comprises a pharmaceutically acceptable carrier adapted for injection.

In certain embodiments of the intravenous composition according to the embodiments above, the intralipid-iodine absorbs/attaches obstructive or potentially-obstructive particles located within blood vessels and/or other organs in the body, and removes same to thereby open clogged blood vessels.

### DETAILED DESCRIPTION OF THE INVENTION

Intravenous lipid emulsions have been used experimentally since at least the 19^{th} century. An early product marketed in 1957 under the name Lipomul was briefly used in the United States but was subsequently withdrawn due to side effects. Intralipid was invented by the Swedish physician and nutrition researcher Arvid Wretlind, and was approved for clinical use in Sweden in 1962 (Hallberg et al., 1967). In the United States, the Food and Drug Administration initially declined to approve the product due to prior experience with another fat emulsion. It was approved in the United States in 1972.

Tubal disease is the cause of female infertility in approximately 30% of women (Juras et al., 2002) and 10-25% of these are due to proximal tubal obstruction (Elstein et al., 2008). Selective salpingography represent an approach in the diagnosis and treatment of proximal tubal abnormalities. Intralipid is a synthetic product composed of 10% soybean oil, 1.2% egg yolk phospholipids, 2.25% glycerin, and water. When indicated, intralipid is infused 7-10 days prior to embryo transfer. Rates of ongoing pregnancy and live births were higher among women who underwent hysterosalpingography (HSG) with oil contrast than among women who underwent this procedure with water contrast.

It is the first time that intralipid-iodine is suggested to be used for HSG in infertile women.

The common indications for selective salpingography are to differentiate spasm from true obstruction (Thurmond et al., 2000). In addition to that, it allows clarifying findings from an equivocal hysterosalpingogram. Isthmic as well as intamural blockages were included. The tubal obstruction may be due to amorphous materials occluding the tubal lumen, inflammatory changes and adhesions (Papaioannou et al., 2003). The use of selective salpingography and fallopian tube recanalization has revolutionized the diagnosis and treatment of infertility. Diagnostic procedure has been used since 1980 (Thurmond et al., 2000). It consists on opacification of the fallopian tube directly through a catheter placed in the tubal ostium. The objective is to differentiate spasm from true obstruction and to clear it with a catheter and guide wire system.

Pregnancy rates among infertile women have been reported to increase after HSG, but it is unclear whether the type of contrast medium used (oil-based or water-soluble contrast) influences this potential therapeutic effect.

Dreyer et al. (2017) performed a multicenter, randomized trial in 27 hospitals in the Netherlands in which infertile women who were undergoing HSG were randomly assigned to undergo this procedure with the use of oil-based or water-based contrast. Subsequently, couples received expectant management or the women underwent intrauterine insemination. The primary outcome was ongoing pregnancy within 6 months after randomization. Outcomes were analyzed according to the intention-to-treat principle.

A total of 1119 women were randomly assigned to HSG with oil contrast (557 women) or water contrast (562 women). A total of 220 of 554 women in the oil group (39.7%) and 161 of 554 women in the water group (29.1%) had an ongoing pregnancy (rate ratio, 1.37; 95% confidence interval, 1.16 to 1.61; P<0.001), and 214 of 552 women in the oil group (38.8%) and 155 of 552 women in the water group (28.1%) had live births (rate ratio, 1.38; 95% CI, 1.17 to 1.64; P<0.001). Rates of adverse events were low and similar in the two groups.

Rates of ongoing pregnancy and live births were higher among women who underwent HSG with oil contrast than among women who underwent this procedure with water contrast.

HSG, which should be done in the follicular phase of the cycle, evaluates the contour of the uterine cavity, cervical canal, and tubal lumina. Other than being diagnostic, it can prove to be therapeutic. The instrument used to introduce the radio-opaque medium should be chosen to give the least discomfort and to cause no leakage of dye from the cervix. Water-soluble medium is usually used rather than an oil-based medium. Fluoroscopy with image intensification gives the best results. Insufficient dye injection will give an incomplete study. Too much dye injection, especially under pressure, might cause extravasation of the dye into the vascular system or conceal the fimbrial ends of the tubes (Baramki et al., 2005).

To determine whether hysterosalpingography (HSG) increases the conception rate and to compare the therapeutic effectiveness of oil and water soluble contrast media, the histories of 744 women who attended the Yale Infertility Clinic in 1965-69 were reviewed. Exclusion of women who had been infertile less than 1 year before coming to the clinic, in whom a HSG was done outside the clinic, and in whom there was no follow-up resulted in a study population of 460. The pregnancy rate for the study group (49%) was identical to that for the larger population. The study group was divided into women who became pregnant during their clinic attendance or within 1 year of terminating clinic care and women who did not become pregnant in this period. These 2 categories were then subdivided according to whether a HSG was done. To determine the relative effectiveness of different media, the conception rate following HSG in the Yale Clinic, where the iodized oil Ethiodol was used, was compared with that among 63 women who had HSG done by a private practitioner with the water soluble dye Salpix. 221 women were in the Ethiodol HSG group, 121 (55%) of whom conceived. Of the 239 Yale Clinic patients who did not have HSG, 103 (43%) became pregnant. Exclusion of couples with organic factors that could account for infertility resulted in a pregnancy rate of 58% for the HSG group and 47% for the non-HSG group. 25 (40%) of the 63 women in the Salpix group conceived, but exclusion of couples with organic factors lowered the pregnancy rate to 38% in this group. The average length of infertility was 0.5 years longer in the non-HSG group than in the Ethiodol HSG group, but shorter in the Salpix HSG group compared to the Ethiodol group. These results suggest that Ethiodol HSG may enhance fertility. Although the oil media has been criticized on the basis that it may cause granuloma formation or embolization, documented complications in the Yale series were rare. In contrast to earlier studies, unilateral nonpatency on x-ray was not found to affect eventual conception (58% pregnancy rate among women in the Ethiodol HSG group with normal tubes compared with 50% in those with filling of only 1 tube) (Mackey et al., 1971).

Previous studies have suggested increased fertility rates following HSG using oil as compared with aqueous contrast medium. To compare the possible fertility-enhancing effects of two agents used for HSG, this prospective randomized study evaluated the subsequent fertility rates in 121 patients who underwent HSG, in which either oil or aqueous contrast medium was used. After random assignment to either agent, patients were observed for four menstrual cycles after HSG without resorting to any therapy other than clomiphene citrate where indicated. The pregnancy rates for the four cycles after HSG were compared with chi-square analysis in the total study and in the diagnostic subgroups. The subgroup with infertility of unknown cause had a significantly higher pregnancy rate after HSG with oil than after HSG with aqueous contrast medium. No significant difference was seen for any other subgroup or for the overall cohort (Schwabe et al., 1983).

In a prospective randomized study, the number of pregnancies after HSG was estimated in 398 patients who had been infertile for longer than 1 year. Iohexol was used in 101 patients, ioxaglate in 102 patients, diatrizoate meglumine in 97 patients, and ethiodized poppy-seed oil in 98 patients. Ten months after HSG, the patient, referring physician, and/or hospital department was consulted for information about pregnancies. Questionnaires were obtained from the patients who became pregnant during the waiting period of 3 months. No differences in demographic parameters, infertility status, or diagnosis made with HSG were detected among the four contrast media groups. Significantly more patients became pregnant after HSG in the ethiodized poppy-seed oil group than in the three water-soluble contrast media groups (P<0.01). When only intrauterine pregnancies resulting in full-term births were considered, significant differences in pregnancy rates between the oil-soluble and the water-soluble contrast media groups became more obvious. In the group that received ethiodized poppy-seed oil, almost one-third of the infertile women had normal pregnancies and childbirths after HSG (Rasmussen et al., 1991).

Meta-analysis of four randomized clinical trials (RCTs) and six nonrandomized controlled studies evaluated pregnancy rates after the use of oil- or water-soluble contrast media during HSG. Four identified RCTs studied 800 patients and six nonrandomized studies comprised an additional 1,806 patients, all experiencing primary or secondary infertility.

Pregnancy rates were significantly higher in the oil-soluble contrast media group compared with the water-soluble contrast media group in the RCTs. Inclusion of the six nonrandomized studies did not alter this conclusion. This apparent benefit was greatest for patients with unexplained infertility.

Oil-soluble contrast media have a therapeutic effect compared with water-soluble media and this effect is greatest for patients who have been diagnosed as having unexplained infertility. New techniques for the evaluation of tubal patency support the hypothesis that tubal “plugs” may be involved in proximal tubal blockage (Watson et al., 1994).

Ninety-three patients with unilateral or bilateral proximal tubal occlusion confirmed by HSG or laparoscopy underwent FTR with use of water-soluble contrast material alone (n=50) or also had an oil-based agent injected into each tube after recanalization (n=43). Pregnancy rates and outcomes of the two groups were studied retrospectively. With respect to differences between groups, only the body mass index proved to be a significant predictor (oil, 28.4; water, 24.7; P=0.008). Mean age, duration of infertility, type of infertility, and initial diagnosis were comparable. There was a weak trend toward a higher pregnancy rate in the oil-based contrast material group, but it was not significant (P=0.64). The average time to pregnancy was 4.4 months with use of oil-based contrast material, compared to 7.7 months with use of only water-soluble contrast material (P=0.03). The use of an oil-based agent had little effect on the rate of conception, but time to conception was reduced by more than 3 months (Pinto et al., 2003).

HSG is used commonly in the evaluation of infertility and in the diagnosis of anomalies of the uterus and fallopian tubes. There is continued debate over the safety and diagnostic or therapeutic efficacy of water-soluble versus oil-based contrast media.

A 29-year-old woman with secondary infertility underwent HSG with both water-soluble and oil-based contrast. The fallopian tubes appeared normal. Six months later, a plain abdominal radiograph obtained at the occasion of a minor motor vehicle accident revealed evidence of retained loculated pelvic contrast material. Subsequent laparoscopy identified adhesions and cul-de-sac implants strongly suspicious for endometriosis. Biopsy and pathologic study documented lipogranuloma.

Oil-based contrast media instilled into the pelvis at HSG can persist for prolonged periods and create granulomatous lesions mimicking endometriosis. In view of the controversy whether oil-based contrast materials are superior to water-soluble media, the routine use of oil-based contrast media should be considered carefully (Grosskinsky et al., 1994).

HSG has assumed a diagnostic and possibly therapeutic role in the evaluation of the infertile couple. The procedure is done using either an oil-based (OBCM) or a water-based (WBCM) contrast medium. Data from several retrospective studies suggest that higher pregnancy rates may be achieved when OBCM is used. Interpretation of these results, however, may be confounded by various methodologic flaws in study design and comparisons of heterogeneous populations. Letterie and Rose (1990) sought to compare the therapeutic benefit of OBCM and WBCM in a prospective randomized study of infertile patients, controlling for pelvic anatomy by laparoscopic assessment. They used ethiodized oil (Ethiodol) or iothalamate meglumine (Conray 60) for tubal lavage at the time of laparoscopy only in patients with normal pelvic anatomy. Of the 225 patients who had diagnostic laparoscopy in the evaluation of infertility, 40 (18%) had normal pelvic anatomy and an otherwise unremarkable evaluation. Adequate follow-up was available on 29 patients randomized to receive either OBCM (n=15) or WBCM (n=14). A significant difference in pregnancy rates was noted between OBCM (40%) and WBCM (14%) by chi-square analysis. No short- or long-term adverse reactions were noted. Results of this study suggest that in patients with normal pelvic anatomy as assessed laparoscopically, OBCM may offer a therapeutic benefit not evident with WBCM.

Moore et al. (1990) evaluated the effect of different iodinated contrast agents on the fallopian tube and adnexal tissue in 15 rabbits. Ethiodized oil, an oil-soluble agent, was used in five rabbits. The following water-soluble agents were used: iothalamate meglumine 30% (n=3), iothalamate meglumine 60% (n=3), and ioxilan (n=4). The agents were injected through catheters placed in the fallopian tubes. Fallopian tubes and peritoneal cavities were histologically evaluated. The contralateral tube served as a control. Ioxilan and iothalamate meglumine 30% produced no pathologic response in the tube or peritoneal cavity. Iothalamate meglumine 60% was associated with mild inflammatory infiltrate, mucosal edema, giant cell reaction, and periovarian adhesions that were bilateral but more pronounced on the injected side. Use of ethiodized oil resulted in papillary fibrous adhesions on the ovarian surface, and fat granulomas were seen in the periovarian tissues. The safety of oil-based contrast agents for use in HSG is therefore questioned. No significant differences were found among the water-soluble contrast agents.

HSG can be accomplished with either oil or water-soluble contrast medium. This randomized prospective study compared pregnancy rates in women who had HSG with either water- or oil-soluble contrast material and were followed for six months. 15 of 60 (25%) patients who received water-soluble dye conceived compared with 14 of 46 (30%) patients in the oil-soluble group, a statistically insignificant difference. Furthermore, no difference in pregnancy rates within each subgroup of fertility diagnosis was detected. Intravasation was more common in patients administered oil-based contrast materials (6 of 46 versus 1 of 60 patients, P=0.02), although no serious consequences occurred. No difference in the amount of pain as assessed by pain scoring was experienced by patients in each group. The authors conclude that pregnancy rates are similar after HSG with oil- and water-soluble contrast material, during at least the first six months after the procedure (Alper et al., 1986).

Aspects of the immunological relationship between mother and conceptus still remain a mystery, although the recent advances in molecular biology have enlightened some of the parameters that participate in feto-maternal cross-talk during implantation (Gautam et al., 2015). The atypical expression of major histocompatibility complex, the specific roles of some hormones and cytokines, as well as the temporal and spatial distributions of uterine natural killer cells, represent substantive parameters of feto-maternal immunotolerance during implantation (Oreshkova et al., 2012). Although human maternal and fetal immunology is difficult to investigate, aberrant immune responses and an imbalanced cytokine network may be related to infertility, implantation failures after IVF, and recurrent pregnancy losses (Makrigiannakis et al., 2011). Patients with recurrent implantation failure (RIF) should be tested for inherited and acquired thrombophilias. Each patient should be individually assessed and counseled regarding management with low-molecular-weight heparin (LMWH).

Empirical treatment with LMWH, aspirin, or corticosteroids is not effective for women with RIF who have negative thrombophilic tests (Fatemi et al., 2013). If thrombophilic tests are normal, patients should be tested for immunological causes. The findings of a recent study suggest that increases in the percentage of CD56(dim) cells and NK cytotoxicity in peripheral blood may be important contributing factors for both RSA and IVF failure (Karami et al., 2012). Human leukocyte antigen (HLA)-DQA1*0505 sharing or the maternal killer immunoglobulin-like receptor (KIR) repertoire is associated with recurrent spontaneous abortion (RSA) or repeated implantation failure (RIF) (Varla-Leftherioti et al., 2010) and if abnormal, the patient might then benefit from intravenous immunoglobulin (IVIg) therapy (Fatemi et al., 2013). IVIg has been successful in the treatment of recurrent miscarriage and recurrent implantation failure among women with elevated anti-phospholipid antibodies (APA) and/or NK cell activity (Coulam et al., 2012). When the pregnancy outcomes of women with a history of reproductive failure and elevated NK cell cytotoxicity treated with intralipid were compared with women treated with IVIg, no differences were seen. Side-by-side comparison showed that synthetic pre-implantation factor (sPIF) is equally effective to inhibit NK cell toxicity at a lower dose than intravenous gamma immunoglobulin or intralipid treatment currently used (Roussev et al., 2013). sPIF is not yet available commercially, but intralipid infusions are available globally. Intralipid is a synthetic product composed of 10% soybean oil, 1.2% egg yolk phospholipids, 2.25% glycerin, and water. When indicated, IL is infused 7-10 days prior to embryo transfer, and one more time again after a positive pregnancy in women whose NKa is due to an autoimmune causes (antiphospholipid antibodies and/or antithyroid antibodies) (CARE Fertility Forum Index). In cases of alloimmune implantation dysfunction (DQa and/HLA matching between the embryo recipient and the male partner), the same applies, but in this situation, the infusion is repeated at 2-4 week intervals until the 24th week of pregnancy. IL costs about 10 times less than IVIg, is not a blood product, and is without significant side effects (CARE Fertility Forum Index).

It is the first time that intralipid-iodine is suggested to be used for HSG in infertile women.

### REFERENCES

1. D. Hallberg I. et al., Fat emulsions for complete intravenous nutrition, Postgrad. med. J. 1967, 43, 307-316.
2. Juras J et al., Imaging in tubal infertility, Rev Prat 2002, 2:1768-74.
3. Elstein M., Tubal disease and fertility outcome., Reprod Biomed Online 2008, 16:167-9.
4. Thurmond A S et al., A Review of Selective Salpingography and Fallopian Tube Catheterization, RadioGraphics 2000, 20:1759-68
5. Papaioannou S. et al., Diagnostic and therapeutic value of selective salpingography and tubal catheterization in an unselected infertile population, Fertil steril 2003, 79: 613-7.
6. Dreyer K. et al., Oil-Based or Water-Based Contrast for Hysterosalpingography in Infertile Women, N. Engl. J. Med. 2017, 376(21):2043-2052.
7. Baramki T A. Hysterosalpingography, Fertil Steril. 2005 June; 83(6):1595-606.
8. Mackey R A et al., Pregnancy following hysterosalpingography with oil and water soluble dye, Fertil Steril. 1971, 22(8):504-7.
9. Schwabe M G et al., Hysterosalpingography with oil contrast medium enhances fertility in patients with infertility of unknown etiology, Fertil. Steril. 1983, 40(5):604-6.
10. Rasmussen F et al., Therapeutic effect of hysterosalpingography: oil-versus water-soluble contrast media—a randomized prospective study, Radiology, 1991, 179(1): 75-8.
11. Watson A et al., A meta-analysis of the therapeutic role of oil soluble contrast media at hysterosalpingography: a surprising result?, Fertil Steril. 1994, 61(3):470-7.
12. Pinto A B et al., Pregnancy outcomes after fallopian tube recanalization: oil-based versus water-soluble contrast agents, J Vasc Intery Radiol. 2003, 14(1):69-74.
13. Grosskinsky C M et al., Pelvic granulomata mimicking endometriosis following the administration of oil-based contrast media for hysterosalpingography, Obstet Gynecol. 1994, 83(5 Pt 2):890-2.
14. Letterie G S & Rose G S, Pregnancy rates after the use of oil-based and water-based contrast media to evaluate tubal patency, South Med J. 1990, 83(12):1402-3.
15. Moore D E et al., Effects of contrast agents on the fallopian tube in a rabbit model, Radiology. 1990, 176(3):721-4.
16. Alper M M et al., Pregnancy rates after hysterosalpingography with oil- and water-soluble contrast media. Obstet Gynecol, 1986, 68(1):6-9.
17. Gautam N. Allahbadia, Intralipid Infusion is the Current Favorite of Gynecologists for Immunotherapy, J. Obstet. Gynaecol. India. 2015, 65(4): 213-217.
18. Oreshkova T et al., A cross-talk of decidual stromal cells, trophoblast, and immune cells: a prerequisite for the success of pregnancy, Am. J. Reprod. Immunol. 2012; 68(5):366-373.
19. Makrigiannakis A, Petsas G, Toth B, et al. Recent advances in understanding immunology of reproductive failure. J Reprod Immunol. 2011, 90(1):96-104.
20. Fatemi H. et al., Implantation in assisted reproduction: a look at endometrial receptivity, Reprod Biomed Online. 2013, 27(5):530-538.
21. Karami N. et al., Enhancement of peripheral blood CD56(dim) cell and NK cell cytotoxicity in women with recurrent spontaneous abortion or in vitro fertilization failure, J. Reprod. Immunol. 2012, 95(1-2):87-92.
22. Varla-Leftherioti M. et al., HLA-DQA1*0505 sharing and killer immunoglobulin-like receptors in sub fertilecouples: report from the 15th International Histocompatibility Workshop, Tissue Antigens. 2010, 75(6):668-672.
23. Coulam C B et al., Does immunotherapy for treatment of reproductive failure enhance live births?, Am. J. Reprod. Immunol. 2012, 67(4):296-304.
24. Roussev R G et al., Preimplantation factor inhibits circulating natural killer cell cytotoxicity and reduces CD69 expression: implications for recurrent pregnancy loss therapy, Reprod Biomed Online. 2013, 26(1):79-87.
25. CARE Fertility Forum Index. Intralipids—All you need to know. http://www.carefertility.com/ivf/viewtopic.php?t=39200.

### EXAMPLES

### Example 1

### Intralipid for Amniotic Fluid Embolism (AFE)

In 1998 it was first showed that intravenous Intralipid could prevent or improve resuscitation from cardiovascular collapse by severe bupivacaine overdose in rats. Since then published examples now include toxicities related to verapamil, diltiazem, amlodipine, quetiapine and sertraline, haldoperidol, lamotrigine, olanzapine, propranolol, atenolol, nevibolol, doxepin, dosulepin, imipramine, amitriptyline, glyosphate herbicide, flecainide, venlafaxine, moxidectin, and others. Amniotic fluid embolism (AFE) is a rare but potentially catastrophic obstetric emergency. Despite earlier recognition and aggressive treatment, morbidity and mortality rates remain high. An estimated 5%-15% of all maternal deaths in Western countries are due to AFE. The pathophysiology of AFE is not completely understood. AFE most commonly occurs during labor, delivery, or the immediate postpartum period. However, it has been reported to occur up to 48 h postpartum. Pulmonary hypertension and right heart strain/failure may be the result of physical amniotic fluid debris in the pulmonary vasculature or, perhaps more likely, result from circulating pulmonary vasoconstrictive mediators. Therapy with Intralipid in male rats resulted in 100% survival and prevented Pulmonary arterial hypertension-induced right ventricular failure by preserving right ventricular pressure and right ventricular ejection fraction and preventing right ventricular hypertrophy and lung remodeling. In preexisting severe Pulmonary arterial hypertension, Intralipid attenuated most lung and right ventricular abnormalities. The beneficial effects of Intralipid in Pulmonary arterial hypertension seem to result from the interplay of various factors, among which preservation and/or stimulation of angiogenesis, suppression and/or reversal of inflammation, fibrosis and hypertrophy, in both lung and right ventricular, appear to be major contributors. In conclusion, Intralipid not only prevents the development of Pulmonary arterial hypertension and right ventricular failure but also rescues preexisting severe Pulmonary arterial hypertension. Intralipid treatment is a new treatment for AFE (amniotic fluid embolism) which was never suggested before. Animal studies should be done in order to evaluate this new treatment modality.

Weinberg et al. [1] first showed in 1998 that an infusion of a soybean oil emulsion normally used as a total parenteral nutrition solution could prevent (by pretreatment) or improve resuscitation from cardiovascular collapse caused by severe bupivacaine overdose in the intact, anesthetized rat. Subsequent studies from the same laboratory confirmed these findings in isolated rat heart [2] and anesthetized dog [3].

Under the latter experimental model, return of spontaneous circulation after a bupivacaine challenge occurred in all animals receiving lipid, but in none of the saline controls [3]. This study was accompanied by an editorial asking whether lipid might be the long-sought “silver bullet” for local anesthetic systemic toxicity (LAST). Since then, the effectiveness of lipid emulsion infusion in reversing LAST has been confirmed in other laboratories and by systematic analysis [4] in the clinical setting as well.

These publications, along with other animal studies, opened the door to more widespread use of lipid emulsion for emergency treatment of toxicities caused by a range of lipophilic drugs. Notably, published examples now include toxicities related to verapamil, diltiazem, amlodipine, quetiapine and sertraline, haldoperidol, lamotrigine, olanzapine, propranolol, atenolol, nevibolol, doxepin, dosulepin, imipramine, amitriptyline, glyosphate herbicide, flecainide, venlafaxine, moxidectin, and others.

### Material and Method

Tricyclic antidepressant (TCA) toxicity results predominantly from myocardial sodium-channel blockade. Subsequent ventricular dysrhythmias, myocardial depression, and hypotension cause cardiovascular collapse. Animal studies have demonstrated the effectiveness of intravenous lipid-emulsion in treating TCA cardiotoxicity.

Blaber M. S. et al. [5] report a case of dothiepin (tricyclic antidepressant) overdose causing refractory cardiovascular collapse, which seemed to be successfully reversed with lipid-emulsion therapy (Intralipid^{→}; Fresenius, Cheshire, UK).

Lipid emulsions are a potentially novel therapy for reversing cardiotoxicity seen in TCA overdose. Research is required into the role of lipid emulsion in the management of poisoning by oral lipophilic agents.

Lipid infusion is useful in reversing cardiac toxicity of local anesthetics, and recent reports indicate it may be useful in resuscitation from toxicity induced by a variety of other drugs. While the mechanism behind the utility of lipid rescue remains to be fully elucidated, the predominant effect appears to be creation of a “lipid sink”.

French D. et al. [6] tried to determine whether the extraction of drugs by lipid, and hence the clinical efficacy of lipid rescue in toxicological emergencies can be predicted by specific drug properties.

Each drug investigated was added individually to human drug-free serum. Intralipid^{→} was added to this drugcontaining serum, shaken and then incubated at 37° C. The lipid was removed by ultracentrifugation and the concentration of drug remaining in the serum was measured by high-pressure liquid chromatography.

In this in vitro model, the ability of lipid emulsion to bind a drug was largely dependent upon the drug's lipid partition constant. Additionally, using a multiple linear regression model, the prediction of binding could be improved by combining the lipid partition constant with the volume of distribution together accounting for approximately 88% of the variation in the decrease in serum drug concentration with the administration of lipid emulsion.

The lipid partition constant and volume of distribution can likely be used to predict the efficacy of lipid infusion in reversing the cardiac toxicity induced by anesthetics or other medications.

Local anaesthetics may induce cardiac arrest, usually because of rapid absorption from the site of injection or because of an intended intravascular injection. Early central nervous system symptoms usually precede seizures. Cardiac arrhythmias follow the CNS signs. These arrhythmias often resolve with the i.v. bolus injection of 100 to 150 mL of a lipid emulsion (20% Intralipid^{→}. Although long acting local anaesthetics (bupivacaine, ropivacaine, levobupivacaine) are predominantly involved in this cardiac toxicity, lidocaine may also induce cardiac arrhythmias and clinician must be aware of this risk. In case of cardiac arrest, resuscitation manoeuvres are of major importance. They need to be performed immediately and the efficacy of the lipid rescue requires a correct coronary flow to be efficacious. Finally, prevention is the key of a safe injection. It is important to control the dose, to inject slowly, without any excessive pressure and to verify that no blood reflux occurs [7].

Papadopoulou A. et al. [8] hypothesized that by substituting a dye surrogate in place of local anesthetic, they could visually demonstrate dye sequestration by lipid emulsion that would be dependent on both dye lipophilicity and the amount of lipid emulsion used.

They selected 2 lipophilic dyes, acid blue 25 and victoria blue, with log P values comparable to lidocaine and bupivacaine, respectively. Each dye solution was mixed with combinations of lipid emulsion and water to emulate “lipid rescue” treatment at dye concentrations equivalent to fatal, cardiotoxic, and neurotoxic local anesthetic plasma concentrations. The lipid emulsion volumes added to each dye solution emulated equivalent intravenous doses of 100, 500, and 900 mL of 20% Intralipid in a 75-kg adult. After mixing, the samples were separated into a lipid-rich supernatant and a lipid-poor subnatant by heparin flocculation. The subnatants were isolated, and their colors compared against a graduated dye concentration scale.

Lipid emulsion addition resulted in significant dye acquisition by the lipid compartment accompanied by a reduction in the color intensity of the aqueous phase that could be readily observed. The greatest amount of sequestration occurred with the dye possessing the higher log P value and the greatest amount of lipid emulsion.

This study provides a visual demonstration of the lipid sink effect. It supports the theory that lipid emulsion may reduce the amount of free drug present in plasma from concentrations associated with an invariably fatal outcome to those that are potentially survivable.

### Statistical Analysis

Local anesthetic (LA) intoxication with cardiovascular arrest is a potential fatal complication of regional anesthesia. Lipid resuscitation has been recommended for the treatment of LA-induced cardiac arrest. Aim of the study [9] was to compare four different rescue regimens using epinephrine and/or lipid emulsion and vasopressin to treat cardiac arrest caused by bupivacaine intoxication.

Twenty-eight piglets were randomized into four groups (4×7), anesthetized with sevoflurane, intubated, and ventilated. Bupivacaine was infused with a syringe driver via central venous catheter at a rate of 1 mg·kg⁻¹·min⁻¹ until circulatory arrest. Bupivacaine infusion and sevoflurane were then stopped, chest compression was started, and the pigs were ventilated with 100% oxygen. After 1 min, epinephrine 10 μg·kg⁻¹ (group 1), Intralipid^{→}20% 4 ml·kg⁻¹ (group 2), epinephrine 10 μg·kg⁻¹+Intralipid^{→} 4 ml·kg⁻¹ (group 3) or 2 IU vasopressin+Intralipid^{→} 4 ml·kg⁻¹ (group 4) were administered. Secondary epinephrine doses were given after 5 min if required.

Survival was 71%, 29%, 86%, and 57% in groups 1, 2, 3, and 4. Return of spontaneous circulation was regained only by initial administration of epinephrine alone or in combination with Intralipid^{→}. Piglets receiving the combination therapy survived without further epinephrine support. In contrast, in groups 2 and 4, return of spontaneous circulation was only achieved after secondary epinephrine rescue.

In cardiac arrest caused by bupivacaine intoxication, first-line rescue with epinephrine and epinephrine+Intralipid^{→} was more effective with regard to survival than Intralipid^{→} alone and vasopressin+Intralipid^{→} in this pig model [9].

Local anesthetic (LA) intoxication with severe hemodynamic compromise is a potential catastrophic event. Lipid resuscitation has been recommended for the treatment of LA-induced cardiac arrest. However, there are no data about effectiveness of Intralipid for the treatment of severe cardiovascular compromise prior to cardiac arrest. Aim of this study was to compare effectiveness of epinephrine and Intralipid for the treatment of severe hemodynamic. Piglets were compromise owing to bupivacaine intoxication, anesthetized with sevoflurane, intubated, and ventilated. Bupivacaine was infused with a syringe driver via a central venous catheter at a rate of 1 mg·kg⁻¹·min⁻¹ until invasively measured mean arterial pressure (MAP) dropped to 50% of the initial value. Bupivacaine infusion was then stopped, and epinephrine 3 μg kg⁻¹ (group 1), Intralipid^{→} 20% 2 ml·kg⁻¹ (group 2), or Intralipid^{→} 20% 4 ml·kg⁻¹ (group 3) was immediately administered. Twenty-one piglets (3×7), were recorded. All animals in group 1 (100%) but only four of seven (57%) piglets in groups 2 and 3, respectively, survived. Normalization of hemodynamic parameters (HR, MAP) and ET (CO₂) was fastest in group 1 with all piglets achieving HR and MAP values. hemodynamic compromise owing to bupivacaine intoxication in piglets, firstline rescue with epinephrine was more effective than Intralipid with regard to survival as well as normalization of hemodynamic parameters and ET (CO₂) [10].

Intravenous lipid emulsion (ILE) has been proposed as a rescue therapy for severe local anesthetic drugs toxicity, but experience is limited with other lipophilic drugs. An 18-year-old healthy woman was admitted 8 h after the voluntary ingestion of sustained-release diltiazem (3600 mg), with severe hypotension refractory to fluid therapy, calcium salts, and high-dose norepinephrine (6.66 μg/kg/min). Hyperinsulinemic euglycemia therapy was initiated and shortly after was followed by a protocol of ILE (intralipid 20%, 1.5 ml/kg as bolus, followed by 0.25 ml/kg over 1 h). The main finding attributed to ILE was an apparent rapid decrease in insulin resistance, despite a prolonged serum diltiazem elimination half-life. Diltiazem is a lipophilic cardiotoxic drug, which could be sequestered in an expanded plasma lipid phase. The mechanism of action of ILE is not known, including its role in insulin resistance and myocardial metabolism in calcium-channel blocker poisoning [11].

There is increasing evidence for the use of Intralipid in the management of acute local anaesthetic toxicity. This is supported by the recent Association of Anaesthetists of Great Britain and Ireland (AAGBI) guidelines for the management of local anaesthetic toxicity. Acute hospitals in England and Wales were surveyed to determine the proportion that currently stocked Intralipid, the locations of stocks within the hospital, guidelines related to its use and previous use in the last 12 months. The majority of hospitals surveyed stocked Intralipid in multiple locations, although not in all areas using high volumes of local anaesthetics. Guidelines were typically in place, although these were often local rather than those from the AAGBI. Use in the last 12 months was uncommon, but typically information was not available on indications for its use. More systematic data collection is required on the safety and efficacy of Intralipid in the management of acute drug toxicity [12].

Intralipid therapy has been used successfully as “rescue therapy” in several cases of overdose. West P. L. et al. [13] present a case of iatrogenic lipid emulsion overdose because of a dosing error.

A 71-year-old female overdosed on 27 tablets of 5 mg amlodipine. Although initially stable in the Emergency Department, she became hypotensive, oliguric, and respiratory failure developed despite medical therapy. The primary treating team felt that meaningful recovery was unlikely to occur without rapid improvement in clinical status, and 12.5 h after presentation, Intralipid rescue therapy was initiated. A protocol for Intralipid specifying a maximum infusion of 400 mL of 20% lipid emulsion was faxed, but the infusion was continued until 2 L of lipid emulsion was infused. There were no detectable adverse hemodynamic effects of the Intralipid infusion. After this time, laboratory values were difficult to obtain. Three hours after the infusion, a metabolic panel was obtained from ultracentrifuged blood showing hyponatremia. A white blood cell (WBC) was obtained from a complete blood count (CBC) performed 22 h after the infusion, hemoglobin and hematocrit could not be obtained from this blood. A platelet count was obtained by smear estimate. Hematocrits were obtained from centrifuged blood and appeared elevated. No oxygenation could be obtained on blood gas. The patient's family chose to withdraw care on hospital day 2 and no further laboratory draws were obtained. Amlodipine was 1500 ng/mL (ref 3-11 ng/mL).

Lipid emulsion overdose caused no detectable acute adverse hemodynamic effects. The following laboratory values were unobtainable immediately after infusion: white blood cell count, hemoglobin, hematocrit, platelet count, and a metabolic panel of serum electrolytes. Ultracentrifugation of blood allowed for detection of a metabolic panel 3 h after the infusion. Centrifuged hematocrits appeared to be higher than expected.

Lipid infusion reverses systemic local anesthetic toxicity. The acceptable upper limit for lipid administration is unknown and has direct bearing on clinical management. Hiller D. B. et al. [14] hypothesize that high volumes of lipid could have undesirable effects and sought to identify the dose required to kill 50% of the animals (LD(50)) of large volume lipid administration.

Intravenous lines and electrocardiogram electrodes were placed in anesthetized, male Sprague-Dawley rats. Twenty percent lipid emulsion (20, 40, 60, or 80 mL/kg) or saline (60 or 80 mL/kg), were administered over 30 mins; lipid dosing was assigned by the Dixon “up-anddown” method. Rats were recovered and observed for 48 h then euthanized for histologic analysis of major organs. Three additional rats were administered 60 mL/kg lipid emulsion and euthanized at 1, 4, and 24 h to identify progression of organ damage.

The maximum likelihood estimate for LD(50) was 67.72 (SE, 10.69) mL/kg. Triglycerides were elevated immediately after infusion but returned to baseline by 48 h when laboratory abnormalities included elevated amylase, aspartate aminotransferase, and serum urea nitrogen for all lipid doses. Histologic diagnosis of myocardium, brain, pancreas, and kidneys was normal at all doses. Microscopic abnormalities in lung and liver were observed at 60 and 80 mL/kg; histopathology in the lung and liver was worse at 1 h than at 4 and 24 h.

The LD(50) of rapid, high volume lipid infusion is an order of magnitude greater than doses typically used for lipid rescue in humans and supports the safety of lipid infusion at currently recommended doses for toxin-induced cardiac arrest. Lung and liver histopathology was observed at the highest infused volumes.

### Results

Cave G. and Harvey M. [15] evaluate the efficacy of lipid emulsion as antidotal therapy outside the accepted setting of local anesthetic toxicity.

Literature was accessed through PubMed, OVID (1966-February 2009), and EMBASE (1947-February 2009) using the search terms “intravenous” and [“fat emulsion” or “lipid emulsion” or “Intralipid”] and [“toxicity” or “resuscitation” or “rescue” or “arrest” or “antidote”]. Additional author and conference publication searches were undertaken. Publications describing the use of lipid emulsion as antidotal treatment in animals or humans were included.

Fourteen animal studies, one human study, and four case reports were identified. In animal models, intravenous lipid emulsion (ILE) has resulted in amelioration of toxicity associated with cyclic antidepressants, verapamil, propranolol, and thiopentone. Administration in human cases has resulted in successful resuscitation from combined bupropion/lamotrigine-induced cardiac arrest, reversal of sertraline/quetiapine-induced coma, and amelioration of verapamiland beta blocker-induced shock.

Management of overdose with highly lipophilic cardiotoxic medications should proceed in accord with established antidotal guidelines and early poisons center consultation. Data from animal experiments and human cases are limited, but suggestive that ILE may be helpful in potentially lethal cardiotoxicity or developed cardiac arrest attributable to such agents. Use of lipid emulsion as antidote remains a nascent field warranting further preclinical study and systematic reporting of human cases of use.

Previous investigators have demonstrated amelioration of lipid-soluble drug toxidromes with infusion of lipid emulsions. Clomipramine is a lipid-soluble tricyclic antidepressant with significant cardiovascular depressant activity in human overdose. Harvey M. and Cave G. [16] compare resuscitation with Intralipid versus sodium bicarbonate in a rabbit model of clomipramine toxicity.

Thirty sedated and mechanically ventilated New Zealand White rabbits were infused with clomipramine at 320 mg/kg per hour. At target mean arterial pressure of 50% initial mean arterial pressure, animals were rescued with 0.9% NaCl 12 mL/kg, 8.4% sodium bicarbonate 3 mL/kg, or 20% Intralipid 12 mL/kg. Pulse rate, mean arterial pressure, and QRS duration were sampled at 2.5-minute intervals to 15 minutes. In the second phase of the experiment, 8 sedated and mechanically ventilated rabbits were infused with clomipramine at 240 mg/kg per hour to a mean arterial pressure of 25 mm Hg. Animals received either 2 mL/kg 8.4% sodium bicarbonate or 8 mL/kg 20% Intralipid as rescue therapy. External cardiac compression and intravenous adrenaline were administered in the event of cardiovascular collapse.

Mean difference in mean arterial pressure between Intralipid and saline solution-treated groups was 21.1 mm Hg (95% confidence interval [CI] 13.5 to 28.7 mm Hg) and 19.5 mm Hg (95% CI 10.5 to 28.9 mm Hg) at 5 and 15 minutes, respectively. Mean difference in mean arterial pressure between Intralipidand bicarbonate-treated groups was 19.4 mm Hg (95% CI 18.8 to 27.0 mm Hg) and 11.5 mm Hg (95% CI 2.5 to 20.5 mm Hg) at 5 and 15 minutes. The rate of change in mean arterial pressure was greatest in the Intralipid-treated group at 3 minutes (6.2 mm Hg/min [95% CI 3.8 to 8.6 mm Hg/min]. In the second phase of the experiment spontaneous circulation was maintained in all Intralipid-treated rabbits (n=4). All animals in the bicarbonate-treated group developed pulseless electrical activity and proved refractory to resuscitation at 10 minutes (n=4, P=0.023).

In this rabbit model, Intralipid infusion resulted in more rapid and complete reversal of clomipramine-induced hypotension compared with sodium bicarbonate. Additionally, Intralipid infusion prevented cardiovascular collapse in a model of severe clomipramine toxicity.

### Discussion

Amniotic fluid embolism (AFE) is a rare but potentially catastrophic obstetric emergency. Despite earlier recognition and aggressive treatment, morbidity and mortality rates remain high. An estimated 5%-15% of all maternal deaths in Western countries are due to AFE [17].

Recent retrospective reviews of population-based hospital databases in Canada [18] and the United States [19] found AFE incidences of 6.1-7.7 cases per 100,000 births.

Early studies revealed mortality rates as high as 61%-86%, but more recent estimates suggest a case fatality of 13%-26% [19-21].

First reported by Meyer in 1926 [22], and then later identified as a syndrome in 1941 by Steiner and Lushbaugh [23].

The pathophysiology of AFE is not completely understood. AFE most commonly occurs during labor, delivery, or the immediate postpartum period. However, it has been reported to occur up to 48 h postpartum [24]. Once thought to be the result of an actual embolic obstruction of the pulmonary vasculature by components of amniotic fluid, AFE might result from immune activation and present as an anaphylactoid process. AFE likely involves a spectrum of severity from a subclinical process to a catastrophic event. Early recognition and prompt and aggressive resuscitative efforts enhance the probability of maternal and neonatal survival.

Three phases in the clinical course of AFE have been described. The first or immediate phase is often characterized by altered mental status, respiratory distress, peripheral oxygen desaturation, and hemodynamic collapse. The second phase involves coagulopathy and hemorrhage and occurs in an estimated 4%-50% of patients with presumed AFE. Although older studies of AFE required either sudden, unresuscitatable maternal death or the subsequent development of disseminated intravascular coagulation (DIC) for inclusion in the AFE database, it is now recognized that DIC does not develop in all cases of AFE. Tissue injury and end-organ system failure comprise the last phase of AFE. Clinical findings will vary depending on the organ system(s) predominantly affected. Ventilation-perfusion mismatching as a result of pulmonary vascular constriction at the onset of AFE may explain sudden hypoxia and respiratory arrest [25].

Pulmonary hypertension and right-heart strain/failure may be the result of physical amniotic fluid debris in the pulmonary vasculature or, perhaps more likely, result from circulating pulmonary vasoconstrictive mediators. The mechanisms for myocardial dysfunction that lead to early hypotension are multifactorial. Proposed explanations include myocardial failure in response to sudden pulmonary hypertension, a direct myocardial depressant effect of humoral mediators in amniotic fluid, deviation of the intraventricular septum due to right ventricular dilation, and/or ischemic myocardial injury from hypoxemia [26-28].

Pulmonary arterial hypertension (PAH) is characterized by pulmonary vascular remodeling leading to right ventricular (RV) hypertrophy and failure. Intralipid (ILP), a source of parenteral nutrition for patients, contains γ-linolenic acid and soy-derived phytoestrogens that are protective for lungs and heart. Umar S. et al. [29] investigated the therapeutic potential of ILP in preventing and rescuing monocrotaline-induced PAH and RV dysfunction. PAH was induced in male rats with monocrotaline (60 mg/kg). Rats then received daily ILP (1 mL of 20% ILP per day IP) from day 1 to day 30 for prevention protocol or from day 21 to day 30 for rescue protocol. Other monocrotaline-injected rats were left untreated to develop severe PAH by day 21 or RV failure by approximately day 30. Saline or ILP-treated rats served as controls. Significant increase in RV pressure and decrease in RV ejection fraction in the RV failure group resulted in high mortality. Therapy with ILP resulted in 100% survival and prevented PAH-induced RV failure by preserving RV pressure and RV ejection fraction and preventing RV hypertrophy and lung remodeling. In preexisting severe PAH, ILP attenuated most lung and RV abnormalities. The beneficial effects of ILP in PAH seem to result from the interplay of various factors, among which preservation and/or stimulation of angiogenesis, suppression and/or reversal of inflammation, fibrosis and hypertrophy, in both lung and RV, appear to be major contributors. In conclusion, ILP not only prevents the development of PAH and RV failure but also rescues preexisting severe PAH [29].

### Conclusion

Joseph Eldor, Vladimir Kotlovker Intralipid treatment is a new treatment for AFE which was never suggested before. Animal studies should be done in order to evaluate this new treatment modality.

### REFERENCES

1. G. L. Weinberg, T. VadeBoncouer, G. A. Ramaraju, M. F. Garcia-Amaro and M. J. Cwik, “Pretreatment or Resuscitation with a Lipid Infusion Shifts the Dose-Response to Bupivacaine-Induced Asystole in Rats,” Anesthesiology, Vol. 88, No. 4, 1998, pp. 1071-1075. doi:10.1097/00000542-199804000-00028
2. G. L. Weinberg, R. Ripper, P. Murphy, L. B. Edelman, W. Hoffman, G. Strichartz and D. L. Feinstein, “Lipid Infusion Accelerates Removal of Bupivacaine and Recovery from Bupivacaine Toxicity in the Isolated Rat Heart,” Regional Anesthesia and Pain Medicine, Vol. 31, No. 4, 2006, pp. 296-303.
3. G. Weinberg, R. Ripper, D. L. Feinstein and W. Hoffman, “Lipid Emulsion Infusion Rescues Dogs from Bupivacaine-Induced Cardiac Toxicity,” Regional Anesthesia and Pain Medicine, Vol. 28, No. 3, 2003, pp. 198-202.
4. C. Jamaty, B. Bailey, A. Larocque, E. Notebaert, K. Sanogo and J. M. Chauny, “Lipid Emulsions in the Treatment of Acute Poisoning: A Systematic Review of Human and Animal Studies,” Clinical Toxicology, Vol. 48, No. 1, 2010, pp. 1-27
5. M. S. Blaber, J. N. Khan, J. A. Brebner and R. McColm, “Lipid Rescue for Tricyclic Antidepressant Cardiotoxicity,” The Journal of Emergency Medicine, 2012, in press. doi:10.1016/j.jemermed.2011.09.010
6. D. French, C. Smollin, W. Ruan, A. Wong, K. Drasner and A. H. Wu, “Partition Constant and Volume of Distribution as Predictors of Clinical Efficacy of Lipid Rescue for Toxicological Emergencies,” Clinical Toxicology, Vol. 49, No. 9, 2011, pp. 801-809. doi:10.3109/15563650.2011.617308
7. J. X. Mazoit, “Cardiac Arrest and Local Anaesthetics,” La Presse Médicale, 2012, in press. doi:10.1016/j.lpm.2012.04.016
8. A. Papadopoulou, J. W. Willers, T. L. Samuels and D. R. Uncles, “The Use of Dye Surrogates to Illustrate Local Anesthetic Drug Sequestration by Lipid Emulsion: A Visual Demonstration of the Lipid Sink Effect,” Regional Anesthesia and Pain Medicine, Vol. 37, No. 2, 2012, pp. 183-187.
9. J. Mauch, 0. M. Jurado, N. Spielmann, R. Bettschart-Wolfensberger and M. Weiss, “Resuscitation Strategies from Bupivacaine-Induced Cardiac Arrest,” Pediatric Anesthesia, Vol. 22, No. 2, 2012, pp. 124-129. doi:10.1111/j.1460-9592.2011.03688.x
10. J. Mauch, 0. M. Jurado, N. Spielmann, R. Bettschart-Wolfensberger and M. Weiss, “Comparison of Epinephrine vs Lipid Rescue to Treat Severe Local Anesthetic Toxicity—An Experimental Study in Piglets,” Pediatric Anesthesia, Vol. 21, No. 11, 2011, pp. 1103-1108. doi:10.1111/j.1460-9592.2011.03652.x
11. V. Montiel, T. Gougnard and P. Hantson, “Diltiazem Poisoning Treated with Hyperinsulinemic Euglycemia Therapy and Intravenous Lipid Emulsion,” European Journal of Emergency Medicine, Vol. 18, No. 2, 2011, pp. 121-123.doi:10.1097/MEJ.0b013e32834130ab
12. P. Hamann, P. I. Dargan, N. Parbat, H. Ovaska and D. M. Wood, “Availability of and Use of Intralipid (Lipid Rescue Therapy, Lipid Emulsion) in England and Wales,” The Journal of Emergency Medicine, Vol. 27, No. 8, 2010, pp. 590-592. doi:10.1136/emj.2009.083352
13. P. L. West, N. J. McKeown and R. G. Hendrickson, “Iatrogenic Lipid Emulsion Overdose in a Case of Amlodipine Poisoning,” Clinical Toxicology, Vol. 48, No. 4, 2010, pp. 393-396. doi:10.3109/15563651003670843
14. D. B. Hiller, G. D. Gregorio, K. Kelly, R. Ripper, L. Edelman, R. Boumendjel, K. Drasner and G. L. Weinberg, “Safety of High Volume Lipid Emulsion Infusion: A First Approximation of LD50 in Rats,” Regional Anesthesia and Pain Medicine, Vol. 35, No. 2, 2010, pp. 140-144. doi:10.1097/AAP.0b013e3181c6f5aa
15. G. Cave and M. Harvey, “Intravenous Lipid Emulsion as Antidote beyond Local Anesthetic Toxicity: A Systematic Review,” Academic Emergency Medicine, Vol. 16, No. 9, 2009, pp. 815-824. doi:10.1111/j.1553-2712.2009.00499.x
16. M. Harvey and G. Cave, “Intralipid Outperforms Sodium Bicarbonate in a Rabbit Model of Clomipramine Toxicity,” Annals of Emergency Medicine, Vol. 49, No. 2, 2007, pp. 178-185doi:10.1016/j.annemergmed.2006.07.016
17. A. Conde-Agudelo and R. Romero, “Amniotic Fluid Embolism: An Evidence-Based Review,” American Journal of Obstetrics & Gynecology, Vol. 201, No. 5, 2009, p. 445. doi:10.1016/j aj og.2009.04.052
18. M. S. Kramer, J. Rouleau, T. F. Baskett and K. S. Joseph, “Maternal Health Study Group of the Canadian Perinatal Surveillance System: Amniotic-Fluid Embolism and Medical Induction of Labour: A Retrospective, PopulationBased Cohort Study,” Lancet, Vol. 368, No. 9545, 2006, pp. 1444-1448
19. H. A. Abenhaim, L. Azoulay, M. S. Kramer and L. Leduc, “Incidence and Risk Factors of Amniotic Fluid Embolisms: A Population-Based Study on 3 Million Births in the United States,” American Journal of Obstetrics & Gynecology, Vol. 199, No. 1, 2008, p. 49. doi:10.1016/j.ajog.2007.11.061
20. M. Knight, D. Tuffnell, P. Brocklehurst, P. Spark and J. J. Kurinczuk, “UK Obstetric Surveillance System: Incidence and Risk Factors for Amniotic-Fluid Embolism,” Obstetrics & Gynecology, Vol. 115, No. 5, 2010, pp. 910-917.
21. A. F. Gei, R. B. Vadhera and G. D Hankins, “Embolism during Pregnancy: Thrombus, Air, and Amniotic Fluid,” Anesthesiology Clinics of North America, Vol. 21, No. 1, 2003, pp. 165-182. doi:10.1016/S0889-8537(02)00052-4
22. J. R. Meyer, “Embolia Pulmonar Amnio Caseosa,” Brasil Medico, Vol. 2, 1926, pp. 301-303.
23. P. E. Steiner and C. C. Lushbaugh, “Maternal Pulmonary Embolism by Amniotic Fluid as a Cause of Obstetric Shock and Unexpected Deaths in Obstetrics,” Journal of the American Medical Association, Vol. 255, No. 16, 1986, pp. 2187-2203. doi:10.1001/jama.1986.03370160085032
24. A. Conde-Agudelo and R. Romero, “Amniotic Fluid Embolism: An Evidence-Based Review,” American Journal of Obstetrics & Gynecology, Vol. 201, No. 5, 2009, p. 445. doi:10.1016/j.ajog.2009.04.052
25. S. L. Clark, G. D. Hankins, D. A. Dudley, G. A. Dildy and T. F. Porter, “Amniotic Fluid Embolism: Analysis of the National Registry,” American Journal of Obstetrics & Gynecology, Vol. 172, No. 4, 1995, pp. 1158-1167.
26. A. O'Shea and S. Eappen, “Amniotic Fluid Embolism,” International Anesthesiology Clinics, Vol. 45, No. 1, 2007, pp. 17-28. doi:10.1097/AIA.0b013e31802b8853
27. R. D. Stanten, L. I. Iverson, T. M. Daugharty, S. M. Lovett, C. Terry and E. Blumenstock, “Amniotic Fluid Embolism Causing Catastrophic Pulmonary Vasoconstriction: Diagnosis by Transesophageal Echocardiogram and Treatment by Cardiopulmonary Bypass,” Obstetrics & Gynecology, Vol. 102, No. 3, 2003, pp. 496-498. doi:10.1016/S0029-7844(03)00161-3
28. C. F. James, N. G. Feinglass, D. M. Menke, S. F. Grinton and T. J. Papadimos, “Massive Amniotic Fluid Embolism: Diagnosis Aided by Emergency Transesophageal Echocardiography,” International Journal of Obstetric Anesthesia, Vol. 13, No. 4, 2004, pp. 279-283. doi:10.1016/j.ijoa.2004.03.008
29. S. Umar, R. D. Nadadur, J. Li, F. Maltese, P. Partownavid, A. van der Laarse and M. Eghbali, “Intralipid Prevents and Rescues Fatal Pulmonary Arterial Hypertension and Right Ventricular Failure in Rats,” Hypertension, Vol. 58, No. 3, 2011, pp. 512-518. doi:10.1161/HYPERTENSIONAHA.110.168781

### Example 2

### Intralipid Treatment: Is it Only the Tip of an Iceberg? a New Suggestion: Bone Cement Implantation Syndrome (BCIS)

On 1998 it was first showed that intravenous intralipid could prevent or improve resuscitation from cardiovascular collapse by severe bupivacaine overdose in rats. Since then published examples now include toxicities related to verapamil, diltiazem, amlodipine, quetiapine and sertraline, haldoperidol, lamotrigine, olanzapine, propranolol, atenolol, nevibolol, doxepin, dosulepin, imipramine, amitriptyline, glyosphate herbicide, flecainide, venlafaxine, moxidectin, and others. Intralipid treatment is a new treatment for Bone cement implantation syndrome (BCIS) which was never suggested before. Animal studies should be done in order to evaluate this new treatment modality.

Pretreatment or Resuscitation with a Lipid Infusion

Intralipid® 10% (10% i.v fat emulsion) (a 10% intravenous fat emulsion) is a sterile, non-pyrogenic fat emulsion prepared for intravenous administration as a source of calories and essential fatty acids. it is made up of 10% soybean oil, 1.2% egg yolk phospholipids, 2.25% glycerin, and water for injection. in addition, sodium hydroxide has been added to adjust the ph so that the final product ph is 8. ph range is 6 to 8.9.

The soybean oil is a refined natural product consisting of a mixture of neutral triglycerides of predominantly unsaturated fatty acids. the major component fatty acids are linoleic (44-62%), oleic (19-30%), palmitic (7-14%),

Purified egg phosphatides are a mixture of naturally occurring phospholipids which are isolated from the egg yolk.

Intralipid® 10% (10% i.v fat emulsion) (a 10% intravenous fat emulsion) has an osmolality of approximately 300 mosmol/kg water (which represents 260 mosmol/liter of emulsion) and contains emulsified fat particles of

The total caloric value, including fat, phospholipid and glycerin, is 1.1 kcal per ml of intralipid 10% (10% i.v fat emulsion). the phospholipids present contribute 47 milligrams or approximately 1.5 mmol of phosphorus per 100 ml, of the emulsion.

Weinberg et al., [1] first showed in 1998 that an infusion of a soybean oil emulsion normally used as a total parenteral nutrition solution could prevent (by pretreatment) or improve resuscitation from cardiovascular collapse caused by severe bupivacaine overdose in the intact, anesthetized rat. Subsequent studies from the same laboratory confirmed these findings in isolated rat heart [2] and anesthetized dog [3]. Under the latter experimental model, return of spontaneous circulation after a bupivacaine challenge occurred in all animals receiving lipid, but in none of the saline controls [3]. This study was accompanied by an editorial asking whether lipid might be the long-sought “silver bullet” for local anesthetic systemic toxicity (LAST). Since then, the effectiveness of lipid emulsion infusion in reversing LAST has been confirmed in other laboratories and by systematic analysis [4] in the clinical setting as well.

Lipid infusion is useful in reversing cardiac toxicity of local anesthetics, and recent reports indicate it may be useful in resuscitation from toxicity induced by a variety of other drugs. While the mechanism behind the utility of lipid rescue remains to be fully elucidated, the predominant effect appears to be creation of a “lipid sink”.

French D et al., [5] tried to determine whether the extraction of drugs by lipid, and hence the clinicals efficacy of lipid rescue in toxicological emergencies can be predicted by specific drug properties.

Each drug investigated was added individually to human drug-free serum. Intralipid® was added to this drug-containing serum, shaken and then incubated at 37° C. The lipid was removed by ultracentrifugation and the concentration of drug remaining in the serum was

In this in vitro model, the ability of lipid emulsion to bind a drug was largely dependent upon the drug's lipid partition constant. Additionally, using a multiple linear regression model, the prediction of binding could be improved by combining the lipid partition constant with the volume of distribution together accounting for approximately 88% of the variation in the decrease in serum drug concentration with the administration of lipid emulsion.

The lipid partition constant and volume of distribution can likely be used to predict the efficacy of lipid infusion in reversing the cardiac toxicity induced by anesthetics or other medications.

Local anaesthetics may induce cardiac arrest, usually because of rapid absorption from the site of injection or because of an intended intravascular injection. Early central nervous system symptoms usually precede seizures. Cardiac arrhythmias follow the CNS signs. These arrhythmias often resolve with the i.v. bolus injection of 100 to 150 mL of a lipid emulsion (20% Intralipid(®)). Although long acting local anaesthetics (bupivacaine, ropivacaine, levobupivacaine) are predominantly involved in this cardiac toxicity, lidocaine may also induce cardiac arrhythmias and clinician must be aware of this risk. In case of cardiac resuscitation manoeuvres are of major importance. They need to be performed immediately and tharrest, e efficacy of the lipid rescue requires a correct coronary flow to be efficacious. Finally, prevention is the key of a safe injection. It is important to control the dose, to inject slowly, without any excessive pressure and to verify that no blood reflux occurs [6].

Reversing Other Drugs Toxicity

These publications, along with other animal studies, opened the door to more widespread use of lipid emulsion for emergency treatment of toxicities caused by a range of lipophilic drugs. Notably, published examples now include toxicities related to verapamil, diltiazem, amlodipine, quetiapine and sertraline, haldoperidol, lamotrigine, olanzapine, propranolol, atenolol, nevibolol, doxepin, dosulepin, imipramine, amitriptyline, glyosphate herbicide, flecainide, venlafaxine, moxidectin, and others.

Tricyclic antidepressant (TCA) toxicity results predominantly from myocardial sodium-channel blockade. Subsequent ventricular dysrhythmias, myocardial depression, and hypotension cause cardiovascular collapse. Animal studies have demonstrated the effectiveness of intravenous lipid-emulsion in treating TCA cardiotoxicity.

Blaber M S et al., [7] report a case of dothiepin (tricyclic antidepressant) overdose causing refractory cardiovascular collapse, which seemed to be successfully reversed with lipid-emulsion therapy (Intralipid®; Fresenius, Cheshire, UK).

Lipid emulsions are a potentially novel therapy for reversing cardiotoxicity seen in TCA overdose. Research is required into the role of lipid emulsion in the management of poisoning by oral lipophilic agents.

### The Lipid Sink Effect

Papadopoulou A et al., [8] hypothesized that by substituting a dye surrogate in place of local anesthetic, they could visually demonstrate dye sequestration by lipid emulsion that would be dependent on both dye lipophilicity and the amount of lipid emulsion used.

They selected 2 lipophilic dyes, acid blue 25 and Victoria blue, with log P values comparable to lidocaine and bupivacaine, respectively. Each dye solution was mixed with combinations of lipid emulsion and water to emulate “lipid rescue” treatment at dye concentrations equivalent to fatal, cardiotoxic, and neurotoxic local anesthetic plasma concentrations. The lipid emulsion volumes added to each dye solution emulated equivalent intravenous doses of 100, 500, and 900 mL of 20% Intralipid in a 75-kg adult. After mixing, the samples were separated into a lipid-rich supernatant and a lipid-poor subnatant by heparin flocculation. The subnatants were isolated, and their colors compared against a graduated dye concentration scale.

Lipid emulsion addition resulted in significant dye acquisition by the lipid compartment accompanied by a reduction in the color intensity of the aqueous phase that could be readily observed. The greatest amount of sequestration occurred with the dye possessing the higher log P value and the greatest amount of lipid emulsion.

This study provides a visual demonstration of the lipid sink effect. It supports the theory that lipid emulsion may reduce the amount of free drug present in plasma from concentrations associated with an invariably fatal outcome to those that are potentially survivable.

Local anesthetic (LA) intoxication with cardiovascular arrest is a potential fatal complication of regional anesthesia. Lipid resuscitation has been recommended for the treatment of LA-induced cardiac arrest. Aim of the study [9] was to compare four different rescue regimens using epinephrine and/or lipid emulsion and vasopressin to treat cardiac arrest caused by bupivacaine intoxication.

Twenty-eight piglets were randomized into four groups (4×7), anesthetized with sevoflurane, intubated, and ventilated. Bupivacaine was infused with a syringe driver via central venous catheter at a rate of 1 mg·kg⁻¹·min⁻¹ until circulatory arrest. Bupivacaine infusion and sevoflurane were then stopped, chest compression was started, and the pigs were ventilated with 100% oxygen. After 1 min, epinephrine 10 μg·kg⁻¹ (group 1), Intralipid(®) 20% 4 ml·kg⁻¹ (group 2), epinephrine 10 μg·kg⁻¹+Intralipid(®) 4 ml·kg⁻¹ (group 3) or 2 IU vasopressin+Intralipid(®) 4 ml·kg⁻¹ (group 4) were administered. Secondary epinephrine doses were given after 5 min if required.

Survival was 71%, 29%, 86%, and 57% in groups 1, 2, 3, and 4. Return of spontaneous circulation was regained only by initial administration of epinephrine alone or in combination with Intralipid(®). Piglets receiving the combination therapy survived without further epinephrine support. In contrast, in groups 2 and 4, return of spontaneous circulation wasonly achieved after secondary epinephrine rescue.

In cardiac arrest caused by bupivacaine intoxication, firstline rescue with epinephrine and epinephrine+Intralipid(®) was more effective with regard to survival than Intralipid(®) alone and vasopressin+Intralipid(®) in this pig model (9).

Local anesthetic (LA) intoxication with severe hemodynamic compromise is a potential catastrophic event. Lipid resuscitation has been recommended for the treatment of LA-induced cardiac arrest. However, there are no data about effectiveness of Intralipid for the treatment of severe cardiovascular compromise prior to cardiac arrest. Aim of this study was to compare effectiveness of epinephrine and Intralipid for the treatment of severe hemodynamic. Piglets were compromise owing to bupivacaine intoxication, anesthetized with sevoflurane, intubated, and ventilated. Bupivacaine was infused with a syringe driver via a central venous catheter at a rate of 1 mg·kg⁻¹·min⁻¹ until invasively measured mean arterial pressure (MAP) dropped to 50% of the initial value. Bupivacaine infusion was then stopped, and epinephrine 3 μg·kg⁻¹ (group 1), Intralipid(®) 20% 2 ml·kg⁻¹ (group 2), or Intralipid 20% 4 ml·kg⁻¹ (group 3) was immediately administered. Twenty-one piglets (3×7), were recorded. All animals in group 1 (100%) but only four of seven (57%) piglets in group 2 and group 3, respectively, survived. Normalization of hemodynamic parameters (HR, MAP) and ET(CO₂) was fastest in group 1 with all piglets achieving HR and MAP values. Hemodynamic compromise owing to bupivacaine intoxication in piglets, first-line rescue with epinephrine was more effective than Intralipid with regard to survival as well as normalization of hemodynamic parameters and ET(CO₂) [10].

Intravenous lipid emulsion (ILE) has been proposed as a rescue therapy for severe local anesthetic drugs toxicity, but experience is limited with other lipophilic drugs. An 18-year-old healthy woman was admitted 8 h after the voluntary ingestion of sustained-release diltiazem (3600 mg), with severe hypotension refractory to fluid therapy, calcium salts, and high-dose norepinephrine (6.66 μg/kg/min). Hyperinsulinemic euglycemia therapy was initiated and shortly after was followed by a protocol of ILE (intralipid 20%, 1.5 ml/kg as bolus, followed by 0.25 ml/kg over 1 h). The main finding attributed to ILE was an apparent rapid decrease in insulin resistance, despite a prolonged serum diltiazem elimination half-life. Diltiazem is a lipophilic cardiotoxic drug, which could be sequestered in an expanded plasma lipid phase. The mechanism of action of ILE is not known, including its role in insulin resistance and myocardial metabolism in calcium-channel blocker poisoning [11].

### Guidelines for the Management of Local Anaesthetic Toxicity

There is increasing evidence for the use of Intralipid in the management of acute local anaesthetic toxicity. This is supported by the recent Association of Anaesthetists of Great Britain and Ireland (AAGBI) guidelines for themanagement of local anaesthetic toxicity. Acute hospitals in England and Wales were surveyed to determine the proportion that currently stocked Intralipid, the locations of stocks within the hospital, guidelines related to its use and previous use in the last 12 months. The majority of hospitals surveyed stocked Intralipid in multiple locations, although not in all areas using high volumes of local anaesthetics. Guidelines were typically in place, although these were often local rather than those from the AAGBI. Use in the last 12 months was uncommon, but typically information was not available on indications for its use. More systematic data collection is required on the safety and efficacy of Intralipid in the management of acute drug toxicity [12].

Intralipid therapy has been used successfully as “rescue therapy” in several cases of overdose. West P L et al., [13] present a case of iatrogenic lipid emulsion overdose because of a dosing error: “A 71-year-old female overdosed on 27 tablets of 5 mg amlodipine. Although initially stable in the Emergency Department, she became hypotensive, oliguric, and respiratory failure developed despite medical therapy. The primary treating team felt that meaningful recovery was unlikely to occur without rapid improvement in clinical status, and 12.5 h after presentation, intralipid rescue therapy was initiated. A protocol for intralipid specifying a maximum infusion of 400 mL of 20% lipid emulsion was faxed, but the infusion was continued until 2 L of lipid emulsion was infused. There were no detectable adverse hemodynamic effects of the intralipid infusion. After this time, laboratory values were difficult to obtain. Three hours after the infusion, a metabolic panel was obtained from ultracentrifuged blood showing hyponatremia. A white blood cell (WBC) was obtained from a complete blood count (CBC) performed 22 h after the infusion, hemoglobin and hematocrit could not be obtained from this blood. A platelet count was obtained by smear estimate. Hematocrits were obtained from centrifuged blood and appeared elevated. No oxygenation could be obtained on blood gas. The patient's family chose to withdraw care on hospital day 2 and no further laboratory draws were obtained. Amlodipine was 1,500 ng/mL (ref 3-11 ng/mL).”

### Lipid Emulsion Overdose

Lipid emulsion overdose caused no detectable acute adverse hemodynamic effects. The following laboratory values were unobtainable immediately after infusion: white blood cell count, hemoglobin, hematocrit, platelet count, and a metabolic panel of serum electrolytes. Ultracentrifugation of blood allowed for detection of a metabolic panel 3 h after the infusion. Centrifuged hematocrits appeared to be higher than expected.

Lipid infusion reverses systemic local anesthetic toxicity. The acceptable upper limit for lipid administration is unknown and has direct bearing on clinical management. Hiller D B et al., [14] hypothesize that high volumes of lipid could have undesirable effects and sought to identify the dose required to kill 50% of the animals (LD(50)) of large volume lipid administration.

Intravenous lines and electrocardiogram electrodes were placed in anesthetized, male Sprague-Dawley rats. Twenty percent lipid emulsion (20, 40, 60, or 80 mL/kg) or saline (60 or 80 mL/kg), were administered over 30 mins; lipid dosing was assigned by the Dixon “up-and-down” method. Rats were recovered and observed for 48 hrs then euthanized for histologic analysis of major organs. Three additional rats were administered 60 mL/kg lipid emulsion and euthanized at 1, 4, and 24 hrs to identify progression of organ damage.

The maximum likelihood estimate for LD(50) was 67.72 (SE, 10.69) mL/kg. Triglycerides were elevated immediately after infusion but returned to baseline by 48 hrs when laboratory abnormalities included elevated amylase, aspartate aminotransferase, and serum urea nitrogen for all lipid doses. Histologic diagnosis of myocardium, brain, pancreas, and kidneys was normal at all doses. Microscopic abnormalities in lung and liver were observed at 60 and 80 mL/kg; histopathology in the lung and liver was worse at 1 hr than at 4 and 24 hrs.

The LD(50) of rapid, high volume lipid infusion is an order of magnitude greater than doses typically used for lipid rescue in humans and supports the safety of lipid infusion at currently recommended doses for toxin-induced cardiac arrest. Lung and liver histopathology was observed at the highest infused volumes.

Cave G and Harvey M [15] evaluate the efficacy of lipid emulsion as antidotal therapy outside the accepted setting of local anesthetic toxicity.

Intralipid rescue: 1966-2009

Literature was accessed through PubMed, OVID (1966-February 2009), and EMBASE (1947-February 2009) using the search terms “intravenous” AND [“fat emulsion” OR “lipid emulsion” OR “Intralipid”] AND [“toxicity” OR “resuscitation” OR “rescue” OR “arrest” OR “antidote”]. Additional author and conference publication searches were undertaken. Publications describing the use of lipid emulsion as antidotal treatment in animals or humans were included.

Fourteen animal studies, one human study, and four case reports were identified. In animal models, intravenous lipid emulsion (ILE) has resulted in amelioration of toxicity associated with cyclic antidepressants, verapamil, propranolol, and thiopentone. Administration in human cases has resulted in successful resuscitation from combined bupropion/lamotrigine-induced cardiac arrest, reversal of sertraline/quetiapine-induced coma, and amelioration of verapamil- and beta blocker-induced shock.

Management of overdose with highly lipophilic cardiotoxic medications should proceed in accord with established antidotal guidelines and early poisons center consultation. Data from animal experiments and human cases are limited, but suggestive that ILE may be helpful in potentially lethal cardiotoxicity or developed cardiac arrest attributable to such agents. Use of lipid emulsion as antidote remains a nascent field warranting further preclinical study and systematic reporting of human cases of use.

Previous investigators have demonstrated amelioration of lipid-soluble drug toxidromes with infusion of lipid emulsions. Clomipramine is a lipid-soluble tricyclic antidepressant with significant cardiovascular depressant activity in human overdose. Harvey M and Cave G [16] compare resuscitation with Intralipid versus sodium bicarbonate in a rabbit model of clomipramine toxicity.

Thirty sedated and mechanically ventilated New Zealand White rabbits were infused with clomipramine at 320 mg/kg per hour. At target mean arterial pressure of 50% initial mean arterial pressure, animals were rescued with 0.9% NaCl 12 mL/kg, 8.4% sodium bicarbonate 3 mL/kg, or 20% Intralipid 12 mL/kg. Pulse rate, mean arterial pressure, and QRS duration were sampled at 2.5-minute intervals to 15 minutes. In the second phase of the experiment, 8 sedated and mechanically ventilated rabbits were infused with clomipramine at 240 mg/kg per hour to a mean arterial pressure of 25 mm Hg. Animals received either 2 mL/kg 8.4% sodium bicarbonate or 8 mL/kg 20% Intralipid as rescue therapy. External cardiac compression and intravenous adrenaline were administered in the event of cardiovascular collapse.

Mean difference in mean arterial pressure between Intralipid- and saline solution-treated groups was 21.1 mm Hg (95% confidence interval [CI] 13.5 to 28.7 mm Hg) and 19.5 mm Hg (95% CI 10.5 to 28.9 mm Hg) at 5 and 15 minutes, respectively. Mean difference in mean arterial pressure between Intralipid- and bicarbonate-treated groups was 19.4 mm Hg (95% CI 18.8 to 27.0 mm Hg) and 11.5 mm Hg (95% CI 2.5 to 20.5 mm Hg) at 5 and 15 minutes. The rate of change in mean arterial pressure was greatest in the Intralipid-treated group at 3 minutes (6.2 mm Hg/min [95% CI 3.8 to 8.6 mm Hg/min] In the second phase of the experiment spontaneous circulation was maintained in all Intralipid-treated rabbits (n=4). All animals in the bicarbonate-treated group developed pulseless electrical activity and proved refractory to resuscitation at 10 minutes (n=4, P=0.023).

In this rabbit model, Intralipid infusion resulted in more rapid and complete reversal of clomipramineinduced hypotension compared with sodium bicarbonate. Additionally, Intralipid infusion prevented cardiovascular collapse in a model of severe clomipramine toxicity.

### Bone Cement Implantation Syndrome (BCIS)

Bone cement implantation syndrome (BCIS) is characterised by hypoxia, hypotension and loss of consciousness occurring early after bone cementation. The haemodynamic perturbations during BCIS have not been extensively studied, particularly not in patients with femoral neck fracture. Kotyra M et al., [17] evaluated the effects of cemented hemiarthroplasty, in these patients, on pulmonary haemodynamics, right ventricular performance, intrapulmonary shunting and physiological dead space.

Fifteen patients undergoing cemented hemiarthroplasty because of femoral neck fracture were included. Surgery was performed under total intravenous anaesthesia in the lateral position. All patients were catheterised with a radial and pulmonary artery catheter, for continuous measurements of mean arterial pressure (MAP), pulmonary arterial pressure (PAP), cardiac output, mixed venous oxygen saturation, right ventricular end-diastolic volume (RVEDV) and right ventricular ejection fraction (RVEF). Haemodynamic measurements and blood gas analyses were performed after induction of anaesthesia, during surgical stimulation before and immediately after bone cementation and prosthesis insertion, 10 and 20 min after insertion and during skin closure.

After bone cementation and prosthesis insertion, MAP (−10%), cardiac index (−10%) and stroke volume index (−10%) decreased, while PAPs (10-15%) and the pulmonary vascular resistance index (45%) increased. RVEF decreased by 10-20%, while the RVEDV index increased by 10%. Pulmonary haemodynamic and RV variables changed progressively with time, while intra-pulmonary shunting and physiological dead space increased immediately after prosthesis insertion and then returned to baseline.

Cemented hemiarthroplasty in patients with femoral neck fracture causes a pronounced pulmonary vasoconstriction and an impairment of RV function accompanied by pulmonary ventilation/perfusion abnormalities.

Acute hypotension, hypoxemia, cardiac arrhythmias, cardiac arrest, (or a combination of these), and sudden death are well-recognized complications of the cemented hip arthroplasty procedure. Collectively, these are known as the bone cement implantation syndrome (BCIS). The endogenous cannabinoids, anandamide (ANA) and 2-arachidonylglycerol (2-AG), are reported to be strong vasodilators and play a role in the hypotension associated with hemorrhagic and septic shock. In the present study [18], a potential role for the endogenous cannabinoids in influencing hemodynamic variables in BCIS was investigated. Thirty-five patients (35 hips) entered a prospective, randomized clinical trial. The patients were divided into two groups. Group 1 comprised 16 patients who had the component inserted using a conventional cementing technique, whereas group 2 consisted of 19 patients who had the femoral component inserted without cement. Blood samples were taken at six consecutive time points: before anesthesia, after reaming the femur, 2 min after insertion of stems with or without cement into the femur, and 10 min, 20, and 30 min after stem insertion. In group 1 (with cement), the mean levels of ANA and 2-AG significantly increased after stem insertion. In a comparison of each group after stem insertion, mean ANA and 2-AG levels in group 1 also significantly differed from those in group 2. By contrast, in group 2 (without cement) neither ANA nor 2-AG levels exhibited a significant increase or change at any point in time. In conclusion, it has been shown for the first time that endogenous cannabinoids are candidates for lipid mediators of BCIS [18].

Fallon K M et al., [19] described a case report of a cardiac arrest during a cemented hip arthroplasty procedure. Hemodynamic instability during methylmethacrylate use in arthroplasty surgery can be explained by fat embolization rather than the inherent toxicity of the monomer. A 78-yrold woman required a cemented hemiarthroplasty for a pathologic left subcapital fracture. The patient's past medical history included stable angina, diet-controlled type II diabetes and metastatic breast cancer. During the cementing of the canal and insertion of the femoral prosthesis, desaturation, hypotension and cardiac arrest occurred. The patient underwent a successful intraoperative resuscitation and was transferred to the intensive care unit where she subsequently developed disseminated intravascular coagulopathy. The patient died 24 hr later and autopsy confirmed the cause of death as fat embolization. The deleterious cardiovascular effects of methylmethacrylate have been discussed in the literature. However, clinical evidence supports fat embolization during arthroplasty surgery as a greater determinant of hemodynamic compromise. Surgical precautions are paramount in minimizing the sequelae of Bone Implantation Syndrome and anesthetic treatment consists of supportive care.

Bone cement implantation syndrome is characterized by hypotension, hypoxemia, cardiac arrhythmias, cardiac arrest, or any combination of these complications. It may result from venous embolization that occurs in conjunction with intramedullary hypertension in the femur during insertion of the prosthesis in patients undergoing cemented total hip arthroplasty (THA). Intramedullary hypertension does not occur in patients undergoing noncemented THA. In this study, Ereth M H et al., [20] sought to compare embolization between patients undergoing cemented and noncemented THA and to determine whether this state resulted in cardiorespiratory deterioration. In this prospective investigation of 35 patieundergoing elective THA, they used transesophageal echocardiography and invasive hemodynants mic monitoring, and in 12 of them, they monitored distribution of pulmonary ventilation and perfusion intraoperatively. Embolization was significantly greater after insertion of the prosthesis in patients undergoing cemented than in those undergoing noncemented THA. Cemented THA was also associated with decreased cardiac output and increased pulmonary artery pressure and pulmonary vascular resistance. Increases in ventilation-perfusion mismatching, however, could not be demonstrated 30 minutes after insertion of the femoral prosthesis. Intraoperative monitoring for embolism may help physicians assess patients in whom cardiorespiratory function deteriorates during THA [20].

Intralipid prevents and rescues fatal pulmonary arterial hypertension and right ventricular failure and enhances the inflammatory response to endotoxin

Pulmonary arterial hypertension (PAH) is characterized by pulmonary vascular remodeling leading to right ventricular (RV) hypertrophy and failure. Intralipid (ILP), a source of parenteral nutrition for patients, contains γ-linolenic acid and soy-derived phytoestrogens that are protective for lungs and heart. Umar S. et al., [21] investigated the therapeutic potential of ILP in preventing and rescuing monocrotaline-induced PAH and RV dysfunction. PAH was induced in male rats with monocrotaline (60 mg/kg). Rats then received daily ILP (1 mL of 20% ILP per day IP) from day 1 to day 30 for prevention protocol or from day 21 to day 30 for rescue protocol. Other monocrotaline-injected rats were left untreated to develop severe PAH by day 21 or RV failure by approximately day 30. Saline or ILP-treated rats served as controls. Significant increase in RV pressure and decrease in RV ejection fraction in the RV failure group resulted in high mortality. Therapy with ILP resulted in 100% survival and prevented PAH-induced RV failure by preserving RV pressure and RV ejection fraction and preventing RV hypertrophy and lung remodeling. In preexisting severe PAH, ILP attenuated most lung and RV abnormalities. The beneficial effects of ILP in PAH seem to result from the interplay of various factors, among which preservation and/or stimulation of angiogenesis, suppression and/or reversal of inflammation, fibrosis and hypertrophy, in both lung and RV, appear to be major contributors. In conclusion, ILP not only prevents the development of PAH and RV failure but also rescues preexisting severe PAH [21].

Novel anti-inflammatory effects of insulin have recently been described, and insulin therapy to maintain euglycemia suppresses the plasma levels of free fatty acids (FFA) and increases the survival of critically ill patients. Krogh-Madsen et al., [22] aimed to explore the effect of short-term high levels of plasma FFA on the inflammatory response to a low dose of endotoxin. Fourteen healthy male volunteers underwent the following two trials in a randomized crossover design: 1) continuous infusion of 20% Intralipid [0.7 ml·kg⁻¹·h⁻¹ (1.54 g/kg)] for 11 h, and 2) infusion of isotonic saline for 11 h (control). In each trial, heparin was given to activate lipoprotein lipase, and an intravenous bolus of endotoxin (0.1 ng/kg) was given after 6 h of Intralipid/saline infusion. Blood samples and muscle and fat biopsies were obtained before the Intralipid/saline infusion and before as well as after infusion of an endotoxin bolus. Plasma levels of FFA, triglycerides, and glycerol were markedly increased during the Intralipid infusion. Endotoxin exposure induced an increase in plasma levels of TNF-alpha, IL-6, and neutrophils and further stimulated gene expression of TNF-alpha and IL-6 in both skeletal muscle and adipose tissue. The systemic inflammatory response to endotoxin was significantly pronounced during Intralipid infusion. Short-term hyperlipidemia enhances the inflammatory response to endotoxin, and skeletal muscle and adipose tissue are capable of producing essential inflammatory mediators after endotoxin stimulation.

### Is Intralipid Approved by the FDA for Local Anesthesia-Induced Cardiovascular Collapse or Resuscitation.

As with any FDA-labeled medication, the individual Intralipid products carry with them a set of contraindications to use and adverse effects. However, the extent to which these apply in the setting of Local Anesthesia-induced cardiovascular collapse or resuscitation, for which Intralipid is not FDA-approved, remains to be defined. The general contraindication to Intralipid use is the presence of disorders of fat metabolism, other contraindications not published in the product's package insert include egg allergy and acute myocardial infarction. The use of Intralipid is cautioned in patients with anemia, severe liver disease, coagulopathies, pulmonary disease, and in patients at risk for fat embolism. The most common adverse effects from general Intralipid use are those related to contamination of the dministration site and irritation of the veins likely due to other solutions co-infused with Intralipid.

Early or immediate adverse effects of Intralipid include allergic reactions, headache, somnolence, dizziness, diaphoresis, dyspnea, nausea/vomiting, hyperthermia, and hypercoagulability. More delayed adverse effects of Intralipid include thrombocytopenia, jaundice, overloading syndrome, increased liver function tests, leucopenia, hepatomegaly, and splenomegaly; pancreatitis has rarely been associated with Intralipid use. Those adverse effects that may be dose- or rate-related include pulmonary embolus or fat embolus, and pulmonary vasoconstriction may result from bolus administration of Intralipid. There were no adverse effects reported with Intralipid use in the four human case reports after Local Anesthesia-induced cardiovascular collapse, although further investigation is warranted [23].

### Conclusion

Intralipid treatment is a new treatment for BCIS which was never suggested before. Animal studies should be done in order to evaluate this new treatment modality.

### REFERENCES

1. Weinberg G L, VadeBoncouer T, Ramaraju G A, Garcia-Amaro M F, Cwik M J: Pretreatment or resuscitation with a lipid infusion shifts the dose-response to bupivacaine-induced asystole in rats. Anesthesiology 1998, 88:1071-1075. Article
2. Weinberg G L, Ripper R, Murphy P, Edelman L B, Hoffman W, Strichartz G, Feinstein D L: Lipid infusion accelerates removal of bupivacaine and recovery from bupivacaine toxicity in the isolated rat heart. Reg Anesth Pain Med 2006, 31:296-303.
3. Weinberg G, Ripper R, Feinstein D L, Hoffman W: Lipid emulsion infusion rescues dogs from bupivacaine-induced cardiac toxicity. Reg Anesth Pain Med 2003, 28:198-202.
4. Jamaty C, Bailey B, Larocque A, Notebaert E, Sanogo K, Chauny J M: Lipid emulsions in the treatment of acute poisoning: a systematic review of human and animal studies. Clin Toxicol (Phila) 2010, 48:1-27.
5. French D, Smollin C, Ruan W, Wong A, Drasner K, Wu A H: Partition constant and volume of distribution as predictors of clinical efficacy of lipid rescue for toxicological emergencies. Clin Toxicol (Phila) 2011, 49:801-809.
6. Mazoit J X: [Cardiac arrest and local anaesthetics.]. Presse Med 2012. Article|PubMed
7. Blaber M S, Khan J N, Brebner J A, McColm R: “Lipid rescue” for tricyclic antidepressant cardiotoxicity. J Emerg Med 2012, 43:465-467.
8. Papadopoulou A, Willers J W, Samuels T L, Uncles D R: The use of dye surrogates to illustrate local anesthetic drug sequestration by lipid emulsion: a visual demonstration of the lipid sink effect. Reg Anesth Pain Med 2012, 37:183-187.
9. Mauch J, Jurado O M, Spielmann N, Bettschart-Wolfensberger R, Weiss M: Resuscitation strategies from bupivacaine-induced cardiac arrest. Paediatr Anaesth 2012, 22:124-129.
10. Mauch J, Martin Jurado O, Spielmann N, Bettschart-Wolfensberger R, Weiss M: Comparison of epinephrine vs lipid rescue to treat severe local anesthetic toxicity—an experimental study in piglets. Paediatr Anaesth 2011, 21:1103-1108.
11. Montiel V, Gougnard T, Hantson P: Diltiazem poisoning treated with hyperinsulinemic euglycemia therapy and intravenous lipid emulsion. Eur J Emerg Med 2011, 18:121-123.
12. Hamann P, Dargan P I, Parbat N, Ovaska H, Wood D M: Availability of and use of Intralipid (lipid rescue therapy, lipid emulsion) in England and Wales. Emerg Med J 2010, 27:590-592.
13. West P L, McKeown N J, Hendrickson R G: Iatrogenic lipid emulsion overdose in a case of amlodipine poisoning. Clin Toxicol (Phila) 2010, 48:393-396.
14. Hiller D B, Di Gregorio G, Kelly K, Ripper R, Edelman L, Boumendjel R, Drasner K, Weinberg G L: Safety of high volume lipid emulsion infusion: a first approximation of LD50 in rats. Reg Anesth Pain Med 2010, 35:140-144.
15. Cave G, Harvey M: Intravenous lipid emulsion as antidote beyond local anesthetic toxicity: a systematic review. Acad Emerg Med 2009, 16:815-824.
16. Harvey M, Cave G: Intralipid outperforms sodium bicarbonate in a rabbit model of clomipramine toxicity. Ann Emerg Med 2007, 49:178-185, 185 e171-174.
17. Kotyra M, Houltz E, Ricksten S E: Pulmonary haemodynamics and right ventricular function during cemented hemiarthroplasty for femoral neck fracture. Acta Anaesthesiol Scand 2010, 54:1210-1216.
18. Motobe T, Hashiguchi T, Uchimura T, Yamakuchi M, Taniguchi N, Komiya S, Maruyama I: Endogenous cannabinoids are candidates for lipid mediators of bone cement implantation syndrome. Shock 2004, 21:8-12.
19. Fallon K M, Fuller J G, Morley-Forster P: Fat embolization and fatal cardiac arrest during hip arthroplasty with methylmethacrylate. Can J Anaesth 2001, 48:626-629.
20. Ereth M H, Weber J G, Abel M D, Lennon R L, Lewallen D G, Ilstrup D M, Rehder K: Cemented versus noncemented total hip arthroplasty—embolism, hemodynamics, and intrapulmonary shunting. Mayo Clin Proc 1992, 67:1066-1074.
21. Umar S, Nadadur R D, Li J, Maltese F, Partownavid P, van der Laarse A, Eghbali M: Intralipid prevents and rescues fatal pulmonary arterial hypertension and right ventricular failure in rats. Hypertension 2011, 58:512-518.
22. Krogh-Madsen R, Plomgaard P, Akerstrom T, Moller K, Schmitz O, Pedersen B K: Effect of short-term intralipid infusion on the immune response during low-dose endotoxemia in humans. Am J Physiol Endocrinol Metab 2008, 294:E371-379.
23. Felice K, Schumann H: Intravenous lipid emulsion for local anesthetic toxicity: a review of the literature. J Med Toxicol 2008, 4:184-191.

### Example 3

### Combined Spinal Epidural Catheters for Epidural Cooling, Cerebrospinal Fluid Aspiration and Spinal Intralipid Infusion for Treatment of Spinal and Brain Injuries, Diseases and Protection

A new proposal for spinal cord and brain treatment and protection due to injuries and diseases is made herein. It is composed of two 20G nylon catheters with 6 lateral holes arranged circumferentially within 3 cm from the tip and a closed end. One catheter is inserted into the epidural space and the other catheter is inserted into the spinal space in two different lumbar interspaces using an 18G Tuohy needle 90 mm. The epidural catheter is used for cooled saline injection and infusion. The spinal catheter is used for Intralipid spinal injections and CSF aspiration. The proposal is based on the current studies on spinal cord cooling and CSF aspiration as well as on the Intralipid resuscitation properties and lipid brain protection. A study is needed to evaluate the clinical value of this combined approach.

Following a traumatic injury to the central nervous system, a cascade of physiological events leads to neuronal loss including, for example, an inflammatory immune response and excitotoxicity. Despite advances in spinal cord protection, paraplegia continues to be a serious complication of descending and thoraco-abdominal aortic operations. Damage to the nervous system may result from a traumatic injury, such as penetrating trauma or blunt trauma, or a disease or disorder including, but not limited to, Alzheimer's disease, Parkinson's disease, Huntington's disease, amyotrophic lateral sclerosis (ALS), diabetic neuropathy, senile dementia, stroke and ischemia. During the past 9 years, considerable new evidence has accumulated supporting the use of prophylactic hypothermia for traumatic brain injury (TBI). In 1998 it was first showed that intravenous intralipid could prevent or improve resuscitation from cardiovascular collapse by severe bupivacaine overdose in rats. Since then published examples now include toxicities related to verapamil, diltiazem, amlodipine, quetiapine and sertraline, haloperidol, lamotrigine, olanzapine, propranolol, atenolol, nevibolol, doxepin, dosulepin, imipramine, amitriptyline, glyosphate herbicide, flecainide, venlafaxine, moxidectin, and

A new multimodal proposal for spinal cord and brain treatment and protection due to injuries and diseases is made herein.

### CSF Role in Ischemic Injury

In ischemic injury, CSF has a toxic effect of facilitating cerebral edema. While intracellularly excessive water content is directly toxic to the CNS cells, the cerebral edema can also block cerebral blood flow and collateral circulation to damaged nerve tissue, causing “no reflow” phenomenon or “hypoperfusion”. This failure of circulation results in continuing damage to CNS tissue after the interruption of blood flow is reversed leading to ireversible damage. Restoration of blood flow to the affected area of the CNS after a period of complete ischemia as short as six minutes does not result in blood reflow to the affected CNS tissue. After the CNS tissue is injured by an initiating insult, such as ischemia, trauma, the CSF infiltrates the CNS tissue through water channels on the injured cell membrane to cause edema. Swelling of the tissue makes the Virchow-Robin space (also known as the perivascular space or extracellular space) smaller and may even cause it to collapse, thereby compressing the small blood vessels and resulting in an obstruction of the blood flow, such as a “hypoperfusion” or even “no-reflow” phenomenon, which prolongs the original ischemic duration, blocks collateral circulation and induces a feedback loop. As the duration of blood flow interruption increases, the edema spreads throughout the CNS tissue causing additional damage in an ischemic cascade.

In the adult human, the average intra-cranial volume is about 1700 ml. The volume of the brain is approximately 1400 ml; CSF volume ranges from about 52 to 160 ml (mean 140 ml), and blood volume is about 150 ml. Thus the CSF occupies about 10 percent of the intra-cranial and intra-spinal volume.

The choroid plexuses are the main sites of CSF formation. The average rate of CSF formation is about 21 to 22 ml/hr, or approximately 500 ml/day. The CSF as a whole is renewed four or five times daily. CSF formation is related to intracranial pressure. When the intracranial pressure is below about 70 mm H2O, CSF is not absorbed, and production increasrs. CSF is a very dilute aqueous solution with a low colloidal osmotic pressure.

The CSF has a mechanical function. It serves as a kind of water jacket for the spinal cord and brain, protecting them from trauma and acute changes in venous blood pressure. The CSF provides buoyancy and shock absorption, so that brain and spinal cord float in a CSF pool. CSF does not appear to be necessary to brain or spinal cord metabolism. However, during ischemic episodes, CSF has a toxic effect by facilitating cerebral edema and resulting in no-reflow phenomenon after disruption of blood flow to CNS tissue.

In order to prevent cerebral edema, and the irreversible effects that occur after the CNS injuries, the CSF is withdrawn from the affected area of the CNS. It is preferred to completely remove all CSF. However, it is very difficult, almost impossible, mechanically to remove CSF completely from the subarachnoid spaces because the CNS (i.e. brain and spinal cord) contour is very complex with many sulci, gyri and pools.

CSF pressure control has been used for protecting spinal cord during aortic surgery. It is said that controlling the pressure of CSF, in particular, maintaining a pressure lower than the central venous pressure could be advantageous in protecting the spinal cord from injury during aortic surgery. However, such pressure control does not achieve the neuroprotective effect in the case of more general ischemia. Removing CSF from the spinal cord's subarachnoid space is relatively easier (compared with the brain) because of spinal cord's simpler contour. However, simple withdrawal of CSF even under controlled conditions in thoraco-aortic surgery, is not predictably effective protecting CSF tissue.

After the CSF has been withdrawn. The injured CNS tissue is washed with washing solutions. The first washing solution is an emulsion. It should contain oil, an osmotic agent, water and at least one emulsifier. Typically, the emulsion contains up to about 31%-80% oil. Generally a water in oil emulsion is preferred. However, oil in water emulsions have also been effective. Intralipid solutions (10%, 20% and 30%), used clinically for parenteral nutrition, such as those manufactured and distributed by Baxter, Fresenius Kabi, Pharmacia & Upjohn etc. may also be effective. The first washing solution acts as a hook to pull out edematous CSF fluids away from the CNS tissue into the oil. The oil can be any non-toxic, organic liquid. Hydrocarbon oils and silicone oils are effective. Any hydrocarbon oils from plant, animal sources and mineral oil such as soybean oil, cod liver oil, vitamin E oil, canola oil, corn oil, and mixtures of these oils in any concentration ratio may be used [1].

### Traumatic Injury to the Central Nervous System

Following a traumatic injury to the central nervous system, a cascade of physiological events leads to neuronal loss including, for example, an inflammatory immune response and excitotoxicity resulting from the initial impact disrupting the glutamate, acetylcholine, cholinergic, GABAA, and NMDA receptor systems. In addition, the traumatic CNS injury is frequently followed by brain and/or spinal cord edema that enhances the cascade of injury and leads to further secondary cell death and increased patient mortality. Methods are needed for the in vivo treatment of traumatic CNS injuries that are successful at providing subsequent trophic support to remaining central nervous system tissue, and thus enhancing functional repair and recovery, under the complex physiological cascade of events which follow the initial insult [2].

The nervous system comprises the central (CNS) and the peripheral nervous system (PNS). The CNS is composed of the brain, spinal cord and visual system; the PNS consists of all of the other neural elements, namely the nerves and ganglia outside of the brain and spinal cord. Damage to the nervous system may result from a traumatic injury, such as penetrating trauma or blunt trauma, or a disease or disorder including, but not limited to, Alzheimer's disease, Parkinson's disease, Huntington's disease, amyotrophic lateral sclerosis (ALS), diabetic neuropathy, senile dementia, stroke and ischemia. Maintenance of CNS integrity is a complex “balancing act” in which compromises are struck with the immune system. In most tissues, the immune system plays an essential part in protection, repair, and healing. In the CNS, because of its unique immune privilege, immunological reactions are relatively limited. A growing body of evidence indicates that the failure of the mammalian CNS to achieve functional recovery after injury reflects an ineffective dialog between the damaged tissue and the immune system [3].

The predominant mechanism in most cases of traumatic brain injury (TBI) is diffuse axonal injury [4]. While axonal injury is common in all TBI regardless of severity, a shearing of the axons occurs in human diffuse axonal injury (DAI) leading to progressive changes that ultimately may result in the loss of connections between nerve cells. The slow progression of events in DAI continues for up to several weeks after injury creating a window of opportunity for therapeutic intervention. There are approximately 500,000 new cases of TBI in the U.S. each year [5], and the incidence requiring hospitalization is estimated to be approximately 200-225/100,000 population. Currently, it is estimated that brain injuries account for 12% of all hospital admissions in the United States [6]. When compared to spinal cord injury, which accounts for less than 1% of hospital admissions, it is clear that TBI is a medical care problem which has a significant impact financially within the United States. Approximately 30,000-44,000 people will survive a severe TBI with GCS score TBI (GCS#10). Yet with new medical management techniques, less than 10% will remain in a persistent vegetative state. A GCS score of eight or less generally reflects a state of unconsciousness in which the patient demonstrates no eye opening, does not follow simple commands to move muscles, and has vocalizations which are limited to sounds. Such signs are indicative of severe brain injury [7]. Approximately 52,000 to 56,000 people die each year from TBI [8], resulting in direct costs approximated at more than $50 billion annually [9]. The costs of severe TBI to the individual and family are extremely high [10]. Acute medical and rehabilitation bills are often around $100,000 with some considerably higher. The Model Systems Database for Traumatic Brain Injury demonstrates there is a correlation between the average Disability Rating Score and the combined acute care and rehabilitation charges [11]. Those with a severe TBI (GCS score of 6-8) have average combined charges of $110,842, and those with a very severe TBI (GCS score 3-5) have average combined charges of $154,256 [12]. About one-half of all TBIs are transportation related and these patients have some of the highest combined charges for acute care and rehabilitations. This may be related to the mechanism of TBI in high speed motor vehicle crashes, specifically the presence of diffuse axonal injury (DAI) being most prevalent in the midbrain and brain stem areas. Clearly, brain injuries of this severity that occur with high speed acceleration-deceleration injuries, have the highest costs to society. TBI clearly causes more mortality, morbidity and probably more economic loss than HIV infection in the United States. Motor vehicle crashes of all types are responsible for approximately 40%-50% of the TBI admissions recorded in the Model TBI Systems Database. The predominant mechanism of injury is considered to be diffuse axonal injury (DAD. Approximately 30%-40% of the fatal head injuries involve diffuse axonal injury by pathological examination [13]. However, based on betaamyloid precursor protein immunostaining, axonal injury may be present in all cases of fatal head injury. In cases of persistent vegetative states, recently found that all cases had evidence of DAI in magnetic resonance imaging (MRI). Diffuse axonal injury occurs even in the absence of a blow to the head and is more prevalent than previously realized. Even in mild head injury, diffuse axonal injury is present in almost ⅓ of the cases. The defining characteristic of DAI is the morphologic change to the axons which occurs over the course of several days to weeks and the fact that multiple regions of the brain are injured. While a component of DAI is present in blunt or penetrating trauma injury, it is at the periphery of the injury zone and is much less significant than the predominant mechanism of injury. DAI is the major mechanism of injury in high speed acceleration-deceleration injuries associated with motor vehicle crashes. While all four mechanisms of TBI (DAI, blunt trauma, penetrating trauma, axonia) may be involved in such an injury, it is the predominant mechanism of injury under this condition [14].

### Inadvertent Intralipid Spinal-Epidural Injection

A term female infant was admitted to the intensive care unit with the diagnosis of tetralogy of Fallot with critical pulmonary stenosis. On the seventh day of life a long saphenous line was inserted that remained without complications until seven days later when the infant appeared septic. A lumbar puncture demonstrated the presence of intra-lipid in the cerebrospinal fluid that was interpreted as due to migration of the saphenous catheter. The child had an uneventful recovery [15].

A patient had accidently received 300 ml of intralipid total parenteral nutrition solution via his epidural catheter. A 21-yr-old man with ulcerative colitis underwent a total colectomy. Postoperative pain control was begun with intravenous morphine using a patient-controlled analgesia device. The patient was unable to obtain satisfactory pain relief. A lumbar epidural catheter was placed, and he received excellent analgesia after 100 microgram of fentanyl was injected into the epidural space. A continuous epidural infusion of preservative-free morphine at 1.0 mg/h was then begun using a standard intravenous infusion set connected to the epidural catheter tubing. The tubing and intravenous infusion pump was labeled with bright green “EPIDURAL CATHETER” tags. He remained nearly pain-free for the next 24 h. It was then discovered that an intravenous infusion of intralipid total parenteral nutrition solution had been “piggy-backed” onto the intravenous tubing leading to the epidural catheter. The label covering one of the side ports on the infusion set tubing had apparently been lost. Approximately 300 ml of intralipid solution had been infused at a rate of 60 ml/h. The epidural catheter was removed from the patient. The catheter insertion site appeared normal and was not painful to palpation. Daily visits failed to reveal any neurological deficits caused by this unusual infusion. The epidural site remained normal. There is no known reports of a similar accidental epidural intralipid infusion [16].

### Epidural Saline Infusion

A 30-year-old female with a 4-month history of postlumbar puncture headache (PLPHA) resulting from an accidental dural puncture during an attempted epidural anesthetic for cesarean section. Epidural blood patches were attempted at 4 days and 3 months post-lumbar puncture, but were unsuccessful. At 4 months post-lumbar puncture, a 24-h epidural saline infusion relieved the PLPHA for 48 h, but the headache returned. Finally, a second epidural saline infusion was done, followed by an epidural blood patch, which permanently cured the PLPHA. Follow-up to 4 months showed no return of the PLPHA [17].

Concerns have been expressed about the potential danger of an autologous epidural blood patch for the treatment of post-dural puncture headache. The immediate resolution of the headache with a blood patch is attributable to thecal compression raising the CSF pressure. An epidural injection of saline would, in theory, produce the same mass effect, and restore normal CSF dynamics. As saline is a relatively inert and sterile solution, epidural saline bolus or infusion appears to be an attractive alternative. Regimens that have been advocated include: 1) 1.0-1.5 liter of epidural Hartman solution over 24 h, starting on the first day after dural puncture; 2) up to 35 ml·h-1 of epidural saline or Hartman solution for 24-48 h, or after development of the headache; 3) a single 30 ml bolus of epidural saline after development of headache; and 4) 10-120 ml of saline injected as a bolus via the caudal epidural space.

Advocates of an epidural saline bolus or infusion maintain that the lumbar injection of saline raises epidural and intrathecal pressure. Reduction in the leak would allow the dura to repair. However, observations of the pressures produced in the subarachnoid and epidural space show that, despite a large rise in epidural pressure, the consequent rise in subarachnoid pressure maintains the differential pressure across the dura. The pressure rise is also not sustained and is dissipated within 10 min. The saline may induce an inflammatory reaction within the epidural space, promoting closure of the dural perforation. Histological studies have not demonstrated an inflammatory response following epidural Dextran 40 administration, however, in contrast to an autologous blood patch. There is no reason to suppose that epidural saline is more likely to accelerate dural healing through a proinflammatory action than Dextran 40. Thus, there are no studies that are able to demonstrate either a sustained rise in CSF pressure or accelerated closure of the dural perforation after the administration of epidural saline. Whilst there are many case reports describing the success of epidural saline, comparative trials with epidural blood patches have not demonstrated the long-term efficacy of epidural saline placement. It is difficult to conclude from the evidence therefore, that epidural saline administration will restore normal CSF dynamics. The administration of large volumes of epidural saline may result in intraocular haemorrhages through a precipitous rise in intracranial pressure [18].

### Paraplegia—A Serious Complication of Descending and Thoraco-Abdominal Aortic Operations

Despite advances in spinal cord protection, paraplegia continues to be a serious complication of descending and thoraco-abdominal aortic operations. A novel, self-contained catheter designed to cool the spinal cord topically after being threaded into the spinal column. A cooling catheter for this purpose was specifically designed and produced. The catheter has two lumina, one for ingress and one for egress of fluid. The system is self-contained, so that the fluid does not communicate in any way with the spinal fluid. A console device circulates cold fluid through the catheter. The catheter was tested in 5 adult sheep, with direct monitoring of core body temperature and spinal cord temperature in both active cooling and passive re-warming cycles. In testing in 4 sheep (five attempted implants, with one failure), the catheter worked without problem, producing effective cooling of the spinal cord, from a mean temperature of 36.8° C. (core temperature) to 30.5° C. (spinal temperature) (p<0.0001). In no case did post-mortem examination or histology reveal any evidence of damage to the spinal cord from hypothermia. Temperature rose toward body temperature after cessation of active cooling. Effective topical cooling of the spinal cord can be achieved via a specially designed, self-contained cooling catheter placed into the intra-thecal space. This catheter holds promise for spinal cord protection in aortic surgery. Also, this catheter may be useful as well in mitigating injury to the spinal cord in cases of traumatic spinal column injury [19].

Paraplegia or paraparesis after otherwise successful thoracic or thoraco-abdominal aortic reconstruction is a devastating complication for both patient and physician. Various strategies have been developed to minimize the incidence of neurological complications after aortic surgery. The incidence of spinal cord ischemia and subsequent neurological complications has been correlated with 1) the duration and severity of ischemia, 2) failure to establish a spinal cord blood supply, and 3) reperfusion injury. Preoperative identification of the arteria radicularis magna, the artery of Adamkiewicz, facilitates identification of critical intercostal vessels for reimplantation, resulting in reestablishing spinal cord blood flow. Techniques for monitoring spinal cord function using evoked potentials have been developed, and surgical techniques have evolved to reduce the duration of ischemia. Furthermore, sequentially sacrificing all the intercostal arteries while maintaining collateral circulation to the cord has produced good outcomes. The severity of ischemia can be minimized by using cerebrospinal fluid drainage, hypothermia, distal bypass, managing the blood pressure, and adjunctive pharmacological therapy. Reperfusion injury can be reduced with the use of antioxidant therapy. Recent advances in endovascular stent grafting have reduced the incidence of postoperative spinal complications, especially among high-risk patients [20].

Hypothermia for Prevention of Paraplegia Associated with Thoracic Aortic Surgery

The most dreaded complication associated with thoracic aortic surgery remains paraplegia caused by spinal cord ischemia. The existence of this problem has been known since the early days of aortic surgery and much research, both clinical and experimental, has been devoted to the understanding and prevention of spinal cord injury. Intraoperative and pen-operative adjuncts have included both pharmacological and surgical approaches. Varying degrees of success have been reported for each new approach, with some remedies coming and going in and out of fashion as continued research either supports or refutes their effectiveness. Rather than finding any one therapy that completely removes the threat of peri-operative spinal cord injury, incremental improvements in surgical and anesthetic regimens have greatly reduced the incidence of this complication to an acceptable level.

Among the earliest adjuncts used in the battle against paraplegia was hypothermia. In studies dating back 50 years, Pontius and colleagues demonstrated that systemic hypothermia successfully allowed prolongation of the spinal cord ischemic interval in laboratory dogs [1]. For many years, the combination of moderate hypothermia, coupled with rapid surgical techniques, remained the mainstay of protection in aortic surgery. As more complex and extensive operations were attempted, profound hypothermic and circulatory arrest approaches were introduced, but for less comprehensive repairs this was thought to be overkill and was selectively used.

Hypothermic techniques, even when applied systemically in moderate or mild amounts, can adversely affect the conduct of the surgery. Prolonged operating times due to cooling and warming, the need for heaters and coolers in the circuits, and increased bleeding tendencies have all given pause to surgeons unfamiliar with these problems. Because of these issues, investigations were turned to eliminating the need for systemic hypothermia and limiting the cooling efforts to those tissues most directly affected, namely the spinal cord. Locally applied cooling blankets and appliances were found not to be effective due to the rapid rewarming by surrounding tissues and interference with the sterile fields of the procedure. Administration of iced saline solutions directly into the isolated aorta was shown to effect cooling of the spinal cord but, as one might expect, this was abandoned due to its complexity.

A more direct approach began to be explored experimentally; by installing a perfusion and drainage catheter system into the epidural or intrathecal spaces, localized sustained regional cooling could be generated. The necessity to perform a laminectomy to install this cooling apparatus as well as the direct installation of iced fluids into the epidural space made it unsuitable for aortic surgery, but alternative localized approaches continue to be explored [21].

Epidural Cooling Catheter to Protect the Spinal Cord from Ischemia During Aortic Surgery

Using swine, Mori et al. [22] investigated whether epidural placement of a cooling catheter rather than infusing iced saline solution could protect the spinal cord from ischemia during aortic surgery.

Fourteen domestic pigs were divided into two groups of 7 each. Each underwent epidural catheter placement preceding 30 minutes of aortic cross-clamping distal to the origin of the left subclavian artery. In group 1, cold water was circulated continuously through the lumen of the catheter connected to an external unit. In group 2, animals received catheter placement without cooling. Spinal cord somatosensory evoked potentials were recorded. Neurologic status involving hind limbs was graded sequentially after surgery. At aortic crossclamping, spinal temperature in group 1 (31.7° C.±0.6° C.) was significantly lower than in group 2 (37.8° C.±0.4° C.; p<0.0001). No significant elevation of intrathecal pressure accompanied cooling with the catheter (group 1, 8.1±1.7 mm Hg; group 2, 8.0±1.5 mm Hg). Mean duration of total loss of potentials was significantly shorter in group 1 (7.4±3.8 minutes) than group 2 (19.7±7.3 minutes; p=0.0002). Pigs in group 1 exhibited better hind limb function recovery (mean Tarlov score, 4.7±0.5) than group 2 (0.6±0.8; p=0.0017). Group 1 showed normal histologic characteristics, whereas group 2 showed loss of motor neurons in the ventral horns. Epidural cooling catheter without iced saline infusion can cool the spinal cord without elevating intrathecal pressure, protecting the cord against ischemia [23].

Cambria et al. [23] summarized their experience with epidural cooling (EC) to achieve regional spinal cord hypothermia and thereby decrease the risk of spinal cord ischemic injury during the course of descending thoracic aneurysm (TA) and thoraco-abdominal aneurysm (TAA) repair.

During the interval July 1993 to December 1995, 70 patients underwent TA (n=9, 13%) or TAA (n=61) (type I, 24 [34%], type II, 11 [15%], type III, 26 [37%]) repair using the EC technique. The latter was accomplished by continuous infusion of normal saline (4 degrees C.) into a T11-12 epidural catheter; an intrathecal catheter was placed at the L3-4 level for monitoring of cerebrospinal fluid temperature (CSFT) and pressure (CSFP). All operations (one exception, atrio-femoral bypass) were performed with the clamp-and-sew technique, and 50% of patients had preservation of intercostal vessels at proximal or distal anastomoses (30%) or by separate inclusion button (20%). Neurologic outcome was compared with a published predictive model for the incidence of neurologic deficits after TAA repair and with a matched (Type IV excluded) consecutive, control group (n=55) who underwent TAA repair in the period 1990 to 1993 before use of EC.

EC was successful in all patients, with a 1442±718 ml mean (range, 200 to 3500 ml) volume of infusate; CSFT was reduced to a mean of 24 degrees±3 degrees C. during aortic cross-clamping with maintenance of core temperature of 34±0.8 degrees C. Mean CSFP increased from baseline values of 13±8 mm Hg to 31±6 mm Hg during cross-clamp. Seven patients (10%) died within 60 days of surgery, but all survived long enough for evaluation of neurologic deficits. The EC group and control group were well-matched with respect to mean age, incidence of acute presentations/aortic dissection/aneurysm rupture, TAA type distribution, and aortic cross-clamp times. Two lower extremity neurologic deficits (2.9%) were observed in the EC patients and 13 (23%) in the control group (p<0.0001). Observed and predicted deficits in the EC patients were 2.9% and 20.0% (p=0.001), and for the control group 23% and 17.8% (p=0.48). In considering EC and control patients (n=115), variables associated with postoperative neurologic deficit were prolonged (>60 min) visceral aortic cross-clamp time (relative risk, 4.4; 95% CI, 1.2 to 16.5; p=0.02) and lack of epidural cooling (relative risk, 9.8; 95% CI, 2 to 48; p=0.005).

EC is a safe and effective technique to increase the ischemic tolerance of the spinal cord during TA or TAA repair. When used in conjunction with a clamp-and-sew technique and a strategy of selective intercostal reanastomosis, EC has significantly reduced the incidence of neurologic deficits after TAA repair.

Despite advances in spinal cord protection, paraplegia continues to be a serious complication of descending and thoraco-abdominal aortic operations. Moomiaie et al. [24] devised and tested a novel, self-contained catheter designed to cool the spinal cord topically after being threaded into the spinal column. A cooling catheter for this purpose was specifically designed and produced. The catheter has two lumina, one for ingress and one for egress of fluid. The system is self-contained, so that the fluid does not communicate in any way with the spinal fluid. A console device circulates cold fluid through the catheter. The catheter was tested in 5 adult sheep, with direct monitoring of core body temperature and spinal cord temperature in both active cooling and passive re-warming cycles. In testing in 4 sheep (five attempted implants, with one failure), the catheter worked without problem, producing effective cooling of the spinal cord, from a mean temperature of 36.8 degrees C. (core temperature) to 30.5 degrees C. (spinal temperature) (p<0.0001). In no case did post-mortem examination or histology reveal any evidence of damage to the spinal cord from hypothermia. Temperature rose toward body temperature after cessation of active cooling. Effective topical cooling of the spinal cord can be achieved via a specially designed, self-contained cooling catheter placed into the intra-thecal space. This catheter holds promise for spinal cord protection in aortic surgery. Also, this catheter may be useful as well in mitigating injury to the spinal cord in cases of traumatic spinal column injury.

Salzano et al. [25] tested in pigs the hypothesis that regional deep hypothermia of the spinal cord achieved by cerebrospinal fluid cooling will protect against ischemic injury during thoracic aortic cross-clamping. Eight control animals underwent aortic cross-clamping at the distal aortic arch and just above the diaphragm for 30 minutes. Eight experimental animals had placement of two subarachnoid perfusion catheters through laminectomies at T4 and the lower lumbar region. The subarachnoid space was perfused with normal saline solution at 6 degrees C. delivered by gravity infusion, with infusion rates adjusted to maintain cord temperatures at less than 20 degrees C. After 30 minutes of aortic cross-clamping, the infusion was stopped and the cord allowed to warm to body temperature. Hind limb neurologic function was graded by Tarlov's scale. All of the animals in the control group had complete hind limb paraplegia (Tarlov grade 0) postoperatively. Seven of the 8 animals in the experimental group had preservation of hind limb motor function (Tarlov grade 2), and 1 animal had complete hind limb paraplegia (Tarlov grade 0) (p=0.002, Fisher's exact test). It was concluded that regional deep hypothermia of the spinal cord in pigs does provide some protection from ischemic injury during thoracic aortic cross-clamping. Clinically this may be a useful adjunct for prevention of paraplegia during thoracic aortic operations.

It is reported that hypothermia has some protective effect against ischemia of the spinal cord during thoraco abdominal aneurysm repair. However, it has not been elucidated clinically whether regional spinal cord hypothermia by epidural perfusion cooling is effective and safe. Tabayashi et al. [26] assessed the effect and safety of perfusion cooling of the epidural space during most or all of descending thoracic or thoracoabdominal aneurysm repair. From January 1998 to December 2007, a total of 102 patients with a mean age of 61 years underwent replacement of most or all of the descending thoracic aorta or thoraco-abdominal aorta with the aid of mild hypothermia via epidural perfusion cooling and cerebrospinal fluid (CSF) drainage. Risk factors for spinal cord injury and hospital death were analyzed using univariate and multivariate analyses. The actuarial survival rate was calculated by the Kaplan-Meier method. The mean lowest CSF temperature was 23.3 degrees C. during epidural perfusion cooling. The mean temperature difference between the nasopharynx and CSF was 8.4 degrees C. The incidence of spinal cord injury was 3.9% (4/102), and that of hospital death was 5.9% (6/102). There was no significant risk factor associated with spinal cord injury. Type III aneurysm and postoperative cerebrovascular accident, respiratory failure, liver failure, and infection were predictors of hospital death. The actuarial survival rates at 3 and 5 years were 82.1% and 75.9%, respectively. Epidural perfusion cooling is a safe method to employ in clinical situations.

The Effect of Intrathecal Tetracaine on the Neurological Sequelae of Spinal Cord Ischemia and Reperfusion with Aortic Occlusion

Spinal cord ischemia and resultant paraplegia are devastating sequelae in up to 40% of patients undergoing repair of thoraco-abdominal aneurysms. Breckwoldt et al. [27] investigated the effect of intrathecal tetracaine on the neurological sequelae of spinal cord ischemia and reperfusion with aortic occlusion. Cocaine-derived anesthetics (lidocaine and its analogues) have been shown to decrease neuronal cell metabolism and also have specific neuronal membrane stabilizing effects. New Zealand white rabbits were anesthetized and spinal cord ischemia was then induced by infrarenal aortic occlusion. Animals were divided into six treatment groups. Tetracaine (groups 2 and 4) or normal saline solution (group 5) was administered intrathecally before aortic cross-clamping. Groups 1 and 3 functioned as controls. Group 6 animals received intravenous thiopental. Rabbits were classified as either neurologically normal or injured (paralyzed or paretic). Among controls, 25 minutes of aortic occlusion produced varied neurological sequelae (group 1, 3/6 injured, 50%) whereas 30 minutes resulted in more consistent injury (group 3, 5/6 injured, 83%). All rabbits that received intrathecal saline solution were paralyzed (group 5, 4/4 injured, 100%). Animals treated with intrathecal tetracaine and aortic occlusion of 30 minutes (group 4) showed significantly better preservation of neurological function (6/7 normal, 86%) than controls and saline-treated animals (groups 3 and 5). All animals treated with intrathecal tetracaine and aortic occlusion for 25 minutes (group 2) showed no signs of injury (5/5 normal, 100%), but this was not significant versus controls (group 1). Intravenous thiopental (group 6, 5/5 injured, 100%) had no beneficial effect. Intrathecal tetracaine significantly and dramatically abrogated the neurological injury secondary to spinal cord ischemia and reperfusion after aortic occlusion at 30 minutes in the rabbit model.

### The Use of Prophylactic Hypothermia for Traumatic Brain Injury

During the past 9 years, considerable new evidence has accumulated supporting the use of prophylactic hypothermia for traumatic brain injury (TBI). Studies can be divided into 2 broad categories: studies with protocols for cooling for a short, predetermined period (e.g., 24-48 h), and those that cool for longer periods and/or terminate based on the normalization of intracranial pressure (ICP). There have been no systematic reviews of hypothermia for TBI that include this recent new evidence.

This analysis followed the recommendations of the Cochrane Handbook for Systematic Reviews of Interventions and the QUOROM (quality of reporting of meta-analyses) statement. Fox et al. [28] developed a comprehensive search strategy to identify all randomized controlled trials (RCTs) comparing therapeutic hypothermia with standard management in TBI patients. They searched Embase, MEDLINE, Web of Science, the Cochrane Central Register of Controlled Trials, the Cochrane Database of Systematic Reviews, ProceedingsFirst and PapersFirst. Additional relevant articles were identified by hand-searching conference proceedings and bibliographies. All stages of study identification and selection, quality assessment and analysis were conducted according to prospectively defined criteria. Study quality was determined by assessment of each study for the use of allocation concealment and outcome assessment blinding. Studies were divided into 2 a priori-defined subgroups for analysis based on cooling strategy: short term (≤48 h), and long term or goal-directed (>48 h and/or continued until normalization of ICP). Outcomes included mortality and good neurologic outcome (defined as Glasgow Outcome Scale score of 4 or 5). Pooling of primary outcomes was completed using relative risk (RR) and reported with 95% confidence intervals (CIs).

Of 1709 articles, 12 studies with 1327 participants were selected for quantitative analysis. Eight of these studies cooled according to a long-term or goal-directed strategy, and 4 used a short-term strategy. Summary results demonstrated lower mortality (RR 0.73, 95% CI 0.62-0.85) and more common good neurologic outcome (RR 1.52, 95% CI 1.28-1.80). When only short-term cooling studies were analyzed, neither mortality (RR 0.98, 95% CI 0.75-1.30) nor neurologic outcome (RR 1.31, 95% CI 0.94-1.83) were improved. In 8 studies of long-term or goal-directed cooling, mortality was reduced (RR 0.62, 95% CI 0.51-0.76) and good neurologic outcome was more common (RR 1.68, 95% CI 1.44-1.96).

The best available evidence to date supports the use of early prophylactic mild-to-moderate hypothermia in patients with severe TBI (Glasgow Coma Scale score ≤8) to decrease mortality and improve rates of good neurologic recovery. This treatment should be commenced as soon as possible after injury (e.g., in the emergency department after computed tomography) regardless of initial ICP, or before ICP is measured. Most studies report using a temperature of 32 degrees-34 degrees C. The maximal benefit occurred with a long-term or goaldirected cooling protocol, in which cooling was continued for at least 72 hours and/or until stable normalization of intracranial pressure for at least 24 hours was achieved.

Peterson et al. [29] conducted an updated meta-analysis of the effects of hypothermia therapy on mortality, favorable neurologic outcome, and associated adverse effects in adults with traumatic brain injury (TBI) for use by Brain Trauma Foundation (BTF)/American Association of Neurological Surgeons (AANS) task force to develop evidence-based treatment guidelines. The data sources relied on hand searches of four previous goodquality systematic reviews, which all conducted electronic searches of primarily MEDLINE (OVID), EMBASE, and Cochrane Library. An independent, supplemental electronic search of MEDLINE was undertaken as well (last searched June 2007). Only English-language publications of randomized controlled trials of therapeutic hypothermia in adults with TBI were selected for analysis. Two reviewers independently abstracted data on trial design, patient population, hypothermia and cointervention protocols, patient outcomes, and aspects of methodological quality. Pooled relative risks (RR) and associated 95% confidence intervals (CIs) were calculated for each outcome using random-effects models. In the current study, only 13 trials met eligibility criteria, with a total of 1339 randomized patients. Sensitivity analyses revealed that outcomes were influenced by variations in methodological quality. Consequently, main analyses were conducted based on eight trials that demonstrated the lowest potential for bias (n=781). Reductions in risk of mortality were greatest (RR 0.51; 95% CI 0.33, 0.79) and favorable neurologic outcomes much more common (RR 1.91; 95% CI 1.28, 2.85) when hypothermia was maintained for more than 48 h. However, this evidence comes with the suggestion that the potential benefits of hypothermia may likely be offset by a significant increase in risk of pneumonia (RR 2.37; 95% CI 1.37, 4.10). In sum, the present study's updated metaanalysis supports previous findings that hypothermic therapy constitutes a beneficial treatment of TBI in specific circumstances. Accordingly, the BTF/AANS guidelines task force has issued a Level III recommendation for optional and cautious use of hypothermia for adults with TBI.

### Lipid Neuroprotection

The harmony and function of the complex brain circuits and synapses are sustained mainly by excitatory and inhibitory neurotransmission, neurotrophins, gene regulation, and factors, many of which are incompletely understood. A common feature of brain circuit components, such as dendrites, synaptic membranes, and other membranes of the nervous system, is that they are richly endowed in docosahexaenoic acid (DHA), the main member of the omega-3 essential fatty acid family. DHA is avidly retained and concentrated in the nervous system and known to play a role in neuroprotection, memory, and vision. Only recently has it become apparent why the surprisingly rapid increases in free (unesterified) DHA pool size take place at the onset of seizures or brain injury. This phenomenon began to be clarified by the discovery of neuroprotectin D1 (NPD1), the first-uncovered bioactive docosanoid formed from free DHA through 15-lipoxygenase-1 (15-LOX-1). NPD1 synthesis includes, as agonists, oxidative stress and neurotrophins. The evolving concept is that DHA-derived docosanoids set in motion endogenous signaling to sustain homeostatic synaptic and circuit integrity. NPD1 is anti-inflammatory, displays inflammatory resolving activities, and induces cell survival, which is in contrast to the pro-inflammatory actions of the many of omega-6 fatty acid family members. Bazan et al. [30] highlighted studies relevant to the ability of DHA to sustain neuronal function and protect synapses and circuits in the context of DHA signalolipidomics. DHA signalolipidomics comprises the integration of the cellular/tissue mechanism of DHA uptake, its distribution among cellular compartments, the organization and function of membrane domains containing DHA phospholipids, and the precise cellular and molecular events revealed by the uncovering of signaling pathways regulated by docosanoids endowed with prohomeostatic and cell survival bioactivity. Therefore, this approach offers emerging targets for prevention, pharmaceutical intervention, and clinical translation involving DHAmediated signaling.

The significance of the selective enrichment in omega-3 essential fatty acids in photoreceptors and synaptic membranes of the nervous system has remained, until recently, incompletely understood. While studying mechanisms of cell survival in neural degeneration, Palacios-Pelaez et al. [31] discovered a docosanoid synthesized from unesterified docosahexaenoic acid (DHA) by a 15-lipoxygenase (15-LOX), which they called neuroprotectin D1 (NPD1; 10R,17S-dihydroxy-docosa-4Z,7Z,11E,13E,15E,19Z hexaenoic acid). This lipid mediator is a docosanoid because it is derived from the 22 carbon (22C) precursor DHA, unlike eicosanoids, which are derived from the 20 carbon (20C) arachidonic acid (AA) family member of essential fatty acids. They discovered that NPD1 is promptly made in response to oxidative stress, as a response to brain ischemia-reperfusion, and in the presence of neurotrophins. NPD1 is neuroprotective in experimental brain damage, in oxidative-stressed retinal pigment epithelial (RPE) cells, and in human brain cells exposed to amyloid-beta (Abeta) peptides. They thus envision NPD1 as a protective sentinel, one of the very first defenses activated when cell homeostasis is threatened by imbalances in normal neural function. They provide here, in three sections, recent experimental examples that highlight the specificity and potency of NPD1 spanning beneficial bioactivity during initiation and early progression of neurodegeneration: 1) during retinal signal phototransduction, 2) during brain ischemia-reperfusion, and 3) in Alzheimer's disease (AD) and stressed human brain cell models of AD. From this experimental evidence, they conclude that DHA-derived NPD1 regulation targets upstream events of brain cell apoptosis, as well as neuro-inflammatory signaling, promoting and maintaining cellular homeostasis, and restoring neural and retinal cell integrity.

Deficiency in docosahexaenoic acid (DHA) is associated with impaired visual and neurological development, cognitive decline, macular degeneration, and other neurodegenerative diseases. DHA is concentrated in phospholipids of the brain and retina, with photoreceptor cells having the highest DHA content of all cell membranes. The discovery that neuroprotectin D1 (NPD1; 10R, 17S-dihydroxy-docosa-4Z,7Z,11E,13E,15Z,19Z-hexaenoic acid) is a bioactive mediator of DHA sheds light on the biological importance of this fatty acid. In oxidative stress-challenged human retinal pigment epithelial (RPE) cells, human brain cells, or brain ischemia-reperfusion, NPD1 synthesis is enhanced as a response for sustaining homeostasis. Thus, neurotrophins, Abeta peptide (Abeta42), calcium ionophore A23187, interleukin-1beta (IL-1beta), or DHA supply enhances NPD1 synthesis. NPD1, in turn, upregulates the antiapoptotic proteins of the Bcl-2 family and decreases the expression of proapoptotic Bcl-2 family members. In human neural cells, DHA attenuates Abeta42 secretion, resulting in concomitant formation of NPD1. NPD1 repressed Abeta42-triggered activation of proinflammatory genes and upregulated the antiapoptotic genes encoding Bcl-2, Bcl-x1, and Bfl-1(A1) in human brain cells in culture. Overall, NPD1 signaling regulates brain and retinal cell survival via the induction of antiapoptotic and neuroprotective gene-expression programs that suppress Abeta42-induced neurotoxicity and other forms of cell injury. These in turn support homeostasis during brain and retinal aging, counteract inflammatory signaling, and downregulate events that support the initiation and progression of neurodegenerative disease [32].

Maciá-Botejara et al. [33] studied the changes occurring in brain lipid composition after the administration of total parenteral nutrition (TPN) by comparing two lipid emulsions, one with long-chain triacylglycerols (LCT) and the other with long-chain and medium-chain triacylglycerols (MCT/LCT 50%/50%).

They used 21 young New Zealand rabbits divided into three groups of seven animals each. Two groups were subjected to TPN for 7 d, with each group receiving using one of two different lipid emulsions: Intralipid 20% (group LCT) and Lipofundin MCT/LCT 20% (group MCT/LCT). The third control group received an oral diet and underwent the same surgical procedure with the administration of intravenous saline solution. The energy administered in the TPN formulas was non-protein 100 kcal·kg⁻¹·d⁻¹, with 40% corresponding to fats.

There were modest increases in plasma cholesterol and triacylglycerols. In the brain tissue, there was a decrease of phosphatidylcholine in animals with TPN, which was greater in group LCT. There were no significant differences in the overall percentage distribution of brain fatty acids among the groups.

The lipid emulsions administered in TPN, especially those prepared exclusively with LCT, cause changes in the brain lipid polar fractions of young rabbits.

### Lipid Resuscitation Therapy

On 1998 it was first showed that intravenous intralipid could prevent or improve resuscitation from cardiovascular collapse by severe bupivacaine overdose in rats.

Since then published examples now include toxicities related to verapamil, diltiazem, amlodipine, quetiapine and sertraline, haloperidol, lamotrigine, olanzapine, propranolol, atenolol, nevibolol, doxepin, dosulepin, imipramine, amitriptyline, glyosphate herbicide, flecainide, venlafaxine, moxidectin, and others.

Lipid resuscitation therapy using intravenous lipid emulsion (IVLE) for drug overdoses has gained widespread use. However, there is little information regarding its adverse effects.

Grunbaum et al. [34] performed lipemic interference studies on typical automated platforms to investigate the potential of lipid resuscitation therapy to interfere with the reliability and turnaround time of analytes that would be of interest in acute intoxications. They also tested methods to minimize interferences.

Serum pools were supplemented with increasing concentrations of Intralipid-20%(^{→}) (0%-30%). Analyses were performed on Beckman-Coulter DXC800 and DXI and Roche Modular-P. Analytes demonstrating significant interference were re-measured after centrifugation (14,000×g for 10 minutes).

Triglyceride and glycerol-blanked triglyceride concentrations were similar in IVLE-free samples. However, with addition of IVLE, concentrations were markedly different (139 vs. 76 mmol/L). There was no appreciable interference on the troponin-I, sodium, potassium, chloride, calcium, bicarbonate or urea assays. Albumin and magnesium assays demonstrated significant interference. Amylase, lipase, phosphate, creatinine, total protein, ALT, CK and bilirubin became unmeasurable in IVLEsupplemented samples. Whereas glucose measurement by potentiometry was free of interference, colorimetric methodology was error prone. Centrifugation removed >90% of glycerol-blanked triglyceride (max=5.8 mmol/L), dramatically reducing lipid interferences.

IVLE results in appreciable analytical interferences at concentrations demonstrated in lipid resuscitation therapy. Of particular concern is the marked interference on glucose and magnesium, which may result in unsuccessful and potentially harmful interventions. Major implications for patient care include reporting of incorrect results and delays in the reporting of time-sensitive results. Whenever possible, blood samples should be collected prior to initiating lipid therapy. Interferences can be minimized by brief centrifugation at relatively low speeds on equipment readily available in most core labs.

Li et al. [35] have recently shown that post-ischemic administration of intralipid protects the heart against ischemia-reperfusion injury. They compared the cardioprotective effects of intralipid with cyclosporine-A, a potent inhibitor of the mitochondrial permeability transition pore opening.

In vivo rat hearts or isolated Langendorff-perfused mouse hearts were subjected to ischemia followed by reperfusion with intralipid (0.5%, 1% and 2% ex vivo, and 20% in vivo), cyclosporine-A (0.2 μM, 0.8 μM, and 1.5 μM ex vivo and 10 mg/kg in vivo), or vehicle. The hemodynamic function, infarct size, calcium retention capacity, mitochondrial superoxide production, and phosphorylation levels of protein kinase B (Akt)/glycolgen synthase kinase-3β (GSK-3β) were measured. The values are mean±SEM.

Administration of intralipid at reperfusion significantly reduced myocardial infarct size compared with cyclosporine-A in vivo (infarct size/area at risk) %: 22.9%±2.5% vs. 35.2%±3.5%; p=0.030, n=7/group). Post-ischemic administration of intralipid at its optimal dose (1%) was more effective than cyclosporine-A (0.8 μM) in protecting the ex vivo heart against ischemia-reperfusion injury, as the rate pressure product at the end of reperfusion was significantly higher (mmHg·beats/min: 12,740±675 [n=7] vs. 9203±10,781 [n=5], p=0.024), and the infarct size was markedly smaller (17.3±2.9 [n=7] vs. 29.2±2.7 [n=5], p=0.014). Intralipid was as efficient as cyclosporine-A in inhibiting the mitochondrial permeability transition pore opening (calcium retention capacity=280±8.2 vs. 260.3±2.9 nmol/mg mitochondria protein in cyclosporine-A, p=0.454, n=6) and in reducing cardiac mitochondrial superoxide production. Unlike intralipid, which increased phosphorylation of Akt (6-fold) and GSK-3β (5-fold), cyclosporine-A had no effect on the activation of these prosurvival kinases.

Although intralipid inhibits the opening of the mitochondrial permeability transition pore as efficiently as cyclosporine-A, intralipid is more effective in reducing the infarct size and improving the cardiac functional recovery.

Pretreatment or Resuscitation with a Lipid Infusion

Intralipid^{→} 10% (10% i.v fat emulsion) (a 10% intravenous fat emulsion) is a sterile, non-pyrogenic fat emulsion prepared for intravenous administration as a source of calories and essential fatty acids. It is made up of 10% soybean oil, 1.2% egg yolk phospholipids, 2.25% glycerin, and water for injection. In addition, sodium hydroxide has been added to adjust the ph so that the final product ph is 8. ph range is 6 to 8.9.

The soybean oil is a refined natural product consisting of a mixture of neutral triglycerides of predominantly unsaturated fatty acids. The major component fatty acids are linoleic (44%-62%), oleic (19%-30%), palmitic (7%-14%), linolenic (4%-11%) and stearic (1.4%-5.5%).

Purified egg phosphatides are a mixture of naturally occurring phospholipids which are isolated from the egg yolk.

Intralipid^{→} 10% (10% i.v fat emulsion) (a 10% intravenous fat emulsion) has an osmolality of approximately 300 mosmol/kg water (which represents 260 mos-mol/liter of emulsion) and contains emulsified fat particles of approximately 0.5 micron size.

The total caloric value, including fat, phospholipid and glycerin, is 1.1 kcal per ml of intralipid 10% (10% i.v fat emulsion). The phospholipids present contribute 47 milligrams or approximately 1.5 mmol of phosphorus per 100 ml, of the emulsion.

Weinberg et al. [36] first showed in 1998 that an infusion of a soybean oil emulsion normally used as a total parenteral nutrition solution could prevent (by pretreatment) or improve resuscitation from cardiovascular collapse caused by severe bupivacaine overdose in the intact, anesthetized rat. Subsequent studies from the same laboratory confirmed these findings in isolated rat heart [37] and anesthetized dog [38].

Under the latter experimental model, return of spontaneous circulation after a bupivacaine challenge occurred in all animals receiving lipid, but in none of the saline controls [38]. This study was accompanied by an editorial asking whether lipid might be the long-sought “silver bullet” for local anesthetic systemic toxicity (LAST). Since then, the effectiveness of lipid emulsion infusion in reversing LAST has been confirmed in other laboratories and by systematic analysis [39] and in the clinical setting as well.

Lipid infusion is useful in reversing cardiac toxicity of local anesthetics, and recent reports indicate it may be useful in resuscitation from toxicity induced by a variety of other drugs. While the mechanism behind the utility of lipid rescue remains to be fully elucidated, the predominant effect appears to be creation of a “lipid sink”.

French D et al. [40] tried to determine whether the extraction of drugs by lipid, and hence the clinical efficacy of lipid rescue in toxicological emergencies can be predicted by specific drug properties.

Each drug investigated was added individually to human drug-free serum. Intralipid^{→} was added to this drugcontaining serum, shaken and then incubated at 37° C. The lipid was removed by ultracentrifugation and the concentration of drug remaining in the serum was measured by high-pressure liquid chromatography.

In this in vitro model, the ability of lipid emulsion to bind a drug was largely dependent upon the drug's lipid partition constant. Additionally, using a multiple linear regression model, the prediction of binding could be improved by combining the lipid partition constant with the volume of distribution together accounting for approximately 88% of the variation in the decrease in serum drug concentration with the administration of lipid emulsion.

The lipid partition constant and volume of distribution can likely be used to predict the efficacy of lipid infusion in reversing the cardiac toxicity induced by anesthetics or other medications.

Local anaesthetics may induce cardiac arrest, usually because of rapid absorption from the site of injection or because of an intended intravascular injection. Early central nervous system symptoms usually precede seizures. Cardiac arrhythmias follow the CNS signs. These arrhythmias often resolve with the i.v. bolus injection of 100 to 150 mL of a lipid emulsion (20% Intralipid^{→}). Although long acting local anaesthetics (bupivacaine, ropivacaine, levobupivacaine) are predominantly involved in this cardiac toxicity, lidocaine may also induce cardiac arrhythmias and clinician must be aware of this risk. In case of cardiac arrest, resuscitation manoeuvres are of major importance. They need to be performed immediately and the efficacy of the lipid rescue requires a correct coronary flow to be efficacious. Finally, prevention is the key of a safe injection. It is important to control the dose, to inject slowly, without any excessive pressure and to verify that no blood reflux occurs [41].

### Intralipid Reversing Other Drugs Toxicity

These publications, along with other animal studies, opened the door to more widespread use of lipid emulsion for emergency treatment of toxicities caused by a range of lipophilic drugs. Notably, published examples now include toxicities related to verapamil, diltiazem, amlodipine, quetiapine and sertraline, haloperidol, lamotrigine, olanzapine, propranolol, atenolol, nevibolol, doxepin, dosulepin, imipramine, amitriptyline, glyosphate herbicide, flecainide, venlafaxine, moxidectin, and others.

Tricyclic antidepressant (TCA) toxicity results predominantly from myocardial sodium-channel blockade. Subsequent ventricular dysrhythmias, myocardial depression, and hypotension cause cardiovascular collapse. Animal studies have demonstrated the effectiveness of intravenous lipid-emulsion in treating TCA cardiotoxicity.

Blaber M S et al. [42] report a case of dothiepin (tricyclic antidepressant) overdose causing refractory cardiovascular collapse, which seemed to be successfully reversed with lipid-emulsion therapy (Intralipid^{→}; Fresenius, Cheshire, UK).

Lipid emulsions are a potentially novel therapy for reversing cardiotoxicity seen in TCA overdose. Research is required into the role of lipid emulsion in the management of poisoning by oral lipophilic agents.

### The Lipid Sink Effect

Papadopoulou A et al. [43] hypothesized that by substituting a dye surrogate in place of local anesthetic, they could visually demonstrate dye sequestration by lipid emulsion that would be dependent on both dye lipophilicity and the amount of lipid emulsion used.

They selected 2 lipophilic dyes, acid blue 25 and Victoria blue, with log P values comparable to lidocaine and bupivacaine, respectively. Each dye solution was mixed with combinations of lipid emulsion and water to emulate “lipid rescue” treatment at dye concentrations equivalent to fatal, cardiotoxic, and neurotoxic local anesthetic plasma concentrations. The lipid emulsion volumes added to each dye solution emulated equivalent intravenous doses of 100, 500, and 900 mL of 20% Intralipid in a 75-kg adult. After mixing, the samples were separated into a lipid-rich supernatant and a lipid-poor subnatant by heparin flocculation. The subnatants were isolated, and their colors compared against a graduated dye concentration scale.

Lipid emulsion addition resulted in significant dye acquisition by the lipid compartment accompanied by a reduction in the color intensity of the aqueous phase that could be readily observed. The greatest amount of sequestration occurred with the dye possessing the higher log P value and the greatest amount of lipid emulsion.

This study provides a visual demonstration of the lipid sink effect. It supports the theory that lipid emulsion may reduce the amount of free drug present in plasma from concentrations associated with an invariably fatal outcome to those that are potentially survivable.

Local anesthetic (LA) intoxication with cardiovascular arrest is a potential fatal complication of regional anesthesia. Lipid resuscitation has been recommended for the treatment of LA-induced cardiac arrest. Aim of the study [34] was to compare four different rescue regimens using epinephrine and/or lipid emulsion and vasopressin to treat cardiac arrest caused by bupivacaine intoxication.

Twenty-eight piglets were randomized into four groups (4×7), anesthetized with sevoflurane, intubated, and ventilated. Bupivacaine was infused with a syringe driver via central venous catheter at a rate of 1 mg·kg⁻¹·min⁻¹ until circulatory arrest. Bupivacaine infusion and sevoflurane were then stopped, chest compression was started, and the pigs were ventilated with 100% oxygen. After 1 min, epinephrine 10 μg·kg⁻¹ (group 1), Intralipid^{→}20% 4 ml·kg⁻¹ (group 2), epinephrine 10 μg·kg⁻¹+Intralipid^{→} 4 ml·kg⁻¹ (group 3) or 2 IU vasopressin+Intralipid^{→} 4 ml·kg⁻¹ (group 4) were administered. Secondary epinephrine doses were given after 5 min if required.

Survival was 71%, 29%, 86%, and 57% in groups 1, 2, 3, and 4. Return of spontaneous circulation was regained only by initial administration of epinephrine alone or in combination with Intralipid. Piglets receiving the combination therapy survived without further epinephrine support. In contrast, in groups 2 and 4, return of spontaneous circulation was only achieved after secondary epinephrine rescue.

In cardiac arrest caused by bupivacaine intoxication, first-line rescue with epinephrine and epinephrine+Intralipid^{→} was more effective with regard to survival than Intralipid^{→} alone and vasopressin+Intralipid^{→} in this pig model [44].

Local anesthetic (LA) intoxication with severe hemodynamic compromise is a potential catastrophic event. Lipid resuscitation has been recommended for the treatment of LA-induced cardiac arrest. However, there are no data about effectiveness of Intralipid for the treatment of severe cardiovascular compromise prior to cardiac arrest. Aim of this study was to compare effectiveness of epinephrine and Intralipid for the treatment of severe Hemodynamic compromise owing to bupivacaine intoxication, anesthetized Piglets were with sevoflurane, intubated, and ventilated. Bupivacaine was infused with a syringe driver via a central venous catheter at a rate of 1 mg·kg⁻¹·min⁻¹ until invasively measured mean arterial pressure (MAP) dropped to 50% of the initial value. Bupivacaine infusion was then stopped, and epinephrine 3 μg·kg⁻¹ (group 1), Intralipid^{→} 20% 2 ml·kg⁻¹ (group 2), or Intralipid 20% 4 ml·kg⁻¹ (group 3) was immediately administered. Twenty-one piglets (3×7), were recorded. All animals in group 1 (100%) but only four of seven (57%) piglets in group 2 and group 3, respectively, survived. Normalization of hemodynamic parameters (HR, MAP) and ET(CO₂) was fastest in group 1 with all piglets achieving HR and MAP values. hemodynamic compromise owing to bupivacaine intoxication in piglets, first-line rescue with epinephrine was more effective than Intralipid with regard to survival as well as normalization of hemodynamic parameters and ET(CO₂) [45].

Intravenous lipid emulsion (ILE) has been proposed as a rescue therapy for severe local anesthetic drugs toxicity, but experience is limited with other lipophilic drugs. An 18-year-old healthy woman was admitted 8 h after the voluntary ingestion of sustained-release diltiazem (3600 mg), with severe hypotension refractory to fluid therapy, calcium salts, and high-dose norepinephrine (6.66 μg/kg/min). Hyperinsulinemic euglycemia therapy was initiated and shortly after was followed by a protocol of ILE (intralipid 20%, 1.5 ml/kg as bolus, followed by 0.25 ml/kg over 1 h). The main finding attributed to ILE was an apparent rapid decrease in insulin resistance, despite a prolonged serum diltiazem elimination half-life. Diltiazem is a lipophilic cardiotoxic drug, which could be sequestered in an expanded plasma lipid phase. The mechanism of action of ILE is not known, including its role in insulin resistance and myocardial metabolism in calcium-channel blocker poisoning [46].

### Guidelines for the Management of Local Anaesthetic Toxicity

There is increasing evidence for the use of Intralipid in the management of acute local anaesthetic toxicity. This is supported by the recent Association of Anaesthetists of Great Britain and Ireland (AAGBI) guidelines for the management of local anaesthetic toxicity. Acute hospitals in England and Wales were surveyed to determine the proportion that currently stocked Intralipid, the locations of stocks within the hospital, guidelines related to its use and previous use in the last 12 months. The majority of hospitals surveyed stocked Intralipid in multiple locations, although not in all areas using high volumes of local anaesthetics. Guidelines were typically in place, although these were often local rather than those from the AAGBI. Use in the last 12 months was uncommon, but typically information was not available on indications for its use. More systematic data collection is required on the safety and efficacy of Intralipid in the management of acute drug toxicity [47].

Intralipid therapy has been used successfully as “rescue therapy” in several cases of overdose. West P L et al. [48] present a case of iatrogenic lipid emulsion overdose because of a dosing error:

“A 71-year-old female overdosed on 27 tablets of 5 mg amlodipine. Although initially stable in the Emergency Department, she became hypotensive, oliguric, and respiratory failure developed despite medical therapy. The primary treating team felt that meaningful recovery was unlikely to occur without rapid improvement in clinical status, and 12.5 h after presentation, intralipid rescue therapy was initiated. A protocol for intralipid specifying a maximum infusion of 400 mL of 20% lipid emulsion was faxed, but the infusion was continued until 2 L of lipid emulsion was infused. There were no detectable adverse hemodynamic effects of the intralipid infusion. After this time, laboratory values were difficult to obtain. Three hours after the infusion, a metabolic panel was obtained from ultracentrifuged blood showing hyponatremia. A white blood cell (WBC) was obtained from a complete blood count (CBC) performed 22 h after the infusion, hemoglobin and hematocrit could not be obtained from this blood. A platelet count was obtained by smear estimate. Hematocrits were obtained from centrifuged blood and appeared elevated. No oxygenation could be obtained on blood gas. The patient's family chose to withdraw care on hospital day 2 and no further laboratory draws were obtained. Amlodipine was 1500 ng/mL (ref 3-11 ng/mL).”

### Lipid Emulsion Overdose

Lipid emulsion overdose caused no detectable acute adverse hemodynamic effects. The following laboratory values were unobtainable immediately after infusion: white blood cell count, hemoglobin, hematocrit, platelet count, and a metabolic panel of serum electrolytes. Ultracentrifugation of blood allowed for detection of a metabolic panel 3 h after the infusion. Centrifuged hematocrits appeared to be higher than expected.

Lipid infusion reverses systemic local anesthetic toxicity. The acceptable upper limit for lipid administration is unknown and has direct bearing on clinical management. Hiller D B et al. [49] hypothesize that high volumes of lipid could have undesirable effects and sought to identify the dose required to kill 50% of the animals (LD(50)) of large volume lipid administration.

Intravenous lines and electrocardiogram electrodes were placed in anesthetized, male Sprague-Dawley rats. Twenty percent lipid emulsion (20, 40, 60, or 80 mL/kg) or saline (60 or 80 mL/kg), were administered over 30 mins; lipid dosing was assigned by the Dixon “up-anddown” method. Rats were recovered and observed for 48 hrs then euthanized for histologic analysis of major organs. Three additional rats were administered 60 mL/kg lipid emulsion and euthanized at 1, 4, and 24 hrs to identify progression of organ damage.

The maximum likelihood estimate for LD(50) was 67.72 (SE, 10.69) mL/kg. Triglycerides were elevated immediately after infusion but returned to baseline by 48 hrs when laboratory abnormalities included elevated amylase, aspartate aminotransferase, and serum urea nitrogen for all lipid doses. Histologic diagnosis of myocardium, brain, pancreas, and kidneys was normal at all doses. Microscopic abnormalities in lung and liver were observed at 60 and 80 mL/kg; histopathology in the lung and liver was worse at 1 hr than at 4 and 24 hrs.

The LD(50) of rapid, high volume lipid infusion is an order of magnitude greater than doses typically used for lipid rescue in humans and supports the safety of lipid infusion at currently recommended doses for toxin-induced cardiac arrest. Lung and liver histopathology was observed at the highest infused volumes.

### Intralipid Rescue: 1966-2009

Cave G and Harvey M [50] evaluate the efficacy of lipid emulsion as antidotal therapy outside the accepted setting of local anesthetic toxicity.

Literature was accessed through PubMed, OVID (1966-February 2009), and EMBASE (1947-February 2009) using the search terms “intravenous” AND [“fat emulsion” OR “lipid emulsion” OR “Intralipid”] AND [“toxicity” OR “resuscitation” OR “rescue” OR “arrest” OR “antidote”]. Additional author and conference publication searches were undertaken. Publications describing the use of lipid emulsion as antidotal treatment in animals or humans were included.

Fourteen animal studies, one human study, and four case reports were identified. In animal models, intravenous lipid emulsion (ILE) has resulted in amelioration of toxicity associated with cyclic antidepressants, verapamil, propranolol, and thiopentone. Administration in human cases has resulted in successful resuscitation from combined bupropion/lamotrigine-induced cardiac arrest, reversal of sertraline/quetiapine-induced coma, and amelioration of verapamiland beta blocker-induced shock.

Management of overdose with highly lipophilic cardiotoxic medications should proceed in accord with established antidotal guidelines and early poisons center consultation. Data from animal experiments and human cases are limited, but suggestive that ILE may be helpful in potentially lethal cardiotoxicity or developed cardiac arrest attributable to such agents. Use of lipid emulsion as antidote remains a nascent field warranting further preclinical study and systematic reporting of human cases of use.

Previous investigators have demonstrated amelioration of lipid-soluble drug toxidromes with infusion of lipid emulsions. Clomipramine is a lipid-soluble tricyclic antidepressant with significant cardiovascular depressant activity in human overdose. Harvey M and Cave G [51] compare resuscitation with Intralipid versus sodium bicarbonate in a rabbit model of clomipramine toxicity.

Thirty sedated and mechanically ventilated New Zealand White rabbits were infused with clomipramine at 320 mg/kg per hour. At target mean arterial pressure of 50% initial mean arterial pressure, animals were rescued with 0.9% NaCl 12 mL/kg, 8.4% sodium bicarbonate 3 mL/kg, or 20% Intralipid 12 mL/kg. Pulse rate, mean arterial pressure, and QRS duration were sampled at 2.5-minute intervals to 15 minutes. In the second phase of the experiment, 8 sedated and mechanically ventilated rabbits were infused with clomipramine at 240 mg/kg per hour to a mean arterial pressure of 25 mm Hg. Animals received either 2 mL/kg 8.4% sodium bicarbonate or 8 mL/kg 20% Intralipid as rescue therapy. External cardiac compression and intravenous adrenaline were administered in the event of cardiovascular collapse.

Mean difference in mean arterial pressure between Intralipidand saline solution-treated groups was 21.1 mm Hg (95% confidence interval [CI] 13.5 to 28.7 mm Hg) and 19.5 mm Hg (95% CI 10.5 to 28.9 mm Hg) at 5 and 15 minutes, respectively. Mean difference in mean arterial pressure between Intralipidand bicarbonatetreated groups was 19.4 mm Hg (95% CI 18.8 to 27.0 mm Hg) and 11.5 mm Hg (95% CI 2.5 to 20.5 mm Hg) at 5 and 15 minutes. The rate of change in mean arterial pressure was greatest in the Intralipid-treated group at 3 minutes (6.2 mm Hg/min [95% CI 3.8 to 8.6 mm Hg/min]). In the second phase of the experiment spontaneous circulation was maintained in all Intralipid-treated rabbits (n=4). All animals in the bicarbonate-treated group developed pulseless electrical activity and proved refractory to resuscitation at 10 minutes (n=4, p=0.023).

In this rabbit model, Intralipid infusion resulted in more rapid and complete reversal of clomipramine-induced hypotension compared with sodium bicarbonate. Additionally, Intralipid infusion prevented cardiovascular collapse in a model of severe clomipramine toxicity.

### Intralipid Prevents and Rescues Fatal Pulmonary Arterial Hypertension and Right Ventricular Failure and Enhances the Inflammatory Response to Endotoxin

Pulmonary arterial hypertension (PAH) is characterized by pulmonary vascular remodeling leading to right ventricular (RV) hypertrophy and failure. Intralipid (ILP), a source of parenteral nutrition for patients, contains γ-linolenic acid and soy-derived phytoestrogens that are protective for lungs and heart. Umar S. et al. [52] investigated the therapeutic potential of ILP in preventing and rescuing monocrotaline-induced PAH and RV dysfunction. PAH was induced in male rats with monocrotaline (60 mg/kg). Rats then received daily ILP (1 mL of 20% ILP per day IP) from day 1 to day 30 for prevention protocol or from day 21 to day 30 for rescue protocol. Other monocrotaline-injected rats were left untreated to develop severe PAH by day 21 or RV failure by approximately day 30. Saline or ILP-treated rats served as controls. Significant increase in RV pressure and decrease in RV ejection fraction in the RV failure group resulted in high mortality. Therapy with ILP resulted in 100% survival and prevented PAH-induced RV failure by preserving RV pressure and RV ejection fraction and preventing RV hypertrophy and lung remodeling. In preexisting severe PAH, ILP attenuated most lung and RV abnormalities. The beneficial effects of ILP in PAH seem to result from the interplay of various factors, among which preservation and/or stimulation of angiogenesis, suppression and/or reversal of inflammation, fibrosis and hypertrophy, in both lung and RV, appear to be major contributors. In conclusion, ILP not only prevents the development of PAH and RV failure but also rescues preexisting severe PAH [52].

Novel anti-inflammatory effects of insulin have recently been described, and insulin therapy to maintain euglycemia suppresses the plasma levels of free fatty acids (FFA) and increases the survival of critically ill patients. Krogh-Madsen et al. [53] aimed to explore the effect of short-term high levels of plasma FFA on the inflammatory response to a low dose of endotoxin. Fourteen healthy male volunteers underwent the following two trials in a randomized crossover design: 1) continuous infusion of 20% Intralipid [0.7 ml·kg⁻¹·h⁻¹ (1.54 g/kg)] for 11 h, and 2) infusion of isotonic saline for 11 h (control). In each trial, heparin was given to activate lipoprotein lipase, and an intravenous bolus of endotoxin (0.1 ng/kg) was given after 6 h of Intralipid/saline infusion. Blood samples and muscle and fat biopsies were obtained before the Intralipid/saline infusion and before as well as after infusion of an endotoxin bolus. Plasma levels of FFA, triglycerides, and glycerol were markedly increased during the Intralipid infusion. Endotoxin exposure induced an increase in plasma levels of TNFalpha, IL-6, and neutrophils and further stimulated gene expression of TNF-alpha and IL-6 in both skeletal muscle and adipose tissue. The systemic inflammatory response to endotoxin was significantly pronounced during Intralipid infusion. Short-term hyperlipidemia enhances the inflammatory response to endotoxin, and skeletal muscle and adipose tissue are capable of producing essential inflammatory mediators after endotoxin stimulation.

### Is Intralipid Approved by the FDA for Local Anesthesia-Induced Cardiovascular Collapse or Resuscitation.

As with any FDA-labeled medication, the individual Intralipid products carry with them a set of contraindications to use and adverse effects. However, the extent to which these apply in the setting of Local Anesthesiainduced cardiovascular collapse or resuscitation, for which Intralipid is not FDA-approved, remains to be defined. The general contraindication to Intralipid use is the presence of disorders of fat metabolism. Other contraindications not published in the product's package insert include egg allergy and acute myocardial infarction. The use of Intralipid is cautioned in patients with anemia, severe liver disease, coagulopathies, pulmonary disease, and in patients at risk for fat embolism. The most common adverse effects from general Intralipid use are those related to contamination of the administration site and irritation of the veins likely due to other solutions coinfused with Intralipid.

Early or immediate adverse effects of Intralipid include allergic reactions, headache, somnolence, dizziness, diaphoresis, dyspnea, nausea/vomiting, hyperthermia, and hypercoagulability. More delayed adverse effects of Intralipid include thrombocytopenia, jaundice, overloading syndrome, increased liver function tests, leucopenia, hepatomegaly, and splenomegaly; pancreatitis has rarely been associated with Intralipid use. Those adverse effects that may be doseor rate-related include pulmonary embolus or fat embolus, and pulmonary vasoconstriction may result from bolus administration of Intralipid. There were no adverse effects reported with Intralipid use in the four human case reports after Local Anesthesia-induced cardiovascular collapse, although further investigation is warranted [54].

### Conclusions

A new multimodal proposal for spinal cord and brain treatment and protection due to injuries and diseases has been described:

The epidural catheter is used for cooled saline injection and infusion.

The spinal catheter is used for Intralipid spinal injections and CSF aspiration.

The proposal is based on the current studies on spinal cord cooling and CSF aspiration as well as on the Intralipid resuscitation properties and lipid brain protection.

Joseph Eldor A study is needed to evaluate the clinical value of this multimodal spinal and brain treatment and disease prevention.

### REFERENCES

1. Y. M. Wang, “Compositions and Treatment Method for Brain and Spinal Cord Injuries,” U.S. Pat. No. 6,683,066 B2, 2004.
2. S. W. Hoffman, A. L. Kellermann, D. G. Stein, D. W. Wright and D. W. Lowery-North, “Dosage Regimen for the Treatment of a Traumatic Brain Injury with Progesterone,” Patent No. EP 1868614 A2, 2007.
3. M. Eisenbach-Schwartz, E. Yoles and E. Hauben, “Use of Poly-Glu, Tyr for Neuroprotection Therapy of the Cns or Pns,” Patent No. EP 1406650 B1, 2008.
4. J. Whyte and M. Rosenthal, “Rehabilitation of the Patient with Traumatic Brain Injury,” In: J. A. DeLisa, Ed., Rehabilitation Medicine: Principles and Practice, 2nd Edition, Lippincott, Philadelphia, 1993, pp. 825-860.
5. R. F. Frankowski, J. F. Annegers and S. Whitman, “The Descriptive Epidemiology of Head Trauma in the United States,” In: D. P. Becker and J. T. Povlishock, Eds., Central Nervous System Research Status Report, NINCDS; Bethesda, 1985, pp. 33-43.
6. M. E. Sandel and M. Finch, “The Case for Comprehensive Residency Training in Traumatic Brain Injury: A Commentary,” American Journal of Physical Medicine & Rehabilitation, Vol. 72, No. 5, 1993, pp. 325-326. http://dx.doi.org/10.1097/00002060-199310000-00013
7. B. Jennett and M. Bond, “Assessment of Outcome in Severe Brain Damage,” The Lancet, Vol. 305, No. 7905, 1975, pp. 480-484. http://dx.doi.org/10.1016/50140-6736(75)92830-5
8. J. F. Kraus and D. L. McArthur, “Epidemiologic Aspects of Brain Injury,” Neurologic Clinics, Vol. 14, No. 2, 1996, pp. 435-450. http://dx.doi.org/10.1016/S0733-8619(05)70266-8
9. W. Max, E. J. Mackenzie and D. P. Rice, “Head Injuries: Costs and Consequences,” Journal of Head Trauma Rehabilitation, Vol. 6, No. 2, 1991, pp. 76-91. http://dx.doi.org/10.1097/00001199-199106000-00010
10. W. R. McMordie and S. L. Barker, “The Financial Trauma of Head Injury,” Brain Injury, Vol. 2, No. 4, 1988, pp. 357-364. http://dx.doi.org/10.3109/02699058809150908
11. M. R. Bullock, B. G. Lyeth and J. P. Muizelaar, “Current Status of Neuroprotection Trials for Traumatic Brain Injury: Lessons from Animal Models and Clinical Studies,” Neurosurgery, Vol. 45, No. 2, 1999, pp. 207-217. http://dx.doi.org/10.1097/00006123-199908000-00001
12. L. D. Lehmkuhl, K. M. Hall, N. Mann and W. A. Gordon, “Factors That Influence Costs and Length of Stay of Persons with Traumatic Brain Injury in Acute Care and Inpatient Rehabilitation,” Journal of Head Trauma Rehabilitation, Vol. 8, No. 2, 1993, pp. 88-100. http://dx.doi.org/10.1097/00001199-199308020-00010
13. M. Bennett, D. P. OBrien, J. P. Phillips and M. A. Farrell, “Clinicopathologic Observations in 100 Consecutive Patients with Fatal Head Injury Admitted to a Neurosurgical Unit,” Irish Medical Journal, Vol. 88, No. 2, 1995, pp. 60-62.
14. J. M. Meythaler and J. Peduzzi, “Method of Treating Traumatic Brain and Spinal Cord Injuries and Other Neurogenic Conditions Using Non-Steroidal Anti-Inflammatory Drugs and Naturally Occurring Conotoxins,” Patent No. EP 1210100 A4, 2004.
15. F. Odaibo, C. A. Fajardo and C. Cronin, “Recovery of Intralipid from Lumbar Puncture after Migration of Saphenous Vein Catheter,” Archives of Disease in Childhood, Vol. 67, No. 10, 1992, pp. 1201-1203.
16. Y. Koga, N. Iwatsuki, M. Takahashi and Y. Hashimoto, “Intralipid Solution Mistakenly Infused into Epidural Space,” Anesthesia & Analgesia, Vol. 71, No. 6, 1990, pp. 712-713. http://dx.doi.org/10.1213/00000539-199012000-00030
17. R. A. Stevens and N. Jorgensen, “Successful Treatment of Dural Puncture Headache with Epidural Saline Infusion after Failure of Epidural Blood Patch. Case Report,” Acta Anaesthesiologica Scandinavica, Vol. 32, No. 5, 1988, pp. 429-431. http://dx.doi.org/10.1110.1399-6576.1988.tb02760.x
18. D. K. Turnbull and D. B. Shepherd, “Post-Dural Puncture Headache: Pathogenesis, Prevention and Treatment,” British Journal of Anaesthesia, Vol. 91, No. 5, 2003, pp. 718-729. http://dx.doi.org/10.1093/bja/aeg231
19. R. M. A. Moomiaie, J. Ransden, J. Stein, J. Strugar, Q. B. Zhu, J. H. Kim and J. A. Elefteriades, “Cooling Catheter for Spinal Cord Preservation in Thoracic Aortic Surgery,” The Journal of Cardiovascular Surgery, Vol. 48, No. 1, 2007, pp. 103-108.
20. Y. Okita, “Fighting Spinal Cord Complication during Surgery for Thoracoabdominal Aortic Disease,” General Thoracic and Cardiovascular Surgery, Vol. 59, No. 2, 2011, pp. 79-90. http://dx.doi.org/10.1007/s11748-010-0668-x
21. J. D. Galla, “Regional Spinal Cord Cooling Using a Countercurrent Closed-Lumen Epidural Catheter. Invited Commentary,” The Annals of Thoracic Surgery, Vol. 80, No. 5, 2005, pp. 1833-1834.
22. A. Mori, T. Ueda, T. Hachiya, N. Kabei, H. Okano, R. Yozu and T. Sasaki, “An Epidural Cooling Catheter Protects the Spinal Cord Against Ischemic Injury in Pigs,” The Annals of Thoracic Surgery, Vol. 80, No. 5, 2005, pp. 1829-1833. http://dx. doi. org/10.1016/j.athoracsur.2005.04.031
23. R. P. Cambria, J. K. Davison, S. Zannetti, G. L'Italien, D. C. Brewster, J. P. Gertler, A. C. Moncure, G. M. LaMuraglia and W. M. Abbott, “Clinical Experience with Epidural Cooling for Spinal Cord Protection during Thoracic and Thoracoabdominal Aneurysm Repair,” Journal of Vascular Surgery, Vol. 25, No. 2, 1997, pp. 234-241; Discussion 241-243. http://dx.doi.org/10.1016/S0741-5214(97)70365-3
24. R. M. Moomiaie, J. Ransden, J. Stein, J. Strugar, Q. B. Zhu, J. H. Kim and J. A. Elefteriades, “Cooling Catheter for Spinal Cord Preservation in Thoracic Aortic Surgery,” The Journal of Cardiovascular Surgery (Torino), Vol. 48, No. 1, 2007, pp. 103-108.
25. R. P. Salzano Jr., L. H. Ellison, P. F. Altonji, J. Richter and P. J. Deckers, “Regional Deep Hypothermia of the Spinal Cord Protects against Ischemic Injury during Thoracic Aortic Cross-Clamping,” The Annals of Thoracic Surgery, Vol. 57, No. 1, 1994, pp. 65-70; Discussion 71. http://dx.doi.org/10.1016/0003-4975(94)90366-2
26. K. Tabayashi, Y. Saiki, H. Kokubo, G. Takahashi, J. Akasaka, S. Yoshida, M. Hata, K. Niibori, M. Miura and T. Konnai, “Protection from Postischemic Spinal Cord Injury by Perfusion Cooling of the Epidural Space during Most or All of a Descending Thoracic or Thoracoabdominal Aneurysm Repair,” General Thoracic and Cardiovascular Surgery, Vol. 58, No. 5, 2010, pp. 228-234. http://dx.doi.org/10.1007/s11748-009-0495-0
27. W. L. Breckwoldt, C. M. Genco, R. J. Connolly, R. J. Cleveland and J. T. Diehl, “Spinal Cord Protection during Aortic Occlusion: Efficacy of Intrathecal Tetracaine,” The Annals of Thoracic Surgery, Vol. 51, No. 6, 1991, pp. 959-963. http://dx.doi.org/10.1016/0003-4975(91)91015-N
28. J. L. Fox, E. N. Vu, M. Doyle-Waters, J. R. Brubacher, R. Abu-Laban and Z. Hu, “Prophylactic Hypothermia for Traumatic Brain Injury: A Quantitative Systematic Review,” CJEM, Vol. 12, No. 4, 2010, pp. 355-364.
29. K. Peterson, S. Carson and N. Carney, “Hypothermia Treatment for Traumatic Brain Injury: A Systematic Review and Meta-Analysis,” Journal of Neurotrauma, Vol. 25, No. 1, 2008, pp. 62-71. http://dx.doi.org/10.1089/neu.2007.0424
30. N. G. Bazan, A. E. Musto and E. J. Knott, “Endogenous Signaling by Omega-3 Docosahexaenoic Acid-Derived Mediators Sustains Homeostatic Synaptic and Circuitry Integrity,” Molecular Neurobiology, Vol. 44, No. 2, 2011, pp. 216-222. http://dx. doi. org/10.1007/s12035-011-8200-6
31. R. Palacios-Pelaez, W. J. Lukiw and N. G. Bazan, “Omega-3 Essential Fatty Acids Modulate Initiation and Progression of Neurodegenerative Disease,” Molecular Neurobiology, Vol. 41, No. 2-3, 2010, pp. 367-374. http://dx.doi.org/10.1007/s12035-010-8139-z
32. C. Zhang and N. G. Bazan, “Lipid-Mediated Cell Signaling Protects against Injury and Neurodegeneration,” The Journal of Nutrition, Vol. 140, No. 4, 2010, pp. 858-863. http://dx.doi.org/10.3945/jn.109.114884
33. E. Maciá-Botejara, J. M. Moran-Penco, M. T. EspinJaime, F. Botello-Martinez, J. Salas-Martinez, M. J. Caballero-Loscos and M. Molina-Fernandez, “Brain Lipid Composition in Rabbits after Total Parenteral Nutrition with Two Different Lipid Emulsions,” Nutrition, Vol. 29, No. 1, 2013, pp. 313-317. http://dx.doi.org/10.1016/j.nut.2012.07.020
34. A. M. Grunbaum, B. M. Gilfix, S. Gosselin and D. W. Blank, “Analytical Interferences Resulting from Intravenous Lipid Emulsion,” Clinical Toxicology (Phila), Vol. 50, No. 9, 2012, pp. 812-817. http://dx.doi.org/10.3109/15563650.2012.731509
35. J. Li, A. lorga, S. Sharma, J. Y. Youn, R. Partow-Navid, S. Umar, H. Cai, S. Rahman and M. Eghbali, “Intralipid, a Clinically Safe Compound, Protects the Heart against Ischemia-Reperfusion Injury More Efficiently than Cyclosporine-A,” Anesthesiology, Vol. 117, No. 4, 2012, pp. 836-846. http://dx.doi.org/10.1097/ALN.0b013e3182655e73
36. G. L. Weinberg, T. VadeBoncouer, G. A. Ramaraju, M. F. Garcia-Amaro and M. J. Cwik, “Pretreatment or Resuscitation with a Lipid Infusion Shifts the Dose-Response to Bupivacaine-Induced Asystole in Rats,” Anesthesiology, Vol. 88, No. 4, 1998, pp. 1071-1075. http://dx.doi.org/10.1097/00000542-199804000-00028
37. G. L. Weinberg, R. Ripper, P. Murphy, L. B. Edelman, W. Hoffman, G. Strichartz and D. L. Feinstein, “Lipid Infusion Accelerates Removal of Bupivacaine and Recovery from Bupivacaine Toxicity in the Isolated Rat Heart,” Regional Anesthesia and Pain Medicine, Vol. 31, No. 4, 2006, pp. 296-303.
38. G. Weinberg, R. Ripper, D. L. Feinstein and W. Hoffman, “Lipid Emulsion Infusion Rescues Dogs from Bupivacaine-Induced Cardiac Toxicity,” Regional Anesthesia and Pain Medicine, Vol. 28, No. 3, 2003, pp. 198-202.
39. C. Jamaty, B. Bailey, A. Larocque, E. Notebaert, K. Sanogo and J. M. Chauny, “Lipid Emulsions in the Treatment of Acute Poisoning: A Systematic Review of Human and Animal Studies,” Clinical Toxicology (Phila), Vol. 48, No. 1, 2010, pp. 1-27. http://dx.doi.org/10.3109/15563650903544124
40. D. French, C. Smollin, W. Ruan, A. Wong, K. Drasner and A. H. Wu, “Partition Constant and Volume of Distribution as Predictors of Clinical Efficacy of Lipid Rescue for Toxicological Emergencies,” Clinical Toxicology (Phila), Vol. 49, No. 9, 2011, pp. 801-809. http://dx.doi.org/10.3109/15563650.2011.617308
41. J. X. Mazoit, “Cardiac Arrest and Local Anaesthetics,” Presse Medicate, Vol. 42, No. 3, 2013, pp. 280-286.
42. M. S. Blaber, J. N. Khan, J. A. Brebner and R. McColm, “‘Lipid Rescue’ for Tricyclic Antidepressant Cardiotoxicity,” The Journal of Emergency Medicine, Vol. 43, No. 3, 2012, pp. 465-467. http://dx.doi.org/10.1016/j.jemermed.2011.09.010
43. A. Papadopoulou, J. W. Willers, T. L. Samuels and D. R. Uncles, “The Use of Dye Surrogates to Illustrate Local Anesthetic Drug Sequestration by Lipid Emulsion: A Visual Demonstration of the Lipid Sink Effect,” Regional Anesthesia & Pain Medicine, Vol. 37, No. 2, 2012, pp. 183-187. http://dx.doi.org/10.1097/AAP.0b013e318244b2b7
44. J. Mauch, O. M. Jurado, N. Spielmann, R. BettschartWolfensberger and M. Weiss, “Resuscitation Strategies from Bupivacaine-Induced Cardiac Arrest,” Pediatric Anesthesia, Vol. 22, No. 2, 2012, pp. 124-129. http://dx.doi.org/10.1111/j.1460-9592.2011.03688.x
45. J. Mauch, 0. M. Jurado, N. Spielmann, R. BettschartWolfensberger and M. Weiss, “Comparison of Epinephrine vs Lipid Rescue to Treat Severe Local Anesthetic Toxicity—An Experimental Study in Piglets,” Pediatric Anesthesia, Vol. 21, No. 11, 2011, pp. 1103-1108. http://dx.doi.org/10.1111/j.1460-9592.2011.03652.x
46. V. Montiel, T. Gougnard and P. Hantson, “Diltiazem Poisoning Treated with Hyperinsulinemic Euglycemia Therapy and Intravenous Lipid Emulsion,” European Journal of Emergency Medicine, Vol. 18, No. 2, 2011, pp. 121-123. http://dx.doi.org/10.1097/MEJ.0b013e32834130ab
47. P. Hamann, P. I. Dargan, N. Parbat, H. Ovaska and D. M. Wood, “Availability of and Use of Intralipid (Lipid Rescue Therapy, Lipid Emulsion) in England and Wales,” Emergency Medicine Journal, Vol. 27, No. 8, 2010, pp. 590-592. http://dx.doi.org/10.1136/emj.2009.083352
48. P. L. West, N. J. McKeown and R. G. Hendrickson, “Iatrogenic Lipid Emulsion Overdose in a Case of Amlodipine Poisoning,” Clinical Toxicology (Phila), Vol. 48, No. 4, 2010, pp. 393-396. http://dx.doi.org/10.3109/15563651003670843
49. D. B. Hiller, G. Di Gregorio, K. Kelly, R. Ripper, L. Edelman, R. Boumendjel, K. Drasner and G. L. Weinberg, “Safety of High Volume Lipid Emulsion Infusion: A First Approximation of LD50 in Rats,” Regional Anesthesia & Pain Medicine, Vol. 35, No. 2, 2010, pp. 140-144. http://dx.doi.org/10.1097/AAP.0b013e3181c6f5aa
50. G. Cave and M. Harvey, “Intravenous Lipid Emulsion as Antidote beyond Local Anesthetic Toxicity: A Systematic Review,” Academic Emergency Medicine, Vol. 16, No. 9, 2009, pp. 815-824. http://dx.doi.org/10.1111/j.1553-2712.2009.00499.x
51. M. Harvey and G. Cave, “Intralipid Outperforms Sodium Bicarbonate in a Rabbit Model of Clomipramine Toxicity,” Annals of Emergency Medicine, Vol. 49, No. 2, 2007, pp. 178-185. http://dx.doi.org/10.1016/j.annemergmed.2006.07.016
52. S. Umar, R. D. Nadadur, J. Li, F. Maltese, P. Partownavid, A. van der Laarse and M. Eghbali, “Intralipid Prevents and Rescues Fatal Pulmonary Arterial Hypertension and Right Ventricular Failure in Rats,” Hypertension, Vol. 58, No. 3, 2011, pp. 512-518. http://dx.doi.org/10.1161/HYPERTENSIONAHA.110.168781
53. R. Krogh-Madsen, P. Plomgaard, T. Akerstrom, K. Moller, O. Schmitz and B. K. Pedersen, “Effect of ShortTerm Intralipid Infusion on the Immune Response during Low-Dose Endotoxemia in Humans,” American Journal of Physiology—Endocrinology and Metabolism, Vol. 294, 2008, pp. E371-E379. http://dx.doi.org/10.1152/ajpendo.00507.2007
54. K. L. Felice and H. M. Schumann, “Intravenous Lipid Emulsion for Local Anesthetic Toxicity: A Review of the Literature,” Journal of Medical Toxicology, Vol. 4, No. 3, 2008, pp. 184-191. http://dx.doi.org/10.1007/BF03161199

### Example 4

### Combined Intralipid-Urotherapy for Patients with Cancer

The article relates in general to intralipid and its use in cancer therapy. Specifically, it relates to the combined intralipid-urotherapy for treating cancer, and methods of such treatment.

Cancer cells release various antigens, some of which appear in the urine. Combined oral Intralipid and auto-urotherapy is suggested as a new treatment modality for cancer patients. It will provide the intestinal lymphatic system the many tumor antigens against which antibodies may be produced. These antibodies may be transpierced through the blood stream and attack the tumor and its cells.

Intralipid can increase the response to the cancer antigens in the intestinal lymphatic system against which antibodies may be produced.

### The Philosophy of Cancer

Microbes were known long before the germ theory of disease was invented. It was not the discovery of germs that revolutinized medicine, but the invention of a philosophy of medical explanation that permitted germs to be causative agents of disease (1).

Burnet and Thomas (2) postulated that specific cell mediated immunity may have evolved in vertebrates specially for defense against the “enemy within” rather than against infecting microorganisms and parasites. Most human cancers appear to lack truly tumor-specific antigens. The same neoplastic cell can express several different tumor antigens. For example, relatively cross-reacting tumor-specific transplantation antigens have been demonstrated in many chemically induced tumors (3).

Tumor-associated differentiation antigens are shared by neoplastic and embryonic cells (4). The extent to which human patients react immunologically against their cancers has been a subject of much controversy (5). Paul Ehrlich, in 1909, said: “I am convinced that during development and growth malignant cells arise extensively frequently but that in the majority of people they remain latent due to the protective action of the host. I am also convinced that this natural immunity is not due to the presence of antimicrobial bodies but is determined purely by cellular factors. These may be weakened in the older age groups in which cancer is more prevalent” (6).

### Tumor Antigens in Urine

Human melanoma cells express membrane antigens distinct from those of the normal ectodermal counterparts (7). Urinary-tumor-associated antigen (U-TAA) is one such antigen. This high-molecular weight glycoprotein was first described when melanoma urine was found to react with autologous antibody (8). The antigen has since been detected in the urine of 68% of melanoma patients. In addition, high levels of U-TAA are found to correlate positively with disease occurrence in surgically treated patients (9).

Prostatic specific antigen (PSA) has become an important laboratory test in the management of prostate cancer. PSA levels can be as readily obtained from voided urine as from serum samples (10).

Quantitative urinary immunocytology with monoclonal antibody (mab) 486p 3/12 proved to be valuable for diagnostic use in bladder-cancer patients' urine, especially in the followup of patients with superficial bladder carcinoma (11).

Quantitative urinary immunocytology is a general tool to test the diagnostic usefulness of mabs, assuming that normal and malignant cells differ in their quantitative expression of a given antigen. Selective criteria for selecting mabs for diagnostic approaches should ask not for tumor specificity, but for different quantitative expression of antigen in the tissues or cells in question.

Gastric juice oncofetal antigen determination, due to direct shedding of antigens into the fluid around tumor tissues, appears to accurately indicate the presence and degree of gastric mucosal damage and to be to a slight extent influenced by unrelated factors (12). Patients' age, for example, modifies CEA serum levels (13). A monoclonal antibody (mab) against a human colorectal adenocarcinoma cell line has been raised (14), which reacts with sialosylfucosyllactoteraose (15) corresponding to the sialylated blood group antigen Lewis (a). The antigen defined by this antibody, CA50, is elevated in the serum of many patients with gastrointestinal tumors (16), with a sensitivity for gastric cancer ranging from 20% (17) to 65% (18). CA50 (a tumor-associated gangliosidic antigen) levels have been determined by an RIA test in serum, gastric juice and urine of patients undergoing upper gastrointestinal tract endoscopy. Sensitivity and specificity were respectively 23% and 89% for CA50 determination in urines (19).

Soluble forms of membrane proteins such as cytokine receptors or cellular adhesion molecules (CD14, TNF receptor, CD25, IL-6 receptor, IFN-γ-receptor and CD54) have been detected in human body fluids. They may have important functions in immune regulation by blocking receptor/ligand interactions. The human adhesion receptor CD58 (LFA-3) is expressed on most cell types. A soluble form of CD58 (sCD58) was purified from human urine and partially purified from supernatant of the Hodgkin-derived cell line L428 (20).

Urinary organ-specific neoantigen from colorectal cancer patients has been used to make a monoclonal antibody, BAC 18.1 (21). Organ-specific neoantigen originates in the colon and is excreted into the urine, so the BAC 18.1 binding levels in the urine may be a diagnostic aid for colorectal cancer.

The polyamines spermidine, spermine and their diamine precursor putrescine are ubiquitous constituents of mammalian cells that are fundamentally involved in normal, malignant and induced proliferative states. The polyamines and ornithine decarboxylase (ODC), the rate-limiting enzyme of the polyamine metabolism, were found to play an important role in tumor promotion (22). The suggestion that polyamines play an important role in colorectal cancer was confirmed by studies that found elevated polyamine concentrations in blood or urine (23) of patients with colon carcinoma. Sensitivity of urinary polyamines for colon cancer were highest for total spermidine (92.1%), acetylated putrescine (84.5%), total putrescine (84.0%), N1-acetylspermidine (79.3%) and N8-acetylspermidine (78.6%), but in all these cases specificity was lower than 65% (24). In patients with successful curative surgical treatment all preoperatively elevated urinary polyamine concentrations markedly decreased and returned to normal, whereas they were elevated and increased further in patients with proven relapse of the tumor and/or metastases in different organs (24).

The function of the CD44 gene is severely damaged, beginning with the very early pre-invasive stages of tumor development. This can be used as a means of tumor detection and diagnosis both on solid tissue specimens (25) and on exfoliated cells in clinically obtained excreta and body fluids (26). Urine cell lysates obtained from patients with bladder cancer can be discriminated from normal urine lysates (27) using Western blotting with a monoclonal antibody against the standard form of the CD44 protein.

### Immunotherapy

Zbar and Tanaka (28) first reported on animal immunotherapy based on the principle that tumor growth is inhibited at sites of delayed hypersensitivity reactions provoked by antigens unrelated to the tumor. They injected living Mycobacterium bovis (strain BCG) into established intradermal tumors and caused tumor regression and prevented the development of metastases. For optimum therapeutic effect contact between BCG and tumor cells was necessary.

The ability of tumor immune lymphocytes to localize specifically to tumor offers a possibility for therapy which has been utilized over the past several years (29). The rejection of murine tumors expressing tumor-specific transplantation antigens has been shown to be mediated primarily by immune cells (30). Some 6 to 7% of transplant recipients may develop cancer as a consequence of iatrogenic immunosuppression (31). Studies on the ability of patient lymphocytes to lyse tumor cells in short term (2-8 hr) isotope release assays have shown that lymphocytes from cancer patients can generally destroy only tumor cells from the same patient (32-34), unless the effector cells are not cytolytic T cells but, for example, Natural Killer cells or Lymphokine Activated Killer cells, in which case neoplastic cells representing many different types are sensitive.

Immunotherapy is believed to be capable of eliminating only relatively small amounts of neoplastic cells and, therefore, the failure to induce a regression in patients with excessive tumor burden is not unexpected (35,36). One approach of immunotherapy is to “xenogenize” tumor cells by virus infection. Another is to culture tumor infiltrating lymphocytes with interleukin-2 and reinoculate them into the host with cytokines (37). The introduction of recombinant vectors expressing cytokine genes into tumor infiltrating lymphocyte cells (38) or into the tumor cells themselves (39) may enhance the migration of effector immune cells into the tumor with consequent immunomediated control. The considerable heterogeneity in the expression of tumor associated differentiation antigens by cells within the same tumor constitutes a problem for any immunotherapy, since it facilitates the escape of antigen-negative tumor variants.

An alternative approach toward increasing the immune response to tumor-associated differentiation antigens is to treat the host to be immunized so as to abolish a “suppressor” response. Such treatment can be provided in the form of sublethal whole body x-irradiation (40), injection of a drug such as cyclophosphamide (41), or by the administration of certain anti-idiotypic antibodies (42).

Anergy is defined as a state of T lymphocyte unresponsiveness characterized by absence of proliferation, IL-2 production and diminished expression of IL-2R (43,44). Most available data support suppression as a mechanism of oral tolerance (45,46). Immunological suppression is classically demonstrated by the suppression of antigen-specific immune responses by T lymphocytes (47,48).

### Autoantigens

Oral administration of S-antigen (S—Ag), a retinal autoantigen that induces experimental autoimmune uveitis, prevented or markedly diminished the clinical appearance of S—Ag-induced disease as measured by ocular inflammation (49,50).

Gut associated lymphoid tissue has the capacity to generate potent immune responses on one hand, and to induce peripheral tolerance to external antigens on the other (51-53). Both processes require antigen stimulation (53), involve cytokine production (51) and might occur at the same time—the first leading to potent local and systemic immune responses, while the latter leads to systemic antigen-specific nonresponsiveness (54). The generation of acquired immune responses in the small intestine is believed to occur in Peyer's patches (51,55).

Orally fed protein antigens are found in the blood within 1 hr of feeding (56). Peripheral tolerance is not induced locally, but rather is induced systemically upon transfer of intact antigen, or its peptides, into the circulation (57-59). Oral tolerance may be induced by a single feeding of a protein antigen (60,61) or by several intermittent feedings (46,62). In order to test whether feeding on autoantigen could suppress an experimental autoimmune disease, the Lewis rat model of experimental autoimmune encephalomyelitis was studied (63). With increasing dosages of GP-MBP, the incidence and severity of disease was suppressed, as well as proliferative responses of lymph node cells to MBP. Antibody responses to MBP were decreased but not as dramatically as proliferative responses. Thus it appears that oral tolerance to MBP, as to other non-self antigens (45), preferentially suppress cellular immune responses.

It appears that homologous MBP is a more potent oral tolerogen for experimental autoimmune encephalomyelitis than heterologous MBP (64).

Tumor cells may escape immune recognition in immunocompetent hosts by clonal evolution. Attention could be directed to activate the resident immune effectors to break the anergy or tolerance.

### Urotherapy

Subcutaneous urine injections was practiced in 1912 by Duncan (65) from New York under the name of auto-pyotherapy for urinary infections, and in 1919 by Wildbolz (65) from Bern for diagnostic purposes. Cimino (66) from Palermo reported in 1927 on the use of auto uro-therapy for urinary infections. Rabinowitch (67) in 1931 described this auto-urine therapy for gonarthritis. Jausion et al. (68) used this kind of therapy in 1933 for desensitization and endocrinological problems. They treated with auto urotherapy injections patients who suffered from migraine, pruritus, asthma, urticaria, eczema, psoriasis, etc. Day (69) in 1936 treated patients with acute and subacute glomerulonephritis by injection of an autogenous urinary extract. Sandweiss, Saltzstein and Farbman (70) reported in 1938 that an extract from urine of pregnant women has a prophylactic and therapeutic effect on experimental ulcers in dogs. Shortly thereafter the same group noted that an extract from urine of normal women has a similar beneficial effect (71).

In 1926 Seiffert first described the construction of ileal loop conduits for urinary diversion (72). Bricker in the 1950s popularized the use of the ileal loop as a means of supravesical urinary diversion following exenteration for pelvic malignancy in adults (73). Ureterosigmoidostomy as a means of urinary diversion was used widely from 1920 to 1955. It was this type of implant which Hammer first reported in 1929 associated with tumor (74).

Peyer's patches are immunocompetent lymphoid organs which participate in intestinal immune responses (75). Epithelial cells within the crypts of the small bowel are one of the fastest dividing cells in the body and yet they show one of the lowest rate of malignant transformation (76). Stem cells in the mucosa of the small bowel can divide every 8 to 12 hours (77). Tapper and Folkman (78) demonstrated that exposure of intestinal segments to urine causes marked lymphoid depletion in the segments. These studies give additional support to the idea that a lymphocyte suppressive factor exist in urine (79). The continued presence of urine bathing the intestinal mucosa appears to locally inhibit regeneration of the Peyer's patches.

Starkey et al. (80) detected in human urine a material that is biologically and immunologically similar to epidermal growth factor that causes proliferation and keratinization of epidermal tissues.

The increased susceptibility of the colon to cancer associated with the existence of an implanted ureter has been theorized to relate to 3 factors: 1. The role of the urine in the colon (81,82); 2. The mechanical effect of the fecal stream on the stoma (83); and 3. The age of the anastomosis (84). Adenocarcinoma of the colon mucosa is a recognized complication of ureterosigmoidostomy. The tumor, which develops adjacent to the junction of the ureter with the bowel, occurs 500 times as often as in the population at large and, in children so operated, 7,000 times as often as in all persons under age 25. The latency period is 5 to 50 years (81,85-87).

It is common knowledge that malignant tumors may disappear spontaneously although very infrequently (88-90). Usually it is accepted that this could be due at least partly to an immunological reaction (91,92). Renal adenocarcinoma is one of the cancer types in which such spontaneous regressions have been described most frequently (88,90).

Urinary extracts from patients with aplastic anemia (93) and idiopathic thrombocytopenic purpura (94) are capable of stimulating megakaryocyte colony growth in culture, and when injected into rats could also induce thrombocytosis in peripheral blood and megakaryocytosis in the spleens of these animals. Stanley et al. (95) demonstrated that rabbits immunized with human urine concentrates from leukemic patients developed antibody which neutralized the mouse bone marrow colony stimulating factor in human urine and human serum.

### Preconclusion

Henry Sigerist said, more than 50 years ago: “I personally have the feeling that the problem of cancer is not merely a biological and laboratory problem, but it belongs to a certain extent to the realm of philosophy . . . All experiments require certain philosophical preparation. And I have the feeling that in the case of cancer many experiments were undertaken without the necessary philosophical background, and therefore proved useless” (96).

Malignant tumors express antigens that may stimulate and serve as targets for antitumor immunity. Virally induced tumors usually contain integrated proviral genomes in their cellular genomes and often express viral genome-encoded proteins that may stimulate specific host immune responses. Antigens unique to individual tumors that stimulate specific rejection of transplanted tumors have been demonstrated only in experimental animals. Other tumor antigens that potentially can stimulate immune responses are shared by different tumors. These include products of mutated or rearranged oncogenes or tumor-suppressor genes. Tumors may also overexpress tissue differentiation antigens or embryonic antigens, which also have the potential to be recognized by the immune system. The recent identification of tumor antigens recognized by cytotoxic T cells opens up new possibilities for constructing chemically defined antigens for specific immunotherapy. Treatment of malignant tumors in humans by immunologic approaches, although theoretically attractive, has not yet succeeded on a large scale. Important progress in immunotherapy of cancer is emerging with several different treatment modalities (97).

Recent studies have identified new melanoma antigens that are recognised by CD4(+) T cells. Analysis of tumour-specific CD4(+) T-cell responses may lead to the development of optimal anti-cancer vaccines that can induce an orchestrated effort of tumour-specific CD4(+) and CD8(+) T cells in the fight against cancer (98).

T cells play an important role in in vivo rejection of human melanoma. Human melanoma antigens recognized by autologous T cells were identified. These antigens are classified as tissue (melanocyte)-specific proteins, cancer-testis antigens (proteins expressed in normal testis and various cancers), tumor-specific peptides derived from mutations in tumor cells, and others. A variety of mechanisms generating T cell epitopes on tumor cells were discovered. Various clinical observations, including tumor regression observed in adoptive transfer of gp100-reactive T cells suggest that these identified melanoma peptides may function as tumor rejection antigens. Immunodominant common epitopes that could expand melanoma-reactive cytotoxic T lymphocytes (CTLs) in vitro were found in the MART-1 and gp100 antigens. New immunization protocols—including immunization with peptides, recombinant viruses, plasmid DNAs, and dendritic cells pulsed with peptides as well as adoptive transfer of in vitro-generated CTLs by stimulation with antigenic peptides—were developed (phase I clinical trials have been performed in the Surgery Branch of the National Cancer Institute, Bethesda, Md., U.S.A.). Immunization with the gp100(209(210M)) peptide that was modified to have high HLA-A2 binding affinity, along with incomplete Freund's adjuvant and interleukin (IL)-2, resulted in a 42% response rate in patients with melanoma. These immunotherapies need further improvement due to the mechanisms of tumor escape from T cell responses (99).

Most major advances in human cancer immunology and immunotherapy have come from studies in melanoma. We are beginning to understand the immune repertoire of T cells and antibodies that are active against melanoma, with recent glimpses of the CD4(+) T cell repertoire. The view of what the immune system can see is extending to mutations and parts of the genome that are normally invisible (100).

Pancreatic cancer is the fifth leading cause of cancer deaths in the United States with little or no impact from conventional treatment options. Significant advances in understanding basic immunology have renewed interest in using immunotherapy to treat pancreatic cancer. Cancer immunotherapy, including humanized MAbs, cytokines, and potent vaccine strategies, has been successful in animal models and is being evaluated in clinical trials. Gene therapy is also being explored using methods to inactivate oncogenes, replace defective tumor suppressor genes, confer enhanced chemosensitivity to tumor cells, and increase immunogenicity of tumor cells. Angiogenesis, an essential step in the growth and metastasis of pancreatic cancer, has been targeted by many antiangiogenic agents. Several clinical trials have been initiated to evaluate the role of these innovative strategies in patients with pancreatic cancer with increasingly sophisticated correlative studies to learn more about the mechanisms of tumor rejection with these agents. The rapid translation of basic science discoveries to clinical trials should result in the development of new effective treatments for patients with pancreatic cancer (101).

The immune repertoire contains T cells and B cells that can recognize autologous cancer cells. This repertoire is directed against self, and in some cases altered self (mutations). Priming immune responses against self antigens can be difficult. Strategies are presented using altered self to elicit immunity against self in poorly immunogenic tumor models. Mechanisms underlying immunity to self antigens on cancer cells show that the immune system can use diverse strategies for cancer immunity, in both the immunization and the effector phases. CD4+ T cells are typically, but not always, required for immunization. The effector phase of tumor immunity can involve cytotoxic T cells, macrophages with activating Fc receptors, and/or killer domain molecules. This diversity in the effector phase is observed even when immunizing with conserved paralogs. A consequence of tumor immunity is potentially autoimmunity, which may be undesirable. Autoimmunity uses similar mechanisms as tumor immunity, but tumor immunity and autoimmunity can uncouple. These studies open up strategies for active immunization against cancer (102).

### Cancer Antigens

The spectrum of human antigens allows a monitoring of various pathological processes such as autoimmune disorders and tumorigenesis. Serological analysis of cDNA expression libraries (SEREX) is now used to search for new cancer-associated antigens, which are potential diagnostic markers or targets for immunotherapy of cancer (103).

The immune response can effectively hamper the progression of preclinical stages of tumor growth. Medicine in the postgenomic era offers an increasing possibility of detecting healthy individuals at risk of developing cancer who could benefit from tumor-preventive vaccines. The identification of novel tumor antigens that fulfill two conditions will be crucial for the development of cancer immunoprevention. First, an ideal antigen should have a crucial pathogenetic role in tumor growth to avoid the selection of antigen-loss variants. Second, the antigen should be recognizable by the immune system even in MI-IC-loss variants and should therefore be recognized both by antibodies and T cells. Identifying such antigens will also provide new targets for cancer immunotherapy (104).

Cancer/testis (CT) antigens are a category of tumor antigens with normal expression restricted to male germ cells in the testis but not in adult somatic tissues. In some cases, CT antigens are also expressed in ovary and in trophoblast. In malignancy, this gene regulation is disrupted, resulting in CT antigen expression in a proportion of tumors of various types. Since their initial identification by T-cell epitope cloning, the list of CT antigens has been greatly expanded through serological expression cloning (SEREX) and differential mRNA expression analysis, and approximately 20 CT antigens or antigen families have been identified to date. Characteristics commonly shared by CT antigens, aside from the highly tissue-restricted expression profile, include existence as multigene families, frequent mapping to chromosome X, heterogeneous protein expression in cancer, likely correlation with tumor progression, induction of expression by hypomethylation and/or histone acetylation, and immunogenicity in cancer patients. Spontaneous humoral and cell-mediated immune responses have been demonstrated against several CT antigens, including NY-ESO-1, MAGE-A, and SSX antigens. Since CT antigens are immunogenic and highly restricted to tumors, their discovery has led directly to the development of antigen-specific cancer vaccines, and clinical trials with MAGE-A and NY-ESO-1 are in progress (105).

Our understanding of how immune responses are generated and regulated drives the design of possible immunotherapies for cancer patients. Cancer vaccines that are able to induce tumor-specific immune responses in cancer patients are not always followed by tumor rejection. Two possible reasons that might explain this dichotomy of cancer immunology. First, the immune response generated, although detectable, may not be quantitatively sufficient to reject the tumor. Second, the tumor microenvironment may modulate tumor cell susceptibility to the systemic immune response induced by the immunization (106).

Cytolytic T lymphocytes (CTL) play a major role in the recognition and destruction of tumor cells by the immune system. Some of these antigens, including those encoded by the MAGE genes, are absent on all normal cells, and therefore constitute ideal targets for cancer vaccines aimed at increasing the activity of anti-tumor lymphocytes. Such vaccines are currently tested in clinical trials with melanoma patients. These antigens consist of small peptides that are presented by HLA molecules and that result from the degradation of intracellular proteins. This degradation is performed by an intracellular proteolytic complex called the proteasome. Dendritic cells, which in the lymph node are responsible for antigen presentation to the lymphocytes in order to initiate the immune response, are inefficient to produce some peptides because they contain a different proteasome called “immunoproteasome” (107).

One of the most significant advances in the field of modern tumor immunology is the identification of genes encoding tumor-rejection antigens that are recognized by human leukocyte antigen (HLA) class I-restricted and tumor-specific cytotoxic T lymphocytes (CTLs). Several peptides encoded by these genes are now under clinical trial as cancer vaccines, and major tumor regression has been observed in some melanoma patients. These results indicate that identification of the peptides capable of inducing CTLs may provide a new modality of cancer therapy. Itoh et al. (108) investigated tumor-rejection antigens from epithelial cancers, and reported 7 genes encoding tumor-rejection antigens and peptides available for specific immunotherapy of HLA-A26 or -A24 patients with epithelial cancers. Furthermore, they identified more than 10 genes encoding tumor-rejection antigens and peptides available for specific immunotherapy of HLA-A2 patients with epithelial cancers. Therefore these new antigens and peptides could be applicable to the treatment of numerous epithelial cancer patients.

Cytotoxic T-cell responses to shared tumor antigens have been characterized for several tumor types, and the MHC-associated peptides that comprise these antigens have been defined at a molecular level. These provide new tools to determine whether immune responses can be generated with these tumor antigens, and there are data to suggest that such immune responses can be generated. However, it is also clear that tumor cells can evade immune responses directed against some shared antigens, by downregulating expression of MHC or of the antigenic protein(s), as well as by more active methods such as secretion of immunosuppressive cytokines. Awareness of these mechanisms of immune escape will help to direct development of the next generation of tumor vaccines. Targeting unique antigens and modulating the cytokine environment likely will be critical to comprehensive vaccine systems in the future (109).

The adoptive transfer of tumor-infiltrating lymphocytes along with interleukin 2 into autologous patients resulted in the objective regression of tumor in about 30% of patients with melanoma, indicating that these T cells play a role in tumor rejection. To understand the molecular basis of the T cell-cancer cell interaction Wang (110) and others started to search for tumor antigens expressed on cancer cells recognized by T cells. This led to the identification of several major histocompatibility complex (MHC) class I restricted tumor antigens. These tumor antigens have been classified into several categories: tissue-specific differentiation antigens, tumor-specific shared antigens, and tumor-specific unique antigens. Because CD4+ T cells play a central role in orchestrating the host immune response against cancer, infectious diseases, and autoimmune diseases, a novel genetic approach has recently been developed to identify these MHC class II restricted tumor antigens. The identification of both MHC class I and II restricted tumor antigens provides new opportunities for the development of therapeutic strategies against cancer.

In order to enhance cell mediated cytotoxicity, bispecific antibodies (BsAbs), molecules combining two or more antibodies with different antigenic specificities, have been developed as new agents for immunotherapy. Kudo et al. (111) recent studies revealed that simultaneous administration of two kinds of BsAbs (anti-tumor×anti-CD3 plus anti-tumor×anti-CD28) together with lymphokine activated killer cells with a T cell phenotype (T-LAK cells) inhibited growth of human xenotransplanted tumors in severe combined immunodeficient (SCID) mice, while single BsAb was without effect. Three kinds of BsAbs (anti-tumor×anti-CD3, anti-tumor×anti-CD28, anti-tumor×anti-CD2) showed the highest cytotoxicity against tumor cells when given simultaneously with T-LAK cells or peripheral blood mononuclear cells in vitro and in vivo. BsAbs can be preserved for immediate application, while cytotoxic T lymphocytes (CTLs) must be made-to-order, and are time-consuming to prepare. Tumor associated antigens, such as MAGE antigens, SART antigens, MUC1 antigen, c-erbB 2 antigen or cancer/testis antigens can be served to target antigens for BsAb production. By conjugation with antibodies to effector cells (anti-CD3, anti-CD28, anti-CD16, anti-CD64, anti-CD89 or anti-CD2), many kinds of BsAbs can be produced to cover most types of cancers from different organs. Therefore this strategy might be ubiquitously applicable to most malignancies.

Melanogenesis-related proteins play important roles in melanin synthesis and antigenicity of melanomas. Identification of highly expressed melanoma-associated antigens (MAA) that are immunogenic in humans will provide potential targets for cancer vaccines. Melanogenesis-related proteins have been shown to be MAA. Autoantibody responses to these MAA have been shown to react with melanoma cells and melanocytes, and suggested to play a role in controlling melanoma progression. To assess antibody responses to potential melanoma/melanocyte autoantigens, the open-reading frame sequences of tyrosinase, tyrosinase-related protein (TRP)-1, TRP-2, and melanoma-associated glycoprotein antigen family (gp100/pme117) genes were cloned and expressed as recombinant proteins in E. coli (112). Purified recombinant antigens were employed to detect antibodies in sera of melanoma patients and normal healthy donors. By affinity enzyme-linked immunosorbent assay and western blotting, all recombinant antigens were shown to be antigenic. The main subclass of antibody response to these antigens was IgG. Most importantly this study demonstrated anti-TRP-2 and anti-gp100/pme117 IgG responses in melanoma patients. Only one of 23 normal donors had an antibody response to the antigens tested. MAA-specific IgG antibodies in sera were assessed in melanoma patients (n=23) pre- and post-polyvalent melanoma cell vaccine treatment. Polyvalent melanoma cell vaccine treatment enhanced anti-MAA antibody responses; however, only anti-TRP-2 and anti-gp100/pme117 antibody response was enhanced. These studies suggest that four melanogenesis-related proteins are autoimmunogenic and can be used as potential targets for active-specific immunotherapy.

The adoptive transfer of cytotoxic T lymphocytes (CTLs) derived from tumor-infiltrating lymphocytes (TIL) along with interleukin 2 (IL-2) into autologous patients with cancer resulted in the objective regression of tumor, indicating that these CTLs recognized cancer rejection antigens on tumor cells. To understand the molecular basis of T cell-mediated antitumor immunity, several groups started to search for such tumor antigens in melanoma as well as in other types of cancers. A number of tumor antigens were isolated by the use of cDNA expression systems and biochemical approaches. These tumor antigens could be classified into several categories: tissue-specific differentiation antigens, tumor-specific shared antigens, and tumor-specific unique antigens. However, the majority of tumor antigens identified to date are nonmutated, self-proteins. This raises important questions regarding the mechanism of antitumor activity and autoimmune disease. The identification of human tumor rejection antigens provides new opportunities for the development of therapeutic strategies against cancer (113).

### Cancer Vaccines

Multiple novel immunotherapy strategies have reached the stage of testing in clinical trials that were accelerated by recent advances in the characterization of tumor antigens and by a more precise knowledge of the regulation of cell-mediated immune responses. The key steps in the generation of an immune response to cancer cells include loading of tumor antigens onto antigen-presenting cells in vitro or in vivo, presenting antigen in the appropriate immune stimulatory environment, activating cytotoxic lymphocytes, and blocking autoregulatory control mechanisms. This knowledge has opened the door to antigen-specific immunization for cancer using tumor-derived proteins or RNA, or synthetically generated peptide epitopes, RNA, or DNA. The critical step of antigen presentation has been facilitated by the coadministration of powerful immunologic adjuvants, the provision of costimulatory molecules and immune stimulatory cytokines, and the ability to culture dendritic cells. Advances in the understanding of the nature of tumor antigens and their optimal presentation, and in the regulatory mechanisms that govern the immune system, have provided multiple novel immunotherapy intervention strategies that are being tested in clinical trials (114).

The critical role of antigen-specific T cells in cancer immunotherapy has been amply demonstrated in many model systems. Though success of clinical trials still remains far behind expectation, the continuous improvement in our understanding of the biology of the immune response will provide the basis of optimized cancer vaccines and allow for new modalities of cancer treatment. The future will mainly be concerned with allogeneic bone marrow cell transplantation after non-myeloablative conditioning, because this approach could provide a major breakthrough in cancer immunotherapy (115). Concerning active vaccination protocols the following aspects will be addressed: i) the targets of immunotherapeutic approaches; ii) the response elements needed for raising a therapeutically successful immune reaction; iii) ways to achieve an optimal confrontation of the immune system with the tumor and iv) supportive regimen of immunomodulation. Many questions remain to be answered in the field of allogeneic bone marrow transplantation after non-myeloablative conditioning to optimize the therapeutic setting for this likely very powerful tool of cancer therapy.

Active immunotherapy using dendritic cells (DCs) to deliver tumor antigens has generated considerable excitement among oncologists worldwide. Although most tumor antigens used in immunotherapeutic approaches are tumor-associated, often, little is known about the underlying biology of the target. Antigen expression is a prerequisite for tumor formation or maintenance by the use of ‘obligate’ tumor antigens. The prototype for this class of antigens is the p53 tumor antigen, which is mutated in >50% of human malignancies. The direct involvement of p53 in the malignant transformation of tumors makes it an attractive target for immunotherapy. p53-Reactive antibodies have been found in patients with various types of cancer, demonstrating that the human immune system can recognize and respond to tumor-associated p53. Extensive preclinical experimentation has now validated the translation of p53-expressing DCs into a clinical setting. Clinical trials are ongoing to evaluate the safety and antitumor responses elicited by DCs transduced with adenoviral-p53 in cancer patients (116).

Tumor vaccination strategies have been increased over the past years. This increase began with the identification of tumor antigens recognized by the immune system. Better understanding of the immune system and increasing knowledge about the antigen presentation process and the role of dendritic cells have opened new therapeutic possibilities.

DNA vaccines, already successfully used against viral antigens and covering a broad repertoire of epitopes, might also be of advantage in tumor immunotherapy. Design and selection of vectors are of considerable importance for the vaccination. There are three major types of DNA-based recombinant cancer vaccines: DNA from tumor antigens can be used 1) to modify dendritic cells, 2) as ‘naked’ DNA-vaccine or 3) to construct recombinant viral vaccines (117).

It is now clear that many human tumor antigens can be recognised by the immune system. These tumor antigens can be classified into several groups including cancer-testis, differentiation, tissue specific, over-expressed, and viral-associated antigens. In many cases, there is a known molecular basis of carcinogenesis which provides the explanation for the differentiated expression of these antigens in tumors compared with normal cells. Improved understanding of the biology of the immune response, particularly of immune recognition and activation of T-cells, allow better design of vaccines. Pre-clinical comparative studies allow evaluation of optimal vaccine strategies which can then be delivered to the clinic. Currently, a range of cancer vaccines are being tested including those using tumor cells, proteins, peptides, viral vectors, DNA or dendritic cells. Ultimately, this research should give rise to an entirely new modality of cancer treatments (118).

The identification of antigens on tumor cells has led to significant contributions to the field of immunotherapy. One of the most active areas under investigation in cancer immunotherapy is the development of vaccines against melanoma antigens. Induction of immunity against tumor antigens can follow multiple routes using different mechanisms. Crucial to the development of active immunization and other immunotherapies is the discovery and understanding of the molecular identity of antigens and the mechanisms involved in tumor immunity, as well as escape from immunity (119).

Antigenic differences between normal and malignant cells form the basis of clinical immunotherapy protocols. Because the antigenic phenotype varies widely among different cells within the same tumor mass, immunization with a vaccine that stimulates immunity to a broad array of tumor antigens expressed by the entire population of malignant cells is likely to be more efficacious than immunization with a vaccine for a single antigen. One strategy is to prepare a vaccine by transfer of DNA from the patient's tumor into a highly immunogenic cell line. Weak tumor antigens, characteristic of malignant cells, become strongly antigenic if they are expressed by immunogenic cells. In animal models of melanoma and breast cancer, immunization with a DNA-based vaccine is sufficient to deter tumor growth and to prolong the lives of tumor-bearing mice (120).

Berd (121) has devised a novel approach to active immunotherapy based on modification of autologous cancer cells with the hapten, dinitrophenyl (DNP). The treatment program consists of multiple intradermal injections of DNP-modified autologous tumor cells mixed with BCG. Administration of DNP-vaccine to patients with metastatic melanoma induces a unique reaction—the development of inflammation in metastatic masses. Histologically, this consists of infiltration of T lymphocytes, most of which are CD8+. These T cells usually produce gamma interferon in situ. Moreover, they represent expansion of T cell clones with novel T cell receptor structures. Occasionally, administration of DNP-vaccine results in partial or complete regression of measurable metastases. The most common site of regression has been small lung metastases. Administration of DNP-vaccine to patients in the post-surgical adjuvant setting produces a more striking clinical effect. Berd et al. have treated 214 patients with clinically evident stage III melanoma who had undergone lymphadenectomy. With a median follow-up time of 4.4 years (1.8-10.4 years) the 5-year overall survival (OS) rate is 47% (one nodal site=51%, two nodal sites=33%). These results appear to be comparable to those obtained with high dose interferon. More recent studies suggest that this therapeutic approach is also applicable to ovarian cancer. There appear to be no insurmountable impediments to applying this approach to much larger numbers of patients or to developing it as a standard cancer treatment.

Certain anti-idiotypic antibodies that bind to the antigen-combining sites of antibodies can effectively mimic the three-dimensional structures and functions of the external antigens and can be used as surrogate antigens for active specific immunotherapy. Extensive studies in animal models have demonstrated the efficacy of these vaccines for triggering the immune system to induce specific and protective immunity against bacterial, viral and parasitic infections as well as tumors. Several monoclonal anti-idiotype antibodies that mimic distinct human tumor-associated antigens have been developed and characterized. Encouraging results have been obtained in recent clinical trials using these anti-idiotype antibodies as vaccines (122).

Immunization with anti-idiotype (Id) antibodies represents a novel new approach to active immunotherapy. Extensive studies in animal tumor models have demonstrated the efficacy of anti-Id vaccines in preventing tumor growth and curing mice with established tumor. Bhattacharya-Chatterjee et al. (123) have developed and characterized several murine monoclonal anti-Id antibodies (Ab2) which mimic distinct human tumor-associated antigens (TAA) and can be used as surrogate antigens for triggering active anti-tumor immunity in cancer patients.

Immunization with dendritic cells loaded with tumor antigens could represent a powerful method of inducing antitumor immunity. Studies from several laboratories have shown that immunization with dendritic cells pulsed with specific antigens prime cytotoxic T-cells and engender tumor immunity. The majority of cancer patients who lack an identified tumor antigen and/or cannot provide sufficient tumor tissue for antigen preparation are excluded from treatment with cancer vaccines based on using either specific tumor antigens or mixtures of tumor-derived antigens in the form of peptides or proteins isolated from tumor cells. Vaccination with the mRNA content of tumor cells would extend the scope of vaccination to this group of patients as well because RNA can be amplified from very few cancer cells (124).

The adoptive transfer of tumor-infiltrating lymphocytes (TIL) along with interleukin (IL)-2 into autologous patients with cancer resulted in the objective regression of tumor, indicating that T cells play an important role in tumor regression. In the last few years, efforts have been made towards understanding the molecular basis of T-cell-mediated antitumor immunity and elucidating the molecular nature of tumor antigens recognized by T cells. Tumor antigens identified thus far could be classified into several categories: tissue-specific differentiation antigens, tumor-specific shared antigens and tumor-specific unique antigens. CD4+ T cells play a central role in orchestrating the host immune response against cancer, infectious diseases and autoimmune diseases. The identification of tumor rejection antigens provides new opportunities for the development of therapeutic strategies against cancer (125).

Human tumors express a number of protein antigens that can be recognized by T cells, thus providing potential targets for cancer immunotherapy. Dendritic cells (DCs) are rare leukocytes that are uniquely potent in their ability to present antigens to T cells, and this property has prompted their recent application to therapeutic cancer vaccines. Isolated DCs loaded with tumor antigen ex vivo and administered as a cellular vaccine have been found to induce protective and therapeutic anti-tumor immunity in experimental animals. In pilot clinical trials of DC vaccination for patients with non-Hodgkin's lymphoma and melanoma, induction of anti-tumor immune responses and tumor regressions have been observed. Additional trials of DC vaccination for a variety of human cancers are under way, and methods for targeting tumor antigens to DCs in vivo are also being explored. Exploitation of the antigen-presenting properties of DCs thus offers promise for the development of effective cancer immunotherapies (126).

Recently, cancer immunotherapy has emerged as a therapeutic option for the management of cancer patients. This is based on the fact that our immune system, once activated, is capable of developing specific immunity against neoplastic but not normal cells. Increasing evidence suggests that cell-mediated immunity, particularly T-cell-mediated immunity, is important for the control of tumor cells. Several experimental vaccine strategies have been developed to enhance cell-mediated immunity against tumors. Some of these tumor vaccines have generated promising results in murine tumor systems. In addition, several phase I/II clinical trials using these vaccine strategies have shown extremely encouraging results in patients (127).

Animal studies have shown that vaccination with genetically modified tumor cells or with dendritic cells (DC) pulsed with tumor antigens are potent strategies to elicit protective immunity in tumor-bearing animals, more potent than “conventional” strategies that have been tested in clinical settings with limited success. While both vaccination strategies are forms of cell therapy requiring complex and costly ex vivo manipulations of the patient's cells, current protocols using dendritic cells are considerably simpler and would be more widely available. Vaccination with defined tumor antigens presented by DC has obvious appeal. However, in view of the expected emergence of antigen-loss variants as well as natural immunovariation, effective vaccine formulations must contain mixtures of commonly, if not universally, expressed tumor antigens. When, or even if, such common tumor antigens will be identified cannot be, predicted, however. Thus, for the foreseeable future, vaccination with total-tumor-derived material as source of tumor antigens may be preferable to using defined tumor antigens. Vaccination with undefined tumor-derived antigens will be limited, however, by the availability of sufficient tumor tissue for antigen preparation. Because the mRNA content of single cells can be amplified, tumor mRNA, or corresponding cDNA libraries, offer an unlimited source of tumor antigens. DC transfected with tumor RNA were shown to engender potent antitumor immunity in animal studies. Thus, immunotherapy using autologous DC loaded with unfractionated tumor-derived antigens in the form of RNA emerges as a potentially powerful and broadly useful vaccination strategy for cancer patients (128).

### Cancer Immunotherapy

Adoptive immunotherapy—the isolation of antigen-specific cells, their ex vivo expansion and activation, and subsequent autologous administration—is a promising approach to inducing antitumor immune responses. The molecular identification of tumor antigens and the ability to monitor the persistence and transport of transferred cells has provided new insights into the mechanisms of tumor immunotherapy. Recent studies have shown the effectiveness of cell-transfer therapies for the treatment of patients with selected metastatic cancers. These studies provide a blueprint for the wider application of adoptive-cell-transfer therapy, and emphasize the requirement for in vivo persistence of the cells for therapeutic efficacy (129).

There is clear evidence that certain forms of immunotherapy can be successful against certain cancers. However, it would appear that cancerous cells of various origin are exceptionally adept at subverting the immune response. Consequently, it is probable that the most efficacious therapy will be one in which multiple responses of the immune system are activated. There is currently an embarrassment of riches with regard to multiple vaccine strategies in the clinic, although no single method seems to hold the solution (130).

Despite advances in chemotherapy and surgical techniques, patients with cancer often develop local recurrence or metastatic spread. Recent advances in molecular biology, coupled with new insights in tumor immunology, have led to the design of novel antitumor vaccines. Poxviruses are a large family of DNA viruses that provide an effective and safe vector system for vaccine development. The poxvirus strategy has been successfully documented in animal models, and has been used to express both tumor-associated antigens and immune stimulatory molecules (131).

Prostate cancer is the most common malignancy in American men. Metastatic prostate cancer is incurable, with the currently best treatment, androgen ablation, being only palliative. Therefore, there is a need to develop new, more effective therapies against this disease. Multiple immunotherapeutic strategies are being explored for the treatment of prostate cancer, with the hope that such treatment will be more effective and have fewer side effects than current treatment options. Several immunotherapy strategies have been shown to be effective against prostate tumors in animal models, and many of these strategies are beginning to be tested in clinical trials for their efficacy against human prostate cancer. It is likely that effective treatment of prostate cancer will require the use of both immunotherapeutic and traditional approaches in multimodality treatments. In addition, for immunotherapy to be effective against prostate cancer, ways to overcome immune evasion and immunosuppression by the tumor cells will need to be developed (132).

Despite the identification of tumor antigens and their subsequent generation in subunit form for use as cancer vaccines, whole tumor cells remain a potent vehicle for generating anti-tumor immunity. This is because tumor cells express an array of target antigens for the immune system to react against, avoiding problems associated with major histocompatibility complex (MHC)-restricted epitope identification for individual patients. Furthermore, whole cells are relatively simple to propagate and are potentially efficient at contributing to the process of T cell priming. However, whole cells can also possess properties that allow for immune evasion, and so the question remains of how to enhance the immune response against tumor cells so that they are rejected. Scenarios where whole tumor cells may be utilised in immunotherapy include autologous tumor cell vaccines generated from resected primary tumor, allogeneic (MHC-disparate) cross-reactive tumor cell line vaccines, and immunotherapy of tumors in situ. Since tumor cells are considered poorly immunogenic, mainly because they express self-antigens in a non-stimulatory context, the environment of the tumor cells may have to be modified to become stimulatory by using immunological adjuvants. Recent studies have re-evaluated the relative roles of direct and cross-priming in generating anti-tumor immunity and have highlighted the need to circumvent immune evasion (133).

The Wilms tumor gene WT1 is expressed in leukemias and various kinds of solid tumors, including lung and breast cancer, and exerts an oncogenic function in these malignancies, suggesting that WT1 protein is a novel, overexpressed tumor antigen. The WT1 protein, in fact, is an attractive tumor rejection antigen in animal models. Stimulation in vitro of peripheral blood mononuclear cells with HLA-A*2402- and HLA-A*0201-restricted 9-mer WT1 peptides elicits WT1-specific cytotoxic T-lymphocytes (CTLs), and the CTLs kill endogenously WT1-expressing leukemia or solid tumor cells. Furthermore, WT1 immunoglobulin M (IgM) and IgG antibodies are detected in patients with hematopoietic malignancies such as acute myeloid leukemia, chronic myeloid leukemia, and myelodysplastic syndromes, indicating that WT1 protein overexpressed by leukemia cells is indeed immunogenic. Taken together, these results demonstrate that WT1 protein is a promising tumor antigen for cancer immunotherapy against leukemias and various kinds of solid tumors, including lung and breast cancer (134).

In the last few years, a great deal of efforts have been directed towards understanding the molecular basis of T cell-mediated anti-tumor immunity and elucidating the molecular nature of tumor antigens recognized by T cells. Identification of a number of major histocompatibility complex (MHC) class I-restricted melanoma antigens has led to clinical trials aimed at developing effective cancer vaccines. These studies showed some evidence of therapeutic effect on the treatment of cancer, but the exclusive use of CD8+ T cells may not be effective in eradicating tumor. This rekindles interest in the role of CD4+ T cells in antitumor immunity, which play a central role in orchestrating the host immune response against cancer. Thus, Wang et al. (135) have attempted to identify MHC class II-restricted tumor antigens recognized by tumor-specific CD4+ T cells. The identification of tumor rejection antigens provides new opportunities for the development of therapeutic strategies against cancer.

Interleukin (IL)-2 and IL-15 are two cytokine growth factors that regulate lymphocyte function and homeostasis. Early clinical interest in the use of IL-2 in the immunotherapy of renal cell carcinoma and malignant melanoma demonstrated the first efficacy for cytokine monotherapy in the treatment of neoplastic disease. Advances in our understanding of the cellular and molecular biology of IL-2 and its receptor complex have provided rationale to better utilize IL-2 to expand and activate immune effectors in patients with cancer. Exciting new developments in monoclonal antibodies recognizing tumor targets and tumor vaccines have provided new avenues to combine with IL-2 therapy in cancer patients. IL-15, initially thought to mediate similar biological effects as IL-2, has been shown to have unique properties in basic and pre-clinical studies that may be of benefit in the immunotherapy of cancer (136).

Several recent developments have hallmarked progress in tumor immunology and immunotherapy. The use of interleukin-2 (IL-2) in cancer patients demonstrated that an immunological manipulation was capable of mediating the regression of established growing cancers in humans. The identification of the genes encoding cancer antigens and the development of means for effectively immunizing patients against these antigens has opened important new avenues of exploration for the development of effective active and cell-transfer immunotherapies for patients with cancer (137).

A wide range of strategies in cancer immunotherapy has been developed in the last decade, some of which are currently being used in clinical settings. The development of these immunotherapeutical strategies has been facilitated by the generation of relevant transgenic animal models. Since the different strategies in experimental immunotherapy of cancer each aim to activate different immune system components, a variety of transgenic animals have been generated either expressing tumor associated, HLA, oncogenic or immune effector cell molecule proteins (138).

Immunotherapy is in its infancy for many diseases, whether they are neoplastic or autoimmune. The major issues for cancer immunotherapy today involve the definition of molecular targets and the generation of effector mechanisms to attack the targets of interest. Soft tissue sarcomas provide a unique opportunity to examine the immune response against defined antigens. Many types of sarcomas contain tumor-specific chromosomal translocations encoding fusion proteins, which are attractive targets for immunotherapy. Our understanding of the immune system is also coming into clearer focus with the discovery of dendritic cells as powerful natural adjuvants and the teasing out of mechanisms leading to immunity versus tolerance as examples. It is hoped that the intersection of cellular immunology and sarcoma molecular biology will lead to new modalities of therapy for this group of patients with this heterogeneous group of diseases (139).

Studies of the administration of interleukin-2 to patients with metastatic melanoma or kidney cancer have shown that immunological manipulations can mediate the durable regression of metastatic cancer. The molecular identification of cancer antigens has opened new possibilities for the development of effective immunotherapies for patients with cancer. Clinical studies using immunization with peptides derived from cancer antigens have shown that high levels of lymphocytes with anti-tumor activity can be raised in cancer-bearing patients. Highly avid anti-tumor lymphocytes can be isolated from immunized patients and grown in vitro for use in cell-transfer therapies. Current studies are aimed at understanding the mechanisms that enable the cancer to escape from immune attack (140).

The idea that there might be an immune response to cancer has been around for many years. Immunotherapy has a long history, but is only rarely considered as the treatment of choice. Immunotherapy has encountered a number of intrinsic difficulties in cancer, such as the antigenic resemblance between the tumor and normal cells, the rapid kinetic proliferation of tumor cells and their reduced immunogenicity. There are various types of immunotherapy. Aspecific immunotherapy augments the body's immune response without targeting specific tumoral antigens. In adoptive immunotherapy, cells are administered with antitumoral reactivity to mediate neoplasm regression. Specific active immunotherapy is based on the principle that neoplasm cells contain immunogenic sites against which an antitumoral immune response can be induced in an attempt to stimulate the immune system to target specific tumoral antigens. Vaccines against cancer cells are based on a more precise identification of the tumoral antigen components. Passive immunotherapy was limited by the difficulty of obtaining high titering and specificity in early attempts using polyclonal antisera; monoclonal antibodies are currently used alone or in association with radioactive substances and cytotoxic agents. Enormous progress has been made this century in the use of immunotherapy for cancer treatment. It seems likely that the next century will see its increased afficacy, making it one of the possible therapeutic options (141).

Despite major advances in our understanding of adaptive immunity and dendritic cells, consistent and durable responses to cancer vaccines remain elusive and active immunotherapy is still not an established treatment modality. The key to developing an effective anti-tumor response is understanding why, initially, the immune system is unable to detect transformed cells and is subsequently tolerant of tumor growth and metastasis. Ineffective antigen presentation limits the adaptive immune response; however, we are now learning that the host's innate immune system may first fail to recognize the tumor as posing a danger. Recent descriptions of stress-induced ligands on tumor cells recognized by innate effector cells, new subsets of T cells that regulate tumor tolerance and the development of spontaneous tumors in mice that lack immune effector molecules, beckon a reflection on our current perspectives on the interaction of transformed cells with the immune system and offer new hope of stimulating therapeutic immunity to cancer (142).

Immunotherapy approaches to fight cancer are based on the principle of mounting an immune response against a self-antigen expressed by the tumor cells. In order to reduce potential autoimmunity side-effects, the antigens used should be as tumor-specific as possible. A complementary approach to experimental tumor antigen discovery is to screen the human genome in silico, particularly the databases of “Expressed Sequence Tags” (ESTs), in search of tumor-specific and tumor-associated antigens. The public databases currently provide a massive amount of ESTs from several hundreds of cDNA tissue libraries, including tumoral tissues from various types. Vinals et al. (143) described a novel method of EST database screening that allows new potential tumor-associated genes to be efficiently selected. The resulting list of candidates is enriched in known genes, described as being expressed in tumor cells.

The concept of immunotherapy of cancer is more than a century old, but only recently have molecularly defined therapeutic approaches been developed. The identification of tumor antigens can now be accelerated by methods allowing the amplification of gene products selectively or preferentially transcribed in the tumor. However, determining the potential immunogenicity of such gene products remains a demanding task, since major histocompatibility complex (MHC) restriction of T cells implies that for any newly defined antigen, immunogenicity will have to be defined for any individual MHC haplotype. Tumor-derived peptides eluted from MHC molecules of tumor tissue are also a promising source of antigen. Tumor antigens are mostly of weak immunogenicity, because the vast majority is tumor-associated differentiation antigens already ‘seen’ by the patient's immune system. Effective therapeutic vaccination will thus require adjuvant support, possibly by new approaches to immunomodulation such as bispecific antibodies or antibody-cytokine fusion proteins. Tumor-specific antigens, which could be a more potent target for immunotherapy, mostly arise by point mutations and have the disadvantage of being not only tumor-specific, but also individual-specific. Therapeutic vaccination will probably focus on defined antigens offered as protein, peptide or nucleic acid. Irrespective of the form in which the antigen is applied, emphasis will be given to the activation of dendritic cells as professional antigen presenters. Dendritic cells may be loaded in vitro with antigen, or, alternatively, initiation of an immune response may be approached in vivo by vaccination with RNA or DNA, given as such or packed into attenuated bacteria. The importance of activation of T helper cells has only recently been taken into account in cancer vaccination. Activation of cytotoxic T cells is facilitated by the provision of T helper cell-derived cytokines. T helper cell-dependent recruitment of elements of non-adaptive defence, such as leucocytes, natural killer cells and monocytes, is of particular importance when the tumor has lost MHC class I expression. Barriers to successful therapeutic vaccination include: (i) the escape mechanisms developed by tumor cells in response to immune attack; (ii) tolerance or anergy of the evoked immune response; (iii) the theoretical possibility of provoking an autoimmune reaction by vaccination against tumor-associated antigens; and (iv) the advanced age of many patients, implying reduced responsiveness of the senescent immune system (144).

Generating an antitumor immune response in tumor-bearing host has been impaired by several characteristics of both patient and tumor cells. Amongst those requirements is the necessity of generating immune effectors that are specific to tumor cells. The last two decades have seen the description of many so called tumor “specific” antigens. Indeed, strictly specific tumor antigens are scarce. Most antigens are tumor-associated antigens, also shared by normal tissues. Telomerase and its activity have recently been recognized as a major marker of tumoral growth in more than 80% of cancers. Some telomerase subunits might be ideal, if not universal, targets to an antitumor immune response in patients with cancer. Many other major parameters remain to be understood and to be mastered (145).

The survival of patients with cancer has improved steadily but incrementally over the last century, with the advent of effective anticancer treatments such as chemotherapy and radiotherapy. However, the majority of patients with metastatic disease will not be cured by these measures and will eventually die of their disease. New and more effective methods of treating these patients are required urgently. The immune system is a potent force for rejecting transplanted organs or microbial pathogens, but effective spontaneous immunologically induced cancer remissions are very rare. In recent years, much has been discovered about the mechanisms by which the immune system recognizes and responds to cancers. The specific antigens involved have now been defined in many cases. Improved adjuvants are available. Means by which cancer cells overcome immunological attack can be exploited and overcome. Most importantly, the immunological control mechanisms responsible for initiating and maintaining an effective immune response are now much better understood. It is now possible to manipulate immunological effector cells or antigen-presenting cells ex vivo in order to induce an effective antitumour response. At the same time, it is possible to recruit other aspects of the immune system, both specific (e.g. antibody responses) and innate (natural killer cells and granulocytes) (146).

Immunotherapy of cancer is entering into a new phase of active investigation both at the pre-clinical and clinical level. This is due to the exciting developments in basic immunology and tumor biology that have allowed a tremendous increase in our understanding of mechanisms of interactions between the immune system and tumor cells. Clinical approaches are diverse but can now be based on strong scientific rationales. The analysis of the available clinical results suggests that, despite some disappointments, there is room for optimism that both active immunotherapy (vaccination) and adoptive immunotherapy may soon become part of the therapeutic arsenal to combat cancer in a more efficient way (147).

Advancements in the understanding of cellular immunity within the last decade, along with the characterization of tumor antigens, have led to immunotherapeutic approaches for cancer therapy. This mode of treatment is expected to provide more tumor-specific activity, thereby being less toxic to normal cells than standard modalities. Clinical trials are underway throughout the world to determine whether immunotherapy is a practical and viable alternative to conventional cancer therapies. Unlike radiotherapy and chemotherapy, wherein tumor regression is the standard for determining efficacy of the regimens, immunotherapy has to be evaluated by the examination of several immunological parameters within patients (148).

The identification of tumor-associated antigens recognized by cellular or humoral effectors of the immune system has opened new perspectives for cancer therapy. Different groups of cancer-associated antigens have been described as targets for cytotoxic T lymphocytes (CTLs) in vitro and in vivo: 1) cancer-testis (CT) antigens, which are expressed in different tumors and normal testis; 2) melanocyte differentiation antigens; 3) point mutations of normal genes; 4) antigens that are overexpressed in malignant tissues; and 5) viral antigens. Clinical studies with peptides derived from these antigens have been initiated to induce specific CTL responses in vivo. Immunological and clinical parameters for the assessment of peptide-specific reactions have been defined, i.e., delayed-type hypersensitivity (DTH), CTL, autoimmmune, and tumor regression responses. Preliminary results demonstrate that tumor-associated peptides alone elicit specific DTH and CTL responses leading to tumor regression after intradermal injection. Granulocyte-macrophage colony-stimulating factor (GM-CSF) was proven effective in enhancing peptide-specific immune reactions by amplification of dermal peptide-presenting dendritic cells. Long-lasting complete tumor regressions have been observed after induction of peptide-specific CTLs. However, in single cases with disease progression after an initial tumor response, either a loss of the respective tumor antigen targeted by CTLs or of the presenting major histocompatibility complex (MHC) class I allele was detected as a mechanism of immune escape under immunization. Based on these observations, cytokines to enhance antigen and MHC class I expression in vivo are being evaluated to prevent immunoselection. Recently, a strategy utilizing spontaneous antibody responses to tumor-associated antigens (SEREX) has led to the identification of a new CT antigen, NY-ESO-1, which is regarded as one of the most immunogenic antigens known today inducing spontaneous immune responses in 50% of patients with NY-ESO-1-expressing cancers. Clinical studies involving antigenic constructs that induce both antibody and CTL responses will show whether these are more effective for immunotherapy of cancer (149).

Tumors express proteins not commonly found in normal cells, or over-express certain proteins. These may in some cases serve as target antigens for immunological attack. It is therefore essential to improve our understanding of the nature of these target epitopes and the cells which recognize them, in order to develop immunotherapy as a realistic treatment for cancer (150).

Advances in molecular biology have enabled specific antigens present on colorectal cells to be characterized, against which immune responses may be generated. This, in combination with our inability to significantly alter survival from this condition, has resurrected an interest in immunotherapy as a potential treatment option. A number of approaches currently constitute immunotherapeutic options for colorectal cancer. A number of treatment modalities are already in phase III studies, although clearly not all will fulfill their initial promise. Surgeons need to be aware of the advances in this rapidly expanding field, and keep an open mind as to their efficacy (151).

The goal of harnessing the immune system to recognize tumor as “nonself” is not new. Now, thanks to new knowledge and new techniques, however, modalities that seek to activate the host immune system are becoming increasingly feasible as treatments for advanced malignancies (152).

The major impact of recent scientific advances, such as the discovery of genes and gene products, has been to facilitate development of immunotherapies based on the specific stimulation of immune reactions against characterized tumor antigens (153).

Over the last decade, there has been a considerable increase in understanding of immune responses against cancers, the antigenic structures on tumor cells recognised by the immune system, and the development of more effective vaccines. There is, however, very limited understanding of why the immune system most often fails to control tumor growth and progression. In some patients, it is difficult to demonstrate immune responses to their tumors, and it may be assumed that this reflects poor recognition of tumor antigens, induction of anergy in lymphocytes, or suppression of immune responses by tumor-derived factors. In other patients, tumor progression appears to occur despite the presence of antibody or cell-mediated responses. This may indicate selection of tumor cells that have lost tumor antigens or HLA antigens by immune responses against the tumor. Tumor cells may also become resistant to mediators of apoptosis, such as Fas ligand and tumor necrosis factor-related apoptosis-inducing ligand used by lymphocytes to kill tumor cells. It is suggested that development of effective immunotherapy will need to include strategies that take into account these limitations of immune responses and classification of tumors according to the treatment approach most likely to succeed (154).

Heat shock proteins (Hsps), ubiquitous in nature, act as chaperones for peptides and other proteins. They have been implicated in loading immunogenic peptides onto major histocompatibility complex molecules for presentation to T cells. When isolated from tumor cells, Hsps are complexed with a wide array of peptides, some of which serve as tumor-specific antigens. Animal studies have demonstrated that heat shock protein—peptide complexes (HSPPCs) from tumor cells can act as vaccines to prevent or treat tumors. Potent and specific tumor antigens have long been the holy grail in cancer immunotherapy; HSPPCs from tumor cells could become a safe and reliable source of tumor-specific antigens for clinical application (155).

Immunotherapy of mice with preexisting cancers with heat shock protein preparations derived from autologous cancer resulted in retarded progression of the primary cancer, a reduced metastatic load, and prolongation of life-span. Treatment with heat shock protein preparations derived from cancers other than the autologous cancer did not provide significant protection. Spontaneous cancers (lung cancer and melanoma), chemically induced cancers (fibrosarcoma and colon carcinoma), and an ultraviolet radiation-induced spindle cell carcinoma were tested, and the results support the efficacy of autologous cancer-derived heat shock protein-peptide complexes in immunotherapy of cancers without the need to identify specific tumor antigenic epitopes (156).

### Dendritic Cells

The identification of tumor specific antigens has provided important advance in tumor immunology. It is now established that specific cytotoxic T lymphocytes (CTL) and natural killer cells infiltrate tumor tissues and are effector cells able to control tumor growth. However, such a natural antitumor immunity has limited effects in cancer patients. Failure of host defenses against tumor is consecutive to several mechanisms which are becoming targets to design new immunotherapeutic approaches. CTL are critical components of the immune response to human tumors and induction of strong CTL responses is the goal of most current vaccine strategies. Effectiveness of cytokine therapy, cancer vaccines and injection of cells improving cellular immunity have been established in tumor grafted murine models. Clinical trials are underway. Today, interest is particularly focused on cell therapy: injected cells are either “ready to use” effector cells (lymphocytes) or antigen presenting cells able to induce a protective immune reaction in vivo (dendritic cells). The challenge ahead lie in the careful optimization of the most promising strategies in clinical situation (157).

The response of hepatocellular carcinoma (HCC) to therapy is often disappointing and new modalities of treatment are clearly needed. Active immunotherapy based on the injection of autologous dendritic cells (DC) co-cultured ex vivo with tumor antigens has been used in pilot studies in various malignancies such as melanoma and lymphoma with encouraging results. In the study of Ladhams et al. (158), the preparation and exposure of patient DC to autologous HCC antigens and re-injection in an attempt to elicit antitumor immune responses were described. Therapy was given to two patients, one with hepatitis C and one with hepatitis B, who had large, multiple HCC and for whom no other therapy was available. No significant side-effects were observed. The clinical course was unchanged in one patient, who died a few months later. The other patient, whose initial prognosis was considered poor, is still alive and well more than 3 years later with evidence of slowing of tumor growth based on organ imaging.

The characterization of tumor-associated antigens recognized by human T lymphocytes in a major histocompatibility complex (MHC)-restricted fashion has opened new possibilities for immunotherapeutic approaches to the treatment of human cancers. Dendritic cells (DC) are professional antigen presenting cells that are well suited to activate T cells toward various antigens, such as tumor-associated antigens, due to their potent costimulatory activity. The availability of large numbers of DC, generated either from hematopoietic progenitor cells or monocytes in vitro or isolated from peripheral blood, has profoundly changed pre-clinical research as well as the clinical evaluation of these cells. Accordingly, appropriately pulsed or transfected DC may be used for vaccination in the field of infectious diseases or tumor immunotherapy to induce antigen-specific T cell responses. These observations led to pilot clinical trials of DC vaccination for patients with cancer in order to investigate the feasibility, safety, as well as the immunologic and clinical effects of this approach. Initial clinical studies of human DC vaccines are generating encouraging preliminary results demonstrating induction of tumor-specific immune responses and tumor regression. Nevertheless, much work is still needed to address several variables that are critical for optimizing this approach and to determine the role of DC-based vaccines in tumor immunotherapy (159).

Dendritic cells are among the most efficient antigen-presenting cells of our immune system and they play a crucial role in immunity reactions such as the activation of T and B cells and the induction or maintenance of tolerance. New culture methods allow us to generate dendritic cells in sufficient numbers for further studies and for the preparation of antigen-loaded dendritic cells for clinical application in cancer patients. In animal studies immunization with antigen-loaded dendritic cells offered protection from growth of injected tumor cells. In experimental clinical studies in cancer patients with e.g. metastatic renal carcinoma, melanoma and B cell lymphoma some lasting remissions were observed after administration of antigen-loaded dendritic cells. Side effects were minor. Unanswered questions on tumor vaccines with antigen-loaded dendritic cells concern specific matters, such as optimal culture methods and antigen loading, and general matters, such as dose, frequency, duration and route of administration. Also, no method is currently available by which the in vivo immune response can be measured accurately (160).

Research over the last two years has explored a number of possible approaches to applying dendritic cell immunotherapy to the treatment of human cancers. The chosen strategy in clinical situations will vary for individual patients and will depend on the type of tumor, availability of tissue samples and potential source of dendritic cells. The isolation of fractionated tumor peptide from individual patients' tumors for use with autologous stem cell-derived dendritic cells may provide, in at least some cases, an effective and practical approach to cancer immunotherapy. This approach will provide a ‘closed’ system of tumor immunotherapy with all components (dendritic cells, antigen and cytotoxic T lymphocytes) being provided by the patient and applied in a tailor-made fashion to individual patients as an adjuvant to current anti-tumor therapies (161).

Cancer Antibodies

The specificity of antibodies has been harnessed to target cancer cells and the first therapeutic antibodies for use in oncology are now finding application in the clinic. Studies are currently under way to develop new and improved antibodies. Recent developments have been made in the identification of novel targets, including the use of genomic and proteomic technologies. Several methods are also being developed to enhance antibody efficacy (162).

Bi-specific antibodies (BsAbs) combine immune cell activation with tumor cell recognition as a result of which tumor cells are killed by pre-defined effector cells (163).

Antibody-based therapy of human cancers has led to several remarkable outcomes, particularly in the therapy of breast cancer and lymphoma. Many solid tumors have proven less responsive, due in part to difficulties in the tumor-selective delivery of antibodies and potential cytolytic effectors. However, antibodies that directly perturb signaling mechanisms in cells derived from epithelial malignancies have shown benefit; examples include antibodies directed against the extracellular domains of HER2/neu and epidermal growth factor receptor. A long-term goal of immunotherapy has been to induce anti-tumor inflammatory responses that can directly cause tumor regression or induce adaptive responses against tumor-related antigens (164).

Specific targeting of tumor cells may be achieved by using monoclonal antibodies to tumor antigens. Edrecolomab is a mouse-derived monoclonal IGg2A antibody directed against the human tumor-associated C017-1A (or Ep-CAM) antigen, and is the first monoclonal antibody approved for cancer therapy. Encouraging results of several clinical trials were recently reported using edrecolomab for adjuvant therapy after surgery of Duke's C colorectal cancer. Side effects and toxicity profiles compare favorably to conventional regimens of radio- or chemotherapy. Future challenges lie in further improvement of these novel therapeutics, hopefully generating benefit for a larger number of cancer patients (165).

Gangliosides on tumor cells have been suggested as potential target antigens for specific immunotherapy in various types of cancer including small cell lung cancer (SCLC). Brezicka et al. (166) have compared the expression of three gangliosides that have been described as tumor-associated antigens, FucGM1, GM2 and GD3 in SCLC tissue specimens collected at autopsy, using a double-layer immunofluorescence staining method and specific monoclonal antibodies (Mabs) directed against these ganliosides. They found expression of FucGM1, GD3 and GM2 in 70% (n=20), 60% (n=15) and 40% of the tumor cells in all lesions from the same patient (five of eight cases). These results indicate that FucGM1 is a relevant ganglioside antigen in SCLC, and suggest that specific immunotherapy involving more than one ganglioside antigen in SCLC should at least include FucGM1 and GD3.

There is now a considerable body of information documenting the autoimmune consequences of antibodies induced by growing malignancies, or by passively administered and actively induced antibodies, in cancer patients against antigens shared by normal and malignant tissues. This provides a rich source of information addressing the consequences of autoantibodies against a range of antigens. Antibodies against cell-surface or intracellular antigens in the central nervous system (CNS) or on epithelial surfaces of normal tissues do not generally result in autoimmunity, but the same types and titers of antibodies against cell surface antigens in the subepidermal skin, peripheral nerves, blood, or vascular sites such as the spleen and bone marrow readily induce autoimmunity. The blood brain barrier of the CNS and apical antigen expression and the basement membrane in epithelial tissues, may protect these sites from antibody induced damage. Cancer cells, however, are protected by neither unidirectional antigen expression nor basement membranes. Vaccine induced antibodies against a variety of cancer cell surface antigens have been associated with prevention of tumor recurrence in preclinical models and in vaccinated cancer patients, in the absence of demonstrable autoimmunity. This forms the basis for a series of ongoing Phase III trials with single or polyvalent antigen cancer vaccines designed for optimal antibody induction (167).

Immunotherapy of cancer is still mainly an experimental treatment. Some monoclonal antibodies have been approved for adjuvant therapy of cancer in patients, but active immunization strategies have not yet matured to this stage. The fact that vaccination against viral diseases is effective has primed high expectations for successful vaccination against cancer as well. Indeed, in some animal models, therapeutic results could be obtained against short-term established tumors, which paved the way for clinical trials. However, the first results with active immunization in cancer patients were disappointing and this led to a careful examination of current protocols and the search for more effective approaches. Evaluation of the available data suggests that cancer patients may not be comparable in their immune response to cancer vaccines with healthy persons. Furthermore, the tumor seems to be able to develop several immune-escape mechanisms, which either inactivate the specific immune cells or prevent activation of potential effector mechanisms against the tumor (168).

### Genetic Immunotherapy

The establishment of cancer in a host involves at least two major events: the escape of tumor cells from normal growth control and their escape from immunological recognition. Because of this nature of their development, cancer cells seem to be predominatly poorly immunogenic. In contrast to the previous idea that cancer cells express no recognizable antigens, recent progress in the identification and characterization of tumor antigens, as well as the expansion of knowledge on the cellular and molecular mechanisms of antigen recognition by the immune system, have raised the possibility of using immunotherapy to treat certain tumors. Information on these mechanisms has been obtained in three crucial areas: 1) the role of cytokines in the regulation of the immune response, 2) the molecular characterization of tumor antigens in both mouse and human tumors, and 3) the molecular mechanisms of T cell activation and antigen presentation. Such information has provided new insight into tumor immunology and immunotherapy. Furthermore, recombinant DNA technology allows for modification of the genome of mammalian cells for therapeutic purposes in several diseases. Several novel strategies have been developed to derive genetically modified tumor cells and use them as cellular vaccines to induce antitumor immunity in animal tumor models. This combined modality of genetically modified tumor cells and immunotherapy has been termed immunogene therapy of tumors. Crucial to this approach has been the ability to transfer into normal or neoplastic cells genes known to increase the immunogenicity of cells, which subsequently can be used to augment immune reactions in tumor-bearing mice or cancer patients. While there has been success in inducing antitumor immunity in some tumor models, there are difficulties and limitations in the application of these gene-modified tumor cells for the treatment of preexisting tumors (169).

Genetic immunization refers to treatment strategies where gene transfer methods are used to generate immune responses against cancer. Our growing knowledge of the mechanisms regulating the initiation and maintenance of cytotoxic immune responses has provided the rationale for the design of several genetic immunization strategies. Tumor cells have been gene-modified to express immune stimulatory genes and are then administered as tumor vaccines, in an attempt to overcome tumor cell ignorance by the immune system. With the description of well-characterized tumor antigens, multiple strategies have been proposed mainly aimed at optimal tumor antigen presentation by antigen-presenting cells (APC). Among APC, the dendritic cells have been recognized as the most powerful cells in this class, and have become the target for introducing tumor antigen genes to initiate antitumor immune responses. The detailed knowledge of how the immune system can be activated to specifically recognize tumor antigens, and the mechanisms involved in the control of this immune response, provide the basis for modern genetic immunization strategies for cancer treatment (170).

T lymphocytes play a crucial role in the host's immune response to cancer. Although there is ample evidence for the presence of tumor-associated antigens on a variety of tumors, they are seemingly unable to elicit an adequate antitumor immune response. Modern cancer immunotherapies are therefore designed to induce or enhance T cell reactivity against tumor antigens. Vaccines consisting of tumor cells transduced with cytokine genes in order to enhance their immunogenicity have been intensely investigated in the past decade and are currently being tested in clinical trials. With the development of novel gene transfer technologies it has now become possible to transfer cytokine genes directly into tumors in vivo. The identification of genes encoding tumor-associated antigens and their peptide products which are recognized by cytotoxic T lymphocytes in the context of major histocompatibility complex class I molecules has allowed development of DNA-based vaccines against defined tumor antigens. Recombinant viral vectors expressing model tumor antigens have shown promising results in experimental models. This has led to clinical trials with replication-defective adenoviruses encoding melanoma-associated antigens for the treatment of patients with melanoma. An attractive alternative concept is the use of plasmid DNA, which can elicit both humoral and cellular immune responses following injection into muscle or skin. New insights into the molecular biology of antigen processing and presentation have revealed the importance of dendritic cells for the induction of primary antigen-specific T cell responses. Considerable clinical interest has arisen to employ dendritic cells as a vehicle to induce tumor antigen-specific immunity. Advances in culture techniques have allowed the generation of large numbers of immunostimulatory dendritic cells in vitro from precursor populations derived from blood or bone marrow. Experimental immunotherapies which now transfer genes encoding tumor-associated antigens or cytokines directly into professional antigen-presenting cells such as dendritic cells are under evaluation in pre-clinical studies at many centers. Gene therapy strategies, such as in vivo cytokine gene transfer directly into tumors as well as the introduction of genes encoding tumor-associated antigens into antigen-presenting cells hold considerable promise for the treatment of patients with cancer (171).

### Adjuvant Immunotherapy

In the course of a century, tumor immunology has revealed a picture of a very complex immune system involving the recognition and eradication of malignancies. Many tumors evade the immune system, and understanding of tumor escape mechanisms is the key to a successful immunotherapy for cancer. A wide array of tumor immunotherapy modalities have been developed, many of which have reached the phase of clinical trials, with some satisfactory results (172).

Although surgery remains the mainstay for the treatment of most solid tumors, investigators are seeking complementary therapies to eradicate microscopic disease, which causes tumor relapse even after an apparently complete surgical excision. Although adjuvant chemotherapy has achieved some significant results, the control of minimal residual disease is still a challenge for clinicians. Among novel therapeutic approaches, immunotherapy holds promise. This anticancer strategy aims at triggering a highly specific endogenous killing machine against tumor cells. Recent progress in tumor immunology has improved our understanding of host-immune system interactions. In particular, new technologies have fostered the identification of potentially immunogenic tumor antigens that can be used as suitable targets for immune effector cells. After observing immunotherapy-mediated clinical responses in patients with metastatic disease, investigators have started evaluating this anticancer modality in the adjuvant setting (173).

### Conclusion 1

Urotherapy is suggested as a kind of immunotherapy for cancer patients. Unlike the clonal immunotherapy the urine of the cancer patients contain the many tumor antigens which constitute the tumor. Oral auto-urotherapy will provide the intestinal lymphatic system the tumor antigens against which they may produce antibodies due to non-self-recognition. These antibodies may be transpierced through the blood stream and attack the tumor and its cells (174).

Intralipid can increase the response to the cancer antigens in the intestinal lymphatic system against which antibodies may be produced.

### Intralipid Effects on the Immune System

Intravenous lipid emulsions have been used experimentally since at least the 19th century. An early product marketed in 1957 under the name Lipomul was briefly used in the United States but was subsequently withdrawn due to side effects. Intralipid was invented by the Swedish physician and nutrition researcher Arvid Wretlind (175), and was approved for clinical use in Sweden in 1962. In the United States, the Food and Drug Administration initially declined to approve the product due to prior experience with another fat emulsion. It was approved in the United States in 1972.

Recurrent embryo implantation failure (RIF) is a disorder with potentially devastating physiological and psychological manifestations for those affected. Although its prevalence is not uncommon, many of the mechanisms involved still require elucidation. Both organ-specific and systemic autoimmunity are associated with an increased prevalence of recurrent miscarriage and reproductive failure, rendering the role of the maternal immunological system in fertility a key concept. It is believed by some that central to this theme is the maternal cytokine profile, with particularly T-helper (Th) cells. Immune modulating therapies have therefore been mooted as potential therapeutic strategies. Recent reports of high pregnancy rates achievable in women with RIF have added fuel to the debate regarding the effectiveness of intralipid in modulating the immune system. We would like to assess if there is sufficient current evidence of acceptable quality to permit an assumption that intralipid therapy is an effective treatment for women undergoing repeated assisted reproduction cycles. We have concluded that appropriately controlled, large-scale, confirmatory studies are necessary to prove the efficacy of intralipid before it can be recommended for routine use (176).

In vitro investigations have revealed the ability of intralipids to suppress natural killer (NK) cytotoxicity. Evidence from both animal and human studies suggests that intralipid administered intravenously may enhance implantation and maintenance of pregnancy when the patient has an abnormal NK cell level or function.

The aim of this study was to establish the duration and efficacy of Intralipids suppressive effect on NK cell functional activity.

Fifty patients with abnormal NK activity results (NKa) received intralipid 20% i.v. (9 mg/mL total blood volume—corresponds to 2 mL of intralipid 20% diluted in 250 mL saline; or 18 mg/mL—corresponds to 4 mL of intralipid 20% diluted in 250 mL saline) infusions and their NKa were tested periodically. The determination of NK cell function was performed by flow cytometry using K562 cells as targets.

Fifty women with abnormal NKa-testing received intralipid infusions. 39 (78%) showed NKa suppression within the normal range the first week after infusion, 11 (22%), showed suppression, but still above the normal threshold. They received second infusion 2-3 weeks later. In 10, the Nka activity was normalized the following week. Four patients had three intralipid infusions in 2-week periods in between and after the third infusion, and all showed NKa normal activity. In 47 patients the suppressive effect of the Intralipid after the normalization of NKa lasted between 6 and 9 weeks, in two patients this benefit lasted 5 weeks, and in one patient the effect was 4 weeks.

Intralipid is effective in suppressing in vivo abnormal NK-cell functional activity. The results suggest that Intralipid can be used successfully as a therapeutic option to modulate abnormal NK activity in women with reproductive failure (177).

Novel anti-inflammatory effects of insulin have recently been described, and insulin therapy to maintain euglycemia suppresses the plasma levels of free fatty acids (FFA) and increases the survival of critically ill patients. We aimed to explore the effect of short-term high levels of plasma FFA on the inflammatory response to a low dose of endotoxin. Fourteen healthy male volunteers underwent the following two trials in a randomized crossover design: 1) continuous infusion of 20% Intralipid [0.7 ml·kg⁻¹·h⁻¹ (1.54 g/kg)] for 11 h, and 2) infusion of isotonic saline for 11 h (control). In each trial, heparin was given to activate lipoprotein lipase, and an intravenous bolus of endotoxin (0.1 ng/kg) was given after 6 h of Intralipid/saline infusion. Blood samples and muscle and fat biopsies were obtained before the Intralipid/saline infusion and before as well as after infusion of an endotoxin bolus. Plasma levels of FFA, triglycerides, and glycerol were markedly increased during the Intralipid infusion. Endotoxin exposure induced an increase in plasma levels of TNF-alpha, IL-6, and neutrophils and further stimulated gene expression of TNF-alpha and IL-6 in both skeletal muscle and adipose tissue. The systemic inflammatory response to endotoxin was significantly pronounced during Intralipid infusion. Short-term hyperlipidemia enhances the inflammatory response to endotoxin, and skeletal muscle and adipose tissue are capable of producing essential inflammatory mediators after endotoxin stimulation (178).

Because of its oxidative modification during the acute-phase response to an aggression, low density lipoprotein (LDL) can be regarded as a source of lipid mediators that can act both to promote and inhibit inflammation. This can be exemplified by the production of anti-inflammatory oxidized fatty acids and proinflammatory lysophosphatidylcholine (LPC) during LDL oxidation. We have shown previously that oxidized LDL (oxLDL) plays an active role at the interface between innate and adaptive immunity by delivering instructive molecules such as LPC, which promotes mature dendritic cell (DC) generation from differentiating monocytes. It is shown in this study that LPC affects the signaling pathway of peroxisome proliferator-activated receptors (PPARs). LPC-induced DC maturation is associated with complete inhibition of PPARgamma activity and up-regulation of the activity of an uncharacterized nuclear receptor that bind peroxisome proliferator response element. Oxidized fatty acids generated during LDL oxidation are natural ligands for PPARgamma and inhibit oxLDL- and LPC-induced maturation. Inhibition experiments with synthetic PPARgamma ligands suggested a PPARgamma-dependent and independent effect of LPC on DC maturation. Therefore, the relative amount of oxidized fatty acids and LPC influences the immunological functions of oxLDL on DC, in part by regulating the PPAR pathway. By sensing the biochemical composition of lipoprotein particles, the innate immune system may thus identify various endogenous signals that influence the immune response during the acute-phase reaction. The therapeutic emulsion intralipid also blocks LPC action on PPAR activity and DC maturation. Intralipid may thus be an alternative therapeutic strategy for some chronic inflammatory diseases (179).

During the acute phase response, the interplay between high density lipoproteins and low density lipoproteins (LDL) favors transient generation of oxidized LDL with proinflammatory activities. We hypothesized that oxidative modification of LDL is an endogenous signal for the immune system, and we have shown that oxidized LDL promotes mature dendritic cell transition from monocyte, therefore linking the nonspecific acute phase response to adaptive immunity. Lysophosphatidylcholine (LPC) is a major lipid component of oxidized LDL with reported proinflammatory activities. We now report that LPC acts through G protein-coupled receptors on differentiating monocytes to generate mature dendritic cells with the ability to stimulate IL-2 and IFN-gamma production by allogeneic T lymphocytes. LPC is most effective in lipoprotein-deprived serum and can be inhibited by an excess of native LDLs reflecting normal plasma conditions. Therefore, by controlling the balance between native and oxidized lipoproteins and the resulting production of LPC, the acute phase reactants may provide a context of Ag presentation that is transiently favorable to immune activation. Intralipid, a therapeutic lipid emulsion for parenteral nutrition with unexplained immunomodulatory properties, also blocked LPC activity. This opens perspectives for the understanding and treatment of acute and chronic inflammatory diseases (180).

The lipid component of total parenteral nutrition (TPN) has reportedly been associated with trophic effects on the intestinal mucosa and suppressive effects on the immune system.

We have challenged these hypotheses using a 7-day TPN rodent model comparing the effects of isocaloric, isonitrogenous lipid-based (TPN-lipid, 50% of calories as long-chain triacylglycerol) and carbohydrate-based TPN (TPN-CH, 100% of calories as carbohydrates) on mucosal morphology and immune function. Enterally fed animals were included to establish a baseline for immunologic read-outs. The study was performed in healthy, metabolically stable animals to avoid interference by septic or trauma-related stress factors.

Both TPN regimens resulted in a significantly smaller weight gain (TPN-lipid, 29.8+/−4.0 g; TPN-CH, 30.3+/−4.4 g) compared with enterally fed reference animals (49.2+/−3.2 g; p=0.007), with no difference in nitrogen balance between the TPN groups. Mucosal sucrase activity was significantly lower in both TPN groups (TPN-lipid, 8.8+/−1.0×10(−7) katal per gram (kat/g) of protein; CH: 11.9+/−1.6×10(−7) kat/g of protein) compared with enteral feeding (17.4+/−0.9×10(−7) kat/g of protein; ANOVA: p=0.0007). Morphometric analysis of the small intestine revealed no differences between the two TPN groups although a significantly depressed villus height in the TPN-lipid group could be observed in comparison to enterally fed reference rats (TPN-lipid, 0.47+/−0.02; TPN-CH, 0.50+/−0.01; enteral, 0.56+/−0.02 mm; ANOVA: p=0.0298). Light and electron microscopy revealed a normal surface architecture in all three groups of rats. Cellular immune reactivity was evaluated using a novel specific immunization protocol: animals were immunized against OVA 4 weeks before TPN. OVA-induced lymphoproliferative responses and phenotypic data from draining popliteal and mesenteric lymph nodes were evaluated after the different regimens. Results did not differ among the three groups.

In healthy rodents, short-term lipid-based and carbohydrate-based TPN regimens lead to limited mucosal atrophy with preserved surface architecture compared with enteral feeding. However, peripheral and mesenteric cellular immune responsiveness after both TPN regimens remained comparable to enterally fed reference animals. Therefore, mesenteric and systemic cellular immune reactivity does not appear to be impaired by lipid-based or carbohydrate-based TPN (181).

To detect possible interactions between lipid-based total parenteral nutrition (TPN) substrates and mononuclear phagocytes, ultrastructural in vitro and in vivo studies were carried out on material from pigs. Mononuclear phagocytes isolated from peripheral blood, phagocytosed lipid after incubation with 1 mg/ml Intralipid for 24 h. Similarly, lipid was taken up by intravascular macrophages in the lungs and liver after central venous administration of TPN containing 2.3 g/kg body weight/day of Intralipid for 5-7 weeks. Lipid accumulation was almost exclusively found intravascularly in the lungs and liver, and not in macrophages obtained from bronchoalveolar lavage fluid. A morphometric study of the lung capillaries showed that the macrophages in TPN animals had increased in size and number, and occupied a larger portion of the capillary lumina. The macrophages appeared to be activated, but the endothelial lining was well preserved. Free intravascular lipid droplets had a diameter both in vitro and in vivo of about 0.5 micron, indicating good stability of the emulsion. We suggest that the lipid uptake stimulates the macrophages and thereby plays a role in the lung tissue inflammation seen in response to long-term lipid-based TPN in pigs (182).

Intravenous lipid emulsions depress lymphocyte proliferative responses and granulocyte function at concentrations found in the blood circulation during their administration. The effects of Intralipid, a widely used intravenous lipid emulsion, were measured on immunoglobulin production in vitro by pokeweed mitogen-activated lymphocytes as a test of B-cell function. Intralipid decreased IgG, IgM, and IgA production at soybean oil triglyceride concentrations of 2.5-20 mg/ml occurring in the blood circulation during Intralipid infusion. The effects on IgM and IgA production were highest and that on IgG production lowest. Hydrocortisone-sensitive and concanavalin A-inducible suppressor cells were more sensitive to Intralipid than other cell populations. In vivo Ig production may not be equally disturbed, inasmuch as Intralipid concentrations in the lymph nodes and the spleen may be lower than in the blood circulation. However, care should be taken to prevent Intralipid concentrations from becoming high enough to depress immune responses (183).

We studied the effect of Intralipid (IL) in monocyte cultures based on the ability of the cultures to phagocytose and kill Candida albicans and produce the oxidative burst. The IL was taken up by monocytes in cultures, and these cells phagocytosed more Candida organisms than did the control cells [85+/−2.2% in the IL treated (1%) compared to 68+/−2.3% after 1 h in the control]. The percentage of killing of Candida albicans, which had been taken up by the IL-treated monocytes measured after 2 h in culture (48.3+/−6.0%), was no different when compared to control (47.0+/−5.8%). Following ingestion of IL, there was an increase in basal H2O2 production, however, the presence of the IL in the cells had no effect on the expected increase in H2O2 production following stimulation with either phorbol myristate acetate (PMA) or zymosan particles. Compared to untreated cells, a significant increase in the number of monocytes with positive nitroblue tetrazolium staining was observed in monocytes that had ingested IL (when they were stimulated with either PMA or Candida microorganisms). Similar results were obtained in monocyte-derived macrophages (i.e., monocytes in monolayer cultures for 10 days). These findings suggest that the essential monocyte functions of phagocytosis, microbicidal activity, and ability to elicit an oxidative burst are not directly altered by the conventional use of IL in clinical practice (184).

In many surgical departments it has been common practice to give patients with weight loss pre-operative parenteral nutrition before major surgery. The purpose of the present study was to elucidate the value of intravenous pre-operative nutrition in relation to the immune system. The study comprised 10 patients undergoing total gastrectomy. All patients had a weight loss of 15% of body weight or more within 6 months or 10% within 3 months. Before operation they all received parenteral nutrition for 1 week. They all had 1.5 g of protein per kg per day and energy corresponding to the basal metabolic rate+50% as Vamin, Intralipid, and carbohydrate solutions. Before and after this treatment blood samples were taken to estimate neutrophil function (the rate of oxygen consumption and superoxide liberation, phagocytosis and intracellular lysis of Candida albicans, the concentration and consumption rate of ATP during phagocytosis, and chemotaxis) and immune globulins (IgG, IgM, & IgA). Cellular immunity (CMI) was estimated by intradermal application of seven different antigens. We found a significant increase in response to the intradermal antigens (p<0.01) but no difference in any of the parameters expressing leukocyte function or immune globulins (185).

Eight healthy subjects were given Intralipid, a soybean oil emulsion, 20% intravenously for 2 h. During the infusion a significant increase in the nitroblue tetrazolium-reduction of blood monocytes was noted. Preincubation of monocytes in vitro with Intralipid (20 to 100 mg/ml) for 30 min was found to increase the ability of the cells to migrate chemotactically and to phagocytize yeast particles. On the contrary, when neutrophilic granulocytes were preincubated with Intralipid in the same concentrations for 30 min. their nitroblue-tetrazolium-reduction, chemotactic and spontaneous locomotion, as well as their ingestion of yeast particles was depressed (186).

Platinum (Pt) drugs are the most potent and commonly used anti-cancer chemotherapeutics. Nanoformulation of Pt drugs has the potential to improve the delivery to tumors and reduce toxic side effects. A major challenge for translating nanodrugs to clinical settings is their rapid clearance by the reticuloendothelial system (RES), hence increasing toxicities on off-target organs and reducing efficacy. We are reporting that an FDA approved parenteral nutrition source, Intralipid 20%, can help this problem. A dichloro (1, 2-diaminocyclohexane) platinum (II)-loaded and hyaluronic acid polymer-coated nanoparticle (DACHPt/HANP) is used in this study. A single dose of Intralipid (2 g/kg, clinical dosage) is administrated [intravenously (i. v.), clinical route] one hour before i.v. injection of DACHPt/HANP. This treatment can significantly reduce the toxicities of DACHPt/HANP in liver, spleen, and, interestingly, kidney. Intralipid can decrease Pt accumulation in the liver, spleen, and kidney by 20.4%, 42.5%, and 31.2% at 24-hr post nanodrug administration, respectively. The bioavailability of DACHPt/HANP increases by 18.7% and 9.4% during the first 5 and 24 hr, respectively (187).

### Conclusion 2

The use of oral intralipid together with auto urotherapy in patients with cancer is first suggested in the medical literature. Intralipid can increase the response to the cancer antigens in the intestinal lymphatic system against which antibodies may be produced. These antibodies may be transpierced through the blood stream and attack the tumor and its cells.

### REFERENCES

1. Root-Bernstein R S. Causality, complementarity, evolution, and emergent properties. In Iversen O H (ed.): New Frontiers in Cancer Causation. Taylor & Francis 1993, p. 1-14.
2. Burnet F M. The concept of immunological surveillance. Prog Exp Tumor Res 1970; 13:1-27.
3. Hellstrom I et al., Cell-mediated reactivity to antigens shared by Moloney virus induced lymphoma cells (LSTRA) and cells from certain 3-methylcholanthrene induced mouse sarcomas. Int J Cancer 1979; 23:555-64.
4. Hellstrom I et al., Cell-mediated immunity against antigens common to tumor colonic carcinomas and fetal gut epithelium. Int J Cancer 1970; 6:346-51.
5. Hellstrom K E, Hellstrom I. Immunological approach to tumor therapy: Monoclonal antibodies, tumor vaccines, and anti-idiotypes. In Covalently Modified Antigens and Antibodies in Diagnosis and Therapy. Vol. 2. Edited by Quash G A, Rodwell J D. Marcel Dekker, Inc., New York 1989, p. 1.
6. Ehrlich P. In Himmelweit S., ed. The Collected Papers of Paul Ehrlich (translated by P. Alexander). Pergamon Press, Oxford, 1957.
7. Watanabe T et al., Human melanoma antigen AH is an autologous ganglioside related to GD2. J Exp Med 1982; 156:1884-9.
8. Rote N S et al., Tumor-associated antigens detected by autologous sera in urine of patients with solid neoplasms. J Surg Res 1980; 29:18-22.
9. Gupta R K et al., Prognostic significance of urinary antigen analysis by enzyme-linked immunosorbant assay in melanoma patients. Diag Immunol 1983; 1:303-309.
10. DeVere White R W et al., Urinary prostate specific antigen levels: Role in monitoring the response of prostate cancer to therapy. J Urol 1992; 147:947-951.
11. Huland E et al., Comparison of 15 monoclonal antibodies against tumor-associated antigens of transitional cell carcinoma of the human bladder. J Urol 1991; 146:1631-6.
12. Farinati F et al., Gastric juice CEA levels: importance of age and gastric mucosal damage. Europ J Cancer Clin Oncol 1986; 22:527-9.
13. Touitou Y et al., Cumulative effects of age and pathology on plasma carcinoembryonic antigen in an unselected elderly population. Europ J Cancer Clin Oncol 1984; 20:369-374.
14. Lindholm L et al., Monoclonal antibodies against gastro-intestinal tumor-associated antigens isolated as monosialogangliosides. Int Arch Allergy appl Immun 1983; 71:178-181.
15. Mansson J E et al., Chemical structure of carcinoma ganglioside antigens defined by monoclonal antibody CA50 and some allied gangliosides of human pancreatic adenocarcinoma. Biochim Biophys Acta 1985; 834:110-117.
16. Holmgren J et al., Detection by monoclonal antibody of carbohydrate antigen CA50 in serum of patients with carcinoma. Brit Med J 1984; 288:1479-1482.
17. Dienst C et al., CA 19-9, CA50 und CEA bei Pankreas und gastrointestinal Tumoren. Medizin Klin 1987; 82:45-50.
18. Bruhn H D et al., CA 50 im serum von Karzinom-Patienten. Deutsch Med Wochensch 1986; 34:1267-1272.
19. Farinati F et al., CA 50 determination in body fluids: Can we screen patients at risk for gastric cancer? Int J Cancer 1991; 47:7-11.
20. Hoffmann J C et al., A soluble form of the adhesion receptor CD58 (LFA-3) is present in human body fluids. Eur J Immunol 1993; 23:3003-3010.
21. Tobi M et al., Urinary organ specific neoantigen. A potentially diagnostic test for colorectal cancer. Dig Dis Sci 1995; 40:1531-7.
22. Boutwell R K. Evidence that an elevated level of ornithine decarboxylase activity is an essential component of tumor promotion. Adv Polyamine Res 1983; 4:127-133.
23. Lipton A et al., Urinary polyamine levels in patients with gastrointestinal malignancy. Cancer 1975; 36:2351-4.
24. Loser C et al., Polyamines in colorectal cancer. Evaluation of polyamine concentrations in the colon tissue, serum and urine of 50 patients with colorectal cancer. Cancer 1990; 65:958-966.
25. Matsumura Y, Tarin D. Significance of CD44 gene products for cancer diagnosis and disease evaluation. Lancet 1992; 340:1053-8
26. Matsumura Y et al., Non-invasive detection of malignancy by identification of unusual CD44 gene activity in exfoliated cancer cells. Br Med J 1994; 308:619-624.
27. Matsumura Y et al., Unusual retention of introns in CD44 gene transcripts in bladder cancer provides new diagnostic and clinical oncological opportunities. J Pathol 1995; 177:11-20.
28. Zbar B, Tanaka T. Immunotherapy of cancer: regression of tumors after intralesional injection of living Mycobacterium bovis. Science 1971; 172:271-273.
29. Rosenberg S A et al., Use of tumor-infiltrating lymphocytes and interleukin-2 in the immunotherapy of patients with metastatic melanoma. A preliminary report. N Engl J Med 1988; 319:1676-80.
30. Hellstrom K E, Hellstrom I. Cellular immunity against tumor specific antigens. Adv Cancer Res 1969; 12:167-223.
31. Penn I. Tumors of the immunocompromised patient. Ann Rev Med 1988; 39:63-73.
32. Vanky F et al., Lysis of autologous tumor cells by blood lymphocytes activated in autologous mixed lymphocyte tumor cell culture—no correlation with the postsurgical clinical course. Cancer Immunol Immunother 1987; 24:180.
33. Vanky F, Klein E. Specificity of auto-tumor cytotoxicity exerted by fresh, activated and propagated human T lymphocytes. Int J Cancer 1982; 29:547.
34. Knuth A et al., Cytolytic T cell clones against an autologous human melanoma: Specificity study and definition of three antigens by immunoselection. Proc Natl Acad Sci (USA) 1989; 86:2804-8.
35. LoBuglio A F, Neidhart J A. A review of transfer factor immunotherapy in cancer. Cancer 1974; 34:1563-70
36. Levin A S et al., Osteogenic sarcoma. Immunologic parameters before and during immunotherapy with tumor-specific transfer factor. J Clin Invest 1975; 55:487-99.
37. Rosenberg S A et al., Combination immunotherapy for cancer: synergistic antitumor interactions of interleukin-2, alpha interferon and tumor-infiltrating lymphocytes. JNCI 1988; 80:1393-1397.
38. Rosenberg S A et al., Gene transfer into humans: immunotherapy of melanoma using tumor-infiltrating lymphocytes modified by retroviral gene transduction. N Engl J Med 1990; 323:570-578.
39. Russell S J et al., Decreased tumorigenicity of a transplantable rat sarcoma following transfer and expression of an IL-2 cDNA. Int J Cancer 1991; 47:244-252.
40. Hellstrom K E et al., Regression and inhibition of sarcoma growth by interference with a radiosensitive T cell population. J Exp Med 1978; 148:799-804.
41. Estin C D et al., Cyclophosphamide potentiates the antitumor activity of v-p97NY. Cell Immunol 1989; 120:126-31.
42. Nepom G T, Hellstrom K E. Anti-idiotypic antibodies and the induction of specific tumor immunity. Cancer Metast Rev 1987; 6:489-502.
43. Schwartz R H. A cell culture model for T lymphocyte clonal anergy. Science 1990; 248:1349-1356.
44. Jenkins M K, Schwartz R H. Antigen presentation by chemically modified splenocytes induces antigen-specific T cell unresponsiveness in vitro and in vivo. J Exp Med 1987; 165:302-319.
45. Mowat A M. The regulation of immune responses to dietary protein antigens. Immunol Today 1987; 8:93-98.
46. Weiner H L et al., Antigen-driven peripheral immune tolerance. Suppression of organ-specific autoimmune diseases by oral administration of autoantigens. Ann NY Acad Sci 1991; 636:227-232.
47. Sercarz E, Krzych U. The distinctive specificity of antigen-specific suppressor T cells. Immunol Today 1991; 12:111-118.
48. Green D R et al., Immunoregulatory T-cell pathways. Annu Rev Immunol 1983; 1:439-463.
49. Thurau S R et al., Immunological suppression of experimental autoimmune uveitis. Fortschr Ophthalmol 1991; 88:404-407.
50. Nussenblatt R B et al., Inhibition of S-antigen induced experimental autoimmune uveoretinitis by oral induction of tolerance with S-antigen. J immunol 1990; 144:1689-51. Brandtzaeg P. Overview of the mucosal immune system. Curr top Microbiol Immunol 1989; 146:13-28.
52. MesteckyJ, McGhee J R. Oral immunization: Past and present. Curr Top Microbiol Immunol 1989; 146:3-12.
53. Stokes C R. Induction and control of intestinal immune responses. In Newby T J, Stokes C R (eds.): Local immune responses of the gut. Boca Raton, CRC Press, 1984, pp. 97-142.
54. Hanson D G et al., Inhibition of specific immune responses by feeding protein antigens. II. Effects of prior passive and active immunization. J Immunol 1979; 122:2261-2266.
55. Brandtzaeg P et al., Nature and properties of the human gastrointestinal immune system. In Miller K, Nicklin S (eds.): Immunology of the Gastrointestinal Tract. Boca Raton, CRC Press, 1987, pp. 1-88.
56. Peng H J, Turner M W, Strobel S. The generation of a “tolerogen” after the ingestion of ovalbumin is time-dependent and unrelated to serum levels of immunoreactive antigen. Clin Exp Immunol 1990; 81:510-515.
57. Stokes C R et al., Genetic differences in immune exclusion and partial tolerance to ingested antigens. Clin Exp Immunol 1983; 52:678-684.
58. Swarbrick E T et al., Absorption of antigens after oral immunization and the simultaneous induction of specific systemic tolerance. Gut 1979; 20:121-125.
59. Miller A et al., Active suppression vs. clonal anergy following oral or IV administration of MBP in actively and passively induced EAE. Neurology 1992; 42(Suppl 3):301.
60. Lamont A G et al., Oral tolerance in protein deprived mice. II. Evidence of normal ‘gut processing’ of ovalbumin, but suppressor cell deficiency, in deprived mice. Immunology 1987; 61:339-343.
61. Hanson D G et al., Inhibition of specific immune responses by feeding protein antigens. Int Arch Allergy Appl Immunol 1977; 55:526-532.
62. Matthews J B et al., Serum and salivary antibody responses and the development of oral tolerance after oral and intragastric antigen administration. Int Arch Allergy Appl Immunol 1981; 65:107-113.
63. Higgins P, Weiner H L. Suppression of experimental autoimmune encephalomyelitis by oral administration of myelin basic protein and its fragments. J Immunol 1988; 140:440-445.
64. Miller A et al., Suppression of experimental autoimmune encephalomyelitis by oral administration of myelin basic protein. J Neuroimmunol 1992; 39:243-250.
65. Jausion H. Sur l'auto-ouro-therapie. Journal D'Urologie 1935; 39:58-59.
66. Cimino T. Premiers essais de vaccine-proteine-therapie des infections non gonococciques ni tuberculeuses des voies urinaires a l'aide des injections sous-cutanees de Purine purulente du sujet, sterilisee par l'ebullition (ouro-therapie). Rivista Sanitaria 1927; 186.
67. Rabinowitch I M. Auto-urine-therapy in gonarthritis. Vratchebnaia gazeta 1931; 35:677-8.
68. Jausion H et al., L'auto-ouro-therapie. La Presse Medicale 1933; 76:1467-1470.
69. Day H B. Treatment of glomerulonephritis by antigen. Lancet 1936; 1456-9.
70. Sandweiss D J et al., The prevention or healing of experimental ulcer in Mann-Williamson dogs with the Anterior-Pituitary-Like hormone (Antuitrin-S). Am J Dig Dis 1938; 5:24-30.
71. Sandweiss D J et al., The relation of sex hormones to peptic ulcer. Am J Dig Dis 1939; 6:6-12.
72. Seiffert L. Die “Darn-Siphonblase”. Arch fur Klin Chir 1935; 183:569.
73. Bricker E M. Bladder substitution after pelvic evisceration. Surg Clin North Am 1950; 30:1511.
74. Hammer E. Cancer du colon sigmoide dix ans apres implantation des ureteres dune vessie exstrophiee. J Urol Nephrol 1929; 28:260.
75. Miller-Schoop J W, Good R A. Functional studies of Peyer's patches: Evidence for their participation in intestinal immune responses. J Immunol 1975; 144:1757.
76. Barclay T H C, Schapira D V. Malignant tumors of the small bowel. Cancer 1983; 51:878-881.
77. Loeffler M et al., Intestinal cell proliferation. I. A comprehensive model of steady-state proliferation in the crypt. Cell Tissue Kinet 1986; 19:627-645.
78. Tapper D, Folkman J. Lymphoid depletion in ileal loops: Mechanism and clinical implications. J Pediatr Surg 1976; 11:871-880.
79. Wilson W E C et al., Suppression of immunologic responsiveness in uremia. Ann Intern Med 1965; 62:1.
80. Starkey R H, Cohen S, Orth D N. Epidural growth factor: Identification of a new hormone in human urine. Science 1975; 189:800-802.
81. Urdaneta L F et al., Late development of primary carcinoma of the colon following ureterosigmoidostomy: report of three cases and literature review. Ann Surg 1966; 164:503-13.
82. Harguindey S S et al., Ureterosigmoidostomy and cancer: new observations (letter). Ann Intern Med 1975; 83:833.
83. Rivard J Y et al., Colonic neoplasms following ureterosigmoidostomy. J Urol 1975; 113:781-6.
84. Carswell J J III et al., Neoplasia at the site of ureterosigmoidostomy. J Urol 1976; 115:750-2.
85. Lasser A, Acosta A E. Colonic neoplasms complicating ureterosigmoidostomy. Cancer 1975; 35:1218-22.
86. S ooriyaarachchi G S et al., Neoplasms of the large bowel following ureterosigmoidostomy. Arch Surg 1977; 112:1174-7.
87. Eraklis A J, Folkman M J. Adenocarcinoma at the site of ureterosigmoidostomies for exstrophy of the bladder. J Pediatr Surg 1978; 13:730-4.
88. Everson T. Spontaneous regression of cancer. Ann NY Acad Sci 1964; 114:721-35.
89. Stephenson H et al., Host immunity and spontaneous regression of cancer evaluated by computerized data reduction study. Surg Gynecol Obstet 1971; 133:649-55.
90. Cole W. Spontaneous regression of cancer: The metabolic triumph of the host? Ann NY Acad Sci 1974; 230:111-41.
91. Burnet F. Immunological aspects of malignant disease. Lancet 1967; 11:1171-4.
92. Droller M. Immunotherapy and genitourinary neoplasia. Urol Clin N Am 1980; 7:831-46.
93. Enomoto K et al., Thrombopoiesis and megakaryocyte colony stimulating factor in the urine of patients with aplastic anemia. Br J Haematol 1980; 45:551-556.
94. Kawakita M et al., Thrombopoiesis and megakaryocyte colony stimulating factors in the urine of patients with idiopathic thrombocytopenic purpura. Br J Haematol 1981; 48:609-615
95. Stanley E R et al., Antibody production to the factor in human urine stimulating colony formation in vitro by bone marrow cells. Br J Haematol 1970; 18:585-590.
96. Galdston I. The ideological basis of discovery. Bull Hist Med 1939; 7:729-735.
97. Shu S et al., Tumor immunology. JAMA. 1997 Dec. 10; 278(22):1972-81.
98. Cerundolo V. T cells work together to fight cancer. Curr Biol. 1999 Sep. 23; 9(18):R695-7.
99. Kawakami Y. New cancer therapy by immunomanipulation: development of immunotherapy for human melanoma as a model system. Cornea. 2000 May; 19(3 Sunni): S2-6.
100. Houghton A N et al., Immunity against cancer: lessons learned from melanoma. Curr Opin Immunol. 2001 April; 13(2):134-40.
101. Lieberman S M et al., Innovative treatments for pancreatic cancer. Surg Clin North Am. 2001 June; 81(3):715-39.
102. Turk M J, Wolchok J D, Guevara-Patino J A, Goldberg S M, Houghton A N. Multiple pathways to tumor immunity and concomitant autoimmunity. Immunol Rev. 2002 October; 188:122-35.
103. Koroleva E P et al., Serological study of a repertoire of human cancer antigens and autoantigens. Mol Biol (Mosk). 2004 March-April; 38(2):233-8.
104. Lollini P L, Forni G. Cancer immunoprevention: tracking down persistent tumor antigens. Trends Immunol. 2003 February; 24(2):62-6.
105. Scanlan M J et al., Cancer/testis antigens: an expanding family of targets for cancer immunotherapy. Immunol Rev. 2002 October; 188:22-32.
106. Perez-Diez A, Marincola F M. Immunotherapy against antigenic tumors: a game with a lot of players. Cell Mol Life Sci. 2002 February; 59(2):230-40.
107. van den Eynde B. Identification of cancer antigens of relevance for specific cancer immunotherapy. Bull Mem Acad R Med Belg. 2001; 156(10-12):548-55.
108. Itoh K et al., Human tumor-rejection antigens and peptides from genes to clinical research. Nippon Geka Gakkai Zasshi. 2000 September; 101(9):612-7.
109. Slingluff C L. Targeting unique tumor antigens and modulating the cytokine environment may improve immunotherapy for tumors with immune escape mechanisms. Cancer Immunol Immunother. 1999 October; 48(7):371-3.
110. Wang R F. Human tumor antigens: implications for cancer vaccine development. J Mol Med. 1999 September; 77(9):640-55.
111. Kudo T et al., Specific targeting immunotherapy of cancer with bispecific antibodies. Tohoku J Exp Med. 1999 August; 188(4):275-88.
112. Huang S K et al., Antibody responses to melanoma/melanocyte autoantigens in melanoma patients. J Invest Dermatol. 1998 October; 111(4):662-7.
113. Wang R F. Tumor antigens discovery: perspectives for cancer therapy. Mol Med. 1997 November; 3(11):716-31.
114. Ribas A et al., Current developments in cancer vaccines and cellular immunotherapy. J Clin Oncol. 2003 Jun. 15; 21(12):2415-32.
115. Zoller M. Immunotherapy of cancer by active vaccination: does allogeneic bone marrow transplantation after non-myeloablative conditioning provide a new option? Technol Cancer Res Treat. 2003 June; 2(3):237-60.
116. Chada S et al., Development of vaccines against self-antigens: the p53 paradigm. Curr Opin Drug Discov Devel. 2003 March; 6(2):169-73.
117. Pecher G. DNA-based tumor vaccines. Onkologie. 2002 December; 25(6):528-32.
118. Dermime S et al., Cancer vaccines and immunotherapy. Br Med Bull. 2002; 62:149-62.
119. Perales M A, Wolchok J D. Melanoma vaccines. Cancer Invest. 2002; 20(7-8):1012-26.
120. Cohen E P. DNA-based vaccines for the treatment of cancer—an experimental model. Trends Mol Med. 2001 April; 7(4):175-9.
121. Berd D. Autologous, hapten-modified vaccine as a treatment for human cancers. Vaccine. 2001 Mar. 21; 19(17-19):2565-70.
122. Bhattacharya-Chatterjee M et al., The anti-idiotype vaccines for immunotherapy. Curr Opin Mol Ther. 2001 February; 3(1):63-9.
123. Bhattacharya-Chatterjee M et al., Anti-idiotype vaccine against cancer. Immunol Lett. 2000 Sep. 15; 74(1):51-8.
124. Mitchell D A, Nair S K. RNA transfected dendritic cells as cancer vaccines. Curr Opin Mol Ther. 2000 April; 2(2):176-81.
125. Wang R F, Rosenberg S A. Human tumor antigens for cancer vaccine development. Immunol Rev. 1999 August; 170:85-100.
126. Timmerman J M, Levy R. Dendritic cell vaccines for cancer immunotherapy. Annu Rev Med. 1999; 50:507-29.
127. Chen C H, Wu T C. Experimental vaccine strategies for cancer immunotherapy. J Biomed Sci. 1998 July-August; 5(4):231-52.
128. Gilboa E, Nair S K, Lyerly H K. Immunotherapy of cancer with dendritic-cell-based vaccines. Cancer Immunol Immunother. 1998 April; 46(2):82-7.
129. Dudley M E, Rosenberg S A. Adoptive-cell-transfer therapy for the treatment of patients with cancer. Nat Rev Cancer. 2003 September; 3(9):666-75.
130. Whelan M et al., Cancer immunotherapy: an embarrassment of riches? Drug Discov Today. 2003 Mar. 15; 8(6):253-8.
131. Kwak H et al., Poxviruses as vectors for cancer immunotherapy. Curr Opin Drug Discov Devel. 2003 March; 6(2):161-8.
132. Markiewicz M A, Kast W M. Advances in immunotherapy for prostate cancer. Adv Cancer Res. 2003; 87:159-94.
133. Ward S et al., Immunotherapeutic potential of whole tumour cells. Cancer Immunol Immunother. 2002 September; 51(7):351-7.
134. Sugiyama H. Cancer immunotherapy targeting WT1 protein. Int J Hematol. 2002 August; 76(2): 127-32.
135. Wang R F et al., T cell-mediated immune responses in melanoma: implications for immunotherapy. Crit Rev Oncol Hematol. 2002 July; 43(1):1-11.
136. Fehniger T A et al., Interleukin-2 and interleukin-15: immunotherapy for cancer. Cytokine Growth Factor Rev. 2002 April; 13(2):169-83.
137. Rosenberg S A. Progress in the development of immunotherapy for the treatment of patients with cancer. J Intern Med. 2001 December; 250(6):462-75.
138. McLaughlin P M et al., Cancer immunotherapy: insights from transgenic animal models. Crit Rev Oncol Hematol. 2001 October; 40(1):53-76.
139. Maki R G. Soft tissue sarcoma as a model disease to examine cancer immunotherapy. Curr Opin Oncol. 2001 July; 13(4):270-4.
140. Rosenberg S A. Progress in human tumour immunology and immunotherapy. Nature. 2001 May 17; 411(6835):380-4.
141. Bertolaccini L, Olivero G. Cancer immunotherapy. A future therapeutical choice? Minerva Chir. 2001 April; 56(2):183-91.
142. Smyth M J et al., A fresh look at tumor immunosurveillance and immunotherapy. Nat Immunol. 2001 April; 2(4):293-9.
143. Vinals C et al., Using in silico transcriptomics to search for tumor-associated antigens for immunotherapy. Vaccine. 2001 Mar. 21; 19(17-19):2607-14.
144. Matzku S, Zoller M. Specific immunotherapy of cancer in elderly patients. Drugs Aging. 2001; 18(9):639-64.
145. Rousseau R, Soria J C. Telomerase, a universal target in immunotherapy strategies against tumor? Bull Cancer. 2000 December; 87(12):895-901.
146. Davis I D. An overview of cancer immunotherapy. Immunol Cell Biol. 2000 June; 78(3): 179-95.
147. Bremers A J, Parmiani G. Immunology and immunotherapy of human cancer: present concepts and clinical developments. Crit Rev Oncol Hematol. 2000 April; 34(1):1-25.
148. Shankar G, Salgaller M L. Immune monitoring of cancer patients undergoing experimental immunotherapy. Curr Opin Mol Ther. 2000 February; 2(1):66-73.
149. Knuth A, Jager D, Jager E. Cancer immunotherapy in clinical oncology. Cancer Chemother Pharmacol. 2000; 46 Suppl:S46-51.
150. Pawelec G et al., Prerequisites for the immunotherapy of cancer. Cancer Immunol Immunother. 1999 July; 48(4):214-7.
151. Maxwell-Armstrong C A et al., Immunotherapy for colorectal cancer. Am J Surg. 1999 April; 177(4): 344-8.
152. Lum L G. T cell-based immunotherapy for cancer: a virtual reality? CA Cancer J Clin. 1999 March-April; 49(2):74-100, 65.
153. Rosenberg S A. A new era of cancer immunotherapy: converting theory to performance. CA Cancer J Clin. 1999 March-April; 49(2):70-3, 65.
154. Hersey P. Impediments to successful immunotherapy. Pharmacol Ther. 1999 February; 81(2):111-9.
155. Przepiorka D, Srivastava P K. Heat shock protein—peptide complexes as immunotherapy for human cancer. Mol Med Today. 1998 November; 4(11):478-84.
156. Tamura Y, Peng P, Liu K, Daou M, Srivastava P K. Immunotherapy of tumors with autologous tumor-derived heat shock protein preparations. Science. 1997 Oct. 3; 278(5335): 117-20.
157. Catros-Quemener V, Bouet F, Genetet N. Antitumor immunity and cellular cancer therapies. Med Sci (Paris). 2003 January; 19(1):43-53.
158. Ladhams A et al., Treatment of non-resectable hepatocellular carcinoma with autologous tumor-pulsed dendritic cells. J Gastroenterol Hepatol. 2002 August; 17(8):889-96.
159. Meidenbauer N et al., Dendritic cells for specific cancer immunotherapy. Biol Chem. 2001 April; 382(4):507-20.
160. Punt C J et al., Immunology in medical practice. XXV. Use of dendritic cells in the immunotherapy of cancer. Ned Tijdschr Geneeskd. 1999 Nov. 27; 143(48):2408-14.
161. Hermans I F et al., The emerging role of the dendritic cell in novel cancer therapies. N Z Med J. 1998 Apr. 10; 111(1063):111-3.
162. Trikha M, Yan L, Nakada M T. Monoclonal antibodies as therapeutics in oncology. Curr Opin Biotechnol. 2002 December; 13(6):609-14.
163. Withoff S et al., Bi-specific antibody therapy for the treatment of cancer. Curr Opin Mol Ther. 2001 February; 3(1):53-62.
164. Weiner L M, Adams G P. New approaches to antibody therapy. Oncogene. 2000 Dec. 11; 19(53):6144-51.
165. Abicht A, Lochmuller H. Technology evaluation: edrecolomab, Centocor Inc. Curr Opin Mol Ther. 2000 October; 2(5):593-600.
166. Brezicka T et al., Reactivity of monoclonal antibodies with ganglioside antigens in human small cell lung cancer tissues. Lung Cancer. 2000 April; 28(1):29-36.
167. Livingston P O et al., Autoimmune and antitumor consequences of antibodies against antigens shared by normal and malignant tissues. J Clin Immunol. 2000 March; 20(2):85-93.
168. Velders M P et al., Active immunization against cancer cells: impediments and advances. Semin Oncol. 1998 December; 25(6):697-706.
169. Yoshizawa H et al., Cancer immunogene therapy. Arch Immunol Ther Exp (Warsz). 2001; 49(5):337-43.
170. Ribas A et al., Genetic immunotherapy for cancer. Oncologist. 2000; 5(2):87-98.
171. Tuting T, Storkus W J, Lotze M T. Gene-based strategies for the immunotherapy of cancer. J Mol Med. 1997 July; 75(7):478-91.
172. Bremers A J et al., Tumour immunotherapy: the adjuvant treatment of the 21st century? Eur J Surg Oncol. 2000 June; 26(4):418-24.
173. Mocellin S et al., Adjuvant immunotherapy for solid tumors: from promise to clinical application. Cancer Immunol Immunother. 2002 December; 51(11-12):583-95.
174. Eldor J. Urotherapy for patients with cancer. Med Hypotheses. 1997 April; 48(4):309-15.
175. http://pmj.bmj.com/content/43/498/307.full.pdf
176. Shreeve N, Sadek K. Intralipid therapy for recurrent implantation failure: new hope or false dawn? J Reprod Immunol. 2012 January; 93(1):38-40.
177. Roussev R G et al., Duration of intralipid's suppressive effect on NK cell's functional activity. Am J Reprod Immunol. 2008 September; 60(3):258-63.
178. Krogh-Madsen R et al., Effect of short-term intralipid infusion on the immune response during low-dose endotoxemia in humans. Am J Physiol Endocrinol Metab. 2008 February; 294(2):E371-9.
179. Coutant F et al., Sensing environmental lipids by dendritic cell modulates its function. J Immunol. 2004 Jan. 1; 172(1):54-60.
180. Coutant F et al., Mature dendritic cell generation promoted by lysophosphatidylcholine. J Immunol. 2002 Aug. 15; 169(4):1688-95.
181. Gross T et al., Intralipid-based short-term total parenteral nutrition does not impair small intestinal mucosa-related cellular immune reactivity in the healthy rat. JPEN J Parenter Enteral Nutr. 2000 November-December; 24(6):337-44.
182. Aksnes J et al., Intravascular lung macrophages play an essential role in lipid entrapment and the inflammatory tissue reaction seen after long-term lipid-based parenteral nutrition in pigs. An ultrastructural study. APMIS. 1996 June; 104(6):429-36.
183. Salo M. Inhibition of immunoglobulin synthesis in vitro by intravenous lipid emulsion (Intralipid). JPEN J Parenter Enteral Nutr. 1990 September-October; 14(5):459-62.
184. Padeh S et al., Effect of intralipid on the phagocytic and microbicidal capacity of human monocytes in culture. J Pediatr Gastroenterol Nutr. 1987 July-August; 6(4):575-80.
185. Rasmussen A et al., The effect of preoperative nutrition on the immune system. Clin Nutr. 1985 August; 4(3):175-8.
186. Wiernik A et al., The effect of intralipid on mononuclear and polymorphonuclear phagocytes. Am J Clin Nutr. 1983 February; 37(2):256-61.
187. Liu L et al., A new approach to reduce toxicities and to improve bioavailabilities of platinum-containing anti-cancer nanodrugs. Sci Rep. 2015 Jun. 3; 5:10881

### Example 5

### Intralipid Treatment for Post Operative Delirium

Postoperative delirium (POD) is a common and serious adverse event in the elderly patient and is associated with significant morbidity and mortality. A new treatment for POD by intravenous Intralipid injection in the recovery room is first suggested in the medical literature.

### Post Operative Delirium

Delirium is defined by either the Diagnostic and Statistical Manual of Mental Disorders, Fifth Edition (DSM-5) [1] or by the 10th revision of the International Statistical Classification of Diseases and Related Health Problems (ICD 10, Table 3) [2]. Delirium is an acute and fluctuating alteration of mental state of reduced awareness and disturbance of attention. POD (Post Operative Delirium) often starts in the recovery room and occurs up to 5 days after surgery [3-5]. One investigation [4] found that many patients with POD on the peripheral ward already had POD in the recovery room.

More than 230 million surgical procedures are performed each year worldwide, of which more than 80 million are in Europe [6-8]. In Europe, the in-hospital mortality rate up to a maximum of 60 days is 3% after elective surgery and nearly 10% after emergency surgery [7]. In addition to mortality, postoperative cognitive impairments such as POD and postoperative cognitive dysfunction (POCD) impose a huge burden on individuals and society [9]. The incidence of POD is dependent on perioperative and intraoperative risk factors [10]. Therefore, the incidence of POD varies within a broad range [11,12]. For example, a meta-analysis of 26 studies of POD reported an incidence of 4.0 to 53.3% in hip fracture patients and 3.6 to 28.3% in elective patients [13].

Delirium is one of the most common complications following hip fracture surgery in older people. This study identified pre- and pen-operative factors associated with the development of post-operative delirium following hip fracture surgery.

Published and unpublished literature were searched to identify all evidence reporting variables on patient characteristics, on-admission, intra-operative and post-operative management assessing incident delirium in older people following hip fracture surgery. Pooled odds ratio (OR) and mean difference of those who experienced delirium compared to those who did not were calculated for each variable. Evidence was assessed using the Downs and Black appraisal tool and interpreted using the GRADE approach.

A total of 6704 people (2090 people with post-operative delirium) from 32 studies were analyzed. There was moderate evidence of nearly a two-times greater probability of post-operative-delirium for those aged 80 years and over (OR: 1.77; 95% CI: 1.09, 2.87), whether patients lived in a care institution pre-admission (OR: 2.65; 95% CI: 1.79, 3.92), and a six-times greater probability of developing post-operative delirium with a pre-admission diagnosis of dementia (OR: 6.07, 95% CI: 4.84, 7.62). There was no association with intra-operative variables and probability of delirium.

Clinicians treating people with a hip fracture should be vigilant towards post-operative-delirium if their patients are older, have pre-existing cognitive impairment and poorer overall general health. This is also the case for those who experience post-operative complications such as pneumonia or a urinary tract infection [14].

Post-operative cerebral dysfunction includes delirium, usually occurring early and reversible, and post-operative cognitive disorders, usually occurring later and prolonged. This is a frequent complication in patients older than 75 years old. The two neurological pictures are often inter-related. The pathophysiology of both entities is similar and related to post-operative neuro-inflammation; therefore onset may occur independently of any surgical complication. Post-operative cerebral dysfunction is a serious organic complication. Reduction of inflammation represents the most logical preventive measure but currently there are no studies that show this to be effective. Prevention therefore means combining several minor measures, elements that fit well into programs of enhanced post-operative recovery after surgery. Diminished pre-operative cognitive status being a major risk factor, pre-operative rehabilitation combining nutritional, physical and cognitive support can be helpful [15].

Postoperative delirium is a common and serious adverse event in the elderly patient and is associated with significant morbidity and mortality. It is of great importance to identify patients at risk for delirium, in order to focus preventive strategies. The aim of this article is to systematically review current available literature on pre-operative risk factors for delirium after vascular surgery.

A systematic literature search was conducted using PubMed and EMBASE, using the MeSH terms and key words “delirium”, “surgery” and “risk factor”. Studies were retained for review after meeting strict inclusion criteria that included only prospective studies evaluating risk factors for delirium in patients who had elective vascular surgery. Diagnosis of delirium needed to be confirmed using the Diagnostic and Statistical Manual of Mental Disorders (DSM) or ICD-10.

Fifteen articles were selected for inclusion, incidence of delirium across the studies ranged from 5% to 39%. Many factors have been associated with increased risk of delirium, including age, cognitive impairment, comorbidity, depression, smoking, alcohol, visual and hearing impairment, ASA-score, biochemical abnormalities, operative strategies and blood loss.

Delirium is a common complication after elective vascular surgery in elderly. The highest delirium incidence was observed after open aortic surgery as well as after surgery for critical limb ischemia. A picture starts to form of which predisposing factors lead to increased risk of delirium. The leading risk factors consistently identified in this systematic review were advanced age and cognitive impairment. Multi-disciplinary specialist-led interventions in the preoperative phase could decrease incidence and severity of delirium and should be focused on identified highrisk patients [16].

This study [17] investigates the relationship between cognitive dysfunction or delirium detected in the early postsurgical phase and the 1-year mortality among 514 hip fracture hospitalized older persons. Patients with early cognitive dysfunction or delirium experienced a 2-fold increased mortality risk. Early post-operative cognitive dysfunction and delirium are negative prognostic factors for mortality. Premorbid cognitive impairment and dementia in older individuals negatively affect functional recovery after hip fracture. Additionally, postoperative delirium is an established risk factor for negative outcomes among hip fracture patients. While the majority of hip fracture patients experience minor post-surgical cognitive dysfunction, the prognostic value of this phenomenon is unknown. Therefore, we investigated the relationship between minor cognitive dysfunction or delirium detected in the early postsurgical phase and the 1-year mortality after index hip fracture.

We enrolled 514 patients with hip fracture (77.4% women), aged 65 years or older (mean age 83.1±7.3 years), who underwent surgical hip fracture repair. Patients were assessed daily from the second to the fourth post-operative day and at 3, 6, and 12 months thereafter. All participants underwent comprehensive assessment, including detection of delirium by using the confusion assessment method and evaluation of cognitive function by using mini-mental state examination (MMSE; score range 0 to 30, with lower scores indicating poorer performance). In the absence of delirium, post-surgical cognitive dysfunction was defined as having low performance on MMSE. Vital status of 1 year after the index fracture and date of death were gathered from local registries.

The observed 1-year mortality rate was 14.8%. Men were more likely to die than women within 1 year of the index fracture (p<0.01). Compared to participants with better cognitive performance, those with MMSE<24, as well as those with delirium in the post-operative phase, showed a significantly higher 1-year mortality rate (23.3 versus 17.9 and 8.1%, respectively). Independent of age and sex, post-operative cognitive dysfunction as well as delirium was both associated with a 2-fold increased mortality risk.

The presence of minor cognitive dysfunction in the early post-surgical phase is a negative prognostic factor for mortality among elderly hip fracture patients. The burden of minor cognitive dysfunction is likely superimposed on that of delirium in subgroups of frail patients [17].

Perioperative cerebral hypoperfusion/ischemia is a major inciting factor of post-operative-delirium, which is coupled with adverse outcome in elderly patients. Cerebral oximetry enables noninvasive assessment of the regional cerebral oxygen saturation rSO2. This study aimed to investigate whether pen operative rSO2 variations were linked to delirium in elderly patients after spinal surgery.

Postoperative delirium was assessed for 48 h post-operative in 109 patients aged over 60 years without a prior history of cerebrovascular or psychiatric diseases by the Confusion Assessment Method for the intensive care unit and the intensive care delirium screening checklist. The rSO2 values immediately before and throughout surgery were acquired. The preoperative cognitive functions, patient characteristics, and perioperative data were recorded.

During the 48-h postoperative period, 9 patients (8%) exhibited delirium. The patients with delirium showed similar pen operative rSO2 values as those without, in terms of the median lowest rSO2 values (55% vs. 56%; P=0.876) and incidence (22%, both) and duration of decline of rSO2<80% of the baseline values. The serially assessed hemodynamic variables, hematocrit levels, and blood gas analysis variables were also similar between the groups, except for the number of hypotensive events per patient, which was higher in the patients with delirium than in those without (4, interquartile range [IQR] 3 to 6 vs. 2, IQR: lto 3; P=0.014). The degree and duration of decrease of the pen operative rSO2 measurements were not associated with delirium in elderly patients after spinal surgery [18].

Three-dimensional Arterial Spin Labeling (ASL) MRI was performed before surgery in a cohort of 146 prospectively enrolled subjects ≥70 years old scheduled to undergo elective surgery. We investigated the prospective association between ASL-derived measures of cerebral blood flow (CBF) before surgery with postoperative delirium incidence and severity using whole-brain and globally normalized voxel-wise analysis. We also investigated the cross-sectional association of CBF with patients' baseline performance on specific neuropsychological tests, and with a composite general cognitive performance measure (GCP). Out of 146 subjects, 32 (22%) developed delirium. We found no significant association between global and voxel-wise CBF with delirium incidence or severity. We found the most significant positive associations between CBF of the posterior cingulate and precuneus and the Hopkins Verbal Learning Test-Revised total score, Visual Search and Attention Test (VSAT) score and the GCP composite. VSAT score was also strongly associated with right parietal lobe CBF. ASL can be employed in a large, well-characterized older cohort to examine associations between CBF and age-related cognitive performance. Although ASL CBF measures in regions previously associated with preclinical Alzheimer's Disease were correlated with cognition, they were not found to be indicators of baseline pathology that may increase risk for delirium [19].

Oxidative stress may be involved in occurrence of postoperative delirium (POD) and cognitive dysfunction (POCD). 8-iso-Prostaglandin F2α (8-iso-PGF2α), an isoprostane derived from arachidonic acid via lipid peroxidation, is considered a gold standard for measuring oxidative stress. The present study aimed to investigate the ability of postoperative plasma 8-iso-PGF2α levels to predict POD and POCD in elderly patients undergoing hip fracture surgery.

Postoperative plasma 8-iso-PGF2α levels of 182 patients were measured by an enzyme-linked immunosorbent assay. We assessed the relationships between plasma 8-iso-PGF2α levels and the risk of POD and POCD using a multivariate analysis.

Plasma 8-iso-PGF2α levels and age were identified as the independent predictors for POD and POCD. Based on areas under receiver operating characteristic curve, the predictive values of 8-iso-PGF2α were obviously higher than those of age for POD and POCD. In a combined logistic-regression model, 8-iso-PGF2α significantly enhanced the areas under curve of age for prediction of POD and POCD. Postoperative plasma 8-iso-PGF2α levels may have the potential to predict POD and POCD in elder patients undergoing hip fracture surgery [20].

Risk factors for delirium following cardiac surgery are incompletely understood. The aim of this study was to investigate whether intra-operative pathophysiological alterations and therapeutic interventions influence the risk of post-operative delirium.

This retrospective cohort study was performed in a 12-bed cardio-surgical intensive care unit (ICU) of a university hospital and included patients consecutively admitted after cardiac surgery during a 2-month period. The diagnosis of delirium was made clinically using validated scores. Comparisons between patients with and without delirium were performed with non-parametric tests. Logistic regression was applied to identify independent risk factors. Results are given as number (percent) or median (range).

Of the 194 consecutive post-cardiac surgery patients, 50 (26%) developed delirium during their ICU stay. Univariate analysis revealed that significant differences between patients with and without delirium occurred in the following intra-operative variables: duration of cardiopulmonary bypass (184 [72-299] vs. 113 (37-717) minutes, p<0.001), lowest mean arterial pressure (50 [30-70] vs. 55 [30-75] mmHg, p=0.004), lowest haemoglobin level (85 [56-133] vs. 98 [53-150] g/L, p=0.005), lowest body temperature (34.5 [24.4-37.2] vs. 35.1 [23.9-37.2] ° C., p=0.035), highest noradrenaline support (0.11 [0.00-0.69] vs. 0.07 [0.00-0.42] μg/kg/minute, p=0.001), and frequency of red blood cell transfusions (18 [36%] vs. 26 [18%], p=0.018) and platelet transfusions (23 [46%] vs. 24 [17%], p<0.001). Only platelet transfusions remained an independent risk factor in the multivariate analysis (p<0.001).

In patients undergoing cardiac surgery, various intra-operative events, such as transfusion of platelets, were risk factors for the development of a post-operative delirium in the ICU. Further research is needed to unravel the underlying mechanisms [21].

In this study, [22] aimed to determine the risk factors and the incidence of delirium in patients who were followed postoperatively in our surgical intensive care unit for 24 h using the confusion assessment method (CAM).

After obtaining approval from the ethics committee, 250 patients were included in the study. Patients who were operated under general anaesthesia or regional anaesthesia and followed in the surgical intensive care unit were evaluated by the Ramsay Sedation Scale on the first postoperative day. CAM was applied to the patients who had a Ramsey Sedation Score of ≤4. Patients' age, gender, American Society of Anesthesiologists (ASA) scores, preoperative risk factors, type of anaesthesia, operation time, intra-operative procedures, pain scores evaluated by the visual analogue scale (VAS) and postoperative analgesia methods were recorded.

The incidence of delirium was found to be 18.4%. The average age of patients who developed delirium was greater than the others (68.8±12.7 and 57.6±12, p=0.001, respectively). It was observed that a one-unit increase in the ASA score resulted in a 3.3-fold increase in the risk of delirium. The incidence of delirium in patients undergoing regional anaesthesia was 34.6%, whereas it was 16.5% in patients receiving general anaesthesia (p=0.024). The existence of preoperative diabetes mellitus (DM) and chronic obstructive pulmonary disease (COPD) was shown to improve the development of delirium (p&ly; 0.05). Delirium incidence was significantly higher in patients who were administered meperidine for postoperative analgesia (p=0.013). The VAS scores of patients who developed delirium were found to be significantly higher (p=0.006).

As a result, we found that older age, high ASA score, preoperative DM and COPD are important risk factors for the development of delirium. Regional anaesthesia, high postoperative pain scores and meperidine use were observed to be associated with the development of delirium. In the postoperative period, addition of CAM, a simple measurement technique, to the daily follow-up forms can provide the early recognition of delirium, which is often underdiagnosed. We think that identification and prevention of effective risk factors have the primary importance for postoperative delirium [22].

Delirium after cardiac surgery is a major problem. The exact mechanisms behind delirium are not understood. Potential pathways of delirium include neurotransmitter interference, global cognitive disorder, and neuroinflammation. Several predisposing and precipitating risk factors have been identified for postoperative delirium. The development of delirium following cardiac surgery is associated with worse outcomes in the perioperative period. Multiple interventions are being explored for the prevention and treatment of delirium. Studies investigating the potential roles of biomarkers in delirium as well as pharmacological interventions to reduce the incidence and duration of delirium are necessary to mitigate this negative outcome [23].

Perhaps the most frequently described mechanism of brain injury in CABG surgery is based on the recognition that microemboli are generated by the surgeon manipulating the heart and aorta, through cardiotomy suctioning, and by the cardiopulmonary bypass circuit itself. Microemboli can be detected intraoperatively as high-intensity transient signals by transcranial Doppler sonography. They have the potential to lodge in cerebral microvasculature, impairing blood supply to the brain and thus cerebral oxygenation. Several phases during cardiac surgery have been associated with increased risk of embolic showers. Aortic cannulation and clamping (during application of cardiopulmonary bypass) increase the high-intensity transient signal rate, particularly if there is extensive atheroma in the ascending aorta [24]. It is not surprising, therefore, that most (81%) microemboli are generated at the point of aortic cross-clamp release [25]. Retaining the shed mediastinal blood with cardiotomy suckers provides an additional source of lipid emboli and other fragments [26].

### Conclusion

A new treatment for POD by intravenous Intralipid injection in the recovery room is first suggested in the medical literature.

### REFERENCES

1. American Psychiatric Association (2013) Diagnostic and statistical manual of mental disorders (Dsm-51). (5th edn) Amer Psychiatric Pub Inc, USA.
2. International Statistical Classification of Diseases and Related Health Problems 10th Revision.
3. Olin K, Eriksdotter Jönhagen M, Jansson A, Herrington M K, Kristiansson M, et al. (2005) Postoperative delirium in elderly patients after major abdominal surgery. Br J Surg 92(12): 1559-1564.
4. Sharma P T, Sieber F E, Zakriya K J, Ronald W, Jian M D, et al. (2005) Recovery room delirium predicts postoperative delirium after hip-fracture repair. Anesthesia & Analgesia 101(4): 1215-1220.
5. Radtke F M, Franck M. Schneider M. Seeling M, Heinz A, et al. (2008) Comparison of three scores to screen for delirium in the recovery room. Br J Anaesth 101(3): 338-343.
6. (2015) Eurostat-Your key to European statistics.
7. Pearse R M, Moreno R P, Bauer P, Pelosi P, Metnitz P, et al. (2012) Mortality after surgery in Europe: a 7 day cohort study. Lancet 380(9847): 1059-1065,
8. Weiser T G, Regenbogen S E, Thompson K D, Haynes A B, Lipsitz S R, et al. (2008) An estimation of the global volume of surgery: a modelling strategy based on available data. Lancet 372(9633): 139-144,
9. Saczynski J S, Marcantonio E R, Quach L, Fong T G, Gross A, et al. (2012) Cognitive trajectories after postoperative delirium. N Engl J Med 367(1): 30-39.
10. Multer N, Lingehall H C, Gustafson Y (2013) Delirium after cardiac surgery: incidence and risk factors. Interact Cardiovasc Thorac Surg 17: 790-796.
11. Dasgupta M, Dumbrell A C (2006) Preoperative risk assessment for delirium after noncardiac surgery: a systematic review. J Am Geriatr Soc 54(10): 1578-1589.
12. Dyer C B, Ashton C M, Teasdale T A (1995) Postoperative delirium. A review of 80 primary data-collection studies. Arch Intern Med 155(5): 461-465.
13. Bruce A J, Ritchie C W, Blizard R, Lai R, Raven P (2007) The incidence of delirium associated with orthopedic surgery: a meta-analytic review. Int Psychogeriatr 19(2): 197-214.
14. Smith T O, Cooper A, Peryer G, Griffiths R, Fox C, et al. (2017) Factors predicting incidence of post-operative delirium in older people following hip fracture surgery: a systematic review and meta-analysis. Int J Geriatr Psychiatry.
15. Benhamou D, Brouquet A (2016) Postoperative cerebral dysfunction in the elderly: Diagnosis and prophylaxis. J Visc Surg 153(6): S27-S32.
16. Raats Steunenberg S L, D Lange D C, Van Der Laan L (2016) Risk factors of post-operative delirium after elective vascular surgery in the elderly: A systematic review. Int J Surg 35: 1-6.
17. Ruggiero C, Bonamassa Pelini L, Priolerta I, Cianferotti L, et al. (2017) Early post-surgical cognitive dysfunction is a risk factor for mortality among hip fracture hospitalized older persons. Osteoporos Int 28(2): 667-675
18. Sob 5, Shim J K, Song J W, Kim K N, Noh H Y, et al. (2016) Postoperative Delirium in Elderly Patients Undergoing Major Spinal Surgery: Role of Cerebral Oximetry. Neurosurg Anesthesiol.
19. Hshieh Dai W. Cavallari M, Guttmann C R, Meier D S, et al. (2016) Cerebral blood flow MRI in the nondemented elderly is not predictive of post-operative delirium but is correlated with cognitive performance. J Cereb Blood Flow Metab.
20. Zheng Y B, Ruan G M, Fu J X, Su Z L, Cheng P, et al. (2016) Postoperative plasma 8-iso-prostaglandin F2cr. levels are associated with delirium and cognitive dysfunction in elderly patients after hip fracture surgery. Clin Chim Acta 455: 149-153.
21. Rudiger A, Begdeda H, Babic D, Krüger B, Seifert B, et al. (2016) Intra-operative events during cardiac surgery are risk factors for the development of delirium in the ICU. Crit Care 20: 264.
22. Bilge E Ü, Kaya M, Şenel G Ö, Ünver S (2015) The Incidence of Delirium at the Postoperative Intensive Care Unit in Adult Patients. Turk J Anaesthesiol Reanim 43(4): 232-239.
23. O Neal J B, Shaw A D (2016) Predicting, preventing, and identifying delirium after cardiac surgery. Perioper Med (Lond) 5: 7.
24. Mackensen G B, Ti L K, Phillips Bute B G, Mathew J P, Newman M F, et ai. (2003) Neurologic Outcome Research Group (NORG). Cerebral embolization during cardiac surgery: impact of aortic atheroma burden. Br J Anaesth 91(5): 656-661.
25. Reinsfeit B. Ricksten S E, Zetterberg H. Blennow K. Fredén Lindqvist, et al. (2012) Cerebrospinal fluid markers of brain injury, inflammation, and blood-brain barrier dysfunction in cardiac surgery. Ann Thorac Surg 94(2): 549-555.
26. Brooker R F, Brown W R, Moody D M, Hammon J W, Reboussin D M, et ai. (1998) Cardiotomy suction: a major source of brain lipid emboli during cardiopulmonary bypass. Ann Thorac Surg 65(6): 1651-1655.

### Example 6

### Can Intralipid Infusion Open a Coronary Occlusion Causing Acute Myocardial Infarction

Coronary heart disease remains the leading cause of morbidity and mortality in Western countries. The best hope of salvaging viable myocardium after a coronary occlusion is by rapid reperfusion of the ischemic myocardium, either by thrombolysis or primary percutaneous coronary intervention. The use of intravenous intralipid infusion instead of thrombolysis or primary percutaneous coronary intervention is first suggested in the medical literature

### Thrombolysis or Primary Percutaneous Coronary Intervention or Intralipid Infusion?

Coronary heart disease remains the leading cause of morbidity and mortality in Western countries. The best hope of salvaging viable myocardium after a coronary occlusion is by rapid reperfusion of the ischemic myocardium, either by thrombolysis or primary percutaneous coronary intervention. Although reperfusion restores blood flow, oxygen, and nutrients to the cardiac muscle, it also has the potential to induce reperfusion injury.

Postconditioning of the heart with brief episodes of reperfusion/occlusion at the onset of reflow has been shown to limit infarct size. However, this approach is not practical for patients treated with thrombolytic agents and therefore a more generic pharmacologic postconditioning is still needed. The ideal pharmacologic candidates need to be safe and effective when administered during the first few minutes of reperfusion by inducing cellular protection or enhancing myocardial tolerance to ischemia/reperfusion injury. Several drugs have yielded encouraging results in animals and a few have been tested in humans; however, none of these modalities has been widely accepted.

Lipids and in particular polyunsaturated fatty acids have received special cardiovascular research attention because polyunsaturated fatty acid-rich diets are associated with a decreased risk of coronary artery disease. Acute application of polyunsaturated fatty acids to cardiomyocytes has also been shown to shorten action potential duration and this could account for the antiarrhythmic mechanism of the polyunsaturated fatty acids. Intralipid (Sigma, St. Louis, Mo.) is a brand name for the first safe fat emulsion for human use; Intralipid 20% is an emulsion of soybean oil (20%), egg yolk phospholipids (1.2%), and glycerol (2.2%). Intralipid has been widely used in patients who need total parenteral nutrition and as a vehicle for different drugs such as propofol. It has been shown recently that postischemic administration of Intralipid protects the isolated rat heart against ischemia/reperfusion injury. However, the molecular mechanism in which Intralipid mediates cardioprotection is completely unknown [1].

Intralipid, a brand name for the first safe fat emulsion for human use, has been shown to be cardioprotective. However, the mechanism of this protection is not known. The authors investigated the molecular mechanism(s) of Intralipid-induced cardioprotection against ischemia/reperfusion injury, particularly the role of glycogen synthase kinase-3β (GSK-3β) and mitochondrial permeability transition pore in this protective action.

In vivo rat hearts or isolated Langendorff-perfused mouse hearts were subjected to ischemia followed by reperfusion with Intralipid (1% in ex vivo and one bolus of 20% in in vivo) or vehicle. The hemodynamic function, infarct size, threshold for the opening of mitochondrial permeability transition pore, and phosphorylation levels of protein kinase B (Akt)/extracellular signal regulating kinase (ERK)/GSK-3β were measured.

Administration of Intralipid at the onset of reperfusion resulted in approximately 70% reduction in infarct size in the in vivo rat model. Intralipid also significantly improved functional recovery of isolated Langendorff-perfused mouse hearts as the rate pressure product was increased from 2,999±863 mmHg*beats/min in the control group to 13,676±611 mmHg*beats/min (mean±SEM) and the infarct size was markedly smaller (18.3±2.4% vs. 54.8±2.9% in the control group, P<0.01). The Intralipid-induced cardioprotection was fully abolished by LY294002, a specific inhibitor of PI3K, but only partially by PD98059, a specific ERK inhibitor. Intralipid also increased the phosphorylation levels of Akt/ERK1/glycogen synthase kinase-3β by eightfold, threefold, and ninefold, respectively. The opening of mitochondrial permeability transition pore was inhibited by Intralipid because calcium retention capacity was higher in the Intralipid group (274.3±8.4 nM/mg vs.

168.6±9.6 nM/mg in the control group).

Postischemic treatment with Intralipid inhibits the opening of mitochondrial permeability transition pore and protects the heart through glycogen synthase kinase-3β via PI3K/Akt/ERK pathways (1).

It was recently shown that postischemic administration of intralipid protects the heart against ischemia-reperfusion injury. Here we compared the cardioprotective effects of intralipid with cyclosporine-A, a potent inhibitor of the mitochondrial permeability transition pore opening.

In vivo rat hearts or isolated Langendorff-perfused mouse hearts were subjected to ischemia followed by reperfusion with intralipid (0.5%, 1% and 2% ex-vivo, and 20% in vivo), cyclosporine-A (0.2 μM, 0.8 μM, and 1.5 μM ex-vivo and 10 mg/kg in vivo), or vehicle. The hemodynamic function, infarct size, calcium retention capacity, mitochondrial superoxide production, and phosphorylation levels of protein kinase B (Akt)/glycogen synthase kinase-313 (GSK-3(3) were measured. The values are mean±SEM.

Administration of intralipid at reperfusion significantly reduced myocardial infarct size compared with cyclosporine-A in vivo (infarct size/area at risk) %: 22.9±2.5% vs. 35.2±3.5%; P=0.030, n=7/group). Postischemic administration of intralipid at its optimal dose (1%) was more effective than cyclosporine-A (0.8 μM) in protecting the ex vivo heart against ischemia-reperfusion injury, as the rate pressure product at the end of reperfusion was significantly higher (mmHg·beats/min: 12,740±675 [n=7] vs. 9,203±10,781 [n=5], P=0.024), and the infarct size was markedly smaller (17.3±2.9 [n=7] vs. 29.2±2.7 [n=5], P=0.014). Intralipid was as efficient as cyclosporine-A in inhibiting the mitochondrial permeability transition pore opening (calcium retention capacity=280±8.2 vs. 260.3±2.9 nmol/mg mitochondria protein in cyclosporine-A, P=0.454, n=6) and in reducing cardiac mitochondrial superoxide production. Unlike intralipid, which increased phosphorylation of Akt (6-fold) and GSK-3β (5-fold), cyclosporine-A had no effect on the activation of these prosurvival kinases.

Although intralipid inhibits the opening of the mitochondrial permeability transition pore as efficiently as cyclosporine-A, intralipid is more effective in reducing the infarct size and improving the cardiac functional recovery [2].

It was recently demonstrated that the heart of late pregnant (LP) rodents is more prone to ischemia/reperfusion (I/R) injury compared to non-pregnant rodents. Lipids, particularly polyunsaturated fatty acids, have received special attention in the field of cardiovascular research. Here, we explored whether Intralipid (ITLD) protects the heart against I/R injury in LP rodents and investigated the mechanisms underlying this protection.

In-vivo female LP rat hearts or ex-vivo isolated Langendorff-perfused LP mouse hearts were subjected to ischemia followed by reperfusion with PBS or ITLD (one bolus of 5 mg/kg of 20% in in-vivo and 1% in ex-vivo). Myocardial infarct size, mitochondrial calcium retention capacity, genome-wide expression profiling, pharmacological inhibition and co-immunoprecipitation were performed. One bolus of ITLD at reperfusion significantly reduced the in-vivo myocardial infarct size in LP rats (23.3±2% vs. 55.5±3.4% in CTRL, p<0.01). Postischemic administration of ITLD also protected the LP hearts against I/R injury ex-vivo. ITLD significantly increased the threshold for the opening of the mitochondrial permeability transition pore in response to calcium overload (nmol-calcium/mg-mitochondrial protein: 290±17 vs. 167±10 in CTRL, p<0.01) and significantly increased phosphorylation of STAT3 (1.8±0.08 vs. 1±0.16 in CTRL, p<0.05) and GSK-3β (2.63±0.55 vs.1±0.0.34 in CTRL, p<0.05). The ITLD-induced cardioprotection was fully abolished by Stattic, a specific inhibitor of STAT3. Transcriptome analysis revealed caveolin 2 (Cav2) was significantly upregulated by ITLD in hearts of LP rats under I/R injury. Co-immunoprecipitation experiments showed that Cav2 interacts with STAT3. ITLD protects the heart in late pregnancy against I/R injury by inhibiting the mPTP opening through Cav2/STAT3/GSK-3β pathway [3].

Recent studies have demonstrated that intralipid (ILP) conferred myocardial protection against ischemia-reperfusion (IR) injury through activation of reperfusion injury salvage kinase (RISK) pathway. As RISK signal has been shown to be impaired in hypertrophied myocardium, we investigated whether ILP-induced cardiac protection was maintained in hypertrophied rat hearts. Transverse aortic constriction was performed on male Sprague-Dawley rats to induce left ventricular hypertrophy, then sham-operated or hypertrophied rat hearts were isolated and perfused retrogradely by the Langendorff for 30 min (equilibration) followed by 40 min of ischemia and then 120 min of reperfusion. The isolated hearts received 15-min episode of 1% ILP separated by 15 min of washout or three episodes of 5-min ischemia followed by 5-min reperfusion before ischemia. The hemodynamics, infarct size, apoptosis, phosphorylated protein kinase B (p-Akt), phosphorylated extracellular regulated protein kinase 1/2 (ERK1/2), phosphorylated glycogen synthase kinase (GSK3β), Bcl-2, phosphorylated Bad, and Bax were determined. We found that ILP significantly improved left ventricular hemodynamics and reduced infarct size and the number of TUNEL (terminal deoxynucleotidyl transferase dUTP nick end labeling)-positive cells in the sham-operated rat hearts exposed to IR. However, such myocardial infarct-sparing effect of ILP was completely blocked by phosphatidylinositol-3-kinase inhibitor wortmannin, but only partially by mitogen-activated protein kinase kinase inhibitor PD98059 in sham-operated hearts. Intralipd upregulated the phosphorylation of Akt, extracellular regulated protein kinase 1/2 (ERK1/2), and their downstream target of GSK3β and antiapoptotic Bcl-2 expression in healthy rat hearts. Nonetheless, ILP failed to improve left ventricular hemodynamics and reduced infarct size and apoptosis and increase the phosphorylated Akt, ERK1/2, GSK3β, and antiapoptotic Bcl-2 in hypertrophied myocardium. In contrast, ischemic preconditioning increased the phosphorylation of Akt, ERK1/2 and GSK3β, improved heart pump function, and reduced myocardial necrosis in sham-operated hearts, a phenomenon partially attenuated by ventricular hypertrophy. Interestingly, GSK inhibitor SB216763 conferred cardioprotection against IR injury in sham-operated hearts, but failed to exert cardioprotection in hypertrophied myocardium. Our results indicated that ventricular hypertrophy abrogated ILP-induced cardioprotection against IR injury by alteration of RISK/GSK3β signal [4].

Elevated low-density lipoprotein cholesterol and triglycerides are major risk factors for coronary artery disease. However, fatty acids from triglycerides are a major energy source, low-density lipoprotein cholesterol is critical for cell membrane synthesis, and both are critical for cell survival. This study was designed to clarify the relationship between lipid profile, morbidity as assessed by Killip classification, and 30-day mortality in patients with acute myocardial infarction.

Seven hundred twenty-four patients with acute myocardial infarction in the coronary care program of the Bureau of Health Promotion were analyzed. Low-density lipoprotein cholesterol and triglyceride levels were significantly lower in high-Killip (III+IV) patients compared with low-Killip (I+II) patients and in those who died compared with those who survived beyond 30 days (both p<0.001). After adjustment for risk factors, low-density lipoprotein cholesterol less than 62.5 mg/dL and triglycerides less than 110 mg/dL were identified as optimal threshold values for predicting 30-day mortality and were associated with hazard ratios of 1.65 (95% CI, 1.18-2.30) and 5.05 (95% CI, 1.75-14.54), and the actual mortality rates were 23% in low low-density lipoprotein, 6% in high low-density lipoprotein, 14% in low triglycerides, and 3% in high triglycerides groups, respectively. To test the synergistic effect, high-Killip patients with triglycerides less than 62.5 mg/dL and low-density lipoprotein cholesterol less than 110 mg/dL had a 10.9-fold higher adjusted risk of mortality than low-Killip patients with triglycerides greater than or equal to 62.5 mg/dL and low-density lipoprotein cholesterol greater than or equal to 110 mg/dL (p<0.001). The lipid paradox also improved acute myocardial infarction short-term outcomes prediction on original Killip and thrombolytic in myocardial infarction scores.

Low low-density lipoprotein cholesterol, low triglycerides, and high Killip severity were associated with significantly higher 30-day in-hospital mortality in patients presenting with acute myocardial infarction. The initial lipid profile of patients with acute myocardial infarction may therefore hold prognostic value [5].

Emulsified Isoflurane (EIso)

Volatile anesthetic postconditioning reduces myocardial infarct size against ischemia/reperfusion (I/R) injury. We tested the hypothesis that emulsified isoflurane (EIso) administrated after ischemia exerts cardioprotection in a rat model of myocardial I/R. Male SD rats underwent 30-min coronary occlusion followed by 3-h reperfusion except for sham rats. All vehicles were administrated intravenously at reperfusion onset for 30 min. In the first study, 56 rats were given saline (CON), 30% intralipid (IL) and 1, 2, 4, 8 or 16 mL/kg EIso for infarct size measurement. In a second study, 32 rats were randomized to four groups and administrated saline in sham (sham) and control (CON) groups, 30% intralipid in IL group and 2 mL/kg emulsified isoflurane in EIso group. Cardiomyocytic enzyme activity was determined. Myocardial mitochondria and cytosol were isolated to determine mitochondrial energy metabolism, cytochrome c release, mitochondrial membrane potential (ΔΨm) and opening of the mitochondrial permeability transition pore (mPTP). Morphologic changes in mitochondria were observed by transmission electron microscopy. Compared with CON and IL, 2, 4 and 8 mL/kg EIso limited infarct size (P<0.01). Serum levels of cardiac enzyme leakage were reduced in EIso-treated hearts compared with CON (P<0.01 or P<0.05). EIso preserved the ultrastructure of mitochondria, protected against mPTP opening, decreased cytochrome c release and preserved ATP production and ΔΨm. In conclusion, EIso is effective in reducing infarct size and in preserving mitochondrial function after ischemia and reperfusion injury [6].

The purpose of this study was to investigate whether adding emulsified isoflurane to St Thomas cardioplegia solution could enhance the cardiac protection after cardioplegic arrest in rats. Thirty isolated heart preparations (male Sprague-Dawley rats) were randomly divided into 3 groups (n=10/group) according to the different cardioplegia solutions being given: St Thomas solution mixed with emulsified isoflurane (containing 2.8% of isoflurane, group EI), St Thomas solution mixed with emulsified Intralipid (Huarui Pharmacy, Wuxi, Jiangsu, China) (group EL), and St Thomas solution alone (group St). In the 35-minute normothermic ischemia period, infusion of cardioplegia solution was repeated every 15 minutes. After the 35-minute ischemia period, the heart was perfused with Krebs-Henseleit buffer for another 2 hours.

The functional parameters of the heart were monitored throughout the experiments. The coronary effluent was collected for measuring the activity of CK-MB 30 minutes after reperfusion, and the infarct size was assessed at the end of reperfusion. The infarct size in group EI (24%+/−4%) was reduced when compared with that in group EL (31%+/−8%, p<0.05) and group St (43%+/−9%, p<0.001). The CK-MB activity in group EI was decreased significantly when compared with that in group EL and group St (p<0.05). The functional recovery in group EI also was improved. Compared with standard St Thomas solution alone, adding 30% Intralipid alone also significantly reduced the infarct size and the CK-MB leakage and improved the recovery of the mechanical function. St Thomas cardioplegia solution supplemented with emulsified isoflurane enhanced its cardioprotection in an isolated heart ischemia reperfusion injury model in rats [7].

Pretreatment with volatile anesthetics has been demonstrated to exert cardioprotective effects. The purpose of this study was to examine the effect of emulsified isoflurane, a new formulation of isoflurane in lipid emulsion, administered intravenously in an ischemia and reperfusion model of myocardial injury. Thirty-two Sprague Dawley rats of both sexes were subjected to 30 min of myocardial ischemia followed by 180 min of reperfusion. Each was assigned to one of four pretreatment groups to receive an isovolumetric intravenous infusion of saline: control group, 30% intralipid group, 8% emulsified isoflurane 2 ml kg-lgroup, and sham group (each group, n=8). The vehicles were administered at a constant rate for 30 min and then discontinued 30 min before left anterior descending coronary artery occlusion. The cardioprotective effects were examined by determining hemodynamics, infarct size, enzyme activity, and cardiomyocytic apoptosis.

Pretreatment with emulsified isoflurane 2 ml kg-1(P=0.000) significantly reduced infarct size (22.6+/−2.2%) compared with control (34.8+/−2.3%) and 30% intralipid (31.1+/−2.9%). When compared with the control and intralipid groups, emulsified isoflurane increased Bcl-2 expression while decreasing Bax and Caspase-3 expression and enhancing Bcl-2/Bax ratios. The apoptotic index in the emulsified isoflurane treatment group showed a significant reduction compared with that in the control group (P=0.000) and the intralipid group (P=0.001). In addition, the serum levels of lactate dehydrogenase and creatine kinase were markedly reduced in the emulsified isoflurane treatment group compared with the control and intralipid groups (lactate dehydrogenase, P=0.015 vs. control; creatine kinase, P=0.000 vs. control and intralipid).

These data support a cardioprotective effect of intravenous emulsified isoflurane against myocardial ischemia and reperfusion injury, which are mediated, at least in part, by the inhibition of apoptosis and cell damage [8]. It has been shown that inhaled isoflurane limits the size of myocardial infarcts. The aim of the present study was to examine the effects of emulsified isoflurane on cardiac function and myocardial apoptosis in an ischaemia model of myocardial injury. In the first study, 48 rats were randomly allocated to six groups (n=8 in each): control (saline); emulsified isoflurane (EIso) at 1, 2 or 4 mL/kg; 30% intralipid (vehicle for EIso); and sham operated. Rats received isovolumetric intravenous infusions for 30 min and then, 30 min after cessation of the infusion, 90 min coronary occlusion. Haemodynamics and myocardial infarct size were measured. In the second study, another 48 rats were randomized into six groups (n=8 in each). After 90 min ischaemia, rats were killed for histopathological study, immunohistochemical evaluation and apoptosis measurement. Pretreatment with 2 and 4 mL/kg EIso significantly attenuated decreases in left ventricular systolic pressure and dP/dt(max), and increases in left ventricular end-diastolic pressure and −dP/dp(max), and alleviated myocardial injury compared with the control, intralipid and 1 mL/kg EIso groups (P<0.05). Infusion of 1 mL/kg EIso and intralipid had no effect on haemodynamics, infarct size or histological variables. Expression of Bcl-2 was increased, whereas expression of Bax and caspase 3 was decreased, after preconditioning with 2 and 4 mL/kg EIso (P<0.05). The apoptotic index in the 2 and 4 mL/kg Eiso-treated groups was reduced compared with that in the control and intralipid groups (P<0.01). In conclusion, EIso ameliorates cardiac dysfunction, attenuates myocardial damage and inhibits apoptosis after ischaemia, which may be attributed, in part, to diminished expression of apoptosis-related protein [9].

To evaluate the protective effects of 8% emulsified isoflurane after myocardial ischemia-reperfusion injury and its mechanism in rabbits. Twenty-four male adult New Zealand white rabbits were anesthetized with intravenous injection of 30 mg/kg pentobarbital followed by 5 mg×kg-1×h-1 infusion. All rabbits were subjected to 30 minutes of left anterior descending coronary artery (LAD) occlusion and 3 hours of subsequent reperfusion. Before LAD occlusion, the rabbits were randomly allocated into three groups for preconditioning treatment (eight for each group). The control group (C group) received intravenously 0.9% NaCl for 30 minutes. The emulsified isoflurane group (EI group) received 8% emulsified isoflurane intravenously till 0.64% end-tidal concentration for 30 minutes that was followed by a 15-minute washout period.

The Intralipid group (IN group) received 30% Intralipid for 30 minutes. The infarcted area, plasma malondialdehyde (MDA) content, superoxide dismutase activity (SOD) and nitrite concentration after 3-hour myocardial perfusion were recorded simultaneously.

For the myocardial ischemia-reperfusion injury animals, the infarcted size in the EI group was significantly reduced (91.9%+/−8%) as compared with control group (39%+/−6%, t=5.19, P<0.01). The plasma SOD activity and nitrite concentration in EI group were significantly higher than those in control group (t=2.82, t=8.46, P<0.05), but MDA content was lower in EI group than that in control group (t=2.56, P<0.05).

The results indicate that emulsified isoflurane has a cardioprotection effect against ischemia-reperfusion injury. This beneficial effect of emulsified isoflurane is probably through NO release and consequently by increase in antioxidation of myocardium [10]. In this study, we examined the cardioprotective effects of parental emulsified isoflurane compared with inhaled isoflurane.

Thirty-two rabbits were subjected to 30 min of myocardial ischemia induced by temporary ligation of the left anterior descending coronary artery followed by 3 h of reperfusion. Before left anterior descending coronary artery occlusion, the rabbits were randomly allocated into one of four groups (eight for each group): group C, no ischemia preconditioning treatment; group IS, inhaled isoflurane 1.1% end-tidal; group EI, a continuous infusion of 8% emulsified isoflurane to an end-tidal concentration of 0.64%; and group IN, a continuous infusion of 30% Intralipid started 30 min. Treatments were started 30 min before ischemia followed by a 15 min washout period for isoflurane groups. Myocardial infarct volume, lactate dehydrogenase, and creatine kinase levels were measured and changes in mitochondrial ultrastructure assessed after 3 h myocardial reperfusion.

Myocardial infarct size 3 h after reperfusion was lower in groups IS and EI compared with groups C and IN (20%+/−8%, 18%+/−8%, 39%+/−6%, and 34%+/−9%, respectively, P<0.01). There were no differences in myocardial infarct size between groups IS and EI or between groups C and IN. Plasma lactate dehydrogenase and creatine kinase levels were lower in group IS (456+/−58 U/L and 1725+/−230 U/L) and group EI (451+/−54 U/L and 1686+/−444 U/L) 3 h after myocardial reperfusion compared with groups C (676+/−82 U/L and 2373+/−529 U/L; P<0.01). Mitochondrial ultrastructure changes were less pronounced in groups IS and EI compared with group C.

Our results indicate that, in rabbits, i.v. emulsified isoflurane provides similar myocardial protection against ischemia-reperfusion injury as inhaled isoflurane [11].

The Intralipid Sink Effect

Papadopoulou A et al. [12] hypothesized that by substituting a dye surrogate in place of local anesthetic, they could visually demonstrate dye sequestration by lipid emulsion that would be dependent on both dye lipophilicity and the amount of lipid emulsion used.

They selected 2 lipophilic dyes, acid blue 25 and Victoria blue, with log P values comparable to lidocaine and bupivacaine, respectively. Each dye solution was mixed with combinations of lipid emulsion and water to emulate “lipid rescue” treatment at dye concentrations equivalent to fatal, cardiotoxic, and neurotoxic local anesthetic plasma concentrations. The lipid emulsion volumes added to each dye solution emulated equivalent intravenous doses of 100, 500, and 900 mL of 20% Intralipid in a 75-kg adult. After mixing, the samples were separated into a lipid-rich supernatant and a lipid-poor subnatant by heparin flocculation. The subnatants were isolated, and their colors compared against a graduated dye concentration scale.

Lipid emulsion addition resulted in significant dye acquisition by the lipid compartment accompanied by a reduction in the color intensity of the aqueous phase that could be readily observed. The greatest amount of sequestration occurred with the dye possessing the higher log P value and the greatest amount of lipid emulsion. This study provides a visual demonstration of the lipid sink effect. It supports the theory that lipid emulsion may reduce the amount of free drug present in plasma from concentrations associated with an invariably fatal outcome to those that are potentially survivable.

Local anesthetic (LA) intoxication with cardiovascular arrest is a potential fatal complication of regional anesthesia. Lipid resuscitation has been recommended for the treatment of LA-induced cardiac arrest. Aim of the study [13] was to compare four different rescue regimens using epinephrine and/or lipid emulsion and vasopressin to treat cardiac arrest caused by bupivacaine intoxication.

Twenty-eight piglets were randomized into four groups (4×7), anesthetized with sevoflurane, intubated, and ventilated. Bupivacaine was infused with a syringe driver via central venous catheter at a rate of 1 mg·kg-1·min-1 circulatory arrest. Bupivacaine infusion and sevoflurane were then stopped, chest compression was started, and the pigs were ventilated with 100% oxygen. After 1 min, epinephrine 10 μg·μkg-1 (group 1) Intralipid(®) 20% 4 ml·kg-1 (group 2), epinephrine 10 μg·kg-1+Intralipid(®) 4 ml·kg-1 (group 3) or 2 IU vasopressin+Intralipid(®) 4 ml·kg-1 (group 4) were administered. Secondary epinephrine doses were given after 5 min if required.

Survival was 71%, 29%, 86%, and 57% in groups 1, 2, 3, and 4. Return of spontaneous circulation was regained only by initial administration of epinephrine alone or in combination with Intralipid(®). Piglets receiving the combination therapy survived without further epinephrine support. In contrast, in groups 2 and 4, return of spontaneous circulation was only achieved after secondary epinephrine rescue.

In cardiac arrest caused by bupivacaine intoxication, first-line rescue with epinephrine and epinephrine+Intralipid(®) was more effective with regard to survival than Intralipid(®) alone and vasopressin+Intralipid(®) in this pig model [14]. Local anesthetic (LA) intoxication with severe hemodynamic compromise is a potential catastrophic event. Lipid resuscitation has been recommended for the treatment of LA-induced cardiac arrest. However, there are no data about effectiveness of Intralipid for the treatment of severe cardiovascular compromise prior to cardiac arrest. Aim of this study was to compare effectiveness of epinephrine and Intralipid for the treatment of severe Hemodynamic compromise owing to bupivacaine intoxication, anesthetized Piglets were with sevoflurane, intubated, and ventilated. Bupivacaine was infused with a syringe driver via a central venous catheter at a rate of 1 mg·kg-1·min-1 until invasively measured mean arterial pressure (MAP) dropped to 50% of the initial value. Bupivacaine infusion was then stopped, and epinephrine 3 μg·μkg-1(group 1), Intralipid(®) 20% 2 ml·kg-1 (group 2), or

Intralipid 20% 4 ml·kg-1 (group 3) was immediately administered. Twenty-one piglets (3×7), were recorded. All animals in group 1 (100%) but only four of seven (57%) piglets in group 2 and group 3, respectively, survived. Normalization of hemodynamic parameters (HR, MAP) and ET(CO₂) was fastest in group 1 with all piglets achieving HR and MAP values. hemodynamic compromise owing to bupivacaine intoxication in piglets, first-line rescue with epinephrine was more effective than Intralipid with regard to survival as well as normalization of hemodynamic parameters and ET(CO₂) [15]. Intravenous lipid emulsion (ILE) has been proposed as a rescue therapy for severe local anesthetic drugs toxicity, but experience is limited with other lipophilic drugs. An 18-year-old healthy woman was admitted 8 h after the voluntary ingestion of sustained-release diltiazem (3600 mg), with severe hypotension refractory to fluid therapy, calcium salts, and high-dose norepinephrine (6.66 μg/kg/min). Hyperinsulinemic euglycemia therapy was initiated and shortly after was followed by a protocol of ILE (intralipid 20%, 1.5 ml/kg as bolus, followed by 0.25 ml/kg over 1 h). The main finding attributed to ILE was an apparent rapid decrease in insulin resistance, despite a prolonged serum diltiazem elimination half-life. Diltiazem is a lipophilic cardiotoxic drug, which could be sequestered in an expanded plasma lipid phase. The mechanism of action of ILE is not known, including its role in insulin resistance and myocardial metabolism in calcium-channel blocker poisoning [16].

### Conclusion

The use of intravenous intralipid infusion instead of thrombolysis or primary percutaneous coronary intervention is first suggested in the medical literature.

### REFERENCES

1. Siamak R, Jingyuan L, Jean C B, Soban U, Andrea I, et al. (2011) Phosphorylation of GSK-3β Mediates Intralipid-induced Cardioprotection against Ischemia/Reperfusion Injury. Anesthesiol 115: 242-253.
2. Li J, Iorga A, Sharma S, Youn J Y, Partow-Navid R, et al. (2012) Intralipid, a clinically safe compound, protects the heart against ischemia-reperfusion injury more efficiently than cyclosporine-A. Anesthesiol 117: 836-846.
3. Li J, Ruffenach G, Kararigas G, Cunningham C M, Motayagheni N, et al. (2017) Intralipid protects the heart in late pregnancy against ischemia/reperfusion injury via Caveolin2/STAT3/GSK-3Î² pathway. J Mol Cell Cardiol 102: 108-116.
4. Ma L L, Ge H W, Kong F J, Qian L B, Hu B C, et al. (2014) Ventricular hypertrophy abrogates intralipid-induced cardioprotection by alteration of reperfusion injury salvage kinase/glycogen synthase kinase 3β signal. Shock 41: 435-442.
5. Cheng K H, Chu C S, Lin T H, Lee K T, Sheu S H, et al. (2015) Lipid paradox in acute myocardial infarction—the association with 30-day in-hospital mortality. Crit Care Med 43: 1255-1264.
6. Hu Z Y, Peng X Y, Liu F, Liu J (2014) Emulsified isoflurane protects rat heart in situ after regional ischemia and reperfusion. Fundam Clin Pharmacol 28:190-198.
7. Huang H, Zhang W, Liu S, Yanfang C, Li T, Liu J (2010) Cardioprotection afforded by St Thomas solution is enhanced by emulsified isoflurane in an isolated heart ischemia reperfusion injury model in rats. J Cardiothorac Vasc Anesth 24: 99-103.
8. Hu Z Y, Luo N F, Liu J (2009) The protective effects of emulsified isoflurane on myocardial ischemia and reperfusion injury in rats. Can J Anaesth 56: 115-125.
9. Hu Z Y, Liu J (2009) Effects of emulsified isoflurane on haemodynamics and cardiomyocyte apoptosis in rats with myocardial ischaemia. Clin Exp Pharmacol Physiol 36: 776-783.
10. Rao Y, Wang Y L, Chen Y Q, Zhang W S, Liu J (2009) Protective effects of emulsified isoflurane after myocardial ischemia-reperfusion injury and its mechanism in rabbits. Chin J Traumatol 12:18-21.
11. Rao Y, Wang Y L, Zhang W S, Liu J (2008) Emulsified isoflurane produces cardiac protection after ischemia-reperfusion injury in rabbits. Anesth Analg 06:1353-1359.
12. Papadopoulou A, Willers J W, Samuels T L, Uncles D R (2012) The Use of Dye Surrogates to Illustrate Local Anesthetic Drug Sequestration by Lipid Emulsion: A Visual Demonstration of the Lipid Sink Effect. Regional Anesthesia & Pain Medicine 37: 183-187.
13. Grunbaum A M, Gilfix B M, Gosselin S, Blank D W (2012) Analytical Interferences Resulting from Intravenous Lipid Emulsion. Clinical Toxicology 50: 812-817.
14. Mauch J, Jurado O M, Spielmann N, Bettschart R W, Weiss M (2012) Resuscitation Strategies from Bupivacaine-Induced Cardiac Arrest, Pediatric Anesthesia 22: 124-129.
15. Mauch J, Jurado O M, Spielmann N, Bettschart R W, Weiss M (2011) Comparison of Epinephrine vs Lipid Rescue to Treat Severe Local Anesthetic Toxicity. An Experimental Study in Piglets. Pediatric Anesthesia 21: 1103-1108.
16. Montiel V, Gougnard T, Hantson P (2011) Diltiazem Poisoning Treated with Hyperinsulinemic Euglycemia Therapy and Intravenous Lipid Emulsion. European Journal of Emergency Medicine 18: 121-123.

### Example 7

### Intralipid Use in Trauma

Hemorrhage is responsible for most deaths that occur during the first few hours after trauma. Current evidence indicates that initial liberal fluid resuscitation strategies may be associated with higher mortality in injured patients. In the pre hospital arena, intravenous fluids have been associated with worse patient outcomes due to increased coagulopathy and time to definitive care. Once in the trauma bay, damage control resuscitation principles apply to the severely injured patient. Large volume crystalloid infusion increases mortality. The best patient outcomes have been found with transfusion of blood products in a ratio that closely mimics whole blood.

Intralipid increased renal blood flow, carotid vascular resistance and mesenteric vascular resistance. In the presence of Intralipid, L-NMA-induced pressor response and systemic, carotid and renal vasoconstriction were more pronounced than in control dogs. Except for the coronary and carotid circulations, Intralipid modulates the NO pathway in cardiac and regional blood flow. Intralipid infusion in trauma patients is first suggested in the medical literature.

The use of Intralipid therapy has been gaining traction as a treatment option for an ever expanding range of toxicities. Veterinary literature has reviewed intravenous lipid emulsion therapy (ILE) [1, 2] and published case reports or studies are available noting efficacy in toxicities including macro cyclic lactones [3, 4], baclofen [5], beta-blockers, calcium channel blockers [6], NSAID [7, 8], bromethalin [9], lidocaine [10], permethrin toxicity [11,12], tricyclic antidepressants [13].

Intravenous lipid emulsion (ILE) in human literature has been reported as a therapy for local anesthetic [14, 15], calcium channel blocker [16, 17], psychotropic medication [18], glyphosatesurfactant herbicide toxicities [19] and even cocaine over dosage [20].

Original work performed by Weinberg noted a response in rats with bupivacaine induced asystole with lipid emulsion [21]. How exactly ILE works is not certain but two theories are considered. The “lipid sink” theory is most commonly considered the primary mode of action. In this theory, the formation of a lipid compartment within the intravascular space can serve as a “sink” into which the lipophilic drug will be drawn into. The drug is then excreted/metabolized. Determination of a drug's lipophilicity may be noted by its log P value. A value >1 indicates lipophilic compound which may move into the temporary lipid phase and be less distributed throughout the body. The formulation of ILE utilized may play a role and supports the “lipid-sink” theory based on one study [22].

This theory has been supported in two case reports that followed plasma ropivacaine [15] and serum verapamil concentrations [17]

An alternate theory is that the lipid provides an energy source for the cardiac myocytes by increasing the availability of FFA. The increase of FFA may also aid in increasing the activation of voltage-gated calcium channels in the myocardium, increasing cytosolic calcium channels This mechanism may be most important in cases of calcium-channel blockade [23, 24].

### Intralipid Use in Rapid Blood Loss

The following were studied in a perimortem mouse model of rapid blood loss: (a) efficacy of a prototypical micellar colloid, Intralipid 20%, (IL20), compared to albumin (b) comparison of intra-arterial and intravenous resuscitation, (c) efficacy of IL20 at a volume 2×the volume of blood removed, and (d) efficacy of oxygenated IL20 after clinical death (CD). CD, the absence of breathing and zero blood pressure (BP), was produced by removing 55% of the blood volume within 3 minutes. After CD, the chest was opened to observe ventricular contraction. IL20, Ringer's lactate (RL), or albumin was infused perimortem.

Without resuscitation CD occurred in 2.85+/−0.40 minutes. Ventricular contraction persisted 20.50+/−1.11 minutes after CD. RL infused immediately after CD restored breathing if given intra-arterially but not intravenously. IL20 was superior to the prototypical colloid, albumin in maintaining the BP. Increasing the volume of IL20 further increased BP. Delayed RI, infusion after CD failed to restore breathing. Delayed resuscitation after CD with oxygenated IL20 restored breathing and BP.

Micellar colloid is superior to the prototypical colloid albumin and can possibly be of use when signs of life are no longer present. In extremis, intra-arterial infusion is superior to intravenous infusion [25].

Fat emulsions, Intralipid 30% and Intralipid 10% were compared in terms of the resulting plasma levels of different lipid components and clinical tolerance in critically-ill patients with multi-injuries. Sixteen criticallyill patients with severe systemic inflammatory response were randomly assigned to two groups, each one comprised of eight patients. Each group was administered the same quantity of fat/Kg/day either Intralipid 30% or Intralipid 10%. The infusion lasted 12 h daily for 6 days.

During the infusion of the fat emulsion, a lower median plasma concentration of triglycerides, phospholipids and free cholesterol was observed in patients who received Intralipid 30% compared with those who received Intralipid 10%. The above observations were sustained 4 h after the termination of the infusion.

Free fatty acids had a higher mean plasma concentration in the group of patients who received Intralipid 30%. There were no differences between the two groups as far as the median plasma concentration of cholesterol and lipoproteins (LDL, HDL, VLDL) are concerned. On the contrary, there was an increase in LpX in the Intralipid 10% group. From the above findings, we draw the conclusion that Intralipid 30% revealed better profiles of different lipid components than Intralipid 10% in critically-ill patients. The new emulsion of higher concentration in triglyceride was proved clinically safe and its use is suggested for critically-ill patients who require total parenteral nutrition [26].

### Intralipid Modulates the NO Pathway in Cardiac and Regional Blood Flow

The commercial propofol preparation in an Intralipid solution causes marked vasodilatation. Both propofol and its solvent seem to stimulate the nitric oxide (NO) pathway. The role of Intralipid in cardiac and regional hemodynamic changes induced by propofol and their respective interactions with the NO pathway was assessed.

Dogs were instrumented to record arterial pressure, heart rate, cardiac output, dP/dt (the first derivative of left ventricular pressure) and vertebral, carotid, coronary, mesenteric, hepatic, portal and renal blood flows. Experimental groups were as follows. Group 1 (control; n=11): N-methyl-L-arginine (L-NMA) 20 mg kg-1 i.v.; Group 2 (n=8): propofol (10 mg ml-1) 4 mg kg-1 i.v. bolus followed by 0.6 mg kg-1 min-1; Group 3 (n=6): Intralipid 0.25 ml kg-1 bolus followed by 0.06 ml kg-1 min-1. After 60 min, L-NMA was injected in Groups 2 and 3.

Propofol induced increases in heart rate, coronary and carotid blood flows, and decreases in systemic vascular resistance and dP/dt. Intralipid increased renal blood flow, carotid vascular resistance and mesenteric vascular resistance. In the presence of Intralipid, L-NMA induced pressor response and systemic, carotid and renal vasoconstriction were more pronounced than in control dogs.

Except for the coronary and carotid circulations, Intralipid modulates the NO pathway in cardiac and regional blood flow [27].

Propofol is a commonly used anesthetic. Despite its favorable safety profile, propofol causes hypotension which can result in end-organ hypo perfusion. Intralipid is a lipid emulsion that has been shown to reverse the vasodilatory effects of propofol in isolated vessels; however, whether these effects are recapitulated in vivo is not known. The objectives of this study were to determine if Intralipid reverses the hypotensive and anesthetic effects of propofol in rats. Under isoflurane anesthesia, male Sprague Dawley rats were instrumented with indwelling catheters for mean arterial pressure (MAP) assessments as well as subdural electrodes for cortical activity assessments by electroencephalography (EEG). Propofol (10 mg/kg IV) caused hypotension (55+/−2% drop in MAP, P<0.001) and Intralipid (4 mL/kg IV) caused greater reversal (80+/−9%) of blood pressures compared to saline (19+/−1%; P<0.001).

Blockade of the autonomic nervous system with chlorisondamine (2.5 mg/kg IV) caused marked hypotension (56+/−3% lowering of MAP, P<0.001) which could be reversed with a constant infusion of phenylephrine (300 μg/kg/hr); under these conditions, propofol nevertheless caused hypotension (12+/−4% lowering of MAP) which was completely reversed by Intralipid. Propofol induced cortical burst suppression was not affected by Intralipid (2+/−3%), saline (−4%) or 20% BSA (−2+/−1%; P=0.27). These results demonstrate that Intralipid reverses propofol-mediated hypotension with minimal effects on its anesthetic profile. Intralipid could be particularly useful as a rescue against propofol in patients prone to hemodynamic instability such as the elderly [28].

Sepsis is one of the most serious complications that can occur during total parenteral nutrition (TPN) procedures. In this experimental study, we investigated the effects of TPN, with or without lipid emulsion, on vascular endothelial damage.

In total, 50 rabbits were used, divided into 5 groups of 10 each. TPN with lipids (group 1), TPN without lipids (group 2), and 0.09% saline (group 3) were given for 10 days via a central venous catheter. Group 4 received no treatment other than placement of a central venous catheter for 10 days. Group 5 was a control group. At the end of day 10, rabbits were sacrificed and tissue samples of liver, kidney, and inferior vena cava were prepared and examined by immunohistochemical methods for vascular cellular adhesion molecule (VCAM)-1 expression.

In tissue sections of liver, kidney, and inferior vena cava, VCAM-1 activity was increased prominently in TPN with and without lipids compared with the control group. VCAM-1 activity in the TPN with lipids group was decreased versus the TPN without lipids group (P>0.05).

The TPN procedure results in vascular endothelial cell damage not only in the vein where the solution is introduced but also in other parts of the vascular system. Even if it is not statistically significant, lipids in the TPN formula may decrease this endothelial cell damage, as shown by immunohistochemistry [29].

Ischemia-reperfusion injury is a determinant in liver injury occurring during surgical procedures, ischemic states, and multiple organ failure. The pre-existing nutritional status of the liver, i.e., fasting, might contribute to the extent of tissue injury. This study investigated whether Intralipid, a solution containing soybean oil, egg phospholipids, and glycerol, could protect ex vivo perfused livers of fasting rats from anoxia-reoxygenation injury.

The portal vein was cannulated, and the liver was removed and perfused in a closed ex vivo system. Isolated livers were perfused with glucose 5.5 and 15 mM, and two different concentrations of Intralipid, i.e., 0.5:100 and 1:100 (v/v) Intralipid 10%:medium (n=5 in each group). The experiment consisted of perfusion for 15 min, warm anoxia for 60 min, and reoxygenation during 60 min. Hepatic enzymes, potassium, glucose, lactate, bilirubin, dienes, trienes, and cytochrome-c were analyzed in perfusate samples. The proportion of glycogen in hepatocytes was determined in biopsies.

Intralipid attenuated transaminases, lactate dehydrogenase, potassium, diene, and triene release in the perfusate (dose-dependent) during the reoxygenation phase when compared with glucose-treated groups. The concentration of cytochrome-c in the medium was the highest in the 5.5-mM glucose group. The glycogen content was low in all livers at the start of the experiment.

Intralipid presents, under the present experimental conditions, a better protective effect than glucose in anoxiareoxygenation injury of the rat liver [30].

### The Effect of Intralipid on Protein Consumption in Severe Burned Patients

Rong et al. [31] investigated the effect of Intralipid on protein consumption in severe burned patients. Sixtyseven non operative patients with severe burns were divided into Intralipid treatment group and non-Intralipid treatment group (control group), and the former was treated with 20% Intralipid (500 ml once a day) from post burn day 4 for 10 consecutive days. Venous blood samples were collected from these patients for testing total protein, albumin, total cholesterol and triglyceride on post burn days 1, 7 and 14, respectively.

The levels of total protein, albumin, total cholesterol and triglyceride were within normal range on post burn day 1 in both groups, and only the albumin level was lowered in the groups on day 7 but at comparable magnitudes (32+/−4.83 vs. 31+/−5.04 g/L, P<0.05). In contrast, the levels of total protein, albumin, total cholesterol and triglyceride were below the normal range on post burn day 14 in both groups, but Intralipid treatment group showed more albumin loss than the control group (28+/−6.46 vs 23+/−7.03 g/L, P<0.01).

Intralipid (20%) provides good energy source to ameliorate albumin loss in severe burned patients [31].

A concentrated fat emulsion (Intralipid 30%) with a phospholipid/triglyceride ratio of 0.04 was tested for clinical tolerance and metabolic effects in the short term parenteral nutrition of septic and trauma critically ill patients and compared with Intralipid 20% (phospholipid/triglyceride ratio of 0.06).

This was a prospective, randomized, multicenter study in the intensive care units in 10 university hospitals, including 90 adult patients in 2 groups: 55 septic and 35 trauma patients. Patients in each group were randomly divided into 2 subgroups according to the fat emulsions administered (1.4 g/kg per day) as part of the calories for at least 6 days of continuous total parenteral nutrition (TPN). One subgroup was treated with 30% long-chain triglycerides (phospholipid/triglyceride ratio: 0.04) and the other with 20% long-chain triglycerides (phospholipid/triglyceride ratio: 0.06). The parenteral nutrition formula was isocaloric and isonitrogenous with 0.25 g of nitrogen/kg per day and 40% of the non protein calories as fat. Clinical tolerance was assessed during the study. At baseline and after 3 and 6 days of TPN, the following biochemical parameters were measured: prealbumin, retinol-binding protein, serum albumin, hematologic, hepatic and renal function variables, triglycerides, phospholipids, total and free cholesterol, nonesterified cholesterol, nonesterified fatty acids, and lipoproteins.

At baseline, no differences in age, gender, severity of the condition [Acute Physiology and Chronic Health Evaluation (APACHE II) score], or clinical chemistry were found between the subgroups. The levels of plasma proteins studied and the renal, hematologic, or hepatic function variables did not vary during the study period. Total cholesterol increased significantly, owing to esterified cholesterol, with 20% long-chain triglyceride in septic patients (baseline: 2.1+/−0.8 mmol/L, day 6: 2.8+/−0.6 mmol/L, p=0.026). In septic patients receiving 20% long-chain triglycerides, plasma triglycerides had a similar behavior (baseline: 1.4+/−0.6 mmol/L, day 3: 2.2+/−0.8 mmol/L, p<0.05). The very-low-density lipoprotein content of cholesterol, triglycerides, and phospholipids showed a tendency to decrease in septic patients treated with 30% long-chain triglycerides (NS). None of the emulsions induced the synthesis of lipoprotein X.

The results indicate that while both fat emulsions used in the TPN of critically ill patients are clinically safe, the 30% long-chain triglyceride fat emulsion with a phospholipid/triglyceride ratio of 0.04 causes fewer lipid metabolic disturbances [32].

A 4-year-old boy with an abdominal trauma had lipiduria following treatment with total intravenous nutrition for 4 days.

Renal function was normal throughout the course and the lipiduria ceased after withdrawal of the intravenous nutrition. The lipids were possibly excreted through the kidneys or entered the urine through a traumatic communication between the lymphatic vessels and the urinary system. Control of renal function and lipiduria after 1 year revealed normal conditions [33].

### Current Fluid Resuscitation and the Vasopressor Support in Severe Trauma Patients

Harrois et al. [34] discuss the fluid resuscitation and the vasopressor support in severe trauma patients.

A critical point is to prevent a potential increase in bleeding by an overly aggressive resuscitative strategy. Indeed, large-volume fluid replacement may promote coagulopathy by diluting coagulation factors. Moreover, an excessive level of mean arterial pressure may induce bleeding by preventing clot formation.

Fluid resuscitation is the first-line therapy to restore intravascular volume and to prevent cardiac arrest.

Thus, fluid resuscitation before bleeding control must be limited to the bare minimum to maintain arterial pressure to minimize dilution of coagulation factors and complications of over fluid resuscitation. However, a strategy of low fluid resuscitation needs to be handled in a flexible way and to be balanced considering the severity of the hemorrhage and the transport time. A target systolic arterial pressure of 80-90 mmHg is recommended until the control of hemorrhage in trauma patients without brain injury. In addition to fluid resuscitation, early vasopressor support may be required to restore arterial pressure and prevent excessive fluid resuscitation. It is crucial to find the best alchemy between fluid resuscitation and vasopressors, to consider hemodynamic monitoring and to establish trauma resuscitative protocols [34].

The ideal strategy for prehospital intravenous fluid resuscitation in trauma remains unclear. Fluid resuscitation may reverse shock but aggravate bleeding by raising blood pressure and haemodilution. We examined the effect of prehospital i.v. fluid on the physiologic status and need for blood transfusion in hypotensive trauma patients after their arrival in the emergency department (ED).

Retrospective analysis of trauma patients (n=941) with field hypotension presenting to a level 1 trauma centre.

Regression models were used to investigate associations between prehospital fluid volumes and shock index and blood transfusion respectively in the emergency department and mortality at 24 h.

A 1 L increase of prehospital i.v. fluid was associated with a 7% decrease of shock index in the emergency department (p<0.001). Volumes of 0.5-1 L and 1-2 L were associated with reduced likelihood of shock as compared to volumes of 0-0.5 L: OR 0.61 (p=0.03) and OR 0.54 (p=0.02), respectively. Volumes of 1-2 L were also associated with an increased likelihood of receiving blood transfusion in ED: OR 3.27 (p<0.001). Patients who had received volumes of >2 L have a much greater likelihood of receiving blood transfusion in ED: OR 9.92 (p<0.001). Mortality at 24 h was not associated with prehospital i.v. fluids.

In hypotensive trauma patients, prehospital i.v. fluids were associated with a reduction of likelihood of shock upon arrival in ED. However, volumes of >1 L were associated with a markedly increased likelihood of receiving blood transfusion in ED. Therefore, decision making regarding prehospital i.v. fluid resuscitation is critical and may need to be tailored to the individual situation. Further research is needed to clarify whether a causal relationship exists between prehospital i.v. fluid volume and blood transfusion. Also, prospective trials on prehospital i.v. fluid resuscitation strategies in specific patient subgroups (e.g. traumatic brain injury and concomitant haemorrhage) are warranted [35].

Feinman et al. [36] explore the recent literature regarding the optimal type and amount of intravenous fluids for the trauma patient from the time of injury through their ICU stay. It discusses damage control principles as well as targeted resuscitation utilizing new technology.

In the prehospital arena, intravenous fluids have been associated with worse patient outcomes due to increased coagulopathy and time to definitive care. Once in the trauma bay, damage control resuscitation principles apply to the severely injured patient. Large volume crystalloid infusion increases mortality. The best patient outcomes have been found with transfusion of blood products in a ratio that closely mimics whole blood. Thrombelastography is a useful adjunct in resuscitation and can help guide the judicious use of blood products. New technology can help providers ascertain when a patient is appropriately resuscitated by determining adequate global and regional perfusion.

During the resuscitation of the acutely injured patient, crystalloids should be limited in favor of blood components.

Damage control principles apply until definitive hemostasis is obtained, at which point the focus should change to targeted resuscitation using traditional global endpoints of resuscitation in conjunction with determinants of regional perfusion [36].

Hemorrhage is responsible for most deaths that occur during the first few hours after trauma. Animal models of trauma have shown that restricting fluid administration can reduce the risk of death; however, studies in patients are difficult to conduct due to logistical and ethical problems. To maximize the value of the existing evidence, we performed a meta-analysis to compare liberal versus restricted fluid resuscitation strategies in trauma patients. Medline and Embase were systemically searched from inception to February 2013.

We selected randomized controlled trials and observational studies that compared different fluid administration strategies in trauma patients. There were no restrictions for language, population, or publication year.

Four randomized controlled trials and seven observational studies were identified from 1,106 references. One of the randomized controlled trials suffered from a high protocol violation rate and was excluded from the final analysis.

The quantitative synthesis indicated that liberal fluid resuscitation strategies might be associated with higher mortality than restricted fluid strategies, both in randomized controlled trials (risk ratio, 1.25; 95% CI, 1.01-1.55; three trials; I(2), 0) and observational studies (odds ratio, 1.14; 95% CI, 1.01-1.28; seven studies; I(2), 21.4%). When only adjusted odds ratios were pooled for observational studies, odds for mortality with liberal fluid resuscitation strategies increased (odds ratio, 1.19; 95% CI, 1.02-1.38; six studies; I (2), 26.3%).

Current evidence indicates that initial liberal fluid resuscitation strategies may be associated with higher mortality in injured patients. However, available studies are subject to a high risk of selection bias and clinical heterogeneity. This result should be interpreted with great caution [37].

Massive transfusion protocol seems to improve outcome in massively bleeding trauma patients, but not pelvic fracture patients. The aim of this study was to evaluate the effect of massive transfusion protocol on the mortality and fluid resuscitation of shocked pelvic fracture patients.

This is a trauma register study from a single hospital. From the trauma registry patients with pelvic fracture, injury severity score >15, admission base excess below −5, age >15 years, blunt trauma, and primary admission from the scene were identified. Patients were divided into two groups: Group 1-pre-massive transfusion protocol (2006-2009) and Group 2-post-massive transfusion protocol (2010-2013). Basic characteristics and intensive care unit length of stay, mortality, and fluid resuscitation data were retrieved from the registry. Standardized mortality ratio was assessed using revised injury severity classification, version II methodology.

Altogether, 102 patients were identified. Group 1 (n=56) and Group 2 (n=46) were comparable in their basic characteristics. The observed mortality was 35.7% and 26.1% in Groups 1 and 2, respectively. The standardized mortality ratio failed to reveal any difference between observed and expected mortality in either group. In the emergency room, the use of crystalloids decreased from 5.3+/−3.4 to 3.3+/−1.8 L (p=0.002) with increased use of fresh frozen plasma (2.9+/−4.4 vs 5.1+/−5.3, p=0.007).

No improvement in the adjusted survival of shocked pelvic fracture patients is apparent after implementation of massive transfusion protocol. Implementation of massive transfusion protocol is associated with a higher use of fresh frozen plasma and improved ratio of fresh frozen plasma:red blood cell toward the targeted 1:1 and decreased use of crystalloids [38].

### Conclusion

Intralipid increased renal blood flow, carotid vascular resistance and mesenteric vascular resistance. In the presence of Intralipid, L-NMA-induced pressor response and systemic, carotid and renal vasoconstriction were more pronounced than in control dogs.

Except for the coronary and carotid circulations, Intralipid modulates the NO pathway in cardiac and regional blood flow.

Intralipid infusion in trauma patients is first suggested in the medical literature.

### REFERENCES

1. Fernandez A L, Lee J A, Rahilly L, et al. (2011) Use of intravenous lipid emulsion as an antidote in Veterinary toxicology. J Vet Emerg Crit Care 21: 309-320.
2. Gwaltney-Brant S, Meadows I (2012) Use of intravenous lipid emulsion for treating certain poisoning cases in small animals. Vet Clie North Am Small Animal Pract 42: 251-262.
3. Clarke D L, Lee J A, Murphy L A, et al. (2011) Use of intravenous lipid emulsion to treat ivermectin toxicosis in a Border Collie. J Am Vet Med Assoc 239: 1328-1333.
4. Epstein S E, Hollingsworth S R (2013) Ivermectin-induced blindness treated with intravenous lips therapy in a dog. J Vet Emerg Crit Care (San Antonio) 23: 58-62.
5. Bates N, Chatterton J, Robbins C, et al. (2013) Lipid infusion in the management of poisoning: a report of 6 canine cases. Vet Rec 172: 339.
6. Maton B L, Simonds E E, Lee J A, et al. (2013) Use of highdose insulin therapy and intravenous lipid Emulsion to treat severe, refractory diltiazem overdose in a dog. J Vet Emerg Care 23: 321-327.
7. Herring J M, McMichael M A, Corsi R, et al. (2015) Intravenous lipid emulsion therapy in three cases of canine naproxen overdose. J Vet Emerg Crit Care (San Antonio) 25: 672-678.
8. Bolfer L, McMichael M, Ngwenyama T R, et al. (2014) Treatment of ibuprofen toxicosis in a dog with IV lipid emulsion. J AM Anim Host Assoc 50: 136-140.
9. Heggem-Perry B, McMichael M, O'Brien M, et al. (2016) Intravenous lipid emulsion therapy for bromethalin toxicity in a dog. J Am Anim Hosp Assoc 52: 265-268.
10. O'Brien T Q, Clark-Price S C, Evans E E, et al. (2010) Infusion of a lipid emulsion to treat lidocaine intoxication in a cat. J Am Vet Med Assoc 237: 1455-1458.
11. Peacock R, Hosgood G, Swindells K L, et al. (2015) Randomized, controlled clinical trial of intravenous lipid emulsion as an adjunctive treatment for permethrin toxicosis in cats. J Vet Emerg Crit Care 25: 597-605.
12. Seitz M A, Burkitt-Creedon J M (2016) Persistent gross lipemia and suspected corneal lipidosis following intravenous lipid therapy in a cat with permethrin toxicosis. J Vet Emerg Crit Care 26: 804-808.
13. Cave G, Harvey M, Shaw T, et al. (2013) Comparison of intravenous lipid emulsion, bicarbonate, and tailored liposomes in rabbit clomipramine toxicity. Acad Emerg Med 20: 1076-1079.
14. Litz R J, Roessel T, Heller A R, et al. (2008) Reversal of central nervous system and cardiac toxicity after local anesthetic intoxication by lipid emulsion injection. Anesth Analg 106: 1575-1577.
15. Mizutani K, Oda Y, Sato H. (2011) Successful treatment of ropivacaine-induced central nervous System toxicity by use of lipid emulsion: effect on total and unbound plasma fractions. J Anesth 25: 442-445.
16. Young A C, Velez L I, Kleinschmidt K C (2009) Intravenous fat emulsion therapy for intentional sustained-release verapamil overdose. Resuscitation 80: 591-593.
17. French D Armenian P, Ryan W, et al. (2011) Serum verapamil concentrations before and after Intralipid therapy during treatment of overdose. Clin Toxicol (Phila) 49: 340-344.
18. Nair F, Paul F K, Protopapas M (2013) Management of near fatal mixed tricyclic antidepressant and selective serotonin reuptake inhibitor overdose with Intralipid 20% emulsion. Aneasth Intensive Care 41: 264-265.
19. Han S K, Jeong J, Yeom S, et al. (2010) Use of a lipid emulsion in a patient with refractory hypotension caused by glyphosatesurfactant herbicide. Clin Toxicol (Phila) 48: 566-568.
20. Jakkala-Saibaba R, Morgan P G, Morton G L (2011) Treatment of cocaine overdose with lipid Emulsion. Anesthesia 66: 1168-1170.
21. Weinberg G L, VadeBoncouer T, Ramaraju G A, et al. (1998) Pretreatment or resuscitation with a Lipid infusion shifts the doseresponse to bupivicaine-induced asystole in rats. Anesthesiology 88: 1071-1075.
22. Mazoit J X, Le Guen R, Beloeil H, et al. (2009) Binding of long-lasting local anesthetics to lipid Emulsions. Anesthesiology 110: 380-386.
23. Turner-Lawrence D E, Kerns W I (2008) Intravenous fat emulsion: a potential novel antidote. J Med Toxicol 4: 109-114.
24. Rothschild L, Bern S, Oswald S, et al. (2010) Intravenous lipid emulsion in clinical toxicology. Scan J Trauma Resusc Emerg Med 18: 51.
25. Simpkins C, Talluri K, Williams M (2016) Intra-Arterial Perimortem Resuscitation Using a Micellar Colloid. Mil Med 181: 253-258.
26. Kalfarentzos F, Kokkinis K, Leukaditi K, et al. (1998) Comparison between two fat emulsions: Intralipid 30 cent vs. Intralipid 10 cent in critically ill patients. Clin Nutr 17: 31-34.
27. Doursout M F, Joseph P M, Liang Y Y, et al. (2002) Role of propofol and its solvent, Intralipid, in nitric oxide-induced peripheral vaso dilatation in dogs. Br J Anaesth 89: 492-498.
28. Stephane Bourque, Sareh Panahil, Rohan Minal1, et al. (2015) Intralipid Reverses Propofol Mediated Hypotension in Rats. The FASEB Journal 29: 624-633.
29. Demircan M, Gurunluoglu K, Karaman A, et al. (2015) Damaging Effects of Total Parenteral Nutrition Formula on Vascular Endothelium. J Pediatr Gastroenterol Nutr 61: 464-468.
30. Stadler M, Nuyens V, Boogaerts J G (2007) Intralipid minimizes hepatocytes injury after anoxiareoxygenation in an ex vivo rat liver model. Nutrition 23: 53-61.
31. Rong X Z, Zhang T, Li Q H, et al. (2006) Effect of Intralipid for ameliorating protein loss in severe burned patients. Nan Fang Yi Ke Da Xue Xue Bao 26: 500-501.
32. Garcia-de-Lorenzo A, Lopez-Martinez J, Planas M, et al. (2003) Safety and metabolic tolerance of a concentrated longchain triglyceride lipid emulsion in critically ill septic and trauma patients. JPEN J Parenter Enteral Nutr 27: 208-215.
33. Nielsen H K, Lybecker H (1991) Lipiduria after Intralipid infusion of a lipid emulsion in a boy with an abdominal trauma. JPEN J Parenter Enteral Nutr 15: 572-573.
34. Harrois A, Hamada S R, Duranteau J (2014) Fluid resuscitation and vasopressors in severe trauma patients. Curr Opin Crit Care 20: 632-637.
35. Geeraedts L M Jr, Pothof L A, Caldwell E, et al. (Prehospital fluid resuscitation in hypotensive trauma patients: do we need a tailored approach? Injury 46: 4-9.
36. Feinman M, Cotton B A, Haut E R (2014) Optimal fluid resuscitation in trauma: type, timing, and Total. Curr Opin Crit Care 20: 366-372.
37. Wang C H, Hsieh W H, Chou H C, et al. (2014) Liberal versus restricted fluid resuscitation strategies in trauma patients: a systematic review and meta-analysis of randomized controlled trials and observational studies. Crit Care Med 42: 954-961.
38. Soderlund T, Ketonen T, Handolin L (2017) Bleeding Pelvic Fracture Patients: Evolution of Resuscitation Protocols. Scand J Surg 1-6.

### Example 8

### Intralipid-Iodine for Hysterosalpingography in Infertile Women

Tubal disease is the cause of female infertility in approximately 30% of women and 10-25% of these are due to proximal tubal obstruction. Selective salpingography represent an approach in the diagnosis and treatment of proximal tubal abnormalities. Intralipid (IL) is a synthetic product composed of 10% soybean oil, 1.2% egg yolk phospholipids, 2.25% glycerin, and water. When indicated, IL is infused 7-10 days prior to embryo transfer (ET), Rates of ongoing pregnancy and live births were higher among women who underwent hysterosalpingography with oil contrast than among women who underwent this procedure with water contrast. It is the first time that intralipid-iodine is suggested to be used for hysterosalpingography in infertile women.

Tubal disease is the cause of female infertility in approximately 30% of women [1] and 10-25% of these are due to proximal tubal obstruction [2]. Selective salpingography represent an approach in the diagnosis and treatment of proximal tubal abnormalities. The common indications for selective salpingography are to differentiate spasm from true obstruction [3]. In addition to that it allows clarifying findings from an equivocal hysterosalpingogram. Isthmic as well as intramural blockages were included. The tubal obstruction may due to amorphous materials occluding the tubal lumen, inflammatory changes and adhesions [4]. The use of selective salpingography and fallopian tube recanalization has revolutionized the diagnosis and treatment of infertility [3]. Diagnostic procedure has been used since 1980 [3]. It consists on opacification of the fallopian tube directly through a catheter placed in the tubal ostium. The objective is to differentiate spasm from true obstruction and to clear it with a catheter and guide wire system.

Pregnancy Rates after Hysterosalpingography

Pregnancy rates among infertile women have been reported to increase after hysterosalpingography, but it is unclear whether the type of contrast medium used (oil-based or water-soluble contrast) influences this potential therapeutic effect.

Dreyer et al. performed a multicenter, randomized trial in 27 hospitals in the Netherlands in which infertile women who were undergoing hysterosalpingography were randomly assigned to undergo this procedure with the use of oil-based or water-based contrast [5]. Subsequently, couples received expectant management, or the women underwent intrauterine insemination. The primary outcome was ongoing pregnancy within 6 months after randomization. Outcomes were analyzed according to the intention-to-treat principle.

A total of 1119 women were randomly assigned to hysterosalpingography with oil contrast (557 women) or water contrast (562 women). A total of 220 of 554 women in the oil group (39.7%) and 161 of 554 women in the water group (29.1%) had an ongoing pregnancy (rate ratio, 1.37; 95% confidence interval [CI], 1.16 to 1.61; P<0.001), and 214 of 552 women in the oil group (38.8%) and 155 of 552 women in the water group (28.1%) had live births (rate ratio, 1.38; 95% CI, 1.17 to 1.64; P<0.001). Rates of adverse events were low and similar in the two groups.

Fluoroscopy with image intensification gives the best results. Insufficient dye injection will give an incomplete study. Too much dye injection, especially under pressure, might cause extravasation of the dye into the vascular system or conceal the fimbrial ends of the tubes [6].

Hysterosalpingography (HSG) Increases the Conception Rate
<=““p=”” style=“box-sizing: border-box;”>

Hysterosalpingography (HSG) Using Oil as Compared with Aqueous Contrast Medium

Previous studies have suggested increased fertility rates following hysterosalpingography (HSG) using oil as compared with aqueous contrast medium. To compare the possible fertility-enhancing effects of two agents used for HSG, this prospective randomized study evaluated the subsequent fertility rates in 121 patients who underwent HSG, in which either oil or aqueous contrast medium was used. After random assignment to either agent, patients were observed for four menstrual cycles after HSG without resorting to any therapy other than clomiphene citrate where indicated. The pregnancy rates for the four cycles after HSG were compared with chi-square analysis in the total study and in the diagnostic subgroups. The subgroup with infertility of unknown cause had a significantly higher pregnancy rate after HSG with oil than after HSG with aqueous contrast medium. No significant difference was seen for any other subgroup or for the overall cohort [8].

In a prospective randomized study, the number of pregnancies after hysterosalpingography (HSG) was estimated in 398 patients who had been infertile for longer than 1 year. Iohexol was used in 101 patients, ioxaglate in 102 patients, diatrizoate meglumine in 97 patients, and ethiodized poppy-seed oil in 98 patients. Ten months after HSG, the patient, referring physician, and/or hospital department was consulted for information about pregnancies. Questionnaires were obtained from the patients who became pregnant during the waiting period of 3 months. No differences in demographic parameters, infertility status, or diagnosis made with HSG were detected among the four contrast media groups. Significantly more patients became pregnant after HSG in the ethiodized poppy-seed oil group than in the three water-soluble contrast media groups (P less than.01). When only intrauterine pregnancies resulting in full-term births were considered, significant differences in pregnancy rates between the oil-soluble and the water soluble contrast media groups became more obvious. In the group that received ethiodized poppy-seed oil, almost one third of the infertile women had normal pregnancies and childbirths after HSG [9].

Meta-analysis of four randomized clinical trials (RCTs) and six nonrandomized controlled studies evaluated pregnancy rates after the use of oil- or water-soluble contrast media during HSG.

Four identified RCTs studied 800 patients and six nonrandomized studies comprised an additional 1,806 patients, all experiencing primary or secondary infertility.

Pregnancy rates were significantly higher in the oil soluble contrast media group compared with the water soluble contrast media group in the RCTs. Inclusion of the six nonrandomized studies did not alter this conclusion. This apparent benefit was greatest for patients with unexplained infertility.

Oil-soluble contrast media have a therapeutic effect compared with water-soluble media and this effect is greatest for patients who have been diagnosed as having unexplained infertility. New techniques for the evaluation of tubal patency support the hypothesis that tubal “plugs” may be involved in proximal tubal blockage [10].

Ninety-three patients with unilateral or bilateral proximal tubal occlusion confirmed by hysterosalpingography or laparoscopy underwent FTR with use of water-soluble contrast material alone (n=50) or also had an oil-based agent injected into each tube after recanalization (n=43). Pregnancy rates and outcomes of the two groups were studied retrospectively.

With respect to differences between groups, only the body mass index proved to be a significant predictor (oil, 28.4; water, 24.7; P=0.008). Mean age, duration of infertility, type of infertility, and initial diagnosis were comparable. There was a weak trend toward a higher pregnancy rate in the oil-based contrast material group, but it was not significant (P=0.64). The average time to pregnancy was 4.4 months with use of oil-based contrast material, compared to 7.7 months with use of only water-soluble contrast material (P=0.03).

The use of an oil-based agent had little effect on the rate of conception, but time to conception was reduced by more than 3 months [11].

Safety and Diagnostic or Therapeutic Efficacy of Water-soluble Versus Oil-based Contrast Media

Hysterosalpingography is used commonly in the evaluation of infertility and in the diagnosis of anomalies of the uterus and fallopian tubes. There is continued debate over the safety and diagnostic or therapeutic efficacy of water-soluble versus oil-based contrast media.

A 29-year-old woman with secondary infertility underwent hysterosalpingography with both water-soluble and oil-based contrast. The fallopian tubes appeared normal. Six months later, a plain abdominal radiograph obtained at the occasion of a minor motor vehicle accident revealed evidence of retained loculated pelvic contrast material. Subsequent laparoscopy identified adhesions and cul-de-sac implants strongly suspicious for endometriosis. Biopsy and pathologic study documented lipogranuloma.

Oil-based contrast media instilled into the pelvis at hysterosalpingography can persist for prolonged periods and create granulomatous lesions mimicking endometriosis. In view of the controversy whether oil-based contrast materials are superior to water-soluble media, the routine use of oil-based contrast media should be considered carefully [12].

Hysterosalpingography (HSG) has assumed a diagnostic and possibly therapeutic role in the evaluation of the infertile couple. The procedure is done using either an oil-based (OBCM) or a water-based (WBCM) contrast medium. Data from several retrospective studies suggest that higher pregnancy rates may be achieved when OBCM is used. Interpretation of these results, however, may be confounded by various methodologic flaws in study design and comparisons of heterogeneous populations. Letterie and Rose sought to compare the therapeutic benefit of OBCM and WBCM in a prospective randomized study of infertile patients, controlling for pelvic anatomy by laparoscopic assessment [13]. They used ethiodized oil (Ethiodol) or iothalamate meglumine (Conray 60) for tubal lavage at the time of laparoscopy only in patients with normal pelvic anatomy. Of the 225 patients who had diagnostic laparoscopy in the evaluation of infertility, 40(18%) had normal pelvic anatomy and an otherwise unremarkable evaluation. Adequate follow-up was available on 29 patients randomized to receive either OBCM (n=15) or WBCM (n=14). A significant difference in pregnancy rates was noted between OBCM (40%) and WBCM (14%) by chi-square analysis. No short- or long-term adverse reactions were noted. Results of this study suggest that in patients with normal pelvic anatomy as assessed laparoscopically, OBCM may offer a therapeutic benefit not evident with WBCM [13].

Moore et al. evaluated the effect of different iodinated contrast agents on the fallopian tube and adnexal tissue in 15 rabbits [14]. Ethiodized oil, an oil-soluble agent, was used in five rabbits. The following water-soluble agents were used: iothalamate meglumine 30% (n=3), iothalamate meglumine 60% (n=3), and ioxilan (n=4). The agents were injected through catheters placed in the fallopian tubes. Fallopian tubes and peritoneal cavities were histologically evaluated. The contralateral tube served as a control. Ioxilan and iothalamate meglumine 30% produced no pathologic response in the tube or peritoneal cavity. Iothalamate meglumine 60% was associated with mild inflammatory infiltrate, mucosal edema, giant cell reaction, and periovarian adhesions that were bilateral but more pronounced on the injected side. Use of ethiodized oil resulted in papillary fibrous adhesions on the ovarian surface, and fat granulomas were seen in the periovarian tissues. The safety of oil-based contrast agents for use in hysterosalpingography is therefore questioned. No significant differences were found among the water-soluble contrast agents [14].

Hysterosalpingography can be accomplished with either oil or water-soluble contrast medium. This randomized prospective study compared pregnancy rates in women who had hysterosalpingography with either water- or oil soluble contrast material and were followed for six months. Fifteen of 60 (25%) patients who received water-soluble dye conceived compared with 14 of 46 (30%) patients in the oil-soluble group, a statistically insignificant difference. Furthermore, no difference in pregnancy rates within each subgroup of fertility diagnosis was detected. Intravasation was more common in patients administered oil-based contrast materials (six of 46 versus one of 60 patients, P=0.02), although no serious consequences occurred. No difference in the amount of pain as assessed by pain scoring was experienced by patients in each group. The authors conclude that pregnancy rates are similar after hysterosalpingography with oil- and water-soluble contrast material, during at least the first six months after the procedure [15].

Immunological Relationship Between Mother and Conceptus

Aspects of the immunological relationship between mother and conceptus still remain a mystery, although the recent advances in molecular biology have enlightened some of the parameters that participate in feto-maternal cross-talk during implantation [16]. The atypical expression of major histocompatibility complex (MHC), the specific roles of some hormones and cytokines, as well as the temporal and spatial distributions of uterine natural killer (uNK) cells, represent substantive parameters of feto-maternal immunotolerance during implantation [17]. Although human maternal and fetal immunology is difficult to investigate, aberrant immune responses and an imbalanced cytokine network may be related to infertility, implantation failures after IVF, and recurrent pregnancy losses [18]. Patients with recurrent implantation failure (RIF) should be tested for inherited and acquired thrombophilias. Each patient should be individually assessed and counseled regarding management with low molecular-weight heparin (LMWH). Empirical treatment with LMWH, aspirin, or corticosteroids is not effective for women with RIF who have negative thrombophilic tests [19]. If thrombophilic tests are normal, patients should be tested for immunological causes. The findings of a recent study suggest that increases in the percentage of CD56(dim) cells and NK cytotoxicity in peripheral blood may be important contributing factors for both RSA and IVF failure [20]. Human leukocyte antigen (HLA)-DQA1*0505 sharing or the maternal killer immunoglobulin-like receptor (KIR) repertoire is associated with recurrent spontaneous abortion (RSA) or repeated implantation failure (RIF) [21] and if abnormal, the patient might then benefit from intravenous immunoglobulin (IVIg) therapy [19]. IVIg has been successful in the treatment of recurrent miscarriage and recurrent implantation failure among women with elevated anti-phospholipid antibodies (APA) and/or NK cell activity [22]. When the pregnancy outcomes of women with a history of reproductive failure and elevated NK cell cytotoxicity treated with intralipid were compared with women treated with IVIg, no differences were seen [22]. Sideby-side comparison showed that synthetic pre-implantation factor (sPIF) is equally effective to inhibit NK cell toxicity at a lower dose than intravenous gamma immunoglobulin or intralipid treatment currently used [23]. sPIF is not yet available commercially, but intralipid infusions are available globally. Intralipid (IL) is a synthetic product composed of 10% soybean oil, 1.2% egg yolk phospholipids, 2.25% glycerin, and water. When indicated, IL is infused 7-10 days prior to embryo transfer (ET), and one more time again after a positive pregnancy in women whose NKa is due to an autoimmune cause (antiphospholipid antibodies and/or antithyroid antibodies) [24]. In cases of alloimmune implantation dysfunction (DQa and/HLA matching between the embryo recipient and the male partner), the same applies, but in this situation, the infusion is repeated at 2-4 week intervals until the 24th week of pregnancy [24]. IL costs about 10 times less than IVIg, is not a blood product, and is without significant side effects [24].

### Intralipid

Intravenous lipid emulsions have been used experimentally since at least the 19th century. An early product marketed in 1957 under the name Lipomul was briefly used in the United States but was subsequently withdrawn due to side effects. Intralipid was invented by the Swedish physician and nutrition researcher Arvid Wretlind, and was approved for clinical use in Sweden in 1962 [25]. In the United States, the Food and Drug Administration initially declined to approve the product due to prior experience with another fat emulsion. It was approved in the United States in 1972.

### Conclusion

It is the first time that intralipid-iodine is suggested to be used for hysterosalpingography in infertile women.

### Example 9

### Intralipid Treatment for Alzheimer Disease

Currently, there is no cure for Alzheimer's. But drug and non-drug treatments may help with both cognitive and behavioral symptoms. Researchers are looking for new treatments to alter the course of the disease and improve the quality of life for people with dementia.

Intralipid treatment is first suggested here for the treatment of Alzheimer disease. It should be given intravenously on a monthly basis according to each patient's response. Clinical studies should be done in order to evaluate this new treatment modality

Dr. Aloysius “Alois” Alzheimer

Dr. Aloysius “Alois” Alzheimer was a German psychiatrist and neuropathologist and a colleague of Emil Kraepelin. Alzheimer is credited with identifying the first published case of “presenile dementia”, which Kraepelin would later identify as Alzheimer's disease [1,2].

In 1901, Dr. Alzheimer observed a patient at the Frankfurt Asylum named Auguste Deter. The 51-year-old patient had strange behavioral symptoms, including a loss of short-term memory; she became his obsession over the coming years. Auguste Deter was a victim of the politics of the time in the psychiatric community; the Frankfurt asylum was too expensive for her husband. Mr. Deter made several requests to have his wife moved to a less expensive facility, but Dr. Alzheimer intervened in these requests. Ms. Deter remained at the Frankfurt asylum, where Alzheimer had made a deal to receive her records and brain upon her death [3]. On 8 Apr. 1906, Ms. Deter died, and Dr. Alzheimer had her medical records and brain brought to Munich where he was working in Kraepelin's laboratory. With two Italian physicians, he used the staining techniques of Bielschowsky to identify amyloid plaques and neurofibrillary tangles. These brain anomalies would become identifiers of what later became known as Alzheimer's Disease [4].

### Alzheimer's Disease

Some evidence indicates that disruption of the blood—brain barrier in Alzheimer's disease patients allows blood plasma containing amyloid beta (Aβ) to enter the brain where the Aβ adheres preferentially to the surface of astrocytes [5]. These findings have led to the hypotheses that [1] breakdown of the blood—brain barrier allows access of neuron-binding autoantibodies and soluble exogenous Aβ42 to brain neurons and [2] binding of these autoantibodies to neurons triggers and/or facilitates the internalization and accumulation of cell surface-bound Aβ42 in vulnerable neurons through their natural tendency to clear surface-bound autoantibodies via endocytosis. Eventually the astrocyte is overwhelmed, dies, ruptures, and disintegrates, leaving behind the insoluble Aβ42 plaque. Thus, in some patients, Alzheimer's disease may be caused (or more likely, aggravated) by a breakdown in the blood-brain barrier [6]. AD evolves with widespread loss of neurons and their synapses in such key brain areas as the cerebral cortex, entorhinal area, and hippocampus. At the gross level, this is evident as a general shrinkage of the brain away from the cranial vault and a corresponding dilation of the fluid-filled brain ventricles to fill the void. At the microscopic level, there are several different pathological changes that occur, but one consistent pathological hallmark is the early appearance of amyloid plaques. These plaques are abundant and widely scattered throughout AD-vulnerable brain regions. They contain a 42-amino acid protein fragment, known as amyloid 1-42 (A42), that is derived from the sequential enzymatic cleavage of the much larger amyloid precursor protein. Once produced, A42 has the ability to self-assemble into nondegradeable fibrils that can persist in AD brains long after the neurons in which they accumulated have died [7].

Alzheimer's disease (AD) was first described in 1906 by German psychiatrist Alois Alzheimer, who observed abnormal clumps and tangled bundles of protein in the brain of a patient who experienced memory loss, language difficulties, and abnormal behaviour [8]. The risk of developing AD increases exponentially with age and is the leading cause of dementia and the most common neurodegenerative disease in the elderly; prevalence rates in 65-74 years old are estimated to be 3%, rising to 19% for 75-85 years old, and nearly 50% in those aged over 85 (9). AD is more common among older people but it is not a normal part of ageing. As the global population ages, the prevalence of AD is expected to rise from 36 million to 115 million sufferers by 2050 [9].

It is estimated that over 5% of the U.S. population over 65 and over 15% of the U.S. population over 85 are beset with some form of Alzheimer's disease. It is believed that the principal cause for confinement of the elderly in long term care facilities is due to this disease, and approximately 65% of those dying in skilled nursing facilities suffer from it [10] intravenous lipid emulsion The idea that intravenous lipid emulsion could be used to affect the pharmacokinetics of a drug in circulation was first introduced fifty years ago. It was shown that rats infused lipid emulsion after an injection of the barbiturate thiopental emerged more rapidly from anaesthesia than rats infused the same volume of fat-free solution [11]. Other early studies were published on the effect of lipid emulsion on chlorpromazine availability in rabbits [12], and the effect of lipid emulsion on the elimination of phenytoin [13]. Although the studies show some effect of lipid emulsion, this did not kindle more widespread interest in the subject. The serendipitous discovery of the apparently shielding effect of a large intravenous dose of lipid emulsion against bupivacaine toxicity in rats triggered renewed interest in the field [14]. Additional experimental animal and isolated heart studies were performed [15-16], and although efficacy and safety had not been established by clinical trials, clinicians soon applied lipid therapy to seemingly hopeless cases of severe intoxication [17].

Intravenous lipid emulsion therapy for severe intoxication is a relatively young field. Although a few early studies on the pharmacokinetic effects of intravenous lipid emulsion exist [11-13; 18], its use as a treatment for severe intoxication was proposed as late as 1998 [14]. Since this proposal, no randomized controlled human trials have been published. Thus, the evidence supporting this use of lipid emulsion consists only of animal studies and human case reports of varying quality [20]. Amyloid beta-peptide (Abeta) is a key molecule in Alzheimer disease (AD). Cerebral deposition of Abeta was earlier thought to initiate the pathological cascade of AD, including the formation of senile plaques and neurofibrillary tangles, neuronal loss, and dementia. According to the classical amyloid hypothesis, the aggregation of Abeta into insoluble beta-sheet fibrils plays an important role in its neurotoxicity. However, this hypothesis is paradoxical: The concentrations of Abeta required for fibrillization and neurotoxicity are higher than its physiological concentrations. Cognitive decline in AD patients is not correlated with the levels of senile plaque formation or insoluble Abeta formation; instead it correlates with the levels of synapse loss and the levels of soluble Abeta. These observations suggest the existence of soluble toxic forms of Abeta in AD brains; these forms have recently been identified to be oligomeric assemblies of Abeta. At present, AD is believed to begin with synaptic dysfunction caused by soluble Abeta oligomers. This hypothesis termed the oligomer hypothesis, is based on the following observations: The levels of Abeta oligomers are high in AD brains. Exogenous Abeta oligomers at physiological concentrations cause synaptic and cognitive dysfunction in vivo and synapse loss and neuronal death in vitro. Furthermore, we observed that the E693delta mutation in the amyloid precursor protein found in AD patients causes disease by increasing the formation of Abeta oligomers without inducing the formation of Abeta fibrils or senile plaques. Currently, senile plaque formation is thought to occur in order to protect neurons from the toxicity of diffusible Abeta oligomers by sequestering them into deposits. Thus, soluble Abeta oligomers play a more important role in the etiology of AD insoluble Abeta fibrils [21].

The defining features of Alzheimer disease (AD) include conspicuous changes in both brain histology and behavior. The AD brain is characterized microscopically by the combined presence of 2 classes of abnormal structures, extracellular amyloid plaques and intraneuronal neurofibrillary tangles, both of which comprise highly insoluble, densely packed filaments. The soluble building blocks of these structures are amyloid-β (Aβ) peptides for plaques and tau for tangles. Amyloid-β peptides are proteolytic fragments of

the transmembrane amyloid precursor protein, whereas tau is a brain-specific, axon-enriched microtubule-associated protein. The behavioral symptoms of AD correlate with the accumulation of plaques and tangles, and they are a direct consequence of the damage and destruction of synapses that mediate memory and cognition. Synapse loss can be caused by the failure of live neurons to maintain functional axons and dendrites or by neuron death.

During the past dozen years, a steadily accumulating body of evidence has indicated that soluble forms of Aβ and tau work together, independently of their accumulation into plaques and tangles, to drive healthy neurons into the diseased state and that hallmark toxic properties of Aβ require tau. For instance, acute neuron death, delayed neuron death following ectopic cell cycle reentry, and synaptic dysfunction are triggered by soluble, extracellular Aβ species and depend on soluble, cytoplasmic tau. Therefore, Aβ is upstream of tau in AD pathogenesis and triggers the conversion of tau from a normal to a toxic state, but there is also evidence that toxic tau enhances Aβ toxicity via a feedback loop. Because soluble toxic aggregates of both Aβ and tau can self-propagate and spread throughout the brain by prionlike mechanisms, successful therapeutic intervention for AD would benefit from detecting these species before plaques, tangles, and cognitive impairment become evident and from interfering with the destructive biochemical pathways that they initiate [22].

The increasing prevalence of Alzheimer's disease (AD) and a lack of effective prevention or disease-modifying therapies are global challenges with devastating personal, social and economic consequences. The amyloid β (Aβ) hypothesis posits that cerebral β-amyloidosis is a critical early event in AD pathogenesis. However, failed clinical trials of Aβ-centric drug candidates have called this hypothesis into question. Whereas we acknowledge that the Aβ hypothesis is far from disproven, we here re-visit the links between Aβ, tau and neurodegeneration. We review the genetics, epidemiology and pathology of sporadic AD and give an updated account of what is currently known about the molecular pathogenesis of the disease [23]. The amyloid hypothesis has driven drug development strategies for Alzheimer's disease for over 20 years. We review why accumulation of amyloid-beta (Aβ) oligomers is generally considered causal for synaptic loss and neurodegeneration in AD. We elaborate on and update arguments for and against the amyloid hypothesis with new data and interpretations, and consider why the amyloid hypothesis may be failing therapeutically. We note several unresolved issues in the field including the presence of Aβ deposition in cognitively normal individuals, the weak correlation between plaque load and cognition, questions regarding the biochemical nature, presence and role of Aβ oligomeric assemblies in vivo, the bias of pre-clinical AD models toward the amyloid hypothesis and the poorly explained pathological heterogeneity and comorbidities associated with AD. We also illustrate how extensive data cited in support of the amyloid hypothesis, including genetic links to disease, can be interpreted independently of a role for Aβ in AD. We conclude it is essential to expand our view of pathogenesis beyond Aβ and tau pathology and suggest several future directions for AD research, which we argue will be critical to understanding AD pathogenesis [24].

The aggregation and deposition of amyloid-beta (Abeta) in the brain is thought to be an early event in the pathology of Alzheimer's disease (AD). Many studies have reported the association of Abeta with lipoproteins from plasma suggesting an involvement of lipoprotein particles in Abeta transport. Chylomicron-like lipid emulsions, resembling chylomicrons in composition, size and metabolism were prepared in the presence of [125I] Abeta1-40. Abeta was found to associate significantly with these lipid emulsions during their preparation. The chylomicron-like emulsions containing Abeta were then injected into a lateral ear vein of conscious rabbits and blood sampled at regular intervals up to 30 mins. It was observed that there was no difference in the plasma clearance of [125I] Abeta and that of the 3H-cholesteryl ester, a marker of the emulsion particles, demonstrating that Abeta remains associated with these particles throughout both their lipolysis and tissue uptake. Our results show that Abeta can be metabolised in association with triglyceride rich lipoproteins (TRLs). In addition we report the presence of specific markers of TRLs of hepatic and intestinal origin in human CSF thus suggesting a potential means of cerebral Abeta delivery [25].

Docosahexaenoic acid (DHA), the main n-3 polyunsaturated fatty acid (PUFA) in membranes, is particularly abundant in brain cells. Decreased cerebral concentrations of DHA, resulting from dietary n-3 deficiency, are associated with impaired cognitive function. Because the cellular causes of this impairment are still unknown, we need in vitro models that mimic the variations in n-3/n-6 PUFA seen in vivo. We have compared the PUFA profiles of hamster astrocytes cultured in medium supplemented with long-chain PUFA [DHA and/or arachidonic acid (AA)] with those of brain tissue from hamsters fed an n-6/n-3 PUFA-balanced diet or one lacking n-3 PUFA. Astrocytes were obtained from the brain cortex of newborn hamsters and cultured in minimum essential medium+5% fetal calf serum (FCS) supplemented with DHA and/or AA for 10 days. The astrocytes cultured in medium+FCS had low n-3 PUFA contents, comparable to those of brain tissue from hamsters fed an n-3-deficient diet. We have shown that astrocytes grown in medium supplemented with DHA and/or AA, plus alpha-tocopherol to prevent lipid peroxidation, incorporated large amounts of these long-chain PUFA, so that the n-6/n-3 PUFA compositions of the phosphatidylethanolamine and phosphatidylcholine, the two main classes of membrane phospholipids, were greatly altered. Astrocytes cultured in medium plus DHA had a more physiological n-3 status, grew better, and retained their astrocyte phenotype. Thus astrocytes in culture are likely to be physiologically relevant only when provided with adequate DHA. This reliable method of altering membrane phospholipid composition promises to be useful for studying the influence of n-6/n-3 imbalance on astrocyte function [26].

Rat neural stem cells/neural progenitors (NSC/NP) are generally grown in serum-free medium. In this study, NSC/NP were supplemented with the main long-chain polyunsaturated fatty acids (PUFAs) present in the brain, arachidonic acid (AA), or docosahexaenoic acid (DHA), and were monitored for their growth. Lipid and fatty acid contents of the cells were also determined. Under standard conditions, the cells were characterized by phospholipids displaying a highly saturated profile, and very low levels of PUFAs. When cultured in the presence of PUFAs, the cells easily incorporated them into the phospholipid fraction. We also compared the presence of three membrane proteins in the lipid raft fractions: GFR and connexin 43 contents in the rafts were increased by DHA supplementation, whereas Gbeta subunit content was not significantly modified. The restoration of DHA levels in the phospholipids could profoundly affect protein localization and, consequently, their functionalities [27].

Lipids are the fundamental structural components of biological membranes. For a long time considered as simple barriers segregating aqueous compartments, membranes are now viewed as dynamic interfaces providing a molecular environment favorable to the activity of membrane-associated proteins. Interestingly, variations in membrane lipid composition, whether quantitative or qualitative, play a crucial role in regulation of membrane protein functionalities. Indeed, a variety of alterations in brain lipid composition have been associated with the processes of normal and pathological aging.

Although not establishing a direct cause-and-effect relationship between these complex modifications in cerebral membranes and the process of cognitive decline, evidence shows that alterations in membrane lipid composition affect important physicochemical properties notably impacting the lateral organization of membranes, and thus microdomains. It has been suggested that preservation of microdomain functionality may represent an effective strategy for preventing or decelerating neuronal dysfunction and cerebral vulnerability, processes that are both aggravated by aging. The working hypothesis developed in this review proposes that preservation of membrane organization, for example, through nutritional supplementation of docosahexaenoic acid, could prevent disturbances in and preserve effective cerebral function [28]. To date, no study has been performed to evaluate the antidotal effect of intravenous lipid emulsion on the poisoned patients' level of consciousness and routine metabolic profile tests in non-local anesthetic drug overdose. Taftachi F. et al. [29] aim was to evaluate the effect of intravenous intralipid administration as an antidote on the poisoned patients' Glasgow Coma Scale (GCS), hemodynamic parameters, arterial blood gas analysis, and routine metabolic profile tests (i.e., urea, glucose, sodium, and potassium) in the setting of non-local anesthetic drug overdose.

In this randomized controlled trial, a total of 30 patients with non-local anesthetic drug intoxication were enrolled and randomly assigned into case (n=15) and control (n=15) groups. In the case group, all patients received 10 cc/kg intralipid 10% infusion. The patients in the control group just received the supportive care. Patients' demographic and clinical characteristics and results of their laboratory tests were evaluated at presentation and 6 hours after that. Mean age was 23+/−5 and 28+/−11 years in cases and controls, respectively. There were no significant statistical differences between these two groups regarding age, gender, elapsed time between intubation and extubation, and need for intubation and/or mechanical ventilation (p=0.70 and p=1.00, respectively). Also, systolic blood pressure, pulse rate, mean rate pressure product, respiratory rate, results of arterial blood gas analyses, serum sodium, potassium, urea, and creatinine on presentation and six hours later were not statistically significantly different between the two study groups. However, a significant difference was found between the two groups in terms of GCS difference (p=0.048) and blood glucose six hours after presentation (p=0.04).

In the setting of non-local anesthetic drug overdose, intravenous intralipid infusion can increase GCS and interestingly, decrease the blood glucose. Lipid emulsions are widely used as carriers for hypnotics such as propofol, etomidate, and diazepam. It is assumed that the emulsions alone exert no effect on cellular functions nor influence the pharmacokinetics, pharmacodynamics, or anesthetic and analgetic potency of the hypnotics they carry. To elucidate possible interactions between lipid emulsions and cell membranes, in particular membrane-bound proteins, we investigated the effects of commercially available lipid emulsions on the cell membranes of cultured cortical neurons from the mouse by using the whole-cell configuration of the patch-clamp technique. Of nine lipid emulsions tested, three, i.e., Intralipid, Structolipid, and, to a much lesser extent, Abbolipid, activated membrane currents in the neuronal cells in a dilution-dependent manner. The emulsion-induced currents were not affected by picrotoxin or bicuculline but were inhibited by DL-AP5 and ketamine. The voltage dependence of the currents was influenced by the presence of Mg (2+) in a way that is typical for currents conducted by N-methyl-D-aspartate receptor channels. We conclude that Intralipid, Structolipid, and Abbolipid activate N-methyl-D-aspartate receptor channels in cortical neurons.

Lipid emulsions are widely used as carriers for hypnotics such as propofol, etomidate, or diazepam. We tested nine commercially available lipid emulsions and demonstrate that three of them—Intralipid, Structolipid, and Abbolipid—activate NMDA receptor channels in the membranes of cortical neuronal cells [30] Haywood S C et al. [31] tested the hypothesis that lipids could act as an alternative fuel source in the brain during insulin-induced hypoglycemia. Male Sprague-Dawley rats were subjected to hyperinsulinemic (5 mU·kg-1·min-1 hypoglycemic (approximately 50 mg/dl) clamps. In protocoll, intralipid (IL), a fat emulsion, was infused intravenously to prevent the fall in free fatty acid levels that occurs in response to hyperinsulinemic hypoglycemia. Intravenous lipid infusion did not alter the counterregulatory responses to hypoglycemia. To test whether IL could have central effects in mediating the counterregulatory response to hypoglycemia, in protocol 2 the brains of precannulated rats were intracerebroventricularly (icy) infused with IL or artificial cerebrospinal fluid (aCSF) as control. Unexpectedly, the epinephrine and glucagon response to hypoglycemia was significantly augmented with icy IL infusion. To determine whether central IL infusion could restore defective counter regulation, in protocol 3 rats were made recurrently hypoglycemic (RH) for 3 days and on the 4th day underwent hyperinsulinemic hypoglycemic clamps with icy IL or aCSF infusion. RH rats had the expected impaired epinephrine response to hypoglycemia, and icy IL infusion again significantly augmented the epinephrine response in RH rats to normal. With regard to our experimental model of hypoglycemic counter regulation, we conclude that 1) systemic lipid infusion did not alter the counterregulatory response to hypoglycemia, 2) the icy infusion of lipids markedly increased CSF FFA levels and paradoxically augmented the epinephrine and glucagon responses, and 3) the blunted sympathoadrenal response in recurrently hypoglycemic rats was completely normalized with the icy lipid infusion. It is concluded that, in the setting of insulin-induced hypoglycemia, increased brain lipids can enhance the sympathoadrenal response. Lipid infusion reverses systemic local anesthetic toxicity. The acceptable upper limit for lipid administration is unknown and has direct bearing on clinical management. We hypothesize that high volumes of lipid could have undesirable effects and sought to identify the dose required to kill 50% of the animals (LD (50)) of large volume lipid administration. Intravenous lines and electrocardiogram electrodes were placed in anesthetized, male Sprague-Dawley rats. Twenty percent lipid emulsion (20, 40, 60, or 80 mL/kg) or saline (60 or 80 mL/kg), were administered over 30 mins; lipid dosing was assigned by the Dixon “up-and-down” method. Rats were recovered and observed for 48 hrs then euthanized for histologic analysis of major organs. Three additional rats were administered 60 mL/kg lipid emulsion and euthanized at 1, 4, and 24 hrs to identify progression of organ damage.

The maximum likelihood estimate for LD (50) was 67.72 (SE, 10.69) mL/kg. Triglycerides were elevated immediately after infusion but returned to baseline by 48 hrs when laboratory abnormalities included elevated amylase, aspartate aminotransferase, and serum urea nitrogen for all lipid doses. Histologic diagnosis of myocardium, brain, pancreas, and kidneys was normal at all doses. Microscopic abnormalities in lung and liver were observed at 60 and 80 mL/kg; histopathology in the lung and liver was worse at 1 hr than at 4 and 24 hrs.

The LD (50) of rapid, high volume lipid infusion is an order of magnitude greater than doses typically used for lipid rescue in humans and supports the safety of lipid infusion at currently recommended doses for toxin-induced cardiac arrest. Lung and liver histopathology was observed at the highest infused volumes [32]. Intravenous lipid emulsion has been suggested as treatment for severe intoxications caused by lipophilic drugs, including tricyclic antidepressants. We investigated the effect of lipid infusion on plasma and tissue concentrations of amitriptyline and haemodynamic recovery, when lipid was given after amitriptyline distribution into well-perfused organs. Twenty anaesthetized pigs received amitriptyline intravenously 10 mg/kg for 15 min. Thirty minutes later, in random fashion, 20% Intralipid(®) (Lipid group) or Ringer's acetate (Control group) was infused 1.5 ml/kg for 1 min. followed by 0.25 ml/kg/min. for 29 min. Arterial and venous plasma amitriptyline concentrations and haemodynamics were followed till 75 min. after amitriptyline infusion. Then, frontal brain and heart apex samples were taken for amitriptyline measurements. Arterial plasma total amitriptyline concentrations were higher in the Lipid than in the Control group (p<0.03) from 20 min. on after the start of the treatment infusions. Lipid emulsion reduced brain amitriptyline concentration by 25% (p=0.038) and amitriptyline concentration ratios brain/arterial plasma (p=0.016) and heart/arterial plasma (p=0.011). There were no differences in ECG parameters and no severe cardiac arrhythmias occurred. Two pigs developed severe hypotension during the lipid infusion and were given adrenaline. In conclusion, lipid infusion, given not earlier than after an initial amitriptyline tissue distribution, was able to entrap amitriptyline back into plasma from brain and possibly from other highly perfused, lipid-rich tissues. In spite of the entrapment, there was no difference in haemodynamics between the groups [33]. Malathion is one of the most widely used organophosphate pesticides and herbicides. It has given rise to major clinical problems by its poisoning in all over the world. Malathion also a highly lipophilic agent, and tends to accumulate within lipid-rich tissue like a brain in the body, causing toxicity. Therefore, the study was aimed to investigate if there is a possible beneficial effect of using intralipid fat emulsion (IFE) on the neurotoxicity, and to detect it time-dependently at the beginning, 6th and 12th hours of M intoxication. Forty-eight rats were randomly divided into six groups including: control (C), Lipid (L) group (18.6 mL/kg oral IFE), Malathion (M) group (10 mg/kg oral M), M0L group (IFE treated after immediate from M), M6L group (IFE treated after 6 hours from M), M12L group (IFE treated after 12 hours from M). M group in comparison with all others group, there was an increase in the total oxidant status (TOS) level. M group in comparison with C, L, M0L groups, it was seen significantly decrease in the total antioxidant capacity (TAC) level. Interestingly, M group in comparison with M6L and M12L groups, there was no significant difference among these groups in terms of the TAC levels. Although there was no significant difference among C, L and M0L groups in terms of both TAC and TOS levels, but was significant difference C, L groups in comparison with M6L, M12L groups in terms of TAC levels. C group in comparison with L, M0L, M6L, M12L groups in terms of TOS levels, there was no significant difference. These findings have indicated that IFE seriously reduced TOS levels in all the groups depending on time. Also, M0L group in comparison with M6L and M12L groups, there was significantly increase of the TAC levels. There was no statistically significant difference between M6L and M12L groups. These biochemical results were confirmed with immunohistochemical results.

The study has had some certain evidence that IFE is a promising safe therapy for acutely intoxicated cases by organophosphate. It is much more effective if used at the beginning of organophosphate poisoning. As such, there is no need to avoid using IFE in clinical practice [34].

Chlorpyriphos is one of the most widely used organophosphate (OP) insecticide in agriculture with potential toxicity. Current post-exposure treatments consist of anti-cholinergic drugs and oxime compounds. We studied the effects of intralipid and caffeic acid phenethyl ester (CAPE) on chlorpyriphos toxicity to compose an alternative or supportive treatment for OP poisoning.

Forty-nine rats were randomly divided into seven groups. Chlorpyriphos was administered for toxicity. Intralipid (IL) and CAPE administered immediately after chlorpyriphos. Serum acetylcholinesterase (AChE) level, total oxidant status (TOS), total antioxidant response (TAR), and histologic examination of cerebellum and brain tissue with Hematoxylin-Eosin and immunohistochemical dyes were examined.

Serum enzym levels showed that chlorpyriphos and CAPE inhibited AChE while IL alone had no effect, chlorpyriphos and CAPE intensifies the inhibition effect. Significant difference at AChE levels between the chlorpyriphos+IL and chlorpyriphos+CAPE verified that IL has a protective effect on AChE inhibition. TAR levels were significantly increased in all groups except chlorpyriphos group, TOS levels revealed that CAPE and IL decrease the amount of oxidative stress. Histologic examination revealed that neuronal degeneration was slightly decreased at chlorpyriphos+IL group, but CAPE had a significant effect on protection of neuronal degeneration.

The results of this study gave us three key points. 1) AChE activity is important for diagnosis of OP intoxication but it has no value for determining the neuro-degeneration. 2) CAPE inhibits AChE activity and may increase the muscarinic-nicotinic hyperactivation. Therefore it should not be used for treatment of OP intoxication. 3) IL decreases the severity of neurodegeneration and symptoms of OP intoxication and it can be used as a supportive agent [35].

Intravenous lipid emulsion has been suggested as treatment for local anaesthetic toxicity, but the exact mechanism of action is still uncertain. Controlled studies on the effect of lipid emulsion on toxic doses of local anaesthetics have not been performed in man. In randomized, subject-blinded and two-phase cross-over fashion, eight healthy volunteers were given a 1.5 ml/kg bolus of 20% Intralipid(®) (200 mg/ml) or Ringer's acetate solution intravenously, followed by a rapid injection of lidocaine 1.0 mg/kg. Then, the same solution as in the bolus was infused at a rate of 0.25 ml/kg/min. for 30 min. Electroencephalography (EEG) was recorded, and 5 min. after lidocaine injection, the volunteers were asked to report subjective symptoms. Total and un-entrapped lidocaine plasma concentrations were measured from venous blood samples. EEG band power changes (delta, alpha and beta) after the lidocaine bolus were similar during lipid and during Ringer infusion. There were no differences between infusions in the subjective symptoms of central nervous system toxicity. Lidocaine was only minimally entrapped in the plasma by lipid emulsion, but the mean un-entrapped lidocaine area under concentration-time curve from 0 to 30 min. was clearly smaller during lipid than Ringer infusion (16.4 versus 21.3 mg×min/l, p=0.044). Intravenous lipid emulsion did not influence subjective toxicity symptoms nor affect the EEG changes caused by lidocaine [36].

### Intralipid Treatment for Alzheimer Disease

Lange D B et al. [37] described a case of severe central nervous system toxicity after an overdose of lidocaine by local infiltration in a peritoneal dialysis patient and subsequent treatment of the toxicity with lipid emulsion. A 31-year-old male received an iatrogenic overdose of 1600 mg of lidocaine 2% by infiltration during an attempt to remove and replace a peritoneal dialysis catheter. Within 10 minutes after the last lidocaine injection, the patient exhibited features of local anesthetic toxicity, which included tachycardia, hypertension, shortness of breath, dizziness, and a choking sensation that progressed to hallucinations, dysarthria, and uncoordinated, weak limb movement. Within 10 minutes after administration of a single 1.5-mg/kg intravenous bolus of 1.5 mL/kg [corrected], the patient improved dramatically. After observation overnight in a monitored care setting, the patient was discharged home with no apparent neurologic sequelae. Systemic toxicity due to regional anesthesia with local anesthetic agents such as lidocaine has been well described in the medical literature. The use of lipid emulsion as an antidote to the toxicity of local anesthetics and other lipophilic drugs has been suggested as a valuable intervention in both early, rapidly progressive toxicity, as well as toxicity that is refractory to standard treatment. Patients with advanced chronic kidney disease may be more susceptible to systemic effects of lidocaine due to decreased drug elimination.

Central nervous system toxicity due to an overdose of lidocaine was quickly reversed by intravenous lipid emulsion in our patient [37]. The major component fatty acids in Intralipid are linoleic acid (44-62%), oleic acid (19-30%), palmitic acid (7-14%), a-linolenic acid (4-11%) and stearic acid (1.4-5.5%) [37]. Oxidative stress is a hallmark of many degenerative disorders. The brain is particularly vulnerable to this phenomenon owing to high oxygen consumption, enrichment in polyunsaturated fatty acids (PUFAs) and high levels in redox metal ions [38]. Lipid peroxidation products (LPPs) have been found in brain, cerebrospinal fluid and plasma from patients with Alzheimer's disease (AD)/t [38]. Primary substrates for lipid peroxidation are PUFAs and include ω-6 fatty acids (for example, linoleic acid and arachidonic acid) as well as ω-3 fatty acids (for example, docosahexaenoic acid). Reactive oxygen species are responsible for starting the chain by the production of an unstable lipid radical that is converted to a lipid peroxyl radical, leading to the peroxidation of other fatty acids (propagation). This chain reaction stops (termination) when two radicals react to produce a non-radical species, or as a result of antioxidants (for example, vitamin C and vitamin E) and enzymes of the superoxide dismutase, catalase and peroxidase families [38]. Oxidized PUFAs are further degraded to toxic products, such as 4-hydroxy-2-nonenal (HNE), acrolein and other short-chain aldehydes. Importantly, amyloid-β has been shown to cause oxidative stress through its interaction with transition metal ions, such as Cu2+ and Zn2+, which are enriched in senile plaques [39]. Amyloid-β can reduce these metal ions, thus producing hydrogen peroxide. During this process, amyloid-β becomes oxidized, thereby leading to the crosslinking of some of its residues' side-chains and the formation of aggregate-prone adducts. Alternatively, hydrogen peroxide can be generated catalytically from Cu2+- or Zn2+-bound amyloid-β using other electron donors (for example, PUFAs and cholesterol), a process leading to the generation of toxic LPPs, such as oxysterol and HNE. Finally, amyloid-β itself can be crosslinked by HNE. Key challenges in the field are to understand the role of LPP accumulation in the progression of AD-associated manifestations.

The linoleic acid metabolism was examined in the brain cortex of 4 month-old and 24 month-old rats. After the injection of [1-14C]-linoleate into the lateral ventricle of the brain the animals were sacrificed at 1,3 and 6 hours from the injection. The linoleate (18:2) incorporation into lipids, the presence of fatty acid peroxidation products, as well as the 18:2 transformation into elongated and desaturated derivatives were determined. Both an age-related reduction in linoleate incorporation rate into glycerophospholipids and a decrease in fatty acid turnover were found. Furthermore, in glycerophospholipids from 24 month-old rat brain cortex a higher level of hydroperoxide derivative of linoleate was found as compared to 4 month-old animals, and this damaged fatty acid is eliminated more slowly in aged rats than in adults. Finally, unlike 4 month-old animals, a stimulation of the transformation rate of linoleate into desaturation (6,9,12-C18:3) and elongation (8,11,14, C20:3) products was found in 24 month-old rat brain cortex. On the contrary, as far as arachidonic acid (one of the most important end products of the mechanism of linoleate modification) is concerned, the differences between aged and control animals were small, making it quite difficult to attribute a physiological meaning to this phenomenon [40].

Alzheimer's disease and associated diseases constitute a major public health concern worldwide. Nutrition-based, preventive strategies could possibly be effective in delaying the occurrence of these diseases and lower their prevalence. Arachidonic acid is the second major polyunsaturated fatty acid (PUFA) and several studies support its involvement in Alzheimer's disease. The objective of this review is to examine how dietary arachidonic acid contributes to Alzheimer's disease mechanisms and therefore to its prevention. First, we explore the sources of neuronal arachidonic acid that could potentially originate from either the conversion of linoleic acid, or from dietary sources and transfer across the blood-brain-barrier. In a second part, a brief overview of the role of the two main agents of Alzheimer's disease, tau protein and Aβ peptide is given, followed by the examination of the relationship between arachidonic acid and the disease. Third, the putative mechanisms by which arachidonic acid could influence Alzheimer's disease occurrence and evolution are presented. The conclusion is devoted to what remains to be determined before integrating arachidonic acid in the design of preventive strategies against Alzheimer's disease and other neurodegenerative diseases [41].

Insulin resistance and type 2 diabetes are associated with an increased risk of neurodegenerative diseases. Brain-derived neurotrophic factor (BDNF) regulates neuronal differentiation and synaptic plasticity, and its decreased levels are supposed to play a role in the pathogenesis of Alzheimer's disease and other disorders. The aim of the current study was to estimate the effects of hyperinsulinemia and serum free fatty acids (FFA) elevation on circulating BDNF concentration in humans.

We studied 18 healthy male subjects (mean age 25.6±3.0 years; mean BMI 26.6±4.8 kg/m2. Serum and plasma BDNF concentration was measured in the baseline state and in the 120 and 360 min of euglycemic hyperinsulinemic clamp with or without intralipid/heparin infusion. Furthermore, plasma BDNF was measured in 20 male subjects (mean age 22.7±2.3 years; mean BMI 24.9±1.5 kg/m2 360 min after a high-fat meal. Insulin sensitivity was reduced by ˜40% after 6 h of intralipid/heparin infusion (P<0.001). During both clamps, serum and plasma BDNF followed the same pattern. Hyperinsulinemia had no effect on circulating BDNF. Raising FFA had no effect on circulating BDNF in 120 min; however, it resulted in a significant decrease by 43% in serum and by 35% in plasma BDNF after 360 min (P=0.005 and 0.006, respectively). High-fat meal also resulted in a decrease by 27.8% in plasma BDNF (P=0.04).

Our data show that raising FFA decreases circulating BDNF. This might indicate a potential link between FFA-induced insulin resistance and neurodegenerative disorders [42]. Rodriguez-Navas C et al. [43] analyzed the fatty acid profile of brains and plasma from male and female mice fed chow or a western-style high fat diet (WD) for 16 weeks to determine if males and females process fatty acids differently. Based on the differences in fatty acids observed in vivo, we performed in vitro experiments on N43 hypothalamic neuronal cells to begin to elucidate how the fatty acid milieu may impact brain inflammation.

Using a comprehensive mass spectrometry fatty acid analysis, which includes a profile for 52 different fatty acid isomers, we assayed the plasma and brain fatty acid composition of age-matched male and female mice maintained on chow or a WD. Additionally, using the same techniques, we determined the fatty acid composition of N43 hypothalamic cells following exposure to palmitic and linoleic acid, alone or in combination.

The data demonstrate there is a sexual dimorphism in brain fatty acid content both following the consumption of the chow diet, as well as the WD, with males having an increased percentage of saturated fatty acids and reductions in ω6-polyunsaturated fatty acids when compared to females. Interestingly, we did not observe a sexual dimorphism in fatty acid content in the plasma of the same mice. Furthermore, exposure of N43 cells to the ω6-PUFA linoleic acid, which is higher in female brains when compared to males, reduces palmitic acid-induced inflammation.

The data suggest male and female brains, and not plasma, differ in their fatty acid profile. This is the first time, to our knowledge, lipidomic analyses has been used to directly test the hypothesis there is a sexual dimorphism in brain and plasma fatty acid composition following consumption of the chow diet, as well as following exposure to the WD.

### Blood-Brain Barrier

Treatment strategies for Alzheimer's disease (AD) are still elusive. Thus, new strategies are needed to understand the pathogenesis of AD in order to provide suitable therapeutic measures. Available evidences suggest that in AD, passage across the blood-brain barrier (BBB) and transport exchanges for amyloid-β-peptide (ABP) between blood and the central nervous system (CNS) compartments play an important regulatory role for the deposition of brain ABP. New evidences suggest that BBB is altered in AD. Studies favoring transport theory clearly show that ABP putative receptors at the BBB control the level of soluble isoform of ABP in brain. This is achieved by regulating influx of circulating ABP into brain via specific receptor for advanced glycation end products (RAGE) and gp330/megalin-mediated transcytosis. On the other hand, the efflux of brain-derived ABP into the circulation across the vascular system via BBB is accomplished by low-density receptor-related protein-1 (LRP1). Furthermore, an increased BBB permeability in AD is also likely since structural damage of endothelial cells is quite frequent in AD brain. Thus, enhanced drug delivery in AD is needed to induce neuroprotection and therapeutic success. For this purpose, nanodrug delivery could be one of the available options that require active consideration for novel therapeutic strategies to treat AD cases. This review is focused on these aspects and provides new data showing that BBB plays an important role in AD-induced neurodegeneration and neurorepair [44].

The blood-brain barrier (BBB) is a tightly regulated barrier in the central nervous system. Though the BBB is thought to be intact during neurodegenerative diseases such as Alzheimer's (AD) and Parkinson's disease (PD), recent evidence argues otherwise.

Dysfunction of the BBB may be involved in disease progression, eliciting of peripheral immune response, and, most importantly, altered drug efficacy. In this review, we will give a brief overview of the BBB, its components, and their functions. We will critically evaluate the current literature in AD and PD BBB pathology resulting from insult, neuroinflammation, and neurodegeneration. Specifically, we will discuss alterations in tight junction, transport and endothelial cell surface proteins, and vascular density changes, all of which result in altered permeability. Finally, we will discuss the implications of BBB dysfunction in current and future therapeutics. Developing a better appreciation of BBB dysfunction in AD and PD may not only provide novel strategies in treatment, but will prove an interesting milestone in understanding neurodegenerative disease etiology and progression [45].

It is not clear whether Alzheimer's Disease (AD) is primarily a neurodegenerative disorder or not. A body of evidence suggests that vascular disorder in brains of individuals with AD contributes to the extremes of this disease. This raises a question whether Alzheimer's dementia is secondary to vascular dysfunction in the central nervous system (CNS) and, therefore, the neurodegeneration that follows is a consequence of inadequate cerebral blood flow, altered brain metabolism and failure in physiological functions of brain endothelium which represents a site at the blood-brain barrier (BBB). In this paper the evidence for a primary role of the CNS vascular system in pathogenesis of Alzheimer's dementia is reviewed to show how alterations in transport across the BBB contribute to development of cerebral beta-amyloidosis in AD. In addition, vascularly-based therapeutic strategies to limit the development of beta-amyloidosis and to remove amyloid and plaques from the CNS of AD individuals are discussed [46]. Protection of the brain is strengthened by active transport and ABC transporters. P-glycoprotein (P-gp) at the blood-brain barrier (BBB) functions as an active efflux pump by extruding a substrate from the brain, which is important for maintaining loco-regional homeostasis in the brain and protection against toxic compounds. Importantly, dysfunctional BBB P-gp transport is postulated as an important factor contributing to accumulation of aggregated protein in neurodegenerative disorders such as Alzheimer's disease (AD) and Parkinson's disease (PD). Furthermore, P-gp is a major factor in mediating resistance to brain entry of numerous exogenous compounds, including toxins that can be involved in PD pathogenesis. This review highlights the role of altered P-gp function in the pathogenesis and progression of neurodegenerative disease. Also the implications of alterations in P-gp function for the treatment of these diseases are discussed [47].

The blood-brain barrier (BBB) is a tightly regulated barrier in the central nervous system. Though the BBB is thought to be intact during neurodegenerative diseases such as Alzheimer's (AD) and Parkinson's disease (PD), recent evidence argues otherwise.

Dysfunction of the BBB may be involved in disease progression, eliciting of peripheral immune response, and, most importantly, altered drug efficacy. In this review, we will give a brief overview of the BBB, its components, and their functions. We will critically evaluate the current literature in AD and PD BBB pathology resulting from insult, neuroinflammation, and neurodegeneration. Specifically, we will discuss alterations in tight junction, transport and endothelial cell surface proteins, and vascular density changes, all of which result in altered permeability. Finally, we will discuss the implications of BBB dysfunction in current and future therapeutics. Developing a better appreciation of BBB dysfunction in AD and PD may not only provide novel strategies in treatment, but will prove an interesting milestone in understanding neurodegenerative disease etiology and progression [48].

Although intravenous lipid emulsion (ILE) was first used to treat life-threatening local anesthetic (LA) toxicity, its use has expanded to include both non-local anesthetic (non-LA) poisoning and less severe manifestations of toxicity. A collaborative workgroup appraised the literature and provides evidence-based recommendations for the use of ILE in poisoning.

Following a systematic review of the literature, data were summarized in four publications: LA and non-LA poisoning efficacy, adverse effects, and analytical interferences. Twenty-two toxins or toxin categories and three clinical situations were selected for voting. Voting statements were proposed using a predetermined format. A two-round modified Delphi method was used to reach consensus on the voting statements. Disagreement was quantified using RAND/UCLA Appropriateness Method.

For the management of cardiac arrest, we recommend using ILE with bupivacaine toxicity, while our recommendations are neutral regarding its use for all other toxins. For the management of life-threatening toxicity, (1) as first line therapy, we suggest not to use ILE with toxicity from amitriptyline, non-lipid soluble beta receptor antagonists, bupropion, calcium channel blockers, cocaine, diphenhydramine, lamotrigine, malathion but are neutral for other toxins, (2) as part of treatment modalities, we suggest using ILE in bupivacaine toxicity if other therapies fail, but are neutral for other toxins, (3) if other therapies fail, we recommend ILE for bupivacaine toxicity and we suggest using ILE for toxicity due to other LAs, amitriptyline, and bupropion, but our recommendations are neutral for all other toxins. In the treatment of non-life-threatening toxicity, recommendations are variable according to the balance of expected risks and benefits for each toxin. For LA-toxicity we suggest the use of Intralipid® 20% as it is the formulation the most often reported. There is no evidence to support a recommendation for the best formulation of ILE for non-LAs. The voting panel is neutral regarding ILE dosing and infusion duration due to insufficient data for non-LAs. All recommendations were based on very low quality of evidence.

Clinical recommendations regarding the use of ILE in poisoning were only possible in a small number of scenarios and were based mainly on very low quality of evidence, balance of expected risks and benefits, adverse effects, laboratory interferences as well as related costs and resources. The workgroup emphasizes that dose-finding and controlled studies reflecting human poisoning scenarios are required to advance

knowledge of limitations, indications, adverse effects, effectiveness, and best regimen for ILE treatment [49].

Intralipid emulsion therapy is well-established for the treatment of local-anesthetic systemic toxicities. In recent years, its role has expanded as an important therapeutic agent in the reversal of other types of drug overdoses, including certain types of antipsychotics, antidepressants, antiarrhythmics, and calcium channel blockers. A literature review identified thirty-one case reports including forty-nine separate drug overdose cases involving ten separate drug classes which were successfully reversed with Intralipid. The present clinical case study describes an elderly unresponsive woman refractory to conventional treatments after ingesting a potentially lethal amount of 5.6 grams of diltiazem in a suicide attempt. After treatment with Intralipid over a twenty-four hour period, the patient's hemodynamic and metabolic derangements were corrected and stabilized completely. Intralipid emulsion rescue therapy provides another potential strategy for the reversal of many drug toxicities, most likely by providing a lipid layer safety net for drug overdose by passive diffusion. Clinicians are urged to embrace an expanded role of Intralipid emulsion rescue therapy, not only for local anesthetic drug toxicities, but also for other lipophilic drug overdoses [50].

Caffeine is arguably the most widely used stimulant drug in the world. Here we describe a suicide attempt involving caffeine overdose whereby the patient's severe intoxication was successfully treated with the prompt infusion of Intralipid. A 19-year-old man was found in an agitated state at home by the volunteer emergency team about 1 h after the intentional ingestion of 40 g of caffeine (tablets). His consciousness decreased rapidly, followed quickly by seizures, and electrocardiographic monitoring showed ventricular fibrillation. Advanced life support maneuvers were started immediately, with the patient defibrillated 10 times and administered 5 mg epinephrine in total and 300+150 mg of amiodarone (as well as lidocaine and magnesium sulfate). The cardiac rhythm eventually evolved to asystole, necessitating the intravenous injection of epinephrine to achieve the return of spontaneous circulation. However, critical hemodynamic instability persisted, with the patient's cardiac rhythm alternating between refractory irregular narrow complex tachycardia and wide complex tachycardia associated with hypotension. In an attempt to restore stability we administered three successive doses of Intralipid (120+250+100 mg), which successfully prevented a severe cardiovascular collapse due to a supra-lethal plasma caffeine level (>120 mg/L after lipid emulsion). The patient survived without any neurologic complications and was transferred to a psychiatric ward a few days later. The case emphasizes the efficacy of intravenous lipid emulsion in the resuscitation of patients from non-local anesthetic systemic toxicity. Intralipid appears to act initially as a vehicle that carries the stimulant drug away from heart and brain to less well-perfused organs (scavenging mechanism) and then, with a sufficient drop in the caffeine concentration, possibly as a tonic to the depressed heart [51].

Thrombosis and immune dysfunction are two important complications that result from the administration of parenteral nutrition. Endothelial cells within the vasculature are crucial components necessary for maintenance of normal coagulation and immune function.

We compared the effects of three commercial lipid emulsions (LEs; ClinOleic® [or Clinolipid®], and Omegaven®) differing in the levels of omega-6 polyunsaturated fatty acids, omega-3 polyunsaturated fatty acids, omega-9 monounsaturated fatty acids, and saturated fatty acids upon endothelial cell fatty acid composition using Gas chromatography, endothelial cell integrity by assessing measurement of apoptosis and necrosis using flow cytometry, endothelial cell inflammatory activation by assessing the induction of ICAM-1 by lipopolysaccharide [LPS]), and transcription factor activation (phosphorylation of NF-κB) using western blot analysis.

Gas chromatographic analysis confirmed cellular uptake of the fatty acids within the LEs; furthermore, these fatty acid changes reflected the composition of the oils and egg phosphatides used in the manufacturing of these emulsions. However, the kinetics of fatty acid uptake and processing differed between LEs. Fish oil LE negatively impacted cell viability by doubling the percentage of apoptotic and necrotic cell populations quantified by flow cytometry using Annexin V/Fluorescein and propidium iodide. The soybean oil LE did not alter cell viability, while the olive oil-predominate emulsion improved cell viability. All LEs were capable of suppressing LPS-induced ICAM-1 expression; however, the fish oil LE was more potent than the other emulsions. Fish oil LE supplementation of cells also suppressed LPS-induced phosphorylation of NF-κB, while the soybean oil and olive predominant LE had no effect upon NF-κB phosphorylation. Lipid emulsions are readily incorporated and stored in the form of triacylglycerols. Soybean oil-based, olive oil-predominant and fish-oil based LEs differentially affected endothelial cell integrity. Importantly, these three LEs were capable of suppressing endothelial cell inflammatory response despite their fatty acid content [52]. Membrane currents conducted by the NMDA receptor channels were investigated in cultured cortical neurons and TsA cells transfected with NR1-1a/NR2A subunits of the NMDA receptor. The whole-cell recording technique was used. Current transients evoked by bath application of NMDA for 5 s were characterized by a fast peak and a slow decay to 46.1+/−15.5% of the peak level at the end. When NMDA was applied in combination with various lipid emulsions (Intralipid, ClinOleic, Lipofundin or Abbolipid, the NMDA-induced currents were reduced, although this reduction did not affect the fast peak, it did affect the decay phase. The amount of reduction depended on the concentration of the lipids (in the case of Abbolipid diluted at 1:40, the current at the end of the 5-s drug application was approximately ⅔ of control). When Abbolipid was applied 40 s before NMDA, peak and late current were reduced to approximately ⅔. The effect of current reduction was the same at either of the two chosen membrane potentials (−80 and +40 mV) which indicates that the effect was not mediated by contamination of the emulsions with Mg(2+). The current reduction produced by Abbolipid was about the same in native neuronal cells and in TsA cells expressing the NR1-1a/NR2A subunits. The current reducing effect of the lipid emulsions may add to the anesthetic, analgesic and neuroprotective effects seen with hypnotics administered by way of lipid carriers [53]. Little is known about the impact of circulating lipids on brain processes. Building on evidence that chronic fat consumption stimulates hypothalamic peptides in close association with elevated triglycerides (TG), this study examined whether an acute rise in TG levels induced by fat emulsion can affect these hypothalamic systems. In normal weight rats, ip injection of Intralipid (20%, 5 ml) during the first 4 h after injection produced a robust increase in TG levels and nonesterified fatty acids, but had no impact on glucose, insulin, or leptin levels. This was accompanied by a marked increase in the expression of particular orexigenic peptides, galanin, orexins, and the opioid, enkephalin, which are known to be positively related to fat ingestion. This effect, similarly induced by 4 h of high fat diet consumption, was detected in the paraventricular nucleus (PVN) for galanin, in the perifornical hypothalamus (PFH) for orexins, and in the PVN, PFH, as well as the arcuate nucleus (ARC) for enkephalin. It was not seen, however, for neuropeptide Y and agouti-related protein localized in the ARC, which are unaffected or reduced by dietary fat. This site specificity was confirmed by c-Fos immunostaining, a marker of neuronal activity, which was increased by Intralipid in the PVN and PFH, but not in the ARC, and was detected in 20% of orexin-expressing neurons in the PFH. These findings suggest that circulating lipids, through different mechanisms, may stimulate hypothalamic neurons, which synthesize specific feeding stimulatory peptides that possibly contribute to hyperphagia during consumption of a fat-rich diet [54].

### Conclusion

Intralipid treatment is first suggested here for the treatment of Alzheimer disease. It should be given intravenously on a monthly basis according to each patient's response. Clinical studies should be done in order to evaluate this new treatment modality.

### REFERENCES

1. Alzheimer's association (2017) Treatments for Alzheimer's.
2. Berrios G E (1991) Alzheimer's Disease: A Conceptual History. International Journal of Geriatric Psychiatry 5: 355-365.
3. Engstrom, Eric J (2007) Researching Dementia in Imperial Germany: Alois Alzheimer and the Economies of Psychiatric Practice. Culture, Medicine and Psychiatry 31: 405-413.
4. https://en.wikipedia.org/wiki/Alois_Alzheimer
5. Zipser B D, Johanson J, Gonzalez L, Berzin T M, Tavares R, et al. (2007) Microvascular injury and blood—brain barrier leakage in Alzheimer's disease. Neurobiology of Aging 28: 977-86.
6. Nagele, Robert G (2006) Alzheimer's disease: new mechanisms for an old problem. UMDNJ Research. University of Medicine and Dentistry of New Jersey 7: 2.
7. https://webbeta.archive.org/web/20110917001430/http://www.umdnj.edu/research/publications/fall0 6/4.htm
8. Daviglus M L, Bell C C, Berrettini W (2010) National Institutes of Health state-of-the-science conference statement: preventing Alzheimer disease and cognitive decline. Annals of Internal Medicine 153: 176-181.
9. World Health Organization and Alzheimer's Disease International, Dementia (2012) A Public Health Priority.
10. David M S, Ann M S, Shi D Y, Berislav Z (2000) Method to increase cerebral blood flow in amyloid angiopathy.
11. Russell R L, Westfall B A (1962) Alleviation of barbiturate depression. Anesth Analg 41: 582-585.
12. Krieglstein J, Meffert A, Niemeyer D (1974) Influence of emulsified fat on chlorpromazine availability in rabbit blood. Experientia 30:924-926.
13. Straathof D J, Driessen O, Meijer J W, Van Rees H, Vermeij P, et al. (1984) Influence of Intralipid infusion on the elimination of phenytoin. Arch Int Pharmacodyn Ther 267: 180-186.
14. Weinberg G L, VadeBoncouer T, Ramaraju G A, Garcia-Amaro M F, Cwik M J (1998) Pretreatment or resuscitation with a lipid infusion shifts the dose-response to bupivacaine-induced asystole in rats. Anesthesiology 88: 1071-1075.
15. Weinberg G (2002) Current concepts in resuscitation of patients with local anesthetic cardiac toxicity. Reg Anesth Pain Med 27: 568-575.
16. Cave G, Harvey M (2009) Intravenous lipid emulsion as antidote beyond local anesthetic toxicity: a systematic review. Acad Emerg Med 16: 815-824.
17. Rosenblatt M A, Abel M, Fischer G W, Itzkovich C J, Eisenkraft J B (2006) Successful use of a 20% lipid emulsion to resuscitate a patient after a presumed bupivacaine-related cardiac arrest. Anesthesiology 105: 217-218.
18. Weinberg G, Di Gregorio G, Hiller D, Hewett A, Sirianni A (2009) Reversal of haloperidol-induced cardiac arrest by using lipid emulsion. Ann Intern Med 150: 737-738.
19. Minton N A, Goode A G, Henry J A (1987) The effect of a lipid suspension on amitriptyline disposition. Arch Toxicol 60: 467-469.
20. Cave G, Harvey M, Graudins A (2011) Review article: Intravenous lipid emulsion as antidote: A summary of published human experience. Emerg Med Australas 23: 123-141.
21. Tomiyama T (2010) Involvement of beta-amyloid in the etiology of Alzheimer's disease. Brain Nerve 62: 691-699.
22. Bloom G S (2014) Amyloid-Î² and tau: the trigger and bullet in Alzheimer disease pathogenesis. JAMA Neurol 71: 505-508.
23. Zetterberg H, Mattsson N (2014) Understanding the cause of sporadic Alzheimer's disease. Expert Rev Neurother 14: 621-630.
24. Morris G P, Clark I A, Vissel B (2014) Inconsistencies and controversies surrounding the amyloid hypothesis of Alzheimer's disease. Acta Neuropathol Commun 2:135.
25. James A P, Pal S, Gennat H C, Vine D F, Mamo J C (2003) The incorporation and metabolism of amyloid-beta into chylomicron-like lipid emulsions. J Alzheimers Dis 5: 179-188.
26. Champeil P G, Denis I, Goustard L B, Alessandri J M, Guesnet P, et al. (2004) Astrocytes in culture require docosahexaenoic acid to restore the n-3/n-6 polyunsaturated fatty acid balance in their membrane phospholipids. J Neurosci Res 75: 96-106.
27. Langelier B, Linard A, Bordat C, Lavialle M, Heberden C (2010) Long chain-polyunsaturated fatty acids modulate membrane phospholipid composition and protein localization in lipid rafts of neural stem cell cultures. J Cell Biochem 110: 1356-64.
28. Colin J, Gregory P L, Lanhers M C, Claudepierre T, Corbier C, et al. (2016) Membrane raft domains and remodeling in aging brain. Biochimie 130: 178-187.
29. Taftachi F, Sanaei Z H, Sepehrian B, Zamani N (2012) Lipid emulsion improves Glasgow coma scale and decreases blood glucose level in the setting of acute non-local anesthetic drug poisoning—a randomized controlled trial. Eur Rev Med Pharmacol Sci 1:38-42.
30. Weigt H U, Georgieff M, Beyer C, Föhr KJ (2002) Activation of neuronal N-methyl-D-aspartate receptor channels by lipid emulsions. Anesth Analg 94: 331-337.
31. Haywood S C, Bree A J, Puente E C, Daphna-Iken D, Fisher S J (2009) Central but not systemic lipid infusion augments the counterregulatory response to hypoglycemia. Am J Physiol Endocrinol Metab 297: E50-E60.
32. Hiller D B, Di Gregorio G, Kelly K, Ripper R, Edelman L, et al. (2010) Safety of high volume lipid emulsion infusion: a first approximation of LD50 in rats. Reg Anesth Pain Med 35: 140-144.
33. Heinonen J A, Litonius E, Backman J T, Neuvonen P J, Rosenberg P H (2013) Intravenous lipid emulsion entraps amitriptyline into plasma and can lower its brain concentration—an experimental intoxication study in pigs. Basic Clin Pharmacol Toxicol 113: 193-200.
34. Basarslan S K, Alp H, Senol S, Evliyaoglu O, Ozkan U (2014) Is intralipid fat emulsion a promising therapeutic strategy on neurotoxicity induced by malathion in rats? Eur Rev Med Pharmacol Sci. 18: 471-476.
35. Ozkan U, Osun A, Basarslan K, Senol S, Kaplan I, et al. (2014) Effects of intralipid and caffeic acid phenethyl ester on neurotoxicity, oxidative stress, and acetylcholinesterase activity in acute chlorpyriphos intoxication. Int J Clin Exp Med 7:837-46.
36. Heinonen J A, Litonius E, Salmi T, Haasio J, Tarkkila P, et al. (2015) Intravenous lipid emulsion given to volunteers does not affect symptoms of lidocaine brain toxicity. Basic Clin Pharmacol Toxicol 116: 378-83.
37. Lange D B, Schwartz D, DaRoza G, Gair R (2012) Use of intravenous lipid emulsion to reverse central nervous system toxicity of an iatrogenic local anesthetic overdose in a patient on peritoneal dialysis. Ann Pharmacother 46: e37.
38. http://ecatalog.baxter.com/ecatalog/loadResource.blob?bid=20000307
39. Gilbert D P, Tae W K (2011) Linking lipids to Alzheimer's disease: cholesterol and beyond. Nature Reviews Neuroscience12: 284-296.
40. Terracina L, Brunetti M, Avellini L, De Medio G E, Trovarelli G, Gaiti A (1992) Linoleic acid metabolism in brain cortex of aged rats. The Italian Journal of Biochemistry 41:225-235.
41. Thomas M H, Pelleieux S, Vitale N, Olivier J L (2016) Dietary arachidonic acid as a risk factor for age-associated neurodegenerative diseases: Potential mechanisms. Biochimie 130: 168-177.
42. Karczewska K M, Kowalska I, NikoÅ,ajuk A, Adamska A, ZieliÅska M, et al. (2012) Circulating brain-derived neurotrophic factor concentration is downregulated by intralipid/heparin infusion or high-fat meal in young healthy male subjects. Diabetes Care 35: 358-362.
43. Rodriguez-Navas C, Morselli E, Clegg D J (2016) Sexually dimorphic brain fatty acid composition in low and high fat diet-fed mice. Mol Metab 5: 680-689.
44. Sharma H S, Castellani R J, Smith M A, Sharma A (2012) The blood-brain barrier in Alzheimer's disease: novel therapeutic targets and nanodrug delivery. Int Rev Neurobiol 102: 47-90.
45. Desai B S, Monahan A J, Carvey P M, Hendey B (2007) Blood-brain barrier pathology in Alzheimer's and Parkinson's disease: implications for drug therapy. Cell Transplant 16: 285-299.
46. Zlokovic B V (2002) Vascular disorder in Alzheimer's disease: role in pathogenesis of dementia and therapeutic targets. Adv Drug Deliv Rev 54: 1553-1559.
47. Bartels A L (2011) Blood-brain barrier P-glycoprotein function in neurodegenerative disease. Curr Pharm Des 17: 2771-2777.
48. Desai B S, Monahan A J, Carvey P M, Hendey B (2007) Blood-brain barrier pathology in Alzheimer's and Parkinson's disease: implications for drug therapy. Cell Transplant 16: 285-299.
49. Gosselin S, Hoegberg L C, Hoffman R S (2016) Evidence-based recommendations on the use of intravenous lipid emulsion therapy in poisoning. Clin Toxicol (Phila) 54: 899-923.
50. Muller S H, Diaz J H, Kaye A D (2016) Intralipid Emulsion Rescue Therapy: Emerging Therapeutic Indications in Medical Practice. J La State Med Soc 168: 101-103.
51. Muraro L, Longo L, Geraldini F, Bortot A, Paoli A, et al. (2016) Intralipid in acute caffeine intoxication: a case report. J Anesth 30: 895-899.
52. Harvey K A, Xu Z, Pavlina T M, Zaloga G P, Siddiqui R A (2015) Modulation of endothelial cell integrity and inflammatory activation by commercial lipid emulsions. Lipids Health Dis 18: 14-19.
53. Weigt H, Georgieff M, Beyer C, Georgieff E M, Kuhse J, et al. (2004) Lipid emulsions reduce NMDA-evoked currents. Neuropharmacology 47: 373-380.
54. Chang G Q, Karatayev O, Davydova Z, Leibowitz S F (2004) Circulating triglycerides impact on orexigenic peptides and neuronal activity in hypothalamus. Endocrinology 145: 3904-3912.

### Example 10

### Lipid Emulsion Treatment (LET) of Post Operative Cognitive Dysfunction (POCD)

Postoperative delirium (POD) or Post operative cognitive dysfunction (POCD) is a common and serious adverse event in the elderly patient and is associated with significant morbidity and mortality. A new treatment for POD/POCD by intravenous Intralipid (lipid emulsion) injection in the recovery room was first suggested by Eldor on 2017 (http://medcraveonline.com/JACCOA/JACCOA-07-00273.pdf).

The 4 case reports in this article describing the successful use of lipid emulsions (Smoflipid 20% and Lipidem 20%) are the first case reports of Lipid Emulsion Treatment (LET) of Post operative cognitive dysfunction (POCD) in the medical literature.

All the 4 patients mentioned in this article have given their signed written permission to use their video clips taken in the recovery room for scientific purposes to all the scientific community all over the world.

### Case Reports

### Case 1

82 years old male, hospitalized because of abscess of the muscle on the back (thoracic part). He has type 2 diabetes, common pulmonary infection. The first surgery for abscess drainage was under local anesthesia uneventfully. He was under antibiotic (Vancomycin lgr, Cefriaxon lgr), Acetaminophen lgr, Voltaren (NSAIDS) 50 mg I.M/day, Omeprazol 40 mg/day for 7 days.

He had been under second surgery for larger incision to drain the abscess under balanced general anesthesia with ETT, Propofol 200 mg, Fentanyl 150 mcg, Rocuronium 35 mg. Bridion 200 mg (Sugammadex) was used to reverse the residual muscle relaxant before extubation. The surgery time was 40 min.

After extubation, patient had been agitated, uncooperative, incomprehensible communication. Because he risked to falling and taking the LV line by his own, textile strings were used to tide his arms to bed. He was given 1 vial of Haloperidol i.m, 2.5 mg i.v bolus of midazolam, but the situation had not improved after 60 min.

We decided to start Lipid Emulsion Therapy (LET): 250 ml IV Smoflipid 20% (Fresenius Kabi) over 30 min, and continued the second vial over 120 min thereafter. The effectiveness of LET:

At 30 min after finishing the first 250 ml Smoflipid 20%, patient was calmer, could talk with comprehensible phrases. Because he could not speak Vietnamese, so we asked his relative to communicate with him. He was cooperative, but he was a little bit relentless. The strings were released regarding his demand and lesser risk of unattended behaviors estimated by staffs. The second vial of 250 ml Smoflipid 20% had been continued.

At 60 min, he was cooperative, comprehensible communication, no agitation. The strings were taken out when risk of inappropriate behaviors were lessen.

At 180 min (3 h), he was calm, cooperative, thirty, hungry. We let him sitting up with the care of his relative.

At 240 min, Termination of the second vial of LET, he was nearly normal and had been returned to ward.

Video Clip Case 1: https://youtu.be/eNclUfCqntc

### Case 2

77 years old Male admitted to hospital due to acute gangrenous cholecystitis.

His past medical history was type 2 diabetes, hypertension, coronary ischemic disease, cerebral ischemic attack for 10 years ago, Parkinson. Cerebral CT Scan revealed the occupational region on the left orbit. Thoracic CT Scan also revealed a thoracic aortic aneurysm. FBC; WBC: 19.36 G/1, NEU 92.8%, Glusose: 8.2 mmol/L, Total Bilirubin: 71.72 mmol/L, Direct Bilirubin: 32.4 mmol/L, ASAT: 352 U/L, ALAST: 265 U/L, Na+: 135 mmol/L, K: 4.2 mmol/L, Calcium: 102 mmol/L, CPR: 35.2 mg/L, CK-MB: 20 U/L, Creatinne: 1.36 mg/dL, Urea: 40.15 mg/dL, ECG: ischemic heart disease on the anterior wall. BP: 180/90 mm Hg, HR 90 BPM, BR 16, Temperature 38.5. He was fully conscious, cooperative, no localized neurologic signs, no chest pain.

He was treated by antibiotic (Meropenem lgr I.V) Because his condition was so frail so the radical surgery was not planned. The Percutaneous Transhepatic Biliary Drainage (PTBD) was proposed to relief the symptoms

He was under local anesthesia with sedative (Propofol 50 mg+Fentanyl 100 mcg) with Oxygen cannula. The PTBD procedure was 30 min under the ultrasound and Fluoroscopy.

In the recovery room, his mental status was disoriented, agitated, incomprehensible communication, relentless. He was tied to the bed by strings because of falling risk.

We started LET by i.v infusion 250 ml Lipidem 20%, (B. Braun) over 30 min. After 100 ml Lipidem 20% infused over 10 min, patient was less relentless, calmer, less agitated, communication became easier. The second vial 250 ml of Lipidem 20% was continued in 30 min.

Approximately 120 min after LET, patient was not agitated, cooperative, comprehensible communication, the strings were released.

4 h after LET, his mental status was nearly normal as before having surgery. He was returned to the ward. He was uneventful thereafter.

Video Clip Case 2: https://youtu.be/1Wqz4wNkhPo

### Case 3

81 years old male, benign prostate enlargement hospitalized for Trans Urethral Resection of Prostate (TURP). His past medical story was hypertension, ischemic heart disease. He had closed head trauma 5 years ago, but no mental disorientation thereafter. He was cooperative, non dependent activity in daily life.

The laboratory pre operative tests were non specific. Ionogram: Na 140 mmol/dL, K 3.4 mmol/dL, Cl 107 mmol/dL, Urea 52.73 mmol/dL, Creatinin 1.1 mmol/dL, Glucose 8.8 mmol/dL. ECG revealed the chronic ischemic heart disease, but the conserved heart function (Left Ventricular EF: 72%).

He was under Spinal Anesthesia for TURP. The BP before SA 180/100, HR 85 BPM, SpO2 99%, Midazolam 2 mg I.V for sedation before SA puncture at L3-4, dose was 8 mg Heavy Bupivacaine 0.5%+20 mcg Fentanyl. The surgery time was 20 min, the irrigation solution was 1000 ml per operation, the crystalloid solution given per operation was 500 ml NaCl 0.9%, no vasoconstrictor used.

For post operative: Tramadol 100 mg I.V diluted in 100 ml NaCl 0.9%, interval 8 h; Odansetron 8 mg I.V; Omeprazol 40 mg I.V; NaCl 0.9% 1000 ml for irrigation.

3 h post operation, his mental status was very disorientated, agitated, yelling, screaming. The on duty anesthesiologist had used 2.5 mg Midazolam bolus+Propofol infusion to sedate him. As soon as infusion finished, the mental crisis rebound and 5 mg I.M Haloperidol was given during the night, but was not effective as the Propofol infusion.

On the next morning, his crisis rebound severely, so several nurses had to tie him to the bed.

We diagnosed this acute mental crisis as Post Operative Delirium. There were two possibilities that may cause POD on this patient:the TURP Syndrome and The Serotonin Syndrome (due to Tramadol)

We decided to use LET as challenging therapy. First vial of 250 ml Lipidem 20% intravenously was given over 60 min.

At 30 min after LET, he was calm, less agitated, comprehensible communication

60 min after LET, he was calm, cooperative, comprehensible communication, he was thirsty and asked for drink

120 min after LET, the mental crisis was nearly gone, he was released from the strings, he drank and eat soft foods. The ionogram revealed the Na 131 mmol/dL, K 3.03 mmol/dL, Cl 99 mmol/dL.

240 min (4 h) after LET, he was a little bit more relentless, but cooperative, comprehensible communication. He complained of discomfort due to urine catheter. We decided to use the second vial of 250 ml Lipidem 20% to maintain the effectiveness.

8 h from the LET, patient was calm, oriented, well communicated, comfortable, not complained on urine catheter, he drank, eaten normally.

No mental crisis thereafter.

Video clip case 3: https://youtu.be/ZKOaOqdEPLO

### Case 4

79 years old hypertensive female, hospitalized in emergency for biliary duct infection. Her vital signs were stable, temperature 38.5, BP 130/80, HR 80 BPM. The CT scan revealed the intra and extra hepatic biliary duct dilatation with the stone at the end of common bile duct. FBC: WBC 16 G/l, Neu 88.9%, Total Billirubin 117.2 mmol/L, direct Billirubin 70 mmol/l, AST 89UI/l, ALT 54 UI/L, Glucose 86 mg/dL, Urea 37 mg/dL, K 2.93 mmol/L, Cl 102 mmol/L, Troponin 0.024 ng/ml.

She was treated by antibiotic (Meropenem 1000 mg every 8 h+Metronidazole 500 mg every 8 h) and was indicated for ERCP procedure. (Endoscopic Retrograde Cholangio—Pancreatography)

She was under general anesthesia with ETT. The balanced anesthesia with Propofol, Fentanyl, Rocuronium, Servoran was uneventfull, she was neutralized by Bridion 100 mg (Sugammadex) before extubation. The surgery lasted for 40 mins.

At recovery room, she was relentless, slight agitated, but not disorientated, comprehensible communication, but complained of discomfort at abdomen, not pain. The vital signs were stable. Because she was overweight and risk of falling, taking out the I.V line by her own, so the strings were used to tie her arms to the bed.

We decided to use LET for this situation.

250 ml Lipidem 20% was intravenously infused over 60 min.

After 30 min LET, her mental status seemed better, but not clear, the strings were released for her comfort

After 60 min LET, her mental status was improved significantly. She was less relentless, less agitated, cooperative, better communication. She asked for drink

After 120 min, she was with better communication, cooperative, smiled, “did not remember what had happened before”.

At 180 min, the mental status was nearly the same. We decided to continue the second vial 250 of Lipidem 20% for sustainable effectiveness.

### Discussion

### Post Operative Delirium/Post Operative Cognitive Dysfunction

Delirium is defined by either the Diagnostic and Statistical Manual of Mental Disorders, Fifth Edition (DSM-5) (1) or by the 10th revision of the International Statistical Classification of Diseases and Related Health Problems (ICD 10, Table 3) (2). Delirium is an acute and fluctuating alteration of mental state of reduced awareness and disturbance of attention. POD (Post Operative Delirium) often starts in the recovery room and occurs up to 5 days after surgery (3-5). One investigation (4) found that many patients with POD on the peripheral ward already had POD in the recovery room.

More than 230 million surgical procedures are performed each year worldwide, of which more than 80 million are in Europe (6-8). In Europe, the in-hospital mortality rate up to a maximum of 60 days is 3% after elective surgery and nearly 10% after emergency surgery (7). In addition to mortality, postoperative cognitive impairments such as POD and postoperative cognitive dysfunction (POCD) impose a huge burden on individuals and society (9). The incidence of POD is dependent on perioperative and intraoperative risk factors (10). Therefore, the incidence of POD varies within a broad range (11, 12). For example, a meta-analysis of 26 studies of POD reported an incidence of 4.0 to 53.3% in hip fracture patients and 3.6 to 28.3% in elective patients (13).

Delirium is one of the most common complications following hip fracture surgery in older people. This study identified pre- and peri-operative factors associated with the development of post-operative delirium following hip fracture surgery.

Published and unpublished literature were searched to identify all evidence reporting variables on patient characteristics, on-admission, intra-operative and post-operative management assessing incident delirium in older people following hip fracture surgery. Pooled odds ratio (OR) and mean difference of those who experienced delirium compared to those who did not were calculated for each variable. Evidence was assessed using the Downs and Black appraisal tool and interpreted using the GRADE approach.

A total of 6704 people (2090 people with post-operative delirium) from 32 studies were analysed. There was moderate evidence of nearly a two-times greater probability of post-operative delirium for those aged 80 years and over (OR: 1.77; 95% CI: 1.09, 2.87), whether patients lived in a care institution pre-admission (OR: 2.65; 95% CI: 1.79, 3.92), and a six-times greater probability of developing post-operative delirium with a pre-admission diagnosis of dementia (OR: 6.07, 95% CI: 4.84, 7.62). There was no association with intra-operative variables and probability of delirium.

Clinicians treating people with a hip fracture should be vigilant towards post-operative delirium if their patients are older, have pre-existing cognitive impairment and poorer overall general health. This is also the case for those who experience post-operative complications such as pneumonia or a urinary tract infection (Smith T O et al 2017) (14).

Post-operative cerebral dysfunction includes delirium, usually occurring early and reversible, and post-operative cognitive disorders, usually occurring later and prolonged. This is a frequent complication in patients older than 75 years old. The two neurological pictures are often inter-related. The pathophysiology of both entities is similar and related to post-operative neuro-inflammation; therefore onset may occur independently of any surgical complication. Post-operative cerebral dysfunction is a serious organic complication. Reduction of inflammation represents the most logical preventive measure but currently there are no studies that show this to be effective. Prevention therefore means combining several minor measures, elements that fit well into programs of enhanced post-operative recovery after surgery. Diminished pre-operative cognitive status being a major risk factor, pre-operative rehabilitation combining nutritional, physical and cognitive support can be helpful (Benhamou D, Brouquet A 2016) (15).

Postoperative delirium is a common and serious adverse event in the elderly patient and is associated with significant morbidity and mortality. It is of great importance to identify patients at risk for delirium, in order to focus preventive strategies. The aim of this article is to systematically review current available literature on pre-operative risk factors for delirium after vascular surgery.

A systematic literature search was conducted using PubMed and EMBASE, using the MeSH terms and key words “delirium”, “surgery” and “risk factor”. Studies were retained for review after meeting strict inclusion criteria that included only prospective studies evaluating risk factors for delirium in patients who had elective vascular surgery. Diagnosis of delirium needed to be confirmed using the Diagnostic and Statistical Manual of Mental Disorders (DSM) or ICD-10.

Fifteen articles were selected for inclusion, incidence of delirium across the studies ranged from 5% to 39%. Many factors have been associated with increased risk of delirium, including age, cognitive impairment, comorbidity, depression, smoking, alcohol, visual and hearing impairment, ASA-score, biochemical abnormalities, operative strategies and blood loss.

Delirium is a common complication after elective vascular surgery in elderly. The highest delirium incidence was observed after open aortic surgery as well as after surgery for critical limb ischemia. A picture starts to form of which predisposing factors lead to increased risk of delirium. The leading risk factors consistently identified in this systematic review were advanced age and cognitive impairment. Multi-disciplinary specialist-led interventions in the preoperative phase could decrease incidence and severity of delirium and should be focused on identified high-risk patients (.Raats J W et al. 2016) (16).

This study (17) investigates the relationship between cognitive dysfunction or delirium detected in the early post-surgical phase and the 1-year mortality among 514 hip fracture hospitalized older persons. Patients with early cognitive dysfunction or delirium experienced a 2-fold increased mortality risk. Early post-operative cognitive dysfunction and delirium are negative prognostic factors for mortality.

Premorbid cognitive impairment and dementia in older individuals negatively affect functional recovery after hip fracture. Additionally, post-operative delirium is an established risk factor for negative outcomes among hip fracture patients. While the majority of hip fracture patients experience minor post-surgical cognitive dysfunction, the prognostic value of this phenomenon is unknown. Therefore, we investigated the relationship between minor cognitive dysfunction or delirium detected in the early post-surgical phase and the 1-year mortality after index hip fracture.

We enrolled 514 patients with hip fracture (77.4% women), aged 65 years or older (mean age 83.1±7.3 years), who underwent surgical hip fracture repair. Patients were assessed daily from the second to the fourth post-operative day and at 3, 6, and 12 months thereafter. All participants underwent comprehensive assessment, including detection of delirium by using the confusion assessment method and evaluation of cognitive function by using mini-mental state examination (MMSE; score range 0 to 30, with lower scores indicating poorer performance). In the absence of delirium, post-surgical cognitive dysfunction was defined as having low performance on MMSE. Vital status of 1 year after the index fracture and date of death were gathered from local registries.

The observed 1-year mortality rate was 14.8%. Men were more likely to die than women within 1 year of the index fracture (p<0.01). Compared to participants with better cognitive performance, those with MMSE <24, as well as those with delirium in the post-operative phase, showed a significantly higher 1-year mortality rate (23.3 versus 17.9 and 8.1%, respectively). Independent of age and sex, post-operative cognitive dysfunction as well as delirium was both associated with a 2-fold increased mortality risk.

The presence of minor cognitive dysfunction in the early post-surgical phase is a negative prognostic factor for mortality among elderly hip fracture patients. The burden of minor cognitive dysfunction is likely superimposed on that of delirium in subgroups of frail patients (Ruggiero C et al. 2017) (17).

Perioperative cerebral hypoperfusion/ischemia is a major inciting factor of postoperative delirium, which is coupled with adverse outcome in elderly patients. Cerebral oximetry enables noninvasive assessment of the regional cerebral oxygen saturation (rSO2). This study aimed to investigate whether perioperative rSO2 variations were linked to delirium in elderly patients after spinal surgery.

Postoperative delirium was assessed for 48 hours postsurgery in 109 patients aged over 60 years without a prior history of cerebrovascular or psychiatric diseases by the Confusion Assessment Method for the intensive care unit and the intensive care delirium screening checklist. The rSO2 values immediately before and throughout surgery were acquired. The preoperative cognitive functions, patient characteristics, and perioperative data were recorded.

During the 48-h postoperative period, 9 patients (8%) exhibited delirium. The patients with delirium showed similar perioperative rSO2 values as those without, in terms of the median lowest rSO2 values (55% vs. 56%; P=0.876) and incidence (22%, both) and duration of decline of rSO2<80% of the baseline values. The serially assessed hemodynamic variables, hematocrit levels, and blood gas analysis variables were also similar between the groups, except for the number of hypotensive events per patient, which was higher in the patients with delirium than in those without (4, interquartile range [IQR] 3 to 6 vs. 2, IQR: lto 3; P=0.014).

The degree and duration of decrease of the perioperative rSO2 measurements were not associated with delirium in elderly patients after spinal surgery (Soh S et al 2016) (18).

Three-dimensional Arterial Spin Labeling (ASL) MRI was performed before surgery in a cohort of 146 prospectively enrolled subjects ≥70 years old scheduled to undergo elective surgery. We investigated the prospective association between ASL-derived measures of cerebral blood flow (CBF) before surgery with postoperative delirium incidence and severity using whole-brain and globally normalized voxel-wise analysis. We also investigated the cross-sectional association of CBF with patients' baseline performance on specific neuropsychological tests, and with a composite general cognitive performance measure (GCP). Out of 146 subjects, 32 (22%) developed delirium. We found no significant association between global and voxel-wise CBF with delirium incidence or severity. We found the most significant positive associations between CBF of the posterior cingulate and precuneus and the Hopkins Verbal Learning Test—Revised total score, Visual Search and Attention Test (VSAT) score and the GCP composite. VSAT score was also strongly associated with right parietal lobe CBF. ASL can be employed in a large, well-characterized older cohort to examine associations between CBF and age-related cognitive performance. Although ASL CBF measures in regions previously associated with preclinical Alzheimer's Disease were correlated with cognition, they were not found to be indicators of baseline pathology that may increase risk for delirium (.Hshieh T T et al. 2016) (19).

Oxidative stress may be involved in occurrence of postoperative delirium (POD) and cognitive dysfunction (POCD). 8-iso-Prostaglandin F2α (8-iso-PGF2α), an isoprostane derived from arachidonic acid via lipid peroxidation, is considered a gold standard for measuring oxidative stress. The present study aimed to investigate the ability of postoperative plasma 8-iso-PGF2α levels to predict POD and POCD in elderly patients undergoing hip fracture surgery.

Postoperative plasma 8-iso-PGF2α levels of 182 patients were measured by an enzyme-linked immunosorbent assay. We assessed the relationships between plasma 8-iso-PGF2α levels and the risk of POD and POCD using a multivariate analysis.

Plasma 8-iso-PGF2α levels and age were identified as the independent predictors for POD and POCD. Based on areas under receiver operating characteristic curve, the predictive values of 8-iso-PGF2α were obviously higher than those of age for POD and POCD. In a combined logistic-regression model, 8-iso-PGF2α significantly enhanced the areas under curve of age for prediction of POD and POCD.

Postoperative plasma 8-iso-PGF2α levels may have the potential to predict POD and POCD in elder patients undergoing hip fracture surgery (Zheng Y B e al. 2016) (20).

Risk factors for delirium following cardiac surgery are incompletely understood. The aim of this study was to investigate whether intra-operative pathophysiological alterations and therapeutic interventions influence the risk of post-operative delirium.

This retrospective cohort study was performed in a 12-bed cardiosurgical intensive care unit (ICU) of a university hospital and included patients consecutively admitted after cardiac surgery during a 2-month period. The diagnosis of delirium was made clinically using validated scores. Comparisons between patients with and without delirium were performed with non-parametric tests. Logistic regression was applied to identify independent risk factors. Results are given as number (percent) or median (range).

Of the 194 consecutive post-cardiac surgery patients, 50 (26%) developed delirium during their ICU stay. Univariate analysis revealed that significant differences between patients with and without delirium occurred in the following intra-operative variables: duration of cardiopulmonary bypass (184 [72-299] vs 113 (37-717) minutes, p<0.001), lowest mean arterial pressure (50 [30-70] vs 55 [30-75] mmHg, p=0.004), lowest haemoglobin level (85 [56-133] vs 98 [53-150] g/L, p=0.005), lowest body temperature (34.5 [24.4-37.2] vs 35.1 [23.9-37.2] ° C., p=0.035), highest noradrenaline support (0.11 [0.00-0.69] vs 0.07 [0.00-0.42] μg/kg/minute, p=0.001), and frequency of red blood cell transfusions (18 [36%] vs 26 [18%], p=0.018) and platelet transfusions (23 [46%] vs 24 [17%], p<0.001). Only platelet transfusions remained an independent risk factor in the multivariate analysis (p<0.001).

In patients undergoing cardiac surgery, various intra-operative events, such as transfusion of platelets, were risk factors for the development of a post-operative delirium in the ICU. Further research is needed to unravel the underlying mechanisms (Rudiger A et al. 2016) (21).

In this study, Bilge E U et al. (23) aimed to determine the risk factors and the incidence of delirium in patients who were followed postoperatively in our surgical intensive care unit for 24 h using the confusion assessment method (CAM).

After obtaining approval from the ethics committee, 250 patients were included in the study. Patients who were operated under general anaesthesia or regional anaesthesia and followed in the surgical intensive care unit were evaluated by the Ramsay Sedation Scale on the first postoperative day. CAM was applied to the patients who had a Ramsay Sedation Score of ≤4. Patients' age, gender, American Society of Anesthesiologists (ASA) scores, preoperative risk factors, type of anaesthesia, operation time, intra-operative procedures, pain scores evaluated by the visual analogue scale (VAS) and postoperative analgesia methods were recorded.

The incidence of delirium was found to be 18.4%. The average age of patients who developed delirium was greater than the others (68.8±12.7 and 57.6±12, p=0.001, respectively). It was observed that a one-unit increase in the ASA score resulted in a 3.3-fold increase in the risk of delirium. The incidence of delirium in patients undergoing regional anaesthesia was 34.6%, whereas it was 16.5% in patients receiving general anaesthesia (p=0.024). The existence of preoperative diabetes mellitus (DM) and chronic obstructive pulmonary disease (COPD) was shown to improve the development of delirium (p<0.05). Delirium incidence was significantly higher in patients who were administered meperidine for postoperative analgesia (p=0.013). The VAS scores of patients who developed delirium were found to be significantly higher (p=0.006).

As a result, we found that older age, high ASA score, preoperative DM and COPD are important risk factors for the development of delirium. Regional anaesthesia, high postoperative pain scores and meperidine use were observed to be associated with the development of delirium. In the postoperative period, addition of CAM, a simple measurement technique, to the daily follow-up forms can provide the early recognition of delirium, which is often underdiagnosed. We think that identification and prevention of effective risk factors have the primary importance for postoperative delirium (Bilge E U et al. 2015) (22).

Delirium after cardiac surgery is a major problem. The exact mechanisms behind delirium are not understood. Potential pathways of delirium include neurotransmitter interference, global cognitive disorder, and neuroinflammation. Several predisposing and precipitating risk factors have been identified for postoperative delirium. The development of delirium following cardiac surgery is associated with worse outcomes in the perioperative period. Multiple interventions are being explored for the prevention and treatment of delirium. Studies investigating the potential roles of biomarkers in delirium as well as pharmacological interventions to reduce the incidence and duration of delirium are necessary to mitigate this negative outcome (O'Neal J B, Shaw A D et al. 2016) (23).

Perhaps the most frequently described mechanism of brain injury in CABG surgery is based on the recognition that microemboli are generated by the surgeon manipulating the heart and aorta, through cardiotomy suctioning, and by the cardiopulmonary bypass circuit itself. Microemboli can be detected intraoperatively as high-intensity transient signals by transcranial Doppler sonography. They have the potential to lodge in cerebral microvasculature, impairing blood supply to the brain and thus cerebral oxygenation. Several phases during cardiac surgery have been associated with increased risk of embolic showers. Aortic cannulation and clamping (during application of cardiopulmonary bypass) increase the high-intensity transient signal rate, particularly if there is extensive atheroma in the ascending aorta (24). It is not surprising, therefore, that most (81%) microemboli are generated at the point of aortic cross-clamp release (25). Retaining the shed mediastinal blood with cardiotomy suckers provides an additional source of lipid emboli and other fragments (26).

### Lipid Emulsion—Mitochondrial Sink Effect

Papadopoulou A et al. (27) hypothesized that by substituting a dye surrogate in place of local anesthetic, they could visually demonstrate dye sequestration by lipid emulsion that would be dependent on both dye lipophilicity and the amount of lipid emulsion used.

They selected 2 lipophilic dyes, acid blue 25 and Victoria blue, with log P values comparable to lidocaine and bupivacaine, respectively. Each dye solution was mixed with combinations of lipid emulsion and water to emulate “lipid rescue” treatment at dye concentrations equivalent to fatal, cardiotoxic, and neurotoxic local anesthetic plasma concentrations. The lipid emulsion volumes added to each dye solution emulated equivalent intravenous doses of 100, 500, and 900 mL of 20% Intralipid in a 75-kg adult. After mixing, the samples were separated into a lipid-rich supernatant and a lipid-poor subnatant by heparin flocculation. The subnatants were isolated, and their colors compared against a graduated dye concentration scale.

Lipid emulsion addition resulted in significant dye acquisition by the lipid compartment accompanied by a reduction in the color intensity of the aqueous phase that could be readily observed. The greatest amount of sequestration occurred with the dye possessing the higher log P value and the greatest amount of lipid emulsion.

This study provides a visual demonstration of the lipid sink effect. It supports the theory that lipid emulsion may reduce the amount of free drug present in plasma from concentrations associated with an invariably fatal outcome to those that are potentially survivable.

Local anesthetic (LA) intoxication with cardiovascular arrest is a potential fatal complication of regional anesthesia. Lipid resuscitation has been recommended for the treatment of LA-induced cardiac arrest. Aim of the study (28) was to compare four different rescue regimens using epinephrine and/or lipid emulsion and vasopressin to treat cardiac arrest caused by bupivacaine intoxication.

Twenty-eight piglets were randomized into four groups (4×7), anesthetized with sevoflurane, intubated, and ventilated. Bupivacaine was infused with a syringe driver via central venous catheter at a rate of 1 mg·kg⁻¹·min⁻¹ until circulatory arrest. Bupivacaine infusion and sevoflurane were then stopped, chest compression was started, and the pigs were ventilated with 100% oxygen. After 1 min, epinephrine 10 μg·kg⁻¹ (group 1), Intralipid(®) 20% 4 ml·kg⁻¹ (group 2), epinephrine 10 μg·kg⁻¹+Intralipid(®) 4 ml·kg⁻¹ (group 3) or 2 IU vasopressin+Intralipid(®) 4 ml·kg⁻¹ (group 4) were administered. Secondary epinephrine doses were given after 5 min if required.

Survival was 71%, 29%, 86%, and 57% in groups 1, 2, 3, and 4. Return of spontaneous circulation was regained only by initial administration of epinephrine alone or in combination with Intralipid(®). Piglets receiving the combination therapy survived without further epinephrine support. In contrast, in groups 2 and 4, return of spontaneous circulation was only achieved after secondary epinephrine rescue.

In cardiac arrest caused by bupivacaine intoxication, first-line rescue with epinephrine and epinephrine+Intralipid(®) was more effective with regard to survival than Intralipid(®) alone and vasopressin+Intralipid(®) in this pig model (29).

Local anesthetic (LA) intoxication with severe hemodynamic compromise is a potential catastrophic event. Lipid resuscitation has been recommended for the treatment of LA-induced cardiac arrest. However, there are no data about effectiveness of Intralipid for the treatment of severe cardiovascular compromise prior to cardiac arrest. Aim of this study was to compare effectiveness of epinephrine and Intralipid for the treatment of severe Hemodynamic compromise owing to bupivacaine intoxication, anesthetized Piglets were with sevoflurane, intubated, and ventilated. Bupivacaine was infused with a syringe driver via a central venous catheter at a rate of 1 mg·kg⁻¹·min⁻¹ until invasively measured mean arterial pressure (MAP) dropped to 50% of the initial value. Bupivacaine infusion was then stopped, and epinephrine 3 μg·kg⁻¹ (group 1), Intralipid(®) 20% 2 ml·kg⁻¹ (group 2), or Intralipid 20% 4 ml·kg⁻¹ (group 3) was immediately administered. Twenty-one piglets (3×7), were recorded. All animals in group 1 (100%) but only four of seven (57%) piglets in group 2 and group 3, respectively, survived. Normalization of hemodynamic parameters (HR, MAP) and ET(CO₂) was fastest in group 1 with all piglets achieving HR and MAP values. hemodynamic compromise owing to bupivacaine intoxication in piglets, first-line rescue with epinephrine was more effective than Intralipid with regard to survival as well as normalization of hemodynamic parameters and ET(CO₂) (30).

Intravenous lipid emulsion (ILE) has been proposed as a rescue therapy for severe local anesthetic drugs toxicity, but experience is limited with other lipophilic drugs. An 18-year-old healthy woman was admitted 8 h after the voluntary ingestion of sustained-release diltiazem (3600 mg), with severe hypotension refractory to fluid therapy, calcium salts, and high-dose norepinephrine (6.66 μg/kg/min). Hyperinsulinemic euglycemia therapy was initiated and shortly after was followed by a protocol of ILE (intralipid 20%, 1.5 ml/kg as bolus, followed by 0.25 ml/kg over 1 h). The main finding attributed to ILE was an apparent rapid decrease in insulin resistance, despite a prolonged serum diltiazem elimination half-life. Diltiazem is a lipophilic cardiotoxic drug, which could be sequestered in an expanded plasma lipid phase. The mechanism of action of ILE is not known, including its role in insulin resistance and myocardial metabolism in calcium-channel blocker poisoning (31).

### Linoleic Acid (Main Component in LE)-Brain Mitochondria Interaction

Linoleic acid (LA; 18:2 n-6), the most abundant polyunsaturated fatty acid in the US diet, is a precursor to oxidized metabolites that have unknown roles in the brain. Here, we show that oxidized LA-derived metabolites accumulate in several rat brain regions during CO₂-induced ischemia and that LA-derived 13-hydroxyoctadecadienoic acid, but not LA, increase somatic paired-pulse facilitation in rat hippocampus by 80%, suggesting bioactivity. This study provides new evidence that LA participates in the response to ischemia-induced brain injury through oxidized metabolites that regulate neurotransmission. Targeting this pathway may be therapeutically relevant for ischemia-related conditions such as stroke (32).

Long-chain polyunsaturated fatty acids like conjugated linoleic acids (CLA) are required for normal neural development and cognitive function and have been ascribed various beneficial functions. Recently, oral CLA also has been shown to increase testosterone (T) biosynthesis, which is known to diminish traumatic brain injury (TBI)-induced neuropathology and reduce deficits induced by stroke in adult rats. To test the impact of CLA on cognitive recovery following a TBI, 5-6 month old male Sprague Dawley rats received a focal injury (craniectomy+controlled cortical impact (CCI; n=17)) or Sham injury (craniectomy alone; n=12) and were injected with 25 mg/kg body weight of Clarinol® G-80 (80% CLA in safflower oil; n=16) or saline (n=13) every 48 h for 4 weeks. Sham surgery decreased baseline plasma progesterone (P4) by 64.2% (from 9.5±3.4 ng/mL to 3.4±0.5 ng/mL; p=0.068), T by 74.6% (from 5.9±1.2 ng/mL to 1.5±0.3 ng/mL; p<0.05), 11-deoxycorticosterone (11-DOC) by 37.5% (from 289.3±42.0 ng/mL to 180.7±3.3 ng/mL), and corticosterone by 50.8% (from 195.1±22.4 ng/mL to 95.9±2.2 ng/mL), by post-surgery day 1. CCI injury induced similar declines in P4, T, 11-DOC and corticosterone (58.9%, 74.6%, 39.4% and 24.6%, respectively) by post-surgery day 1. These results suggest that both Sham surgery and CCI injury induce hypogonadism and hypoadrenalism in adult male rats. CLA treatment did not reverse hypogonadism in Sham (P4: 2.5±1.0 ng/mL; T: 0.9±0.2 ng/mL) or CCI-injured (P4: 2.2±0.9 ng/mL; T: 1.0±0.2 ng/mL, p>0.05) animals by post-injury day 29, but rapidly reversed by post-injury day 1 the hypoadrenalism in Sham (11-DOC: 372.6±36.6 ng/mL; corticosterone: 202.6±15.6 ng/mL) and CCI-injured (11-DOC: 384.2±101.3 ng/mL; corticosterone: 234.6±43.8 ng/mL) animals. In Sham surgery animals, CLA did not alter body weight, but did markedly increase latency to find the hidden Morris Water Maze platform (40.3±13.0 s) compared to saline treated Sham animals (8.8±1.7 s). In CCI injured animals, CLA did not alter CCI-induced body weight loss, CCI-induced cystic infarct size, or deficits in rotarod performance. However, like Sham animals, CLA injections exacerbated the latency of CCI-injured rats to find the hidden MWM platform (66.8±10.6 s) compared to CCI-injured rats treated with saline (30.7±5.5 s, p<0.05). These results indicate that chronic treatment of CLA at a dose of 25 mg/kg body weight in adult male rats over 1-month 1) does not reverse craniectomy- and craniectomy+CCI-induced hypogonadism, but does reverse craniectomy- and craniectomy+CCI-induced hypoadrenalism, 2) is detrimental to medium- and long-term spatial learning and memory in craniectomized uninjured rats, 3) limits cognitive recovery following a moderate-severe CCI injury, and 4) does not alter body weight (33).

Oxidative damage of membrane polyunsaturated fatty acids (PUFA) is thought to play a major role in mitochondrial dysfunction related to Parkinson's disease (PD). The toxic products formed by PUFA oxidation inflict further damage on cellular components and contribute to neuronal degeneration. Here, we tested the hypothesis that isotopic reinforcement, by deuteration of the bisallylic sites most susceptible to oxidation in PUFA may provide at least partial protection against nigrostriatal injury in a mouse model of oxidative stress and cell death, the 1-methyl-4-phenyl-1,2,3,6-tetrahydropyridine (MPTP) model. Mice were fed a fat-free diet supplemented with saturated acids, oleic acid and essential PUFA: either normal, hydrogenated linoleic (LA, 18:2n-6) and α-linolenic (ALA, 18:3n-3) or deuterated 11,11-D2-LA and 11,11,14,14-D4-ALA in a ratio of 1:1 (to a total of 10% mass fat) for 6 days; each group was divided into two cohorts receiving either MPTP or saline and then continued on respective diets for 6 days. Brain homogenates from mice receiving deuterated PUFA (D-PUFA) vs. hydrogenated PUFA (H-PUFA) demonstrated a significant incorporation of deuterium as measured by isotope ratio mass-spectrometry. Following MPTP exposure, mice fed H-PUFA revealed 78.7% striatal dopamine (DA) depletion compared to a 46.8% reduction in the D-PUFA cohort (as compared to their respective saline-treated controls), indicating a significant improvement in DA concentration with D-PUFA. Similarly, higher levels of the DA metabolite 3,4-dihydroxyphenylacetic acid (DOPAC) were detected in MPTP-exposure mice administered D-PUFA; however, saline-treated mice revealed no change in DA or DOPAC levels. Western blot analyses of tyrosine hydroxylase (TH) confirmed neuroprotection with D-PUFA, as striatal homogenates showed higher levels of TH immunoreactivity in D-PUFA (88.5% control) vs. H-PUFA (50.4% control) in the MPTP-treated cohorts. In the substantia nigra, a significant improvement was noted in the number of nigral dopaminergic neurons following MPTP exposure in the D-PUFA (79.5% control) vs. H-PUFA (58.8% control) mice using unbiased stereological cell counting. Taken together, these findings indicate that dietary isotopic reinforcement with D-PUFA partially protects against nigrostriatal damage from oxidative injury elicited by MPTP in mice (34).

Arachidonic acid (AA), 5,8,11,14-eicosateraenoic acid is abundant, active and necessary in the human body. In the present study, we reported the neuroprotective effects and mechanism of arachidonic acid on hippocampal slices insulted by glutamate, NaN(3) or H(2)O(2) in vitro. Different types of models of brain injury in vitro were developed by 1 mM glutamate, 10 mM NaN(3) or 2 mM H(2)O(2). After 30 min of preincubation with arachidonic acid or linoleic acid, hippocampal slices were subjected to glutamate, NaN(3) or H(2)O(2), then the tissue activities were evaluated by using the 2,3,5-triphenyltetrazolium chloride method. Endogenous antioxidant enzymes activities (SOD, GSH-PX and catalase) in hippocampal slices were evaluated during the course of incubation. MK886 (5 microM; a noncompetitive inhibitor of proliferator-activated receptor [PPAR]alpha), BADGE (bisphenol A diglycidyl ether; 100 microM; an antagonist of PPARgamma) and cycloheximide (CHX; 30 microM; an inhibitor of protein synthesis) were tested for their effects on the neuroprotection afforded by arachidonic acid. Population spikes were recorded in randomly selected hippocapal slices. Arachidonic acid (1-10 microM) dose dependently protected hippocampal slices from glutamate and H(2)O(2) injury (P<0.01), and arachidonic acid (10 microM) can significantly improve the activities of Cu/Zn-SOD in hippocampal slices after 1 h incubation. In addition, 10 microM arachidonic acid significantly increased the activity of Mn-SOD and catalase, and decreased the activities of Cu/Zn-SOD to control value after 3 h incubation. These secondary changes of SOD during incubation can be reversed by indomethacine (10 microM; a nonspecific cyclooxygenase inhibitor) or AA 861 (20 microM; a 5-lipoxygenase inhibitor). Its neuroprotective effect was completely abolished by BADGE and CHX. These observations reveal that arachidonic acid can defense against oxidative stress by boosting the internal antioxidant system of hippocampal slices. Its neuroprotective effect may be mainly mediated by the activation of PPARgamma and synthesis of new protein in tissue (35).

Free fatty acid (FFA) concentrations in cerebrospinal fluid (CSF) are recognized as markers of brain damage in animal studies. There is, however, relatively little information regarding FFA concentrations in human CSF in normal and pathological conditions. The present study examined FFA concentrations in CSF from 15 patients with traumatic brain injury (TBI) and compared the data with values obtained from 73 contemporary controls. Concentrations of specific FFAs from TBI patients, obtained within 48 h of the insult were significantly greater than those in the control group (arachidonic, docosahexaenoic and myristic, P<0.001; oleic, palmitic, P<0.01; linoleic, P<0.05). Higher concentrations of total polyunsaturated fatty acids (P<0.001) and of arachidonic, myristic and palmitic acids measured individually in CSF (P<0.01) obtained 1 week after the insult were associated with a worse outcome at the time of hospital discharge using the Glasgow Outcome Scale. This preliminary investigation suggests that CSF FFA concentrations may be useful as a predictive marker of outcome following TBI (36).

Elevated levels of free fatty acids (FFA) have been implicated in the pathogenesis of neuronal injury and death induced by cerebral ischemia. This study evaluated the effects of immunosuppressants agents, calcineurin inhibitors and blockade of endoplasmic reticulum (ER) calcium channels on free fatty acid formation and efflux in the ischemic/reperfused (I/R) rat brain. Changes in the extracellular levels of arachidonic, docosahexaenoic, linoleic, myristic, oleic and palmitic acids in cerebral cortical superfusates during four-vessel occlusion-elicited global cerebral ischemia were examined using a cortical cup technique. A 20-min period of ischemia elicited large increases in the efflux of all six FFAs, which were sustained during the 40 min of reperfusion. Cyclosporin A (CsA) and trifluoperazine, which reportedly inhibit the I/R elicited opening of a mitochondrial permeability transition (MPT) pore, were very effective in suppressing ischemia/reperfusion evoked release of all six FFAs. FK506, an immunosuppressant which does not directly affect the MPT, but is a calcineurin inhibitor, also suppressed the I/R-evoked efflux of FFAs, but less effectively than CsA. Rapamycin, a derivative of FK506 which does not inhibit calcineurin, did not suppress I/R-evoked FFA efflux. Gossypol, a structurally unrelated inhibitor of calcineurin, was also effective, significantly reducing the efflux of docosahexaenoic, arachidonic and oleic acids. As previous experiments had implicated elevated Ca(2+) levels in the activation of phospholipases with FFA formation, agents affecting endoplasmic reticulum stores were also evaluated. Dantrolene, which blocks the ryanodine receptor (RyR) channel of the ER, significantly inhibited I/R-evoked release of docosahexaenoic, arachidonic, linoleic and oleic acids. Ryanodine, which can either accentuate or block Ca(2+) release, significantly enhanced ischemia/reperfusion-elicited efflux of linoleic acid, with non-significant increases in the efflux of myristic, arachidonic, palmitic and oleic acids. Xestospongin C, an inhibitor of the inositol triphosphate (IP(3)R) channel, failed to affect I/R-evoked FFA efflux. Thapsigargin, an inhibitor of the Ca(2+)-ATPase ER uptake pump, elicited significant elevations in the efflux of myristic, arachidonic and linoleic acids, in the absence of ischemia. Collectively, the data suggest an involvement of both ER and mitochondrial Ca(2+) stores in the chain of events which lead to PLA(2) activation and FFA formation (37).

Brain extracellular levels of glutamate, aspartate, GABA and glycine increase rapidly following the onset of ischemia, remain at an elevated level during the ischemia, and then decline over 20-30 min following reperfusion. The elevated levels of the excitotoxic amino acids, glutamate and aspartate, are thought to contribute to ischemia-evoked neuronal injury and death. Calcium-evoked exocytotic release appears to account for the initial (1-2 min) efflux of neurotransmitter-type amino acids following the onset of ischemia, with non-vesicular release responsible for much of the subsequent efflux of these and other amino acids, including taurine and phosphoethanolamine. Extracellular Ca(2+)-independent release is mediated, in part by Na(+)-dependent amino acid transporters in the plasma membrane operating in a reversed mode, and by the opening of swelling-induced chloride channels, which allow the passage of amino acids down their concentration gradients. Experiments on cultured neurons and astrocytes have suggested that it is the astrocytes which make the primary contribution to this amino acid efflux. Inhibition of phospholipase A(2) attenuates ischemia-evoked release of both amino and free fatty acids from the rat cerebral cortex indicating that this group of enzymes is involved in amino acid efflux, and also accounting for the consistent ischemia-evoked release of phosphoethanolamine. It is, therefore, possible that disruption of membrane integrity by phospholipases plays a role in amino acid release. Recovery of amino acid levels to pre ischemic levels requires their uptake by high affinity Na(+)-dependent transporters, operating in their normal mode, following restoration of energy metabolism, cell resting potentials and ionic gradients (38).

Free fatty acid (FFA) elevation in the brain has been shown to correlate with the severity of damage in ischemic injury. The etiology of this increase in FFA remains unclear and has been hypothesized to result from phospholipase activation. This study examines the effects of specific phospholipase inhibitors on FFA efflux during ischemia-reperfusion injury. A four-vessel occlusion model of cerebral ischemia was utilized to assess the effects of PLA(2) and PLC inhibitors on FFA efflux from rat cerebral cortex. In addition, FFA efflux from non-ischemic cortices exposed to PLA(2) and PLC was measured. Concentrations of arachidonic, docosahexaenoic, linoleic, myristic, oleic, and palmitic acids in cortical superfusates were determined using high performance liquid chromatography (HPLC). Exposure to the non-selective PLA(2) inhibitor 4-bromophenylacyl bromide (BPB) significantly inhibited FFA efflux during ischemia-reperfusion injury (P<0.01 arachidonic, oleic and palmitic; P<0.05 all others); exposure to the PLC inhibitor U73122 had no observed effect. The effects of the Ca(2+)-dependent PLA(2) inhibitor arachidonyl trifluoromethyl ketone (AACOCF(3)) mirrored the effects of BPB and led to reductions in all FFA levels (P<0.01 arachidonic, oleic and palmitic; P<0.05 all others). Exposure to the secretory PLA(2) inhibitor 3-(3-acetamide-1-benzyl-2-ethyl-indolyl-5-oxy) propane sulfonic acid (LY311727) and to the Ca(2+)-independent PLA(2) inhibitor bromoenol lactone (BEL) had only minimal effects on FFA efflux. Application of both PLA(2) and PLC to non-ischemic cortices resulted in significant increases in efflux of all FFA (P<0.05). The study suggests that FFA efflux during ischemia-reperfusion injury is coupled to activation of Ca(2+)-dependent PLA(2) and provides further evidence of the potential neuroprotective benefit of Ca(2+)-dependent PLA(2) inhibitors in ischemia (39).

### Conclusion

The 4 case reports in this article describing the successful use of lipid emulsions (Smoflipid 20% and Lipidem 20%) are the first case reports of Lipid Emulsion Treatment (LET) of Post operative cognitive dysfunction (POCD) in the medical literature.

### REFERENCES

1. American Psychiatric Association. Diagnostic and statistical manual of mental disorders (Dsm-51), 5th ed. Amer Psychiatric Pub Inc; 2013.
2. International Statistical Classification of Diseases and Related Health Problems 10th Revision. http://apps.who.int/classifications/icd10/browse/2016/en, 2015.
3. Olin K, Eriksdotter-Jonhagen M, Jansson A, et al. Postoperative delirium in elderly patients after major abdominal surgery. Br J Surg 2005; 92:1559-1564.
4. Sharma P T, Sieber F E, Zakriya K J, et al. Recovery room delirium predicts postoperative delirium after hip-fracture repair. Anesth Analg 2005; 101:1215-1220
5. Radtke F M, Franck M, Schneider M, et al. Comparison of three scores to screen for delirium in the recovery room. Br J Anaesth 2008; 101:338-343.
6. Eurostat. Your key to European statistics. 2015; http://ec.europa.eu/eurostat/data/database 7. Pearse R M, Moreno R P, Bauer P, et al. Mortality after surgery in Europe: a 7 day cohort study. Lancet 2012; 380:1059-1065.
8. Weiser T G, Regenbogen S E, Thompson K D, et al. An estimation of the global volume of surgery: a modelling strategy based on available data. Lancet 2008; 372:139-144.
9. Saczynski J S, Marcantonio E R, Quach L, et al. Cognitive trajectories after postoperative delirium. N Engl J Med 2012; 367:30-39.
10. multer N, Lingehall H C, Gustafson Y, et al. Delirium after cardiac surgery: incidence and risk factors. Interact Cardiovasc Thorac Surg 2013; 17:790-796.
11. Dasgupta M, Dumbrell A C. Preoperative risk assessment for delirium after noncardiac surgery: a systematic review. J Am Geriatr Soc 2006; 54:1578-1589.
12. Dyer C B, Ashton C M, Teasdale T A. Postoperative delirium. A review of 80 primary data-collection studies. Arch Intern Med 1995; 155:461-465.
13. Bruce A J, Ritchie C W, Blizard R, et al. The incidence of delirium associated with orthopedic surgery: a meta-analytic review. Int Psychogeriatr 2007; 19:197-214.
14. Smith T O, Cooper A, Peryer G, Griffiths R, Fox C3, Cross J. Factors predicting incidence of post-operative delirium in older people following hip fracture surgery: a systematic review and meta-analysis. Int J Geriatr Psychiatry. 2017 Jan. 17.
15. Benhamou D, Brouquet A. Postoperative cerebral dysfunction in the elderly: Diagnosis and prophylaxis. J Visc Surg. 2016 December; 153(6S):S27-S32.
16. Raats J W, Steunenberg S L, de Lange D C, van der Laan L. Risk factors of post-operative delirium after elective vascular surgery in the elderly: A systematic review. Int J Surg. 2016 November; 35:1-6.
17. Ruggiero C, Bonamassa L, Pelini L, Prioletta I, Cianferotti L, Metozzi A, Benvenuti E, Brandi G, Guazzini A, Santoro G C, Mecocci P, Black D, Brandi M L Early post-surgical cognitive dysfunction is a risk factor for mortality among hip fracture hospitalized older persons. Osteoporos Int. 2017 February; 28(2):667-675.
18. Soh S, Shim J K, Song J W, Kim K N, Noh H Y, Kwak Y L. Postoperative Delirium in Elderly Patients Undergoing Major Spinal Surgery: Role of Cerebral Oximetry. J Neurosurg Anesthesiol. 2016 Aug. 25.
19. Hshieh T T, Dai W, Cavallari M, Guttmann C R, Meier D S, Schmitt E M, Dickerson B C, Press D Z, Marcantonio E R, Jones R N, Gou Y R, Travison T G, Fong T G, Ngo L, Inouye S K, Alsop D C; SAGES Study Group. Cerebral blood flow MRI in the nondemented elderly is not predictive of post-operative delirium but is correlated with cognitive performance. J Cereb Blood Flow Metab. 2016 Jul. 11.
20. Zheng Y B, Ruan G M, Fu J X, Su Z L, Cheng P, Lu J Z. Postoperative plasma 8-iso-prostaglandin F2α levels are associated with delirium and cognitive dysfunction in elderly patients after hip fracture surgery. Clin Chim Acta. 2016 Apr. 1; 455:149-53.
21. Rudiger A, Begdeda H, Babic D, Kruger B, Seifert B, Schubert M, Spahn D R, Bettex D. Intra-operative events during cardiac surgery are risk factors for the development of delirium in the ICU. Crit Care. 2016 Aug. 21; 20:264.
22. Bilge E U, Kaya M, Senel G O, Unver S. The Incidence of Delirium at the Postoperative Intensive Care Unit in Adult Patients. Turk J Anaesthesiol Reanim. 2015 August; 43(4):232-9.
23. O'Neal J B, Shaw A D. Predicting, preventing, and identifying delirium after cardiac surgery. Perioper Med (Lond). 2016 Apr. 26; 5:7.
24. Mackensen G B, Ti L K, Phillips-Bute B G, Mathew J P, Newman M F, Grocott H P. Neurologic Outcome Research Group (NORG). Cerebral embolization during cardiac surgery: impact of aortic atheroma burden. Br J Anaesth. 2003; 91:656-661.
25. Reinsfelt B, Ricksten S E, Zetterberg H, Blennow K, Freden-Lindqvist J, Westerlind A. Cerebrospinal fluid markers of brain injury, inflammation, and blood-brain barrier dysfunction in cardiac surgery. Ann Thorac Surg. 2012; 94:549-555
26. Brooker R F, Brown W R, Moody D M, Hammon J W Jr., Reboussin D M, Deal D D, Ghazi-Birry H S, Stump D A. Cardiotomy suction: a major source of brain lipid emboli during cardiopulmonary bypass. Ann Thorac Surg. 1998; 65:1651-1655.
27. A. Papadopoulou, J. W. Willers, T. L. Samuels and D. R. Uncles, “The Use of Dye Surrogates to Illustrate Local Anesthetic Drug Sequestration by Lipid Emulsion: A Visual Demonstration of the Lipid Sink Effect,” Regional Anesthesia & Pain Medicine, Vol. 37, No. 2,2012, pp. 183-187.
28. A. M. Grunbaum, B. M. Gilfix, S. Gosselin and D. W. Blank, “Analytical Interferences Resulting from Intravenous Lipid Emulsion,” Clinical Toxicology (Phila), Vol. 50, No. 9, 2012, pp. 812-817.
29. J. Mauch, 0. M. Jurado, N. Spielmann, R. BettschartWolfensberger and M. Weiss, “Resuscitation Strategies from Bupivacaine-Induced Cardiac Arrest,” Pediatric Anesthesia, Vol. 22, No. 2, 2012, pp. 124-129.
30. J. Mauch, 0. M. Jurado, N. Spielmann, R. BettschartWolfensberger and M. Weiss, “Comparison of Epinephrine vs Lipid Rescue to Treat Severe Local Anesthetic Toxicity—An Experimental Study in Piglets,” Pediatric Anesthesia, Vol. 21, No. 11, 2011, pp. 1103-1108.
31. V. Montiel, T. Gougnard and P. Hantson, “Diltiazem Poisoning Treated with Hyperinsulinemic Euglycemia Therapy and Intravenous Lipid Emulsion,” European Journal of Emergency Medicine, Vol. 18, No. 2, 2011, pp. 121-123.
32. Hennebelle M, Zhang Z, Metherel A H, Kitson A P, Otoki Y, Richardson C E, Yang J, Lee K S S, Hammock B D, Zhang L, Bazinet R P, Taha A Y. Linoleic acid participates in the response to ischemic brain injury through oxidized metabolites that regulate neurotransmission. Sci Rep. 2017 Jun. 28; 7(1):4342.
33. Geddes R I, Hayashi K, Bongers Q, Wehber M, Anderson I M, Jansen A D, Nier C, Fares E, Farquhar G, Kapoor A, Ziegler T E, VadakkadathMeethal S, Bird I M, Atwood C S. Conjugated Linoleic Acid Administration Induces Amnesia in Male Sprague Dawley Rats and Exacerbates Recovery from Functional Deficits Induced by a Controlled Cortical Impact Injury. PLoS One. 2017 Jan. 26; 12(1):e0169494.
34. Shchepinov M S, Chou V P, Pollock E, Langston J W, Cantor C R, Molinari R J, Manning-Bog A B. Isotopic reinforcement of essential polyunsaturated fatty acids diminishes nigrostriatal degeneration in a mouse model of Parkinson's disease. Toxicol Lett. 2011 Nov. 30; 207(2):97-103.
35. Wang Z J, Liang C L, Li G M, Yu C Y, Yin M. Neuroprotective effects of arachidonic acid against oxidative stress on rat hippocampal slices. Chem Biol Interact. 2006 Nov. 7; 163(3):207-17.
36. Pilitsis J G, Coplin W M, O'Regan M H, Wellwood J M, Diaz F G, Fairfax M R, Michael D B, Phillis J W. Free fatty acids in cerebrospinal fluids from patients with traumatic brain injury. Neurosci Lett. 2003 Oct. 2; 349(2):136-8.
37. Phillis J W, Diaz F G, O'Regan M H, Pilitsis J G. Effects of immunosuppressants, calcineurin inhibition, and blockade of endoplasmic reticulum calcium channels on free fatty acid efflux from the ischemic/reperfused rat cerebral cortex. Brain Res. 2002 Dec. 6; 957(1):12-24.
38. Phillis J W, O'Regan M H. Characterization of modes of release of amino acids in the ischemic/reperfused rat cerebral cortex. Neurochem Int. 2003 September-October; 43(4-5):461-7.
39. Pilitsis J G, Diaz F G, O'Regan M H, Phillis J W. Differential effects of phospholipase inhibitors on free fatty acid efflux in rat cerebral cortex during ischemia-reperfusion injury. Brain Res. 2002 Sep. 27; 951(1):96-106.

### Example 11

### Lipid Emulsion Treatment for Post Spinal Anesthesia Myoclonus

Two case reports of myoclonus of legs post spinal anesthesia treated successfully by IV lipid emulsion are first described in the medical literature.

A review of cases of myoclonus post regional anesthesia (spinal or epidural) are discussed with the hypothesis that the Lipid Emulsion effects are on the mitochondria and the intracellular calcium.

### Case Report No 1

43-years-old, healthy female patient was scheduled for drainage of an abcess on the right buttock. In the past she had given 2 Spinal Anesthesia for Cesarian sections uneventfully. For surgery this time, Spinal Anesthesia performed at L3-4 with 6 mg bupivacaine 0.5% heavy and 20 mcg fentanyl uneventfully. Time of surgery was 30 min. Myoclonic movement of both legs occurred 60 min post-op. She remained fully conscious and had no other local neurological symptoms. Vital signs were stable. LAST was ruled out. She was under close monitoring, but myoclonus was not diminished in 2 hours. Lipid challenging therapy was considered. After an infusion of 250 ml Lipidem 20% over 30 min abnormal movement diminished gradually and disappeared in 60 min. No recurrent of myoclonus episode was noted thereafter.

The patient's myoclonus can be seen in the following video [23]:

### Case Report No 2

A female 34 years-old, 39 weeks of pregnancy. Her precedent had two vaginal delivery (the second time with the assistance of Forceps). She was healthy and had expected a normal delivery but failed. The Cesarian section was performed under Spinal Anesthesia. The 27 Quincke Needle was introduced at L3-4 uneventfully. Dose of SA was 7.5 mg Bupivacaine heavy 0.5% (Aguettant)+25 mcg Fentanyl. The operation lasted 45 min without any medication added. Approximately 100 min postop, patient had myoclonus on the left leg as in the following Video [24].

The movement of the right leg was normal. The sensory feeling of both legs returned nearly normal. Other systems were with no particular problems. No LAST, hemodynamic was stable, she was totally conscious, no shivering, no other neurological signs.

Since the anesthesiologist (TTD) learned from anesthesiologist (TAN) his case through discussions in the group on Facebook, he used LIPIDEM 20% (B. Braun) right away, not wasted time as in anesthesiologist (TAN) case (case no.1). After 5 min of IV Lipid emulsion infusion the myoclonus decreased significantly (see the video after Lipidem infusion) [25]. and gone away in 30 min of the IV lipid emulsion infusion.

### Discussion

### Lipidem

Lipidem is an intralipid like lipid emulsion (using Soybean oil and Egg yolk) with the following composition

Lipoplus®/Lipidem® Composition 1000 ml of emulsion contains: Medium-chain triglycerides: 100.0 g Soybean oil, refined: 80.0 g Omega-3-acid triglycerides: 20.0 g Essential fatty acid content per liter: Linoleic acid (omega-6): 48.0-58.0 g Alpha-linolenic acid (omega-3): 5.0-11.0 g Eicosa-pentaenoic acid and docosahexaenoic acid: 8.6-17.2 g Caloric content per liter: 7,900 kJ≈1,910 kcal. Osmolality: approx. 410 mOsm/kg. Titration acidity or alkalinity (to pH 7.4): less than 0.5 mmol/l NaOH or HCl pH: 6.5-8.5. The other ingredients are glycerol, egg lecithin, allrac-α-Tocopherol, ascorbyl palmitate, sodium oleate, sodium hydroxide (for pH adjustment) and water for injections. B. Braun Melsungen AG Carl-Braun-Straße 134212 Melsungen, Germany.

### Myoclonus

Myoclonus creates significant disability for patients. This symptom or sign can have many different etiologies, presentations, and pathophysiological mechanisms. A thorough evaluation for the myoclonus etiology is critical for developing a treatment strategy. The best etiological classification scheme is a modified version from that proposed by Marsden et al. in 1982. Clinical neurophysiology, as assessed by electromyography and electroencephalography, can be used to classify the pathophysiology of the myoclonus using a neurophysiology classification scheme. If the etiology of the myoclonus cannot be reversed or treated, then symptomatic treatment of the myoclonus itself may be warranted. Unfortunately, there are few controlled studies for myoclonus treatments.

The treatment strategy for the myoclonus is best derived from the neurophysiology classification scheme categories: cortical, cortical-subcortical, subcortical-nonsegmental, peripheral.

A cortical physiology classification is most common. Levetiracetam is suggested as first-line treatment for cortical myoclonus, but valproic acid and clonazepam are commonly used. Cortical-subcortical myoclonus is the physiology demonstrated by myoclonic seizures, such as in primary epileptic myoclonus (e.g., juvenile myoclonic epilepsy). Valproic acid has demonstrated efficacy in such epileptic syndromes with other medications providing an adjunctive role. Clonazepam is used for subcorticalnonsegmental myoclonus, but other treatments, depending on the syndrome, have been used for this physiological type of myoclonus. Segmental myoclonus is difficult to treat, but clonazepam and botulinum toxin are used. Botulinum toxin is used for focal examples of peripheral myoclonus. Myoclonus treatment is commonly not effective and/or limited by side effects [1].

Myoclonus remains a challenging movement phenotype to characterize, evaluate, and treat. A systematic assessment of the temporal sequence, phenomenology, and distribution of movements can assist in the rational approach to diagnosis and management.

Cortical forms of myoclonus are increasingly recognized as primarily cerebellar disorders. A syndrome of orthostatic myoclonus has been recognized by electrophysiology in patients with neurodegenerative disorders, mainly in Alzheimer disease, accounting for impairments in gait and balance previously mischaracterized as normal pressure hydrocephalus or orthostatic tremor. Tyrosine hydroxylase deficiency and Silver-Russell syndrome (uniparental disomy of chromosome 6) have been established as two novel causes of the myoclonus-dystonia syndrome. Mutations in the glycine receptor (GlyR) al-subunit gene (GLRA1) explain the major expression of hyper ekplexia, an inherited excessive startle disorder, but newly identified mutations in GlyR β-subunit (GLRB) and glycine transporter 2 (GlyT2) genes (SLC6A5) account for “minor” forms of this disorder manifested as excessive startle and hypnic jerks. The entity previously known as palatal myoclonus has been reclassified as palatal tremor in recognition of its clinical and electromyographic features and no longer enters the differential diagnosis of myoclonic disorders. Increasing documentation of psychogenic features in patients previously characterized as having propriospinal myoclonus has cast doubts on the existence of this distinctive disorder.

Myoclonus can be a prominent manifestation of a wide range of disorders. Electrophysiologic testing aids in distinguishing myoclonus from other mimics and classifying them according to cortical, subcortical, or spinal origin, which assists the choice of treatment. Despite the lack of randomized clinical trials, levetiracetam appears most effective in patients with cortical myoclonus, whereas clonazepam remains the only first-line therapeutic option in subcortical and spinal myoclonus [2].

Post Spinal/Epidural Anesthesia Myoclonus

Perioperative spinal myoclonus is extremely rare. Many anaesthetists and perioperative practitioners may not diagnose or manage this complication appropriately when it occurs. This case report of unusual acute spinal myoclonus following regional anaesthesia highlights certain aspects of this rare complication that have not previously been published [3].

A series of four consecutive patients who developed acute lower-limb myoclonus following spinal or epidural anaesthesia are described. The case series occurred at three different hospitals and involved four anaesthetists over a 3-year period. Two Caucasian men, aged 90-years-old and 67-years-old, manifested unilateral myoclonus. Two Caucasian women, aged 64-years-old and 53-years-old, developed bilateral myoclonus. Myoclonus was self-limiting in one patient, treated with further regional anaesthesia in one patient and treated with intravenous midazolam in two patients. The overall outcome was good in all patients, with no recurrence or sequelae in any of the patients.

This case series emphasizes that spinal myoclonus following regional anaesthesia is rare, has diverse pathophysiology and can have diverse presentations. The treatment of perioperative spinal myoclonus should be directed at the aetiology. Anaesthetists and perioperative practitioners who are unfamiliar with this rare complication should be reassured that it may be treated successfully with midazolam [3].

We report a case of spinal myoclonus induced by the tip of an intrathecal catheter in a 35-year-old patient with severe, adult-onset, generalized dystonia of unknown cause, treated for 2 years using intrathecal baclofen [3]. One month after a falling episode, the patient developed focal myoclonus of the right proximal leg whenever she stood up from a seated position. The electrophysiologic recordings were compatible with spinal segmental myoclonus, originating at a focus corresponding to the L2-S2 segments. At this site, the tip of the intrathecal catheter was demonstrated by myelography to be in close proximity to the nerve roots and conus medullaris. The myoclonus resolved promptly once the catheter tip was withdrawn. This report represents an unusual complication of intrathecal catheter systems that, if recognized, can lead to prompt therapeutic intervention [4].

A nulliparous woman presented with pre-eclampsia at 39 weeks' gestation. A combined spinal-epidural anaesthesia was employed for Caesarean section, but the spinal component produced no discernible block, so the epidural was topped up with 20 ml ropivacaine 0.75% without problem and surgery was uneventful. A week after delivery she developed twitching of her legs and opisthotonus, that was initially thought to be eclampsia but was subsequently diagnosed as spinal myoclonus. She was treated with oral carbamazepine and diazepam, with improvement over the next 4 days, and discharged home a week later taking oral carbidopa and levodopa. Her symptoms resolved completely 6 months after the initial event [5].

We report a patient who developed paraplegia following percutaneous nephrolithotresis of the left kidney under epidural anaesthesia [6]. The cause of the paraplegia was unknown, but occlusion of the anterior spinal artery or central arteries and arachnoiditis, possibly due to the epidural anaesthesia, may have taken part in the onset and progression of the paralysis. The patient had spinal myoclonus corresponding to the spinal levels where myelomalacia was found by magnetic resonance (MR) imaging [6]. Spinal myoclonus following neuraxial anesthesia is rare. This report describes a case of myoclonus-like involuntary movement that occurred during the recovery from epidural anesthesia for a cesarean delivery. The patient's symptom improved with the administration of benzodiazepine, and the patient recovered with no neurological sequelae. In conclusion, epidural anesthesia can cause spinal myoclonus, which can be treated with a benzodiazepine [7].

Involuntary movement during and after neuraxial anesthesia, such as spinal and epidural anesthesia, is rarely observed. In this report, we describe a case of myoclonus-like involuntary movement of the upper extremities in a patient undergoing a planned repeat cesarean section under spinal anesthesia with bupivacaine that completely subsided after administration of 2 mg of midazolam [8]. The myoclonus-like movement did not recur or cause any apparent neurological side effects [8].

It is presented in this case report a very rare complication after spinal anesthesia to provide subsidies to the management and therapeutic conduct [9]. This is a 63-year old African-Brazilian patient, ASA I, scheduled for transurethral resection of the prostate (TURP). He underwent subarachnoid anesthesia with bupivacaine (15 mg) without adrenaline. Intercurrences were not observed during puncture, and the patient was positioned for surgery. Soon after positioning the patient, he complained of severe pain in the perineum region followed by involuntary tonicclonic movements of the lower limbs. The patient was treated with a benzodiazepine to control the myoclonus without response. This episode was followed by significant agitation and the patient was intubated. He was maintained in controlled ventilation and transferred to the Intensive Care Unit. Despite all biochemical and imaging tests performed, an apparent cause was not detected. The medication was not changed and the same batch of anesthetic had been used in other patients that same day without intercurrences.

After ruling out all possible causes, the diagnosis of spinal myoclonus after spinal anesthesia with bupivacaine was made by exclusion [9]. Spinal myoclonus is an unusual, self-limiting, adverse event that may occur during spinal anesthesia. The exact cause and underlying biochemical mechanism of spinal myoclonus remain unclear. A few cases of spinal myoclonus have been reported after administration of intrathecal bupivacaine. We report a case in which spinal myoclonus recurred after two episodes of spinal anesthesia with bupivacaine at a 1-year interval in a 35-year-old woman [10]. The myoclonus was acute and transient. The patient recovered completely, with no neurologic sequelae [10].

We report a case of spinal myoclonus following cesarean section [11]. The patient was a 34-year-old woman without history of neurologic disorders. In the operating room, after placement of an epidural catheter at T12-L1, bupivacaine 2.4 ml was administered intrathecally via a 25 G needle at L2-3. Epidural administration of ropivacaine (0.13%, 4 ml×hr⁻¹) was started 72 min after spinal anesthesia. The intra and postoperative courses were otherwise uneventful. The patient complained of involuntary jerky movements of her lower legs 195 min after the start of the spinal anesthesia. The sensory level was T12 and she could move her legs on command but could not stop her involuntary movements. The myoclonic movements ceased 150 min later without medication and did not reappear, despite restarting the epidural anesthesia with ropivacaine [11].

Propriospinal myoclonus is a rare disorder characterized by sudden, shock-like, involuntary jerks that arise from the axial muscles and spread both rostrally and caudally to other myotomes through slow polysynaptic pathways. It can be idiopathic or secondary to intrinsic and extrinsic spinal cord lesions; additionally, it can develop as an adverse effect to the administration of several drugs, including neuraxial local anesthetics. This article describes a case of transient propriospinal myoclonus in a 77-year-old woman undergoing surgery for hip replacement who received 12 mg of 0.5% normobaric bupivacaine administered by a 25-G spinal needle [12]. On postoperative day 1, the patient presented with spinal myoclonus, defined by clinical and electrophysiologic studies. Valproate and clonazepam controlled the symptoms, and on day 4 the myoclonus completely disappeared. Few cases of myoclonus induced by intrathecal bupivacaine administration have been reported in the literature, but systematic reviews written to clarify the global incidence and the physiopathology of this complication are still lacking [12].

Focal myoclonus of peripheral origin, i.e., peripheral myoclonus (PM), is a rare disorder. Although PM always accompanies a lesion in the peripheral nerve, supplying the affected muscles, its mechanism remains unclear. Here we present a patient with focal myoclonus of the thigh muscles following a traumatic lesion in the femoral nerve [13]. Lumbar spinal anesthesia, as well as local anesthetic block of the femoral nerve, completely abolished the patient's myoclonus temporarily. This movement was remarkably diminished after a surgical exploration of the wound with the removal of fibrous tissue beneath the scar and liberation of the femoral nerve. This case suggests the contribution of a spinal relay mechanism in the development of PM, in addition to the contribution of a nerve lesion [13].

We report a patient who developed a rare neurological complication of spinal myoclonus possibly caused by an epidural catheter [14]. A 24-yr-old female received laparoscopy and intrauterine curettage under general combined with epidural anesthesia. Spinal myoclonus started about 4 hours after the last epidural drug injection and disappeared 2 hours following removal of the epidural catheter. The patient was discharged without any untoward neurological sequelae [14].

We herein report a case of spinal myoclonus following the administration of epidural anesthesia [15]. A 25-year-old woman underwent lumbar epidural anesthesia because of lumbago and cramps in her left lower limb. She immediately felt a lancinating pain in her left limb during anesthesia at the level of L 4/5 and soon developed myoclonus in her left thigh. The neurological examination revealed rhythmic myoclonus in the left quadriceps and adductor thigh muscles. The myoclonus disappeared after performing a blockade of the left L4 spinal root by using 1.5 ml of 1% lidocaine. An injury to the left L4 nerve root during the epidural anesthesia possibly caused an abnormal transmission of the impulses or ectopic hyperexcitability in the nerve root, which might lead to the disturbance of the spinal inhibitory interneurons and hyperexcitability of the anterior horn cells causing myoclonus. Since she did not demonstrate any muscular weakness, nor sensory loss during the lidocaine block, the 1% lidocaine appeared to block the sympathetic nerves or to suppress the ectopic hyperexcitability. The sympathetic nerves may be involved in the development of her spinal myoclonus [15].

The use of intrathecal diamorphine via an implanted portal system is described for pain control in a patient suffering from vertebral metastatic disease. The complication of myoclonic spasms affecting the lower half of the body occurred after 14 days, when increasing the bolus dose to 40 mg. The spasms lasted for 3 hr and then gradually subsided. Diamorphine was subsequently restarted at a lower dose of 15 mg twice daily. On increasing the dose to 20 mg diamorphine 10 days later, severe distressing myoclonic spasms recurred 20 min post injection. Myoclonus could only be controlled by instituting a local anesthetic intrathecal block. The patient was finally managed with 20 mg diamorphine per day by intrathecal infusion, and the pain was reasonably well controlled for the following 10 weeks without any recurrence of myoclonic spasms [16].

We report a case of periodic leg movements (PLM) observed in an 86-year-old man during either midthoracic epidural anesthesia or spinal anesthesia [17]. The PLM observed were stereotyped (extension of the big toe in combination with partial flexion of the ankle, knee, and hip lasting 3-5s) and repetitive (inter event intervals between jerks were 20-40 s) for about 120 and 30 min respectively. The patient was awake but unaware of the PLM unless reminded. The present case was quite similar to sleep-related (noctural) myoclonus (SRM) in every respect except for its occurrence during wakefulness. SRM is more prevalent in the elderly population but its mechanism remains to be elucidated. Previously, we had reported a case of PLM observed in an elderly man with SRM [18]. In our two cases, PLM were seen only while the local anesthetic was acting on the spinal cord; therefore, these anesthesia-related PLM (ARPLM) may suggest that the spinal cord is involved. In particular, we consider that physiological changes seen commonly during non-rapid-eye-movement sleep and a certain phase of anesthesia, such as suppression of the descending inhibitory pathway, and pyramidal tract dysfunction are relevant to ARPLM. In addition, the concomitant alteration of the blood flow in the leg and changes due to aging of the spinal cord may also be involved [17].

Lipid Emulsion Effects on Mitochondria and Intracellular Calcium

Local anesthetic toxicity is thought to be mediated partly by inhibition of cardiac mitochondrial function. Intravenous (i.v.) lipid emulsion may overcome this energy depletion, but doses larger than currently recommended may be needed for rescue effect. In this randomized study with anesthetized pigs, we compared the effect of a large dose, 4 mL/kg, of i.v. 20% Intralipid® (n=7) with Ringer's acetate (n=6) on cardiovascular recovery after a cardiotoxic dose of bupivacaine [18]. We also examined mitochondrial respiratory function in myocardial cell homogenates analyzed promptly after needle biopsies from the animals. Bupivacaine plasma concentrations were quantified from plasma samples. Arterial blood pressure recovered faster, and systemic vascular resistance rose more rapidly after Intralipid than Ringer's acetate administration (p<0.0001), but Intralipid did not increase cardiac index or left ventricular ejection fraction. The lipid-based mitochondrial respiration was stimulated by approximately 30% after Intralipid (p<0.05) but unaffected by Ringer's acetate. The mean (standard deviation) area under the concentrationtime curve (AUC) of total bupivacaine was greater after Intralipid (105.2(13.6) mg·min/L) than after Ringer's acetate (88.1(7.1) mg·min/L) (p=0.019). After Intralipid, the AUC of the lipid-un-entrapped bupivacaine portion (97.0(14.5) mg·min/L) was 8% lower than that of total bupivacaine (p<0.0001). To conclude, 4 mL/kg of Intralipid expedited cardiovascular recovery from bupivacaine cardiotoxicity mainly by increasing systemic vascular resistance. The increased myocardial mitochondrial respiration and bupivacaine entrapment after Intralipid did not improve cardiac function [18].

Lipid emulsions have been used to treat various drug toxicities and for total parenteral nutrition therapy. Their usefulness has also been confirmed in patients with local anesthetic-induced cardiac toxicity. The purpose of this study was to measure the hemodynamic and composition effects of lipid emulsions and to elucidate the mechanism associated with changes in intracellular calcium levels in myocardiocytes.

We measured hemodynamic effects using a digital analysis system after Intralipid® and Lipofundin® MCT/LCT were infused into hearts hanging in a Langendorff perfusion system [19,20]. We measured the effects of the lipid emulsions on intracellular calcium levels in H9c2 cells by confocal microscopy.

Infusion of Lipofundin® MCT/LCT 20% (1 ml/kg) resulted in a significant increase in left ventricular systolic pressure compared to that after infusing modified Krebs-Henseleit solution (1 ml/kg) (P=0.003, 95% confidence interval [CI], 2.4-12.5). Lipofundin® MCT/LCT 20% had a more positive inotropic effect than that of Intralipid® 20% (P=0.009, 95% CI, 1.4-11.6). Both lipid emulsion treatments increased intracellular calcium levels. Lipofundin® MCT/LCT (0.01%) increased intracellular calcium level more than that of 0.01% Intralipid® (P<0.05, 95% CI, 0.0-1.9).

These two lipid emulsions had different inotropic effects depending on their triglyceride component. The inotropic effect of lipid emulsions could be related with intracellular calcium level [19].

Accidental intravascular or high-dose injection of local anesthetics (LA) can result in serious, potentially life threatening complications. Indeed, adequate supportive measures and the administration of lipid emulsions are required in such complications.

The study's objectives were threefold:

(i) evaluate the myocardial toxicity of levobupivacaine when administered intravenously; (ii) investigate levobupivacaine toxicity on cardiomyocytes mitochondrial functions and cellular structure; (iii) assess the protective effects of a lipid emulsion in the presence or absence of myocardial ischemia. Domestic pigs randomized into two groups of 24 animals each, with either preserved coronary circulation or experimental myocardial ischemia. Six animals from each group received either: (i) single IV injection of saline, (ii) lipid emulsion (Intralipid (®)), (iii) levobupivacaine, (iv) combination levobupivacaine-Intralipid (®).

Serially measured endpoints included: heart rate, duration of the monophasic action potentials (dMAP), mean arterial pressure, and peak of the time derivative of left ventricular pressure (LV dP/dtmax). In addition, the following cardiomyocytes mitochondrial functions were measured: reactive oxygen species (ROS) production, oxidative phosphorylation, and calcium retention capacity (CRC) as well as the consequences of ROS production on lipids, proteins, and DNA. IV injection of levobupivacaine induced sinus bradycardia and reduced dMAP and LV dP/dtmax. At the mitochondrial level, oxygen consumption and CRC were decreased. In contrast, ROS production was increased leading to enhanced lipid peroxidation and structural alterations of proteins and DNA. Myocardial ischemia was associated with global worsening of all changes. Intralipid (®) quickly improved haemodynamics. However, beneficial effects of Intralipid (®) were less clear after myocardial ischemia [20].

Cocaine intoxication leads to over 500,000 emergency department visits annually in the United States and ethanol cointoxication occurs in 34% of those cases. Cardiotoxicity is an ominous complication of cocaine and cocaethylene overdose for which no specific antidote exists. Because infusion of lipid emulsion (Intralipid) can treat lipophilic local anesthetic toxicity and cocaine is an amphipathic local anesthetic, the authors tested whether lipid emulsion could attenuate cocaine cardiotoxicity in vivo [21]. The effects of lipid emulsion were compared with the metabolically inert sulfobutylether-β-cyclodextrin (SBE-β-CD; Captisol) in an isolated heart model of cocaine and cocaethylene toxicity to determine if capture alone could exert similar benefit as lipid emulsion, which exhibits multimodal effects. The authors then tested if cocaine and cocaethylene, like bupivacaine, inhibit lipid-based metabolism in isolated cardiac mitochondria.

For whole animal experiments, Sprague-Dawley rats were anesthetized, instrumented, and pre-treated with lipid emulsion followed by a continuous infusion of cocaine to assess time of onset of cocaine toxicity. For ex vivo experiments, rat hearts were placed onto a nonrecirculating Langendorff system perfused with Krebs-Henseleit solution. Heart rate, left ventricle maximum developed pressure (LVdevP), left ventricle diastolic pressure, maximum rate of contraction (+dP/dtmax), maximum rate of relaxation (−dP/dtmax), rate-pressure product (RPP=heart ratexLVdevP), and line pressure were monitored continuously during the experiment. A dose response to cocaine (10, 30, 50, and 100 μmol/L) and cocaethylene (10, 30, and 50 μmol/L) was generated in the absence or presence of either 0.25% lipid emulsion or SBE-β-CD. Substrate-specific rates of oxygen consumption were measured in interfibrillar cardiac mitochondria in the presence of cocaine, cocaethylene, ecgonine, and benzoylecgonine.

Treatment with lipid emulsion delayed onset of hypotension (140 seconds vs. 279 seconds; p=0.008) and asystole (369 seconds vs. 607 seconds; p=0.02) in whole animals. Cocaine and cocaethylene induced dose dependent decreases in RPP, +dP/dtmax, and −dP/dtmax abs (p<0.0001) in Langendorff hearts; line pressure was increased by cocaine and cocaethylene infusion, but not altered by treatment. Lipid emulsion attenuated cocaine and cocaethylene-induced cardiac depression. SBE-β-CD alone evoked a mild cardio depressant effect (p<0.0001) but attenuated further cocaine- and cocaethylene-induced decrements in cardiac contractility at high concentrations of drug (100 μmol/L; p<0.001). Finally, both cocaine and cocaethylene, but not ecgonine and benzoylecgonine, inhibited lipid-dependent mitochondrial respiration by blocking carnitine exchange (p<0.05).

A commercially available lipid emulsion was able to delay progression of cocaine cardiac toxicity in vivo. Further, it improved acute cocaine- and cocaethylene-induced cardiac toxicity in rat isolated heart while SBE-β-CD was effective only at the highest cocaine concentration. Further, both cocaine and cocaethylene inhibited lipid-dependent mitochondrial respiration. Collectively, this suggests that scavenging-independent effects of lipid emulsion may contribute to reversal of acute cocaine and cocaethylene cardiotoxicity, and the beneficial effects may involve mitochondrial lipid processing [21].

We hypothesized that acute lipid-induced insulin resistance would be attenuated in high-oxidative muscle of lean trained (LT) endurance athletes due to their enhanced metabolic flexibility and mitochondrial capacity [22]. Lean sedentary (LS), obese sedentary (OS), and LT participants completed two hyperinsulinemic euglycemic clamp studies with and without (glycerol control) the coinfusion of Intralipid. Metabolic flexibility was measured by indirect calorimetry as the oxidation of fatty acids and glucose during fasted and insulin-stimulated conditions, the latter with and without lipid oversupply. Muscle biopsies were obtained for mitochondrial and insulin-signaling studies. During hyperinsulinemia without lipid, glucose infusion rate (GIR) was lowest in OS due to lower rates of nonoxidative glucose disposal (NOGD), whereas state 4 respiration was increased in all groups. Lipid infusion reduced GIR similarly in all subjects and reduced state 4 respiration. However, in LT subjects, fat oxidation was higher with lipid oversupply, and although glucose oxidation was reduced, NOGD was better preserved compared with LS and OS subjects. Mitochondrial performance was positively associated with better NOGD and insulin sensitivity in both conditions. We conclude that enhanced mitochondrial performance with exercise is related to better metabolic flexibility and insulin sensitivity in response to lipid overload [22].

### Conclusion

Two case reports of myoclonus of legs post spinal anesthesia treated successfully by IV lipid emulsion are first described in the medical literature.

A review of cases of myoclonus post regional anesthesia (spinal or epidural) are discussed with the hypothesis that the Lipid Emulsion effects are on the mitochondria and the intracellular calcium.

### Example 12

### Lipid Emulsion for Local Anesthesia Reversal (LAR) after Prolonged Spinal/Epidural Anesthesia

A 71-year-old woman was scheduled for total knee replacement on left side. Combined Spinal Epidural Anesthesia (CSEA) was performed at L3-4 and L2-3 separately and was technically uneventful. Epidural Anesthesia (EA) with levobupivacaine 0.1%+fentanyl (2 mcg/ml) was effective at 5 ml/h for postoperative analgesia. Eight hours post-op, she complained of having lost all feeling on the non-operative leg. On examination, she had diminished both touch sensory and motor power on the non-operative leg, but intact on the operative side with VAS at 2/10. Epidural catheter was receded 2 cm and the dose was lowered and paused. Despite of these adjustments after 4 hours, her experience was not improved. It was decided to use lipid emulsion as a challenging therapy. After completion of the lipid therapy, the sensory and movement of the non-operative leg regained normal after 60 min. The severe pain recurred which needed IV morphine titration. The EA of 2-5 ml/h was recontinued and effective for pain control for 3 days without no incident.

It is the first time in the medical literature that Lipid Emulsion is used for the purpose of LAR (Local Anesthesia Reversal) not connected to LAST (Local Anesthetic Systemic Toxicity).

### Case Report

A 71-year-old woman was scheduled for total knee replacement on left side. Past medical history was hypertension, type 2 diabetes, bilateral degenerative knee pain. Combined Spinal Epidural Anesthesia (CSEA) was performed at L3-4 and L2-3 separately and was technically uneventful. Spinal Anesthesia (SA) with 10 mg bupivacaine 0.5% heavy+20 mcg fentanyl was effective for surgery. Epidural Anesthesia (EA) with levobupivacaine 0.1%+fentanyl (2 mcg/ml) was effective at 5 ml/h for postoperative analgesia. She was sent back to ward after free movement of both knees. Eight hours post-op, she complained of having lost all feeling on the non-operative leg. On examination, she had diminished both touch sensory and motor power on the non-operative leg, but intact on the operative side with VAS at 2/10. After Local Anesthetic Systemic Toxicity (LAST) was ruled out, epidural catheter was receded 2 cm and the dose was lowered and paused. Despite of these adjustments after 4 hours, her experience was not improved.

It was decided to use lipid emulsion as a challenging therapy. A vial of 250 ml of Lipidem 20% (B. Braun) was infused over 30 min as regimen in ASRA's checklist for treatment of LAST. After completion of the lipid therapy, the sensory and movement of the non-operative leg regained normal after 60 min. The severe pain recurred which needed IV morphine titration. The EA of 2-5 ml/h was recontinued and effective for pain control for 3 days without no incident.

### Discussion

### LAST

On April 1998 Weinberg G L et al. published in Anesthesiology journal the following article: Pretreatment or resuscitation with a lipid infusion shifts the dose-response to bupivacaine-induced asystole in rats [1]. This article started the Intralipid (lipid infusion) LAST (Local Anesthetic Systemic Toxicity) treatment by Intralipid.

The article conclusion was that “Lipid infusion shifts the dose-response to bupivacaine-induced asystole in rats. Partitioning of bupivacaine into the newly created lipid phase may partially explain this effect. These results suggest a potential application for lipid infusion in treating cardiotoxicity resulting from bupivacaine”.

On February 2018 Neal J M et al. published in Regional Anesthesia Pain Medicine journal the following article: The Third American Society of Regional Anesthesia and Pain Medicine Practice Advisory on Local Anesthetic Systemic Toxicity: Executive Summary 2017 [2].

The article conclusion was: “This interim update summarizes recent scientific findings that have enhanced our understanding of the mechanisms that lead to lipid emulsion reversal of LAST, including rapid partitioning, direct inotropy, and post-conditioning. Since the previous practice advisory, epidemiological data have emerged that suggest a lower frequency of LAST as reported by single institutions and some registries, nevertheless a considerable number of events still occur within the general community. Contemporary case reports suggest a trend toward delayed presentation, which may mirror the increased use of ultrasound guidance (fewer intravascular injections), local infiltration techniques (slower systemic uptake), and continuous local anesthetic infusions. Small patient size and sarcopenia are additional factors that increase potential risk for LAST. An increasing number of reported events occur outside of the traditional hospital setting and involve nonanesthesiologists”.

The LAST was the subject regarding Intralipid and nothing else in all these 20 years of research. Eldor defined this Intralipid (fat emulsion containing soybean oil and egg yolk) as a “Magic Bullet” [3].

The case report mentioned in this article is a kind of a Local Anesthesia Reversal (LAR) by Intralipid. It is the first article mentioning this Intralipid—LAR treatment in the medical literature.

### AFE

There are other theoretical options for use of intralipid. For example:

Lipid emulsion rescue of amniotic fluid embolism induced cardiac arrest: “As a last resort, IV lipid 20% emulsion (1.5 mL/kg) was administered as a bolus [4]. Within 30 to 90 seconds, the patient had return of spontaneous circulation, normal sinus rhythm, and dramatic improvement in left and right ventricular function, shown clearly by TEE. After several minutes, the patient's condition slowly deteriorated once again to asystole, at which time CPR was once again started and a second lipid emulsion bolus (1.5 mL/kg) was administered and followed with an infusion at 0.25 mL/kg/min. Within 30 to 60 seconds, the patient again had a return of spontaneous circulation with normal sinus rhythm. In addition, she also exhibited spontaneous movements of her extremities”.

“Although Eldor et al. were the first to suggest a possible benefit of lipid emulsion therapy in the treatment of AFE, this is the first published instance in which a patient received intravenous lipid emulsion temporally related to the recovery from cardiovascular collapse associated with amniotic fluid embolism [3]. The main limitation is the fact that AFE is a diagnosis of exclusion; however, other differential diagnoses are less likely. There is TEE evidence that shows overall improvement of cardiac function temporally related to administration of lipid emulsion. The patient had return of spontaneous circulation occurring shortly after the administration of lipid emulsion on 2 different occasions after exhausting all other ACLS options, suggesting that lipid emulsion may have been responsible for the successful resuscitation. In addition, after the initial improvement, a relapse occurred, which was treated with a second bolus of lipid emulsion after which the same improvement in clinical and cardiac function occurred. Full neurologic recovery was noted after significantly prolonged cardiovascular collapse with chest compressions (40 min) and exhaustion of other standard ACLS medications. The excellent neurologic recovery emphasizes the importance of high quality and sustained CPR. Furthermore, a possible physiologic mechanism for the cardiopulmonary recovery is presented and is based on scientific models from previous research on the effects of lipid emulsion and its components. This report suggests a possible benefit of lipid emulsion therapy in the treatment of cardiovascular collapse caused by AFE, and further research will be required to elucidate the role of lipid emulsion therapy in the setting of AFE”.

### Spinal/Epidural Lavage

This study was designed to determine whether epidural motor blockade could be reversed by postoperative injections of crystalloid solutions via the epidural catheter [5]. Twenty-seven patients (ASA physical status I, nonlabouring) had epidural anesthesia with 0.75% bupivacaine for elective cesarean delivery. Postoperatively, patients were randomized to receive three 15 mL injections (over 30 min) of crystalloid solutions (normal saline or Ringer's lactate) or no treatment (control) via the epidural catheter. Degree of motor and sensory blockade was evaluated with an investigator blinded to treatment group. Rate of resolution of sensory blockade was not different among groups. However, time for resolution of motor blockade was more than twice as long in the control group than in either treatment group (control=178+/−70 min vs Ringer's lactate=84+/−44 min, normal saline=70+/−38 min, P=0.001). The data suggest that unwanted motor blockade due to epidural anesthesia can be reversed by epidural injections of crystalloid solutions [5].

Prolonged motor and sensory block following epidural anesthesia can be associated with extended postoperative care unit stays and patient dissatisfaction. Previous studies have demonstrated a more rapid motor recovery following the administration of epidural crystalloids in patients who had received plain bupivacaine and lidocaine epidural anesthesia. However, epinephrine is commonly added to local anesthetics to improve the quality and prolong the duration of the epidural block. The objective of this study was to determine the relationship of 0.9% NaCl epidural catheter flush volume (i.e., washout) to the recovery of motor and sensory block in patients undergoing 2% lidocaine with epinephrine epidural anesthesia [6].

A prospective, randomized, double-blind study design was utilized. Thirty-three subjects scheduled for elective gynecologic or obstetrical surgical procedures underwent epidural anesthesia using 2% lidocaine with epinephrine (1:200,000). A T4 dermatome level of analgesia, determined by toothpick prick, was maintained intraoperatively. Following surgery, subjects were randomized to 1 of 3 treatment groups. Group 1 (control, n=11) received no epidural 0.9% NaCl (normal saline [NS]) postoperatively.

Group 2 (15 mL NSx1, n=10) received an epidural bolus of 15 mL NS. Group 3 (15 mL NSx2, n=12) received an epidural bolus of 15 mL NS postoperatively and a second 15 mL NS bolus 15 minutes later. Assessment of motor and sensory block was performed at 15-minute intervals until complete motor and sensory recovery.

Times to partial and full motor and sensory recovery were significantly faster in the epidural NS groups than in the control group. Full motor recovery was more rapid in subjects receiving two 15 mL NS epidural NS boluses (30 mL total) compared with those receiving a single 15-mL NS bolus (108+/−9 min vs 136+/−13 min) and significantly faster than control group subjects (153+/−14 min). Both NSx1 and NSx2 epidural bolus groups experienced significantly reduced times to complete sensory recovery when compared with the control group (NSx1=154+/−13 min, NSx2=153+/−9 min, control 195+/−14 min).

A more rapid recovery of motor and sensory block in patients undergoing 2% lidocaine with epinephrine epidural anesthesia can be achieved with the use of 30 mL NS epidural washout [6].

In this case report, we describe the use of cerebrospinal fluid lavage as a successful treatment of an inadvertent intrathecally placed epidural catheter in a 14-yr-old girl who underwent a combination of epidural anesthesia and general anesthesia for orthopedic surgery [7]. In this case, a large amount of local anesthetic was injected (the total possible intrathecal injection was 200 mg of lidocaine and 61 mg of bupivacaine), resulting in apnea and fixed dilated pupils in the patient at the end of surgery. Twenty milliliters of cerebrospinal fluid was replaced with 10 mL of normal saline and 10 mL of lactated Ringer's solution from the “epidural” catheter. Spontaneous respiration returned 5 min later, and the patient was tracheally extubated after 30 min. No signs of neurological deficit or post dural puncture headache were noted after surgery.

Cerebrospinal lavage may be a helpful adjunct to the conventional supportive management of patients in the event of an inadvertent total spinal [7].

Several investigators have described the phenomena of epidural saline washout using bolus injections. This study was designed to determine whether epidural block could be reversed more effectively by infusion of crystalloid solutions via the epidural catheter.

One hundred male patients scheduled for outpatient surgery were enrolled in this study. After 30 min of 2% prilocaine epidural anesthesia, patients were randomly assigned to receive 45 mL of study solution as follows: (1) normal saline bolus (group NSB); (2) Ringer's lactate bolus (group RLB); (3) normal saline infusion (group NSI); (4) Ringer's lactate infusion (group RLI). Patients in the control group received no washout fluid. Motor, sensory blockade and side effects were compared among 5 groups. Ambulation time is defined as the recovery time.

In the control group, ambulation time (139+/−15 min) was significantly longer than in the washout groups (NSB 90+/−10, RLB 88+/−10, NSI 85+/−8, RLI 91+/−6 minutes) (P<0.001). Two-segment sensory regression time in the control group (86+/−15 min) was significantly longer than in groups NSB, RLB, NSI and RLI (55+/−8, 51+/−4, 58+/−8, and 53+/−10 minutes, respectively) (P<0.001).

We concluded that a more rapid recovery of motor and sensory blockade in patients undergoing epidural anesthesia may be achieved by the use of an epidural washout with either bolus or infusion of 45 mL normal saline or Ringer's lactate [8].

High or total spinal anesthesia commonly results from accidental placement of an epidural catheter in the intrathecal space with subsequent injection of excessive volumes of local anesthetic. Cerebrospinal lavage has been shown to be effective at reversing the effects of high/total spinal anesthesia but is rarely considered in obstetric cases. Here, we describe the use of cerebrospinal lavage to prevent potential complications from high/total spinal anesthesia after unintentional placement of an intrathecal catheter in a labouring obstetric patient [9].

A 34-yr-old female presented to the labour and delivery unit in active labour. Epidural anesthesia was initiated, and after the first bolus dose, the patient experienced lower extremity motor block and shortness of breath. A high spinal was confirmed, and cerebrospinal lavage was performed. In total, 40 mL of cerebrospinal fluid (CSF) were exchanged for an equal volume of normal saline. The patient's breathing difficulties and motor block resolved quickly, and a new epidural catheter was placed after removal of the spinal catheter. Pain control was effective, and the patient delivered a healthy baby.

We show that exchange of CSF for normal saline can be used successfully to manage a high spinal in an obstetric patient [9]. Our results suggest that CSF lavage could potentially be an important and helpful adjunct to the conventional supportive management of obstetric patients in the event of inadvertent high or total spinal anesthesia [9].

### Dental LAR

PM (OraVerse) enables the dentist or dental hygienist (where permitted) to significantly decrease the duration of residual STA in patients where such numbness may prove to be potentially injurious (children, geriatric, and special needs patients), or a negative influence on their quality of life (speaking, eating, negative body image). (Note: As of Aug. 3, 2009, dental hygienists are permitted to administer PM in the following states: Alaska, Arkansas, Hawaii, Idaho, Iowa, Louisiana, Montana, Nevada, New York, North Dakota, Oklahoma, Rhode Island, Tennessee, Utah, and Wisconsin) [10].

This study sought to identify and quantify complications with local anesthetic administration and reversal on consecutive patients seen for comprehensive dental care in a school-based, portable dental clinic, and includes data on the patients seen by the participating portable dental providers. In 923 dental visits where local anesthetic was administered, a standardized form was used to gain further information and identify any complications; this was accompanied by a questionnaire for the student's teacher, in order to quantify the student's distraction and disruption ratings following the dental visit. After statistical analysis of the 923 consecutive cases, the overall complication rate was 5.3%. All of the complications were considered to be mild or moderate, and there were no severe event reports. The complications encountered most frequently (n=49) were associated with self-inflicted soft tissue injury. The results of this study indicate that comprehensive care with local anesthesia delivered by a school-based portable dental clinic has a low risk of complications. Whereas safe administration of dental care is achievable with or without phentolamine mesylate as a local anesthetic reversal agent, its use was determined to improve safety outcomes. Three factors appeared to directly increase the incidence of complications: the administration of an inferior alveolar nerve block, attention deficit disorder, and obesity. Teacher evaluations demonstrated that children receiving care by a portable dental team were able to reorient back to class work and were not disruptive to classmates [11].

Administration of local anesthesia is an integral procedure prior to dental treatments to minimize the associated pain. It is learned that its effect stays more than the time required for the dental procedure to be completed. This prolonged soft tissue anesthesia (STA) can be detrimental, inconvenient, and unnecessary.

Phentolamine mesylate, a Food and Drug Administrationapproved drug essentially serves the purpose of faster recovery from numbness at the site of local anesthesia. This article reviews the development of the drug phentolamine mesylate and its indication as a local anesthetic reversal agent [12]. A literature search for phentolamine mesylate as a STA reversal agent was conducted in PubMed using the terms “dental local anesthesia reversal, phentolamine mesylate” up to March 2014. The search was limited to articles published in English. The search revealed 13 PubMed indexed articles stating the development and application of phentolamine mesylate. Phentolamine mesylate is an important step in the progress of developing patient care as well as an aid to the dental clinician [12].

### Lipid Emulsion for Non-Local Anesthetics Toxicity

The use of intravenous lipid emulsion (ILE) therapy for the treatment of lipophilic drug toxicity is increasing. Despite this, the evidence for its effect in non-local anesthetic toxicity remains sparse. Furthermore, many case reports describe ILE use for substances in which no clear efficacy data exists. The American Academy of Clinical Toxicology established a lipid emulsion workgroup. The aim of this group is to review the available evidence regarding the effect of ILE in non-LA drug poisoning and develop consensus-based recommendations on the use of this therapy.

A systematic review of the literature was performed to capture articles through 15 Dec. 2014. Relevant articles were determined based upon a predefined methodology. Articles involving pre-treatment experiments, pharmacokinetic studies not involving toxicity, and studies not addressing antidotal use of ILE met pre-defined exclusion criteria. Agreement of at least two members of the subgroup was required before an article could be excluded.

The final analysis included 203 articles: 141 for humans and 62 for animals. These include 40 animal experiments and 22 case reports involving animal toxicity. There were three human randomized control trials (RCT): one RCT examined ILE in TCA overdose, one RCT examined ILE in various overdoses, and one study examined ILE in reversal of sedation after therapeutic administration of inhaled anesthesia. One observational study examined ILE in glyphosate overdose. In addition, 137 human case reports or case series were identified. Intravenous lipid emulsion therapy was used in the management of overdose with 65 unique substances.

Despite the use of ILE for multiple substances in the treatment of patients with poisoning and overdose, the effect of ILE in various non-local anesthetic poisonings is heterogenous, and the quality of evidence remains low to very low [13].

### IRE (Intralipid Rescue Evidence)

The quality of “evidence” related to case reports cannot be “low to very low” unless there is “evidence” that the authors fabricated the facts of the case.

We do not think that anyone in any Practice Advisory in any Executive Summary has any proof to delete any evidence from any case report.

This is what makes Intralipid (or any other fat emulsion with soybean oil and egg yolk) a “magic bullet”: “Intravenous lipid emulsion therapy was used in the management of overdose with 65 unique substances”.

Does anyone know of another substance that can reverse the toxicity of 65 unique substances? and this is only the beginning.

### Conclusion

It is the first time in the medical literature that Lipid emulsion is used for the purpose of LAR (Local Anesthesia Reversal) not connected to LAST (Local Anesthetic Systemic Toxicity).

### Example 13

### The First Case Report of Local Anesthesia Reversal (LAR) of the Upper Arm Brachial Plexus Block by Lipid Emulsion

A 25 years old male was diagnosed as having natural stump in left forearm due to detonator. He needed an emergency surgery for cutting damaged tissue and repair of the stump. The patient was anesthetized by Ultrasound guided brachial plexus block through the Supra Clavicular approach with Lidocaine 4 mg/kg combined with Ropivacaine 1 mg/kg and Epinephrine 1/200.000 in total of 30 ml solution. Lipid emulsion (Lipofundin 20%, B. Braun) bolus injection was given 70 min after the Brachial plexus block over 2 min in a dose of 1.5 mg/kg (patient's weight is 60 kg). After 2 min of the Lipid emulsion bolus injection he could move slightly his left arm. Full Local Anesthesia Reversal (LAR) was achieved after 255 min from the Brachial plexus block performance.

It is the first case report in the medical literature of Local Anesthesia Reversal (LAR) of the upper arm Brachial plexus block by Lipid Emulsion.

### Case Report

A 25 years old male was diagnosed as having natural stump in left forearm due to detonator. (Photo: http://www.csen.com/stump1.jpg)(X-ray: http://www.csen.com/stump2.jpg).

He needed an emergency surgery for cutting damaged tissue and repair of the stump. The operation was done on Feb. 16, 2018 at the Military Hospital 103, Vietnam Military Medical University. The patient was anesthetized by Ultrasound guided brachial plexus block through the Supra Clavicular approach with Lidocaine 4 mg/kg combined with Ropivacaine 1 mg/kg and Epinephrine 1/200.000 in total of 30 ml solution.

Onset time of Anesthesia: 5 min.

Anesthesia effect was very good for the surgery.

Duration of surgery: 50 min

Surgery was accomplished in 65 min after the Brachial plexus block. Sensory and motor function of the left hand that were assessed before injection were absent completely.

Lipid emulsion (Lipofundin 20%, B. Braun) bolus injection was given 70 min after the Brachial plexus block over 2 min in a dose of 1.5 mg/kg (patient's weight is 60 kg). The patient was monitored closely in the operating room for 1 hour:

(Video 1: https://www.youtube.com/watch?v=93QrTB--2_o)

After 2 min of the Lipid emulsion bolus injection he could move slightly his left arm. Vital signs were stable after the Lipid emulsion bolus injection.

After 185 min from the lipid emulsion bolus injection the sensory and motor function of the left hand were fully recovered. He could move his left hand without any difficulty. It means that full Local Anesthesia Reversal (LAR) was achieved after 255 min from the Brachial plexus block performance. (Video 2: https://www.youtube.com/watch?v=K2H4h-GLZko&feature=youtu.be)

### Discussion

### Supraclavicular Block

The first percutaneous supraclavicular block was performed in 1911 by German surgeon Diedrich Kulenkampff (1880-1967)[1]. Kulenkampff subjected himself to the supraclavicular block. Later that year, Georg Hirschel (1875-1963) described a percutaneous approach to the brachial plexus from the axilla[2]. By the late 1940s, clinical experience with brachial plexus block in both peacetime and wartime surgery was extensive, and new approaches to this technique began to be described[3]. For example, In 1946, F. Paul Ansbro was the first to describe a continuous brachial plexus block technique. He secured a needle in the supraclavicular fossa and attached tubing connected to a syringe through which he could inject incremental doses of local anesthetic[4]. The subclavian perivascular block was first described by Winnie and Collins in 1964[5]. This approach became popular due to its lower risk of pneumothorax compared to the traditional Kulenkampffapproach. The infraclavicular approach was first developed by Raj. In 1977, Selander described a technique for continuous brachial plexus block using anintravenous catheter secured in the axilla[6].

### Prolonged Blockade

There are a few reports of prolonged blockade following seemingly flawless technique of performing block. Complete recovery in those case reports varied from 40 to 84 hours after the block[?-8]. None of the papers have clearly stated the reason behind the long blockade. Injecting the local anesthetic too close to the nerves and chronic treatment with lithium has been proposed as reasons behind these unusually prolonged blocks. Luduena believed that causes of prolonged blockade are often unknown and if the duration is longer than 24 hours then probability of nerve damage should be considered [9].

### Regular Blockade

The present study compares the effectiveness of 0.25% ropivacaine and 0.25% bupivacaine in 44 patients receiving a subclavian perivascular brachial plexus block for upper extremity surgery. The patients were assigned to two equal groups in this randomized, double-blind study; one group received ropivacaine 0.25% (112.5 mg) and the other, bupivacaine 0.25% (112.5 mg), both without epinephrine. Onset times for analgesia and anesthesia in each of the C-5 through T-1 brachial plexus dermatomes did not differ significantly between the two groups. The mean onset time for analgesia ranged from 11.2 to 20.2 min, and the mean onset time for anesthesia ranged from 23.3 to 48.2 min. The onset of motor block differed only with respect to paresis in the hand, with bupivacaine demonstrating a shorter onset time than ropivacaine. The duration of sensory and motor block also was not significantly different between the two groups. The mean duration of analgesia ranged from 9.2 to 13.0 h, and the mean duration of anesthesia ranged from 5.0 to 10.2 h. Both groups required supplementation with peripheral nerve blocks or general anesthesia in a large number of cases, with 9 of the 22 patients in the bupivacaine group and 8 of the 22 patients in the ropivacaine group requiring supplementation to allow surgery to begin. In view of the frequent need for supplementation noted with both 0.25% ropivacaine and 0.25% bupivacaine, we do not recommend using the 0.25% concentrations of these local anesthetics to provide brachial plexus block [10].

The effects of clonidine and epinephrine, administered into the brachial plexus sheath, were evaluated in 60 patients who underwent surgery of the upper limb. All patients received 40 to 50 ml of 0.25% bupivacaine, injected into the brachial plexus sheath, using the supraclavicular technique. The patients were randomly allocated to two groups so that 30 patients received 150 micrograms clonidine hydrochloride (Group I), and 30 received 200 micrograms epinephrine (Group II). The quality and the duration of analgesia were assessed as well as the possible side-effects. The block produced with the addition of clonidine was longer (994.2+/−34.2 vs 728.3+/−35.8 min) and superior to that with epinephrine (P less than 0.001). No major side-effects were recorded. We conclude that the injection of clonidine into the brachial plexus sheath is an attractive alternative to epinephrine to prolong the duration of analgesia following upper limb surgery under conduction anaesthesia [11].

This study compared the effectiveness of 0.5% ropivacaine and 0.5% bupivacaine for brachial plexus block[12]. Fortyeight patients received a subclavian perivascular brachial plexus block for upper-extremity surgery. One group (n=24) received ropivacaine 0.5% (175 mg) and a second group (n=24) received bupivacaine 0.5% (175 mg), both without epinephrine. Onset times for analgesia and anesthesia in each of the C5 through T1 brachial plexus dermatomes did not differ significantly between groups. Duration of analgesia and anesthesia was long (mean duration of analgesia, 13-14 h; mean duration of anesthesia, 9-11 h) and also did not differ significantly between groups. Motor block was profound, with shoulder paralysis as well as hand paresis developing in all of the patients in both groups. Two patients in each group required supplemental blocks before surgery. Ropivacaine 0.5% and bupivacaine 0.5% appeared equally effective in providing brachial plexus anesthesia[12].

The mixture of 1% lidocaine and 0.2% tetracaine with 1:200,000 epinephrine, so-called “supercaine,” has been used extensively for axillary brachial plexus blockade for several decades. Since the advent of bupivacaine, the supercaine mixture has fallen into relative disuse despite its record of effectiveness and safety. No studies have been done recently to evaluate quality of anesthesia, duration of postoperative analgesia, and degree of patient satisfaction with this mixture when used for axillary brachial plexus blockade. The assumptions were as follows: surgical anesthesia will be adequate, length of postoperative analgesia will be approximately 4 to 9 hours, and patients will be highly satisfied. The specific aim of the present study was to describe the anesthetic characteristics of supercaine. Patients between 18 and 65 years of age received a standard mixture of supercaine, totalling 450-500 mg of lidocaine and 90 to 100 mg of tetracaine. Epinephrine in a solution of 1:200,000 and an 8.4% solution of sodium bicarbonate were added, and the trans arterial technique was used. Patients were contacted on postoperative day 1 to determine the duration of sensory and motor block; overall satisfaction with the block was rated. Data were analyzed with the Statistical Program for the Social Sciences (SPSS, Chicago, Ill.) and Stata (Stata Corp., College Station, Tax) computer programs. The mean+/−SD findings were as follows: duration of sensory block, 465+/−204 minutes; duration of motor block, 473+/−214 minutes; patient satisfaction score, 9+/−1 on a 1 to 10 scale. Data are reported within a 95% confidence interval. Variables examined and compared were not statistically significant. We concluded that the duration of block supports findings reported in the literature, patients equate duration of sensory block with duration of motor block, differences in duration were probably due to levels of provider experience, and patients were extremely satisfied with the anesthetic[13].

Ultrasound-guided supraclavicular brachial plexus block (USSB) provides excellent postoperative analgesia after upper extremity surgery. Dexamethasone and clonidine have been added to local anesthetics to enhance and prolong the duration of analgesia.

The objective of this randomized prospective study is to evaluate the efficacy of dexamethasone, clonidine, or combination of both as adjuvants to ropivacaine on the duration of USSB for postoperative analgesia.

Patients receiving USSB for postoperative pain control for upper extremity surgery were randomized to one of four groups; ropivacaine 0.5%, ropivacaine 0.5% with 4 mg dexamethasone, ropivacaine 0.5% with 100 mcg clonidine, or ropivacaine 0.5% with 4 mg dexamethasone and 100 mcg clonidine. Pain scores, sensory and motor function were evaluated at post anesthesia care unit (PACU), discharge and at 24 h postoperatively.

The duration of sensory and motor blocks were significantly longer in clonidine groups when compared to ropivacaine alone [Sensorial analgesia: ropivacaine alone 13.4±6, Ropivacaine-Clonidine 17.4±6; Ropivacaine-Dexamethasone-Clonidine 18.8±6.2; Motor blocks: Ropivacaine 12±5, Ropivacaine-Clonidine 16.8±5.2, Ropivacaine-Dexamethasone-Clonidine 18.2±5.7]. In clonidine groups, there was significantly prolongation of motor and sensory block when compared to ropivacaine alone group.

The results demonstrated that clonidine significantly prolongs the duration of ropivacaine effects for the postoperative analgesia in patient underwent upper arm surgeries [14].

The duration of effect for axillary plexus block using ropivacaine is highly variable. The available literature does not offer any plausible means of predicting time of block offset for individual patients, making it difficult to give accurate information and plan postoperative analgesics. This study was designed to identify factors influencing axillary plexus block offset time.

A total of 92 patients participated in this prospective double centred observational study. All patients were scheduled for axillary plexus block with ropivacaine 0.75% and subsequent block duration was recorded.

Mean time of axillary plexus block offset was 13.5 hours, with a range of 4.8 to 25.4 hours. No statistical significant differences in offset time was seen with regard to gender, age, body weight, BMI and ASA-classification. A trend for increasing duration of blocks associated with increasing age was observed. No statistically significant difference was identified in block duration between blocks performed with nerve stimulator guidance versus ultrasound guidance. Similarly, neither dose nor volume of ropivacaine 0.75% was identified as a factor influencing block duration.

This prospective study demonstrates a large inter individual variation in time of axillary plexus block offset using ropivacaine 0.75%. The lack of association between offset time and both demographic and block performance factors, makes predictability of individual duration of axillary plexus blocks in clinical practice extremely difficult. We suggest that all patients should be made aware of such variability in duration prior to block placement [15].

On a pharmacologic basis, levobupivacaine is expected to last longer than ropivacaine. However, most reports of these anesthetics for brachial plexus block do not suggest a difference in analgesic effect. The aim of this study is to compare the postoperative analgesic effects of levobupivacaine and ropivacaine when used for treating ultrasound-guided brachial plexus block.

A total of 62 patients undergoing orthopaedic surgery procedures were prospectively enrolled and randomized to receive levobupivacaine (group L, N=31) or ropivacaine (group R, N=31). The duration of analgesia, offset time of motor block, need for rescue analgesics, and sleep disturbance on the night of surgery were recorded. Pain score was recorded on the day of surgery, and on postoperative days 1 and 2.

There was no difference in the time interval until the first request for pain medication comparing the two groups (group L: 15.6 [11.4, 16.8] hours; group R: 12.5 [9.4, 16.0] hours, P=0.32). There was no difference in the duration of motor block (group L: 12.2 [7.6, 14.4] hours; group R: 9.4 [7.9, 13.2] hours, P=0.44), pain score (P=0.92), need for rescue analgesics (group L: 55%; group R: 65%, P=0.6), or rate of sleep disturbance (group L: 61%, group R: 58%, P=1.0) on comparing the two groups.

There was no difference in postoperative analgesia comparing levobupivacaine and ropivacaine when used for brachial plexus block[16].

For any surgery in the upper extremity that does not involve the shoulder, a supraclavicular block is preferred, as it is a safe procedure associated with rapid onset and reliable anaesthesia. Although ropivacaine has been extensively studied for epidural anaesthesia, very few reports exist on its use in supraclavicular brachial plexus block.

This study was conducted to investigate and compare the effectiveness of supraclavicular brachial plexus anaesthesia with two different concentrations of ropivacaine (0.5% and 0.75%) and to compare them with the standard 0.5% bupivacaine [17].

Ninety patients of age 18 to 60 years belonging to American Society of Anaesthesiologists (ASA) status 1 or 2, admitted to Pondicherry Institute of Medical Sciences were chosen for the study and were divided into three groups. Group A received 30 ml of 0.5% bupivacaine, group B received 30 ml of 0.5% ropivacaine and group C received 30 ml of 0.75% ropivacaine into the supraclavicular region, by a nerve-stimulator technique. Onset time of each of the drug was recorded both for the sensory and motor block. Duration of sensory and motor block was recorded along with pen-operative haemodynamic monitoring.

The onset of complete sensory and motor block observed with both ropivacaine groups and bupivacaine was similar (16.85±6.67 min in group A, 17.79±5.03 min in group B and 18.48±6.14 in group C, p>0.05); onset of motor block (21.45±4.45 min in group A, 22.23±4.05 min in group B and 22.33±5.17 in group C, p<0.05). The duration of sensory block with 0.5% bupivacaine was 11.58 hours, with 0.5% ropivacaine was 9.02 hours with 0.75% ropivacaine was 8.87 hours (p<0.001). The duration of motor block with 0.5% bupivacaine was 12.94 hours, with 0.5% ropivacaine was 8.29 hours with 0.75% ropivacaine was 7.89 hours (p<0.001). Multiple comparison test with Bonferroni correction showed there was statistically significant difference in mean duration of sensory block between Group A (0.5% bupivacaine) and Group B (0.5% ropivacaine) and also between Group A (0.5% bupivacaine) and Group C (0.75% ropivacaine). However, there were no statistically significant difference in mean duration of sensory block between Group B (0.5% ropivacaine) and Group C (0.75% ropivacaine). The preoperative, intra operative and postoperative heart rate, systolic & diastolic blood pressure and oxygen saturation were comparable among the three study groups (p>0.05). No side effects were recorded in the study.

The onset of sensory and motor block was similar in all the three groups. However, when compared to bupivacaine group, recovery of motor functions was faster in both the ropivacaine groups. Patients in all the 3 groups did not experience any adverse effects [17].

### Lipid Emulsion Effects on Mitochondria and Intracellular Calcium

Local anesthetic toxicity is thought to be mediated partly by inhibition of cardiac mitochondrial function. Intravenous (i.v.) lipid emulsion may overcome this energy depletion, but doses larger than currently recommended may be needed for rescue effect. In this randomized study with anesthetized pigs, we compared the effect of a large dose, 4 mL/kg, of i.v. 20% Intralipid® (n=7) with Ringer's acetate (n=6) on cardiovascular recovery after a cardiotoxic dose of bupivacaine[18]. We also examined mitochondrial respiratory function in myocardial cell homogenates analyzed promptly after needle biopsies from the animals. Bupivacaine plasma concentrations were quantified from plasma samples. Arterial blood pressure recovered faster and systemic vascular resistance rose more rapidly after Intralipid than Ringer's acetate administration (p<0.0001), but Intralipid did not increase cardiac index or left ventricular ejection fraction. The lipid-based mitochondrial respiration was stimulated by approximately 30% after Intralipid (p<0.05) but unaffected by Ringer's acetate. The mean (standard deviation) area under the concentration-time curve (AUC) of total bupivacaine was greater after Intralipid (105.2 (13.6) mg·min/L) than after Ringer's acetate (88.1 (7.1) mg·min/L) (p=0.019). After Intralipid, the AUC of the lipid-un-entrapped bupivacaine portion (97.0 (14.5) mg·min/L) was 8% lower than that of total bupivacaine (p<0.0001). To conclude, 4 mL/kg of Intralipid expedited cardiovascular recovery from bupivacaine cardiotoxicity mainly by increasing systemic vascular resistance. The increased myocardial mitochondrial respiration and bupivacaine entrapment after Intralipid did not improve cardiac function [18].

Lipid emulsions have been used to treat various drug toxicities and for total parenteral nutrition therapy. Their usefulness has also been confirmed in patients with local anesthetic-induced cardiac toxicity. The purpose of this study was to measure the hemodynamic and composition effects of lipid emulsions and to elucidate the mechanism associated with changes in intracellular calcium levels in myocardiocytes.

Wemeasured hemodynamic effects using a digital analysis system after Intralipid® and Lipofundin® MCT/LCT were infused into hearts hanging in a Langendorff perfusion system[20]. We measured the effects of the lipid emulsions on intracellular calcium levels in H9c2 cells by confocal microscopy.

Infusion of Lipofundin® MCT/LCT 20% (1 ml/kg) resulted in a significant increase in left ventricular systolic pressure compared to that after infusing modified Krebs-Henseleit solution (1 ml/kg) (P=0.003, 95% confidence interval [CI], 2.4-12.5). Lipofundin® MCT/LCT 20% had a more positive inotropic effect than that of Intralipid® 20% (P=0.009, 95% CI, 1.4-11.6). Both lipid emulsion treatments increased intracellular calcium levels. Lipofundin® MCT/LCT (0.01%) increased intracellular calcium level more than that of 0.01% Intralipid® (P<0.05, 95% CI, 0.0-1.9).

These two lipid emulsions had different inotropic effects depending on their triglyceride component. The inotropic effect of lipid emulsions could be related with intracellular calcium level [19].

Accidental intravascular or high-dose injection of local anesthetics (LA) can result in serious, potentially life threatening complications. Indeed, adequate supportive measures and the administration of lipid emulsions are required in such complications. The study's objectives were threefold: (i) evaluate the myocardial toxicity of levobupivacaine when administered intravenously; (ii) investigate levobupivacaine toxicity on cardiomyocytes mitochondrial functions and cellular structure; (iii) assess the protective effects of a lipid emulsion in the presence or absence of myocardial ischemia. Domestic pigs randomized into two groups of 24 animals each, with either preserved coronary circulation or experimental myocardial ischemia. Six animals from each group received either: (i) single IV injection of saline, (ii) lipid emulsion (Intralipid(®)), (iii) levobupivacaine, (iv) combination levobupivacaine-Intralipid(®). Serially measured endpoints included: heart rate, duration of the monophasic action potentials (dMAP), mean arterial pressure, and peak of the time derivative of left ventricular pressure (LV dP/dtmax). In addition, the following cardiomyocytes mitochondrial functions were measured: reactive oxygen species (ROS) production, oxidative phosphorylation, and calcium retention capacity (CRC) as well as the consequences of ROS production on lipids, proteins, and DNA. IV injection of levobupivacaine induced sinus bradycardia and reduced dMAP and LV dP/dtmax. At the mitochondrial level, oxygen consumption and CRC were decreased. In contrast, ROS production was increased leading to enhanced lipid peroxidation and structural alterations of proteins and DNA. Myocardial ischemia was associated with global worsening of all changes. Intralipid(®) quickly improved haemodynamics. However, beneficial effects of Intralipid(®) were less clear after myocardial ischemia [20].

Cocaine intoxication leads to over 500,000 emergency department visits annually in the United States and ethanol cointoxication occurs in 34% of those cases. Cardiotoxicity is an ominous complication of cocaine and cocaethylene overdose for which no specific antidote exists. Because infusion of lipid emulsion (Intralipid) can treat lipophilic local anesthetic toxicity and cocaine is an amphipathic local anesthetic, the authorstested whether lipid emulsion could attenuate cocaine cardiotoxicity in vivo[21]. The effects of lipid emulsion were compared with the metabolically inert sulfobutylether-β-cyclodextrin (SBE-β-CD; Captisol) in an isolated heart model of cocaine and cocaethylene toxicity to determine if capture alone could exert similar benefit as lipid emulsion, which exhibits multimodal effects. The authors then tested if cocaine and cocaethylene, like bupivacaine, inhibit lipid-based metabolism in isolated cardiac mitochondria.

For whole animal experiments, Sprague-Dawley rats were anesthetized, instrumented, and pretreated with lipid emulsion followed by a continuous infusion of cocaine to assess time of onset of cocaine toxicity. For ex vivo experiments, rat hearts were placed onto a nonrecirculating Langendorff system perfused with Krebs-Henseleit solution. Heart rate, left ventricle maximum developed pressure (LVdevP), left ventricle diastolic pressure, maximum rate of contraction (+dP/dtmax), maximum rate of relaxation (−dP/dtmax), rate-pressure product (RPP=heart ratexLVdevP), and line pressure were monitored continuously during the experiment. A dose response to cocaine (10, 30, 50, and 100 μmol/L) and cocaethylene (10, 30, and 50 μmol/L) was generated in the absence or presence of either 0.25% lipid emulsion or SBE-β-CD. Substrate-specific rates of oxygen consumption were measured in interfibrillar cardiac mitochondria in the presence of cocaine, cocaethylene, ecgonine, and benzoylecgonine.

Treatment with lipid emulsion delayed onset of hypotension (140 seconds vs. 279 seconds; p=0.008) and asystole (369 seconds vs. 607 seconds; p=0.02) in whole animals. Cocaine and cocaethylene induced dose-dependent decreases in RPP, +dP/dtmax, and −dP/dtmaxabs (p<0.0001) in Langendorff hearts; line pressure was increased by cocaine and cocaethylene infusion, but not altered by treatment. Lipid emulsion attenuated cocaine- and cocaethyleneinduced cardiac depression. SBE-β-CD alone evoked a mild cardiodepressant effect (p<0.0001) but attenuated further cocaine- and cocaethylene-induced decrements in cardiac contractility at high concentrations of drug (100 μmol/L; p<0.001). Finally, both cocaine and cocaethylene, but not ecgonine and benzoylecgonine, inhibited lipid-dependent mitochondrial respiration by blocking carnitine exchange (p<0.05). A commercially available lipid emulsion was able to delay progression of cocaine cardiac toxicity in vivo. Further, it improved acute cocaine- and cocaethyleneinduced cardiac toxicity in rat isolated heart while SBE-β-CD was effective only at the highest cocaine concentration. Further, both cocaine and cocaethylene inhibited lipid dependent mitochondrial respiration. Collectively, this suggests that scavenging-independent effects of lipid emulsion may contribute to reversal of acute cocaine and cocaethylene cardiotoxicity, and the beneficial effects may involve mitochondrial lipid processing [21].

We hypothesized that acute lipid-induced insulin resistance would be attenuated in high-oxidative muscle of lean trained (LT) endurance athletes due to their enhanced metabolic flexibility and mitochondrial capacity[22]. Lean sedentary (LS), obese sedentary (OS), and

LT participants completed two hyperinsulinemic euglycemic clamp studies with and without (glycerol control) the coinfusion of Intralipid. Metabolic flexibility was measured by indirect calorimetry as the oxidation of fatty acids and glucose during fasted and insulin-stimulated conditions, the latter with and without lipid oversupply. Muscle biopsies were obtained for mitochondrial and insulin-signaling studies. During hyperinsulinemia without lipid, glucose infusion rate (GIR) was lowest in OS due to lower rates of nonoxidative glucose disposal (NOGD), whereas state 4 respiration was increased in all groups. Lipid infusion reduced GIR similarly in all subjects and reduced state 4 respiration. However, in LT subjects, fat oxidation was higher with lipid oversupply, and although glucose oxidation was reduced, NOGD was better preserved compared with LS and OS subjects. Mitochondrial performance was positively associated with better NOGD and insulin sensitivity in both conditions. We conclude that enhanced mitochondrial performance with exercise is related to better metabolic flexibility and insulin sensitivity in response to lipid overload[22].

### Conclusion

It is the first case report in the medical literature of Local Anesthesia Reversal (LAR) of the upper arm Brachial plexus block by Lipid Emulsion.

### Example 14

### Local Anesthesia Reversal (LAR) of Total Spinal Anesthesia (TSA) by Lipofundin (Lipid Emulsion)

These 3 case reports are the first reports in the medical literature regarding the Local Anesthesia Reversal (LAR) of Total Spinal Anesthesia (TSA) by Lipofundin which is similar to Intralipid having soybean oil fatty acids including Linoleic acid.

### Case Report 1

A 22 years old, 158 cm of height, 40 kg weight, female, was under Spinal Anesthesia for tibia reconstruction on the right leg. Past medical history was normal. The patient had a right tibia fracture, scheduled for tibia fixation.

Spinal Anesthesia (SA) was performed at L3-4, with Bupivacaine 0.5% hyperbaric. No sedation was given. Baseline BP 126/83 mmHg, HR 90, SpO2 100% (nasal oxygenation 31/min). SA technique was uneventful and skin incision initiated.

Approximately 5 mins after SA, patient experienced nausea, BP decreased to 90/50, HR 90. The patient got acceleration of 500 ml Crystalloid I.V. 10 min after SA, the patient's consciousness was confused, nausea, (Ramsay Score 1), BP 110/80 mmHg, HR 88, SpO2 98%. 20 Min after SA, patient was in a total coma state (Ramsay Score 6), iris was bilaterally small, BP 89/77, SpO2 98%.

(However, while asking and receiving the permission of the patient to include her videos in the article including her face (a signed permission) that was done a few days later, the patient told us that “In fact she was in the “locking syndrome” in which she heard, understood everything that was happening, but could not open her eyes nor talk (as the situation under muscle relaxant without enough sedative in general anesthesia). After the first bolus of Lipofundin she was awake nearly completely, but after that returned to the previous situation. After the second bolus, she felt much better and could respond to open her eyes as described in the report”.)

The anesthesiologist decided to give 250 ml of Lipofundin 20%. After 60 ml bolus dose, the patient opened her eyes to demand (Ramsay Score 3), but felt tired, nausea, difficult to breath, otherwise hemodynamically was stable (BP 103/74, HR 88, SpO2 98%), Pinprick test lost at neck and upper extremities (C4-5).

Another 60 ml bolus of Lipofundin 20% was added. The patient opened her eyes spontaneously (Ramsay Score 2) for a while, then stayed at Ramsay score 4. Other vital signs were stable: BP 120/77, HR 76, Spontaneous breathing with SpO2 98%. The rest of Lipofundin 20% I.V continued over 30 min. During the incident, surgery was continued and lasted for 50 mins.

At the end of surgery, the Ramsay Score was 3, short of breath decreased, the pinprick test lost at T4-5, vital signs were stable (BP 120/81, HR 78, SpO2 98%) without any vasoconstricting drugs nor respiratory supports (video clip 1).

Video clip 1: https://youtu.be/JmYXsaxlnak

At the recovery room, the patient received a second infusion of 250 ml vial of Lipofundin 20% over 60 mins.

At 10 min: Ramsay Score 3, Pinprick test lost at T6, Bromage Score 0, slight myoclonus on upper extremities noted, vital signs were stable (video clip 2).

Video clip 2: https://youtu.be/Jibckg-tpro

At 30 min: Ramsay Score 2, pinprick test lost at T6, Bromage Score 1. (video clip 3).

Video clip 3: https://youtu.be/UuKq51vWVag

At 60 min: Ramsay Score 2, pinprick test lost at T7-8, Bromage Score 1-2. (video clip 4).

Video clip 4: https://youtu.be/bQS811zsNUY

At 120 min: Ramsay Score 1, pinprick test lost at T 12, Bromage Score 1, no notion of any sign of myoclonus (Table 1)

TABLE 1
RAMSAY Score

| | |
|---|---|
| 1 | Patient is anxious and agitated or restless, or both |
| 2 | Patient is cooperative, oriented, and tranquil |
| 3 | Patient responds to command only |
| 4 | A brisk response to a light glabella tap or loud auditory stimulus |
| 5 | A sluggish response to a light glabella tap or loud auditory stimulus |
| 6 | No response to a light glabella tap or loud auditory stimulus |

### Case Report 2

A 28 years old, 155 cm height, 62 kg weight, healthy parturient with healthy pregnancy was scheduled for Cesarean Section for her second baby. Her previous Cesarean Section was done under Spinal anesthesia 5 years ago and was uneventfully. The laboratory tests were in normal ranges. Spinal Anesthesia was performed at L3-4 in the sitting position using Bupivacaine 10 mg+Fentanyl 25 mcg+Morphine 150 mcg.

Two mins after the SA, BP decreased to 70/40, HR 120, shortness of breath, pale, SpO2 87-90%, (under oxygenation of 5 L/min), the voice was lost.

She was given oxygen via mask 10 L/min, 30 mg Ephedrine to recover BP, but still experienced shortness of breath and lost voice. The anesthesiologist considered these signs as LAST (Local Anesthetic Systemic Toxicity) and decided to treat the patient by LE 20% (Lipofundin 20%). After a bolus of 100 mL, the skin's color improved, SpO2 improved to 100%, BP was stable.

Twenty mins after the Infusion of 250 ml Lipofundin 20%, she regained the voice, no shortness of breath any more, SpO2 100%, BP stable.

The surgery was continued uneventfully for 30 mins. The Apgar score of the newborn was ok.

### Case Report 3

A 28 years old, 165 cm, 70 kg weight, first pregnancy, healthy, full-term parturient was indicated for a Cesarean Section because the fetal head did not descend.

The first Spinal Anesthesia (SA) was performed in the sitting position at L2-3 with a dose of 8 mg Bupivacaine 0.5% heavy+50 mcg Fentanyl.

The first SA failed, so she was given another SA in the sitting position in the same level with a dose of 6 mg Bupivacaine 0.5% heavy.

She was kept in head up position thereafter. Just after lying down on the table, she developed pale, severe dyspnea, BP decreased to 60/30, HR 50, SpO2<80%, sensory touch was lost at the upper extremities. The 500 ml Crystalloid infused rapidly, facial mask ventilation with Oxygen, and 30 mg Ephedrine given I.V to maintain BP (80/60 mmHg). Meanwhile the surgery initiated immediately to take out the neonate.

At this time the anesthesiologist was considering intubation, but he decided to use lipid emulsion therapy because he thought that it was LAST (Local Anesthetic Systemic Toxicity) and not a benign hypotension due to SA.

Since of LE 20% (Lipofundin 20%) was unavailable, the anesthesiologist used Lipofundin 10% instead.

After a rapid IV of 250 ml of Lipofundin 10% (approximately 10 min), the parturient improved completely: full consciousness, normal spontaneous breathing, SpO2 100% with an oxygen face mask, stable BP, HR without using any vasoconstrictor, the sensory loss estimated at T4. At the end of the surgery (approx. 30 mins) the parturient returned to nearly normal state.

### Discussion

### Introduction

When a local anesthetic is injected multiple times into the subarachnoid space, it can lead to an extensive block, termed total spinal anesthesia (TSA). The most common cause is an accidental dural injection during an epidural anesthesia [1].

High neuraxial block is a common cause of anesthesiarelated maternal death. Two-thirds of high blocks were caused by accidental intrathecal injection through a presumed epidural catheter [2].

Typical features of TSA include fall in blood pressure, cessation of respiration, loss of consciousness and even cardiac arrest, which can occur within minutes of injecting a spinal drug [3]. Sometimes symptoms are atypical and difficult to identify [4].

The duration and severity of TSA is related to the type and dose of local anesthetic; for example, 0.75% bupivacaine was shown to have a mean duration of action of 1.511-3 h [5].

The study was part of the Thai Anesthesia Incidents Study (THAI Study) of anesthetic adverse outcomes. Study complications after spinal anesthesia [6].

During the 12-month period (Mar. 1, 2003-Feb. 28, 2004), a prospective multi centered descriptive study was conducted in 20 hospitals comprised of seven university, five tertiary, four general and four district hospitals across Thailand. Anesthesia personnel filled up patient-related, surgical-related, and anesthesia-related variables and adverse outcomes of all consecutive patients receiving anesthesia on a structured data entry form. The data were collected during pre-anesthetic, intra-operative, and 24 hr post-operative period. Adverse event specific forms were used to record when these incidents occurred. Data were reviewed by three independent reviewers and analyzed to identify contributing factors by consensus.

This was registry of 40,271 spinal anesthetics from 172,697 anesthetics. The incidence of total spinal anesthesia, neurological complications, suspected myocardial ischemia, or infarction and oxygen desaturation per 10,000 spinal anesthetics were 3.48 (95% CI 1.66-5.30), 1.49 (95% CI 0.30-2.68), 2.73 (95% CI 1.12-4.35), 0.99 (95% CI 0.39-2.56), and 6.46 (95% CI 3.98-8.94) respectively. This was not different to the incidence in other countries. Risk factors of oxygen desaturation were shorter in height [OR 0.95 (95% CI 0.92-0.97); p<0.0011, higher ASA physical status [OR 3.37 (95% CI 1.98-5.72); p<0.001] and use of propofol [OR 5.22 (95% CI 1.78-15.35); p=0.003]. Other complications such as seizure, anaphylactic or anaphylactoid reaction, drug error, and pulmonary aspiration were scarce. There was no case of mismatched blood transfusion in the present study.

Incidence of total spinal block, neurological complication, and suspected myocardial ischemia or infarction was uncommon. Risk factors of oxygen destruction were shorter in height, higher ASA physical status, and use of propofol. Some events were considered avoidable and preventable [6].

### Effect of TSA on the Heart and Peripheral Blood Flow

We evaluated the effect of total spinal anesthesia (TSA) on the heart rate and peripheral blood flow variations to evaluate if TSA could influence these parameters as indicators of autonomic nervous function (ANF) [7]. Four patients with intractable pain were treated by TSA by administration of local anesthetics (lidocaine or mepivacaine) into the C7-Th1 vertebral interspace. Power spectrum of heart rate before TSA showed three peaks; low (LO-FR, 0.04-0.095 Hz), mid (MID-FR, 0.095-0.15 Hz) and high frequency areas (HI-FR, 0.15-0.3 Hz). Spectral peaks of LO-FR and MID-FR disappeared during TSA (P less than 0.001). HI—FR area decreased to 3.3% of the control level (P less than 0.001). Mean peripheral blood flow was changed to 59.9% of the control level (not significant). However, peripheral blood flow variations of LO-FR decreased to 11.3% of the control level (P less than 0.001) after TSA. Furthermore, the vagal discharge disappeared promptly after TSA in dog. These results suggest that TSA depresses the vagal activity as well as the sympathetic activity innervating the cardiovascular system and therefore, heart rate and peripheral blood flow variations are totally eliminated. Thus, we conclude that heart rate and peripheral blood flow variations can serve as valid markers of ANF activity [7].

To test whether acute denervation alters the vascular effects of dopamine and dobutamine, we anesthetized 16 greyhounds and placed them on total cardiopulmonary bypass (CPB) [8]. Eight dogs received total spinal anesthesia before drug testing; eight dogs were tested in the absence of total spinal anesthesia. During dopamine and dobutamine infusions, venous capacitance [determined by the volume of the CPB venous reservoir (VR)] and mean arterial pressure (MAP) were monitored. The CPB pump flows remained constant throughout our studies. Every dog received six increasing doses of both drugs. In the absence of total spinal anesthesia, both dopamine and dobutamine increased VR (decreased venous capacitance) in a dose dependent manner. Dobutamine decreased MAP in a dose related fashion but dopamine had no significant effect on MAP. After total spinal anesthesia, both dopamine and dobutamine produced greater dose-related increases in VR (i.e., decreases in venous capacitance) than in the absence of spinal anesthesia. Dopamine increased MAP but dobutamine had no significant effect. These data demonstrate how dopamine and dobutamine differ in their effects on the arterial circulation in the presence or absence of spinal anesthesia. The acute denervation of spinal anesthesia altered venous and arterial dose-response relationships of both drugs. Finally, our study demonstrates the effectiveness of dobutamine and, perhaps even more so, dopamine as possible alternatives to ephedrine for the pharmacologic correction of the noncardiac circulatory sequelae of spinal anesthesia [8].

### TSA History

Evans described in 1928 the possible complications of spinal anesthesia [9]. Concerning respiratory paralysis, he wrote: “If respiration should cease, keep cool. Raise the lower jaw, pull the tongue forward and begin artificial respiration at a uniform rate. Mouth to mouth insufflation is the most convenient and efficacious method of artificial respiration”. Twenty years before, in September 1908, before the Congress of the International Society of Surgery, in Brussels, Thomas Jonnesco from Bucharest, described his new method of general spinal anesthesia and reported 14 cases operated upon by his method [10]. Bier, who 10 years ago established the first human surgical spinal anesthesia, rejected it [10]. In a later paper in 1910 Jonnesco wrote: “It is an error to confuse lumbar rachianesthesia, conceived by Corning and popularized by Bier, with my method. As I have many times emphasized, my method is a new one and altogether distinctive, because I have generalized spinal anaesthesia, adopting it to all operations on any part of the body” [11]. Patients given high spinal anesthesia frequently either lapse into what appears to be normal sleep or may actually lose consciousness [12-15]. If patients with high spinal anesthesia are given an inhalational anesthetic such as nitrous oxide-oxygen, very low concentrations of anesthetic gases are required to maintain unconsciousness [16]. Reduction in the strength of nociceptive input may contribute to loss of consciousness by diminishing the strength of arousing stimuli arriving at cortical structures [17]. Studies with C14 labeled lidocaine in dogs have shown that the foramen magnum is not a physiological barrier, for autoradiographs and tissue samples reveal the presence of radioactivity in intracranial parts of the CNS after a relatively modest epidural dose [18]. Total spinal anesthesia has been used as a method of general anesthesia for abdominal surgery [19] and for the treatment of intractable pain [20]. Gillies and Morgan described a patient in whom a total spinal anesthesia resulted after 18 ml of inadvertent subarachnoid injection of 0.5% bupivacaine [21]. Spontaneous respiration was noted 120 minutes later, and consciousness regained after further 65 minutes. Return of respiration after 17 ml 1.5% lignocaine which resulted in total spinal analgesia occurred after 45 minutes and consciousness after further 80 minutes [22]. Four patients with intractable pain were treated by total spinal anesthesia. Power spectral analysis of heart rate and peripheral blood flow variations were studied. Vagal activity was depressed as well as the sympathetic activity innervating the cardiovascular system, so the heart rate and peripheral blood flow variations were totally eliminated [23]. Total spinal block can be elicited even after an epidural test dose like the 36-year-old parturient of Palkar et al. who developed hypotension and extensive sensory and motor block including respiratory paralysis and aphonia after injection via the epidural catheter of 3 ml lidocaine 1.5% (45 mg) with 1:200,000 epinephrine (15 microgram) [24]. The patient remained fully conscious and alert and spontaneous respiration recommenced in five minutes. Three patients were studied to determine the changes in regional skin temperature and blood flow during extensive sympathetic blockade following total spinal anesthesia. The temperature of the truncal area, arm and leg decreased by 1-degree C., whereas the temperature of the hand and foot increased by 3 degrees C. [25]. Total spinal block was induced by 2% lidocaine in adult mongrel dogs. Heart rate, mean arterial pressure, cardiac index and left ventricle dp/dt max decreased significantly [26]. Ephedrine 0.5 mg/Kg elevated HR, MAP, LV dp/dt max and SVR [27]. Total spinal anesthesia blocks the vagus as well as the sympathetic nervous system and decreases heart rate variation, suggesting that neural control of the heart via the autonomic nervous system is abolished after total spinal anesthesia [25]. Matsuki et al. described a patient with primary aldosteronism who was anesthetized by total spinal anesthesia using an epidural catheter inserted at L3-4 into the subarachnoid space [28].

The trachea was intubated after intravenous injection of thiopentone 250 mg and suxamethonium 40 mg, and oxygen 3 liters/minute and nitrous oxide 2 liters/minute inhaled.

The intraoperative course was smooth and intraoperative muscle relaxation excellent. Adrenaline, noradrenaline and dopamine in the plasma remained within normal ranges. Mets et al. described a case of an unplanned version of CSEGA (Combined Spinal Epidural General Anesthesia): A 24-year-old parturient received an epidural analgesia during labor [29]. Then she was scheduled for cesarean section for failure to progress. A total dose of 30 ml 0.5% bupivacaine was administered incrementally via the epidural catheter which resulted in a patchy block that was inadequate for surgery. Twenty minutes after the last injection of epidural local anesthetic a spinal anesthesia was done which resulted in a high block that necessitated tracheal intubation and ventilation. Controlled ventilation maintained with 50% N20 and 0.5% isoflurane in oxygen until delivery of the baby after which the isoflurane was stopped and 70% N20 in oxygen was administered. No further muscle relaxation was required for the remainder of the operation which lasted 45 minutes. The patient was extubated at the end of the operation uneventfully.

### CSF Lavage

High or total spinal anesthesia commonly results from accidental placement of an epidural catheter in the intrathecal space with subsequent injection of excessive volumes of local anesthetic. Cerebrospinal lavage has been shown to be effective at reversing the effects of high/total spinal anesthesia but is rarely considered in obstetric cases. Here, we describe the use of cerebrospinal lavage to prevent potential complications from high/total spinal anesthesia after unintentional placement of an intrathecal catheter in a labouring obstetric patient [30].

A 34-yr-old female presented to the labour and delivery unit in active labour. Epidural anesthesia was initiated, and after the first bolus dose, the patient experienced lower extremity motor block and shortness of breath. A high spinal was confirmed, and cerebrospinal lavage was performed. In total, 40 mL of cerebrospinal fluid (CSF) were exchanged for an equal volume of normal saline. The patient's breathing difficulties and motor block resolved quickly, and a new epidural catheter was placed after removal of the spinal catheter. Pain control was effective, and the patient delivered a healthy baby.

We show that exchange of CSF for normal saline can be used successfully to manage a high spinal in an obstetric patient. Our results suggest that CSF lavage could potentially be an important and helpful adjunct to the conventional supportive management of obstetric patients in the event of inadvertent high or total spinal anesthesia [30].

High spinal block carries significant morbidity and mortality risk. The predisposing factors are modifiable to reduce the incidence but unintentional high block secondary to migrated epidural catheter is still a possibility.

Our patient had received a combined spinal epidural anesthetic for surgery in the lithotomy position with 15 mg of 0.5% hyperbaric bupivacaine deposited intrathecally and repeated hourly boluses of 8 ml/hour of isobaric bupivacaine throughout surgery. The position of epidural catheter was confirmed with a test dose of 3 ml 2% preservative free lignocaine before the first epidural injection. The last epidural medication was 0.25% bupivacaine 6 ml given at the time of skin closure.

Subsequent to this injection, the patient started complaining of heaviness of breath in a low-pitched voice. His vital signs were stable (HR 92 bpm; BP 110/56 mmHg) prior to this episode. Blood pressure decreased to 85/56 mm Hg with a HR of 78 bpm. A colloid 6% HES infusion was started. The possibility of a pulmonary embolism or cranial migration of epidural drug in lithotomy position was considered. The patient could support his airway and low volume tidal breathing was observed clinically. Ventilation was gently assisted with BMV with 100% 02. The epidural catheter showed CSF on aspiration.

The technique of CSF lavage to reverse the high spinal anesthesia was employed. Slowly, CSF was removed in aliquots of 10 ml each time and replaced with 10 ml of isotonic saline. This procedure was repeated for 5 times following strict asepsis and keeping a careful watch on the patient's haemodynamics. Prompt improvements in single breath count from 4 to 12, hand grip and end tidal capnogram of spontaneous breathing was noticed. The sensory level of analgesia regressed from T2 to T4 over 15 mins and T10 by 25 mins. He was administered iv paracetamol 1 gm and tramadol 50 mg to combat pain, which was his next complaint half an hour after CSF lavage. The spinally migrated epidural catheter was removed in lateral position and patient was monitored for next 72 hours for meningeal signs, respiratory or cardiovascular deterioration and post dural puncture headache [31].

### TSA Due to Intercostal Nerve Block and Interscalene Nerve Block and Cervical Epidural Block

Intercostal nerve block is a recognized way of providing analgesia at thoracotomy. There is a rare association between intercostal nerve block and the complication of total spinal anesthesia. This may arise inadvertently by injection into a dural cuff extending outside the intervertebral foramen. We report our experience with a patient who sustained this life-threatening complication [32]. The patient required postoperative ventilation until the neurologic deficits resolved. The operator must be aware that intercostal nerve block runs the rare but potentially fatal risk of total spinal block [32].

We report a catastrophic postoperative complication of a prolonged interscalene block performed under general anaesthesia [33]. The course of the anaesthetic was uneventful, and the patient remained stable during his stay in the recovery area with the operative extremity paralysed and insensate. No further local anaesthetic was administered until later that day when the patient received 10 ml bupivacaine 0.25% through the catheter. Upon completion of the top-up dose, no change in the patient's status was noticed. The patient was next assessed 6.5 h later when he was found dead in his bed. A post-mortem CT scan revealed the catheter to be sited intrathecally, presumably the result of dural sleeve penetration [33].

We present a case of unintentional total spinal anesthesia, which occurred during cervical epidural block [34]. A 34-year-old man with complex regional pain syndrome of the right upper arm was treated with epidural block at C7-T1 interspace. Immediately after test-dose injection of ropivacaine 1.5 ml, he complained of paraesthesia of his upper extremities. He developed difficulty talking and breathing. Subsequently he showed a complete paralysis with the loss of consciousness, respiratory arrest, and bilateral mydriasis. Mandatory ventilation was started, and endotracheal tube was placed. Eighty minutes after the injection of ropivacaine, he recovered consciousness and spontaneous respiration resumed. Checking adequate ventilation, his trachea was extubated. Neurological dysfunction was not seen thereafter. Although test-dose injection is recommended especially in high-risk patients and case of difficulty of epidural space identification, it does not fully prevent complications. For cervical epidural block, local anesthetics should either be given at small doses or not be given as long as a possibility of spinal injection is remaining [34].

### TSA is not LAST

Look on the following checklist for treatment of local anesthetic systemic toxicity {LAST): https://www.asra.com/content/documents/asra_last_checklist_2018.pdf

It is based on The Third American Society of Regional Anesthesia and Pain Medicine Practice Advisory on Local Anesthetic Systemic Toxicity. Executive Summary 2017. Reg Anesth Pain Med 2016; 43: 113-123.

Look on the second page under Risk Reduction (Be sensible):
Second point: “Local anesthetic blood levels are influenced by site of injection and dose”.
Fourth point: “Aspirate the syringe prior to each injection while observing for blood in the syringe or tubing”.

In other words: LAST refers to LA>BLOOD>ST

It has nothing to do with TSA . . . .

Of course, the LA in the blood can have “altered mental status, neurological symptoms or signs of cardiovascular instability . . . . ”

These 3 case reports are the first reports in the medical literature regarding the Local Anesthesia Reversal (LAR) of Total Spinal Anesthesia (TSA) by Lipofundin which is similar to Intralipid having soybean oil fatty acids including Linoleic acid.

### Conclusion

These 3 case reports are the first reports in the medical literature regarding the Local Anesthesia Reversal (LAR) of Total Spinal Anesthesia (TSA) by Lipofundin.

### REFERENCES

1. Hodgkinson R (1981) Total spinal block after epidural injection into an interspace adjacent to an inadvertent dural perforation. Anesthesiology 55: 593-595.
2. Davies J M, Posner K L, Lee L A, Cheney F W, Domino K B (2009) Liability associated with obstetric anesthesia: a closed claims analysis. Anesthesiology 110: 131-139.
3. Gillies I D, Morgan M (1973) Accidental total spinal analgesia with bupivacaine. Anaesthesia 28: 441-445.
4. Siddik-Sayyid S M, Gellad P H, Aouad M T (2012) Total spinal block after spinal anesthesia following ongoing epidural analgesia for cesarean delivery. J Anesth 26: 312-313.
5. Wilkinson G R, Lund P C (1970) Bupivacaine levels in plasma and cerebrospinal fluid following peridural administration. Anesthesiology 33: 482-486.
6. Charuluxananan S, Thienthong S, Rungreungvanich M, Chanchayanon T, Chinachoti T, et al. (2007) The Thai Anesthesia Incidents Study (THAI study) of morbidity after spinal anesthesia: a multi-centered registry of 40,271 anesthetics. J Med Assoc Thai 90: 1150-1160.
7. Goda Y, Kimura T, Goto Y, Kemmotsu O (1989) Power spectral analysis of heart rate and peripheral blood flow variations during total spinal anesthesia. Masui 38: 1275-1281.
8. Butterworth J F, Austin J C, Johnson M D, Berrizbeitia L D, Dance G R, et al. Effect of total spinal anesthesia on arterial and venous responses to dopamine and dobutamine. Anesth Analg 66: 209-214.
9. Evans C H (1928) Possible complications with spinal anesthesia. Their recognition and the measures employed to prevent and to control them. Am J Surgery 5: 581-593.
10. Jonnesco T (1909) Remarks on general spinal analgesia. Br Med J 2: 1396-1401.
11. Jonnesco T (1910) Concerning general rachianesthesia. Am J Surgery 24: 33.
12. Koster H, Kasman L P (1929) Spinal anesthesia for the head, neck and thorax: its relation to respiratory paralysis. Surg Gynecol Obstet 49: 617.
13. Vehrs G R (1934) Spinal anesthesia: Technic and clinical application. St Louis: The C.V. Mosby Co.
14. Jones R G G (1953) A complication of epidural technique. Anaesthesia 8: 242.
15. Huvos M C, Greene N M, Glaser G H (1962) Electroencephalographic studies during acute subtotal denervation in man. Yale J Biol Med 34: 592.
16. Greene N M (1952) Hypotensive spinal anesthesia. Surg Gynecol Obstet 95: 331.
17. Kendig J J (1993) Spinal cord as a site of anesthetic action. Anesthesiology 79: 1161-1162.
18. Bromage P R, Joyal A C, Binney J C (1963) Local anaesthetic drugs: Penetration from the spinal extradural space into the neuraxis. Science 140: 392.
19. Evans T I (1974) Total spinal anaesthesia. Anaesth Intensive Care 2: 158-163.
20. Yamashiro H, Hirano K (1987) Treatment with total spinal block of severe herpetic neuralgia accompanying median and ulnar nerve palsy. Masui 36: 971-975.
21. Gillies I D S, Morgan M (1973) Accidental total spinal analgesia with bupivacaine.
Anaesthesia 28: 441-445.
22. DeSaram M (1956) Accidental total spinal analgesia. A report of three cases. Anaesthesia 11: 77.
23. Goda Y, Kimura T, Goto Y, Kemmotsu O (1989) Power spectral analysis of heart rate and peripheral blood flow variations during total spinal anesthesia. Masui 38: 1275-1281.
24. Palkar N V, Boudreaux R C, Mankad A V (1992) Accidental total spinal block: a complication of an epidural test dose. Can J Anaesth 39: 1058-1060.
25. Kimura T, Goda Y, Kemmotsu O, Shimada Y (1992) Regional differences in skin blood flow and temperature during total spinal anaesthesia. Can J Anaesth 39: 123-127.
26. Kobori M, Negishi H, Masuda Y, Hosoyamada A (1991) Changes in respiratory, circulatory, endocrine, and metabolic systems under induced total spinal block. Masui 40: 1804-1809.
27. Kobori M, Negishi H, Masuda Y, Hosoyamada A (1990) Changes in systemic circulation under induced total spinal block and choice of vasopressors. Masui 39: 1580-1585.
28. Matsuki M, Muraoka M, Oyama T (1988) Total spinal anaesthesia for a Jehovah's
Witness with primary aldosteronism. Anaesthesia 43: 164-165.
29. Mets B, Broccoli E, Brown A R (1993) Is spinal anesthesia after failed epidural anesthesia contraindicated for cesarean section? Anesth Analg 77: 629-631.
30. Ting H Y, Tsui B C (2014) Reversal of high spinal anesthesia with cerebrospinal lavage after inadvertent intrathecal injection of local anesthetic in an obstetric patient. Can J
Anaesth 61: 1004-1007.
31. Ashima S, Kiran G, Padmaja D, Gopinath R (2014) CSF Lavage for high spinal—A technical miracle. BJA: British Journal of Anaesthesia 113.
32. Chaudhri B B, Macfie A, Kirk A J (2009) Inadvertent total spinal anesthesia after intercostal nerve block placement during lung resection. Ann Thorac Surg 88: 283-284.
33. Yanovski B, Gaitini L, Volodarski D, Ben-David B (2012) Catastrophic complication of an interscalene catheter for continuous peripheral nerve blockanalgesia. Anaesthesia 67: 1166-1169.
34. Hara K, Sata T (2006) Unintentional total spinal anesthesia during cervical epidural block with ropivacaine. Masui 55: 1168-1169.

### Example 15

### Upper Arm Local Anesthesia Reversal (LAR) Using Lipofundin (3 Case Reports)

Local anesthesia reversal(LAR) is a new idea based on the history of Local Anesthetic Systemic Toxicity (LAST). If Intralipid and other specific Lipid emulsions can reverse the cardiac toxicity, why it cannot reverse other features connected to the local anesthesia toxicity? It is a very logical idea. The illogical issue is why it took almost 20 years from the first article on Intralipid and bupivacaine to invent it . . . ?

In our 3 cases the first patient “after finishing the lipofundin bolus injection after 4 mins (137 min after the brachial plexus block), she could move slightly her right finger. The patient could move her hand at 34 mins after the lipofundin bolus injection (166 mins after the brachial plexus block), and she could move her forearm 102 mins after the lipofundin bolus injection”.

In the 2nd case “after 80-84 mins of the LE bolus injection (180 min after the brachial plexus block) the patient could move slightly his left thumb. After 184 mins since the lipid emulsion bolus injection (290 mins after the brachial block) the patient could move his hand and arm easily”.

In the 3rd case “85-87 mins after the LE bolus injection (220 min after the brachial plexus block), the patient could move slightly his arm. 128 mins after the lipid emulsion bolus injection (261 mins after the brachial plexus block), the patient could move his hand easily”.

In all the 3 cases the reversal of the sensory and motor blocks were much faster than in the non-LAR cases as described in the medical literature. In that regards the LAR by Intralipid, Lipofundin and other similar lipid emulsions is a new method for postoperative care of patients after brachial plexus block.

### Case Report 1

A 37 years old female was diagnosed an ununion radial bone. She performed a radial bone surgery on 1stMarch 2018 at Military Hospital 103, Vietnam Military Medical University: http://www.csen.com/upper4.JPG

Patient was anesthetized by ultrasound guided brachial plexus block, upper clavicular approach with lidocaine 4 mg/kg combined with ropivacaine 1 mg/kg and epinephrine 1/200.000 in a total volume of 30 ml.

Onset time: 8 mins

Duration of surgery: 100 mins

Anesthesia effect was very good for surgery. Sensory and motor functions of the right hand were assessed before Lipofundin injection and were absent completely after surgery.

Lipid emulsion bolus injection was started at 137 mins after brachial plexus block over 3 mins with the dose of 1.5 mg/kg (patient's weight 65 kg, 158 cm), total −97 ml. Then, continuous infusion with the dose of 0.25 ml/kg/min (162 ml) over 10 mins was done. Patient was monitored closely.

After finishing the lipofundin bolus injection after 4 mins (137 min after the brachial plexus block), the patient could move slightly her right finger. The patient could move her hand at 34 mins after the lipofundin bolus injection (166 mins after the brachial plexus block), and could move her forearm 102 mins after the lipofundin bolus injection. She completely recovered her movement and sensory 127 mins after the lipofundin bolus injection. Vital signs were stable after the lipofundin injections (Table 2).

TABLE 2

| | | | Lipid bolus | | | | | Can | Move | |
|---|---|---|---|---|---|---|---|---|---|---|
| | | | injection | Continuous | Can move | Can | Can | move | her | Complete |
| | | Duration of | 1.5 ml/kg | infusion | slightly her | move | move | her | upper | Recovery of |
| | Brachial | surgery | (97 ml) | in 10 mins | finger | her | her | upper | arm | movement |
| | Plexus | 8 h | over 3 mins | 10 h 30 min to 10 h | 10 h 33 to | hand | forearm | arm | easily | and sensory |
| | block | 21 minAM | (100 mins) | 33 min | 10 h 43 min | 10 h 37 | 11 h 7 | 12 h 25 | 12 h 43 | 12 h 47 |
| After block (mins) | | | 129 | | 137 | 166 | 244 | 262 | 266 | 270 |
| Afterlipofundinbolus | | | 0 | | 4 | 34 | 102 | 120 | 124 | 127 |
| injection (mins) | | | | | | | | | | |

### Videos:

Starting lipofundin bolus injection: https://youtu.be/rTtDOQiih1U

Continuous lipofundin infusion: https://youtu.be/lF4bP5zU4Kg

4 mins after finishing lipofundin bolus injection: https://youtu.be/--TREW5jeTs

13 mins after finishing lipofundin bolus injection: https://youtu.be/HE80oYqxFjc

15 mins after finishing lipofundin bolus injection: https://youtu.be/LcPoQrzwxQM

26 mins after finishing lipofundin bolus injection: https://youtu.be/C2-SOngdgWc

34 mins after finishing lipofundin bolus injection: https://youtu.be/xCwbVNuEZ9U

57 mins after finishing lipofundin bolus injection: https://youtu.be/rlfbMnQNLIM

63 mins after finishing lipofundin bolus injection: https://youtu.be/a6JILCUZQSY

67 mins after finishing lipofundin bolus injection: https://youtu.be/j0ALXiaafzc

72 mins after finishing lipofundin bolus injection: https://youtu.be/91cDOiS3rc4

81 mins after finishing lipofundin bolus injection: https://youtu.be/vUS_GJiRjmk

94 mins after finishing lipofundin bolus injection: https://youtu.be/j0zhtChUsBs

104 mins after finishing lipofundin bolus injection: https://youtu.be/ImSV0dmVpg4

111 mins after finishing lipofundin bolus injection: https://youtu.be/EmVx1UtgiJU

131 mins after finishing lipofundin bolus injection: https://youtu.be/U8ywvu Ny2I

136 mins after finishing lipofundin bolus injection: https://youtu.be/X5IcvySgBMg

139 mins after finishing lipofundin bolus injection: https://youtu.be/cX8Ki4uFhd4 Case report 2

A 19 years old male was diagnosed with two bone fractures in the left forearm due to vehicle accident: http://www.csen.com/upper3.JPG

He underwent an elective two bone surgery on 27th February, 2018 at the Military Hospital 103, Vietnam Military Medical University. The patient was anesthetized by ultrasound guided brachial plexus block, upper clavicular approach with lidocaine 4 mg/kg combined with ropivacaine 1 mg/kg and epinephrine 1/200.000 in total 30 ml of solution.

Onset time: 7 mins.

Duration of surgery: 80 mins.

Anesthesia effect was very good for surgery. Surgery was accomplished in 83 mins after the brachial plexus block. Sensory and motor functions of the left hand were absent completely after the surgery.

Lipid emulsion (Lipofundin) bolus injection was started at point 133 mins after the brachial plexus block over 3 mins with the dose of 1.5 mg/kg (patient's weight is 40 kg, 158 cm), total −60 ml. Afterwards a continuous infusion with the dose of 0.25 ml/kg/min (100 ml/10 mins) over 10 mins was done. The patient was monitored during the LE infusion until the full recovery of the motor and sensory functions.

After 80-84 mins of the LE bolus injection (180 min after the brachial plexus block) the patient could move slightly his left thumb. After 184 mins since the lipid emulsion bolus injection (290 mins after the brachial block) the patient could move his hand and arm easily. Vital signs were stable during and after the LE bolus and infusion(Table 3). Lipofundin: http://www.csen.com/Lipofundin.JPG

TABLE 3

| | | | Lipid bolus | Continuous | | | | |
|---|---|---|---|---|---|---|---|---|
| | Brachial | Duration | injection 1.5 ml/kg | infusion in | Move | Can | Move his | Completely recover |
| | Plexus | of | (90 ml) | 10 mins | slightly | move his | upper | movement and sensory |
| | block | surgery | over 3 mins | 11 h to 11 h | his arm | forearm | arm easily | 15 h (feel pain and need |
| | 9 h 21 min | (80 mins) | 10 h 57 min | 10 min | 12 h 21 | 12 h 39 | 14 h 11 | pain medication) |
| After block | | | 96 | 99 | 180 | 198 | 290 | 339 |
| (mins) | | | | | | | | |
| AfterLE bolus | | | 0 | | 84 | 102 | 184 | 233 |
| injection (mins) | | | | | | | | |

### Videos:

Starting Bolus LE injection: https://youtu.be/bSU1LPPOOZQ

10 mins after bolus LE injection: https://youtu.be/6VkXsLXlsc

46 mins after bolus LE injection: https://youtu.be/B8VgbvF8538

60 mins after bolus LE injection: https://youtu.be/Jc9oCClkHTg

80 mins after bolus LE injection: https://youtu.be/F7mEXJG Sdk

84 mins after bolus LE injection: https://youtu.be/BukCloqzHnU

100 mins after bolus LE injection: https://youtu.be/VpuUV3A8AcA

102 mins after bolus LE injection: https://youtu.be/p1CGcsTHzCM

194 mins after bolus LE injection: https://youtu.be/wfJKDneDj_g

### Case Report 3

A 49 years old male was diagnosed of a complicated two bone fractures in the right forearm due to labour accident: http://www.csen.com/upper2.jpg

The patient performed an elective two bones surgery on 26 Feb. 2018 at the Military Hospital 103, Vietnam Military Medical University. The patient was anesthetized by ultrasound guided brachial plexus block, upper clavicular approach with lidocaine 4 mg/kg combined with ropivacaine 1 mg/kg and epinephrine 1/200.000 in total 30 ml of solution.

Onset time: 8 mins.

Duration of surgery: 110 mins.

Anesthesia effect was very good for the surgery. Surgery was accomplished in 120 mins after the brachial plexus block. Sensory and motor functions of the left hand were absent completely after the surgery.

Lipid emulsion (Lipofundin) bolus injection was started at the point of 133 mins after the brachial plexus block over 3 mins with the dose of 1.5 mg/kg (patient's weigh is 60 kg, 168 cm), total: 90 ml. Then, continuous infusion of Lipofundin with the dose of 0.25 ml/kg/min (150 ml) over 10 mins was done. The patient was monitored during the LAR (Local Anesthesia Reversal).

85-87 mins after the LE bolus injection (220 min after the brachial plexus block), the patient could move slightly his arm. 128 mins after the lipid emulsion bolus injection (261 mins after the brachial plexus block), the patient could move his hand easily. Vital signs were stable during the LAR procedure (Table 4).

TABLE 4

| | | | Lipid bolus | | | | | |
|---|---|---|---|---|---|---|---|---|
| | | | injection | | | | | |
| | | | 1.5 ml/kg | Continuous | | | Move his | Completely |
| | Brachial | Duration | (90 ml) | infusion in | Move | Move his | upper | recover |
| | Plexus | of | over 3 mins | 10 mins | slightly his | forearm | arm | movement and |
| | block | (10 h 56) | surgery | 11 h 10 min to | arm | easily | easily | sensory |
| | 8 h 46 min | 110 mins | 11 h 7 min | 11 h 20 min | 12 h 15 | 12 h 25 | 12 h 40 | 13 h 15 |
| After block | | | 131 | 144 | 199 | 209 | 226 | 261 |
| (mins) | | | | | | | | |
| After LE bolus | | | 0 | 0 | 68 | 78 | 93 | 128 |
| injection | | | | | | | | |
| (mins) | | | | | | | | |

### Videos:

Bolus LE. Injection: https://youtu.be/vrStLYycNrI

78 mins after LE bolus injection: https://youtu.be/SV6-FvF2dvo

93 mins after LE bolus injection: https://youtu.be/i4oCviFJdIA

97 mins after LE bolus injection: https://youtu.be/C1DTrW2fZo4

101 mins after LE bolus injection: https://youtu.be/f6dr2sahf9I

104 mins after LE bolus injection: https://youtu.be/vVnJbN1SOkY

### Discussion

Lipofundin

Composition 1000 ml emulsion contain:

Lipofundin® MCT/LCT 20%

Soybean oil: 100.0 g

Medium Chain Triglycerides: 100.0 g

Linoleic acid: 48-58 g/L

a-Linoleic acid: 5-11 g/L

Glycerol: 25.0 g

Egg yolk phospholipids*: 12.0 g

Sodium Oleate, α-Tocopherol*, Water for injections

Megajoules/l (approx.): 7.99 (1908 kcal)

Milliosmols/l (approx.): 380

pH:6.5-8.5

Lipid emulsions have been used to treat various drug toxicities and for total parenteral nutrition therapy. Their usefulness has also been confirmed in patients with local anesthetic-induced cardiac toxicity. The purpose of this study was to measure the hemodynamic and composition effects of lipid emulsions and to elucidate the mechanism associated with changes in intracellular calcium levels in myocardiocytes[1].

We measured hemodynamic effects using a digital analysis system after Intralipid® and Lipofundin® MCT/LCT were infused into hearts hanging in a Langendorff perfusion system[1]. We measured the effects of the lipid emulsions on intracellular calcium levels in H9c2 cells by confocal microscopy.

Infusion of Lipofundin® MCT/LCT 20% (1 ml/kg) resulted in a significant increase in left ventricular systolic pressure compared to that after infusing modified Krebs-Henseleit solution (1 ml/kg) (P=0.003, 95% confidence interval [CI], 2.4-12.5). Lipofundin® MCT/LCT 20% had a more positive inotropic effect than that of Intralipid® 20% (P=0.009, 95% CI, 1.4-11.6). Both lipid emulsion treatments increased intracellular calcium levels. Lipofundin® MCT/LCT (0.01%) increased intracellular calcium level more than that of 0.01% Intralipid® (P<0.05, 95% CI, 0.0-1.9).

These two lipid emulsions had different inotropic effects depending on their triglyceride component. The inotropic effect of lipid emulsions could be related with intracellular calcium level[1].

Free fatty acid (FFA) oxidation is depressed in the post ischaemic stunned myocardium and recovers in parallel with the normalization of contractile performance. Assuming a causal role for this metabolic disturbance in the pathogenesis of stunning, we questioned whether exogenous administration of high dose triglycerides during reperfusion of post ischaemic myocardium, could improve its functional recovery[2].

Thirteen dogs were chronically instrumented to measure global and regional haemodynamics and to produce a 10 min episode of regional myocardial ischaemia. In 7 dogs, Intralipid 20% was administered i.v. during the reperfusion phase. Contractile recovery of stunned myocardium was compared with control saline treatments. The series were repeated in another 6 animals, but oxfenicine (CPT I inhibitor) preceded Intralipid during reperfusion.

Contractile recovery of stunned myocardium was faster and more extensive when Intralipid was administered during reperfusion than with saline treatment (wall thickening fraction 86+/−6% of pre-ischaemic controls versus 52+/−11% at 90 min post-reperfusion; P<0.05). Oxfenicine pretreatment completely abolished this beneficial effect.

Exogenous administration of triglycerides during reperfusion of post ischaemic myocardium improves functional recovery from stunning. This beneficial effect most likely operates through enhanced FFA availability and/or oxidation since it could be abolished by selective inhibition of the carnitine palmitoyl I enzyme[2].

The authors sought to confirm a chance observation that intravenous lipid treatment increases the dose of bupivacaine required to produce asystole in rats. The authors also measured the partitioning of bupivacaine between the lipid and aqueous phases of a plasma-lipid emulsion mixture[3].

Anesthetized Sprague-Dawley rats were used in pretreatment (protocol 1) and resuscitation (protocol 2) experiments. In protocol 1, animals were pretreated with saline or 10%, 20%, or 30% Intralipid (n=6 for all groups), then received 0.75% bupivacaine hydrochloride at a rate of 10 ml×kg×min⁻¹ to asystole. In protocol 2, mortality was compared over a range of bolus doses of bupivacaine after resuscitation with either saline or 30% Intralipid (n=6 for all groups). The lipid:aqueous partitioning of bupivacaine in a mixture of plasma and Intralipid was measured using radiolabelled bupivacaine.

Median doses of bupivacaine (in milligrams per kilogram) producing asystole in protocol 1 were for 17.7 for saline, 27.6 for 10% Intralipid, 49.7 for 20% Intralipid, and 82.0 for 30% Intralipid (P<0.001 for differences between all groups). Differences in mean+/−SE concentrations of bupivacaine in plasma (in micrograms per millilitre) were significant (P<0.05) for the difference between saline (93.3+/−7.6) and 30% Intralipid (212+/−45). In protocol 2, lipid infusion increased the dose of bupivacaine required to cause death in 50% of animals by 48%, from 12.5 to 18.5 mg/kg. The mean lipid:aqueous ratio of concentrations of bupivacaine in a plasma-Intralipid mixture was 11.9+/−1.77 (n=3).

Lipid infusion shifts the dose-response to bupivacaineinduced asystole in rats. Partitioning of bupivacaine into the newly created lipid phase may partially explain this effect. These results suggest a potential application for lipid infusion in treating cardiotoxicity resulting from bupivacaine[3].

### Local Anesthesia Drug Inhibition of Mitochondrial ATPase

The concentrations of n-butanol and tetracaine required for 50% inhibition of the ATPase activity of F1 particles isolated from bovine heart mitochondria were 160 mM and 1.1 mM, respectively. The results are offered as evidence that the physiological effects of these anesthetics may be due to direct interaction with membrane proteins rather than with the lipids [4].

Local anesthetics and alcohols were found to inhibit mitochondrial electron transport at several points along the chain. The anesthetics employed were the tertiary amines procaine, tetracaine, dibucaine, and chlorpromazine, and the alcohols were n-butanol, n-pentanol, n-hexanol, and benzyl alcohol. Uncoupled sonic sub mitochondrial particles from beef heart and rat liver were studied. We [5]report the following: (1) All of the anesthetics were found to inhibit each of the segments of the electron transport chain assayed; these included cytochrome c oxidase, durohydroquinone oxidase, succinate oxidase, NADH oxidase, succinate dehydrogenase, succinate-cytochrome c oxidoreductase, and NADH-cytochrome c oxidoreductase. (2) NADH oxidase and NADH-cytochrome c oxidoreductase required the lowest concentration of anesthetic for inhibition, and cytochrome c oxidase required the highest concentrations. (3) We conclude that there are several points along the chain at which inhibition occurs, the most sensitive being in the region of Complex I (NADH dehydrogenase). (4) Beef heart sub mitochondrial particles are less sensitive to inhibition than are rat liver particles. (5) Low concentrations of several of the anesthetics gave enhancement of electron transport activity, whereas higher concentrations of the same agents caused inhibition. (6) The concentrations of anesthetics (alcohol and tertiary amine) which gave 50% inhibition of NADH oxidase were lower than the reported concentrations required for blockage of frog sciatic nerve [5].

The following characteristics are reported for mitochondrial ATPase prepared by the chloroform extraction method: (1) The pH optimum for enzyme activity is at 8.0. (2) The neutral anesthetic benzocaine inhibits the enzyme at all pH values. (3) Reciprocal plots of 1/v versus 1/[ATP] show that inhibition by lidocaine, tetracaine, dibucaine, and chlorpromazine is non-competitive; slope and intercept replot are hyperbolic, showing that the inhibition is partial rather than complete[6].

Supra clavicular brachial plexus anesthesia duration

To compare the efficacy of ropivacaine 7.5 mg×ml⁻¹ with bupivacaine 5.0 mg×ml⁻¹ for subclavian perivascular brachial plexus block.

After informed consent, 104 ASA I-III adults participated in a randomized, double-blind, multi-center trial to receive 30 ml of either ropivacaine 7.5 mg×ml⁻¹ or bupivacaine 5.0 mg×ml⁻¹ for subclavian perivascular brachial plexus block prior to upper limb surgery. Onset and duration of sensory and motor block in the distribution of the axillary, median, musculo-cutaneous, radial and ulnar nerves were assessed.

Onset times and duration of sensory and motor block were similar between groups. Mean duration of analgesia for the five nerves was between 11.3 and 14.3 hr with ropivacaine and between 10.3 and 17.1 hr with bupivacaine. Quality of muscle relaxation judged as excellent by the investigators was not significantly different (ropivacaine-35/49, bupivacaine-30/49). The median time to first request for analgesia was comparable between the two groups (11-12 hr). One patient developed a grand mal seizure shortly after receiving bupivacaine and recovered consciousness within 30 min. There were no serious adverse events in the ropivacaine group.

Thirty ml ropivacaine 7.5 mg×ml⁻¹ (225 mg) produced effective and well tolerated brachial plexus block of long duration by the subclavian perivascular route. In this study, the results were similar to those of 30 ml bupivacaine 5.0 mg×ml⁻¹ [7].

We examined the outcomes and levels of patient satisfaction in 202 consecutive cases of ultrasound-guided supraclavicular brachial plexus block (SBPB) in upper limb surgery performed between September 2007 and March 2010[8]. All blocks were performed by orthopaedic surgeons using ultrasound visualisation with a high-frequency linear probe. The probe was placed in the coronal-oblique plane in the supraclavicular fossa, and the puncture was ‘in-plane’ from lateral to medial. Most of the blocks were performed with 0.75% ropivacaine/1% lidocaine (1:1), with or without adrenaline in 1:200,000 dilution. In 201 patients (99.5%) the brachial plexus block permitted surgery without conversion to general anaesthesia. The mean procedure time for block was 3.9 min (2 to 12), the mean waiting time for surgery was 34.1 min (10 to 64), the mean surgical time was 75.2 min (6 to 232), and the mean duration of post-anaesthetic analgesia was 437 min (171 to 992). A total of 20 patients (10%) developed a transient Horner's syndrome. No nerve injury, pneumothorax, arterial puncture or systemic anaesthetic toxicity were recorded. Most patients (96.7%) were satisfied with ultrasound-guided SBPB. This study demonstrates the efficacy and safety of ultrasound-guided SBPB for orthopaedic surgery on the upper limb[8].

### Localanesthesiareversal (LAR)

Local anesthesia reversal (LAR) is a new idea based on the history of Local Anesthetic Systemic Toxicity (LAST). If Intralipid and other specific Lipid emulsions can reverse the cardiac toxicity, why it cannot reverse other features connected to the local anesthesia toxicity? It is a very logicalidea. The illogical issue is why it took almost 20 years from the first article on Intralipid and bupivacaine to invent it ? . . .

In our 3 cases the first patient “after finishing the lipofundin bolus injection after 4 mins (137 min after the brachial plexus block), she could move slightly her right finger. The patient could move her hand at 34 mins after the lipofundin bolus injection (166 mins after the brachial plexus block), and she could move her forearm 102 mins after the lipofundin bolus injection”.

In the 2nd case“after 80-84 mins of the LE bolus injection (180 min after the brachial plexus block) the patient could move slightly his left thumb. After 184 mins since the lipid emulsion bolus injection (290 mins after the brachial block) the patient could move his hand and arm easily”.

In the 3rd case “85-87 mins after the LE bolus injection (220 min after the brachial plexus block), the patient could move slightly his arm. 128 mins after the lipid emulsion bolus injection (261 mins after the brachial plexus block), the patient could move his hand easily”.

In all the 3 cases the reversal of the sensory and motor blocks were much faster than in the non-LAR cases as described in the medical literature. In that regards the LAR by Intralipid, Lipofundin and other similar lipid emulsions is a new method for postoperative care of patients after brachial plexus block.

### Conclusion

Local anesthesia reversal (LAR) is a new idea based on the history of Local Anesthetic Systemic Toxicity(LAST). If Intralipid and other specific Lipid emulsions can reverse the cardiac toxicity, why it cannot reverse other features connected to the local anesthesia toxicity? It is a very logical idea. The illogical issue is why it took almost 20 years from the first article on Intralipid and bupivacaine to invent it?.

### Example 16

### Local Anesthesia Reversal (LAR) of Spinal Anesthesia by Lipid Emulsion (Lipofundin 20%) for Day Case Surgery

A 46 years old Male was diagnosed with penis fracture due to sexual intercourse. A 72 years old Male was diagnosed as having a tumor on the upper pole of the femur. A 41 years old Female had a Cesarean section. All of them underwent spinal anesthesia for their operations. All of them underwent at the end of their operations Local anesthesia reversal (LAR) of Spinal anesthesia by Lipid Emulsion (Lipofundin 20%) using Bolus and Infusion. The local anesthesia reversal was started in approximately 3 minutes after starting the bolus injection and completed in approximately 30 minutes afterwards. This new modality of LAR can make a great change in the use of spinal anesthesia in day case surgery facilities.

### Case Reports

### Case No. 1

A 46 years old Male was diagnosed with penis fracture due to sexual intercourse. He underwent an emergency surgery for penis repair on 21 Feb. 2018 at the Military Hospital 103, Vietnam Military Medical University. The patient was anesthetized by spinal anesthesia with 8 mg of bupivacaine plus 20 mcg fentanyl in the sitting position at the L2-L3 level. The point time of the spinal block was 15 h 35 min.

Onset time: 3 mins.

Anesthesia effect was very good for the surgery.

Dermatome check by the cold test with alcohol gauze was T$ at 10 mins after the spinal block.

Duration of surgery: 30 mins.

Hemodynamic was stable during the surgery.

Surgery was completed at 16 h 15 min (50 mins after the performance of the spinal block).

Lipid emulsion (Lipofundin 20%) bolus injection was started at 16 h 15 mins over 3 mins with the dose of 1.5 mg/kg (patient's weight 68 kg), then by continuous infusion with 0.25 ml/kg/min over 12 mins. Total volume of the Lipid emulsion was 300 ml. The patient was monitored closely during the local anesthesia reversal (LAR) process (Table 5).

TABLE 5

| | 16 h 15 | | | | | | | |
|---|---|---|---|---|---|---|---|---|
| | (Lipid | 16 h 20 | 16 h 32 | | | | | |
| | bolus | Continuous | | | | | | |
| | injection) | infusion | | 16 h 40 | 16 h 55 | 17 h 10 | 17 h 20 | 17 h 30 |
| Bromage in | 3 | 2 | 2 | 2 | 0 | 0 | 0 | |
| the Right leg | | | | | | | | |
| Bromage in | 3 | 3 | 3 | 3 | 2 | 1 | 0 | |
| Left leg | | | | | | | | |
| Sensory | T4 | T4 | T5 | T5 | T7 | T10 | T12 | |
| level | | | | | | | | |
| Min after | 50 | 55 | 63 | 75 | 90 | 105 | 1156 | Full |
| spinal block | | | | | | | | recovery |
| | | | | | | | | of |
| | | | | | | | | sensory |
| | | | | | | | | block |

### Videos:

Starting the bolus of lipofundin 20% injection: https://youtu.be/cXIy1Bq08CA

1 minute after finishing bolus lipofundin injection: https://youtu.be/ic9xoODVqXc

2 minutes after finishing bolus lipofundin injection: https://youtu.be/BcPdXevSKvI

19 minutes after finishing bolus lipofundin injection: https://youtu.be/FJp122JFSEc

28 minutes after finishing bolus lipofundin injection: https://youtu.be/9cxuynCwrvY

32 minutes after finishing bolus lipofundin injection: https://youtu.be/5GuxnVucjzY

34 minutes after finishing bolus lipofundin injection: https://youtu.be/yQuk2EEcNqM

39 minutes after finishing bolus lipofundin injection: https://youtu.be/Rx8dCQPjXuY

42 minutes after finishing bolus lipofundin injection: https://youtu.be/E85Xv2gfGOY

47 minutes after finishing bolus lipofundin injection: https://youtu.be/GygRH6GYbW0

48 minutes after finishing bolus lipofundin injection: https://youtu.be/25eBCScsVbk

50 minutes after finishing bolus lipofundin injection: https://youtu.be/IBacn8MrbJU

52 minutes after finishing bolus lipofundin injection: https://youtu.be/oleWEpuNZ5I

61 minutes after finishing bolus lipofundin injection: https://youtu.be/AKt23S6FPpk

69 minutes after finishing bolus lipofundin injection: https://youtu.be/ipWH95V4e0k

73 minutes after finishing bolus lipofundin injection: https://youtu.be/WC-bKjpAmmg

### Case No. 2

A 72 years old Male was diagnosed as having a tumor on the upper pole of the femur. The patient performed a biopsy on Mar. 9, 2018 at the Military Hospital 103, Vietnam Military Medical University. The patient was anesthetized by spinal anesthesia with 9 mg of bupivacaine plus 20 mcg fentanyl in the sitting position at L2-L3 level. Point time of the spinal block: 9 h 02, AM.

Anesthesia effect was very good for the surgery.

Onset time: 4 mins.

Dermatome check by cold test with alcohol gauze was T4 at 10 mins after the spinal block.

Duration of surgery: 28 mins

Hemodynamic was stable during the surgery.

Surgery was completed at 9 h 40 which means 38 mins after the spinal block.

Lipid emulsion (Lipofundin 20%) bolus injection was started at 9 h 50 mins over 3 mins with the dose of 1.5 mg/kg (patient's weight 70 kg), then continuous infusion with 175 ml over 30 mins (−0.08 mg/kg/min). Total volume of Lipid emulsion (Lipofundin 20%) was 275 ml. The patient was monitored closely in the operating theatre during the lipid emulsion treatment.

Only 3 minutes after finishing the bolus injection the patient could move slightly both legs. The patient got a full recovery of his movement function at 30 minutes after finishing the bolus of Lipofundin 20% injection (Table 2).

### Videos:

Starting bolus injection:

https://youtu.be/bBYHP-iFmZI

https://youtu.be/mVKww1EfP8A

2 minutes after finishing bolus lipofundin injection: https://youtu.be/63-EeqC7WL8

4 minutes after finishing bolus lipofundin injection: https://youtu.be/6kj5OzFNTwQ

5 minutes after finishing bolus lipofundin injection: https://youtu.be/nM3qjveWQOw

6 minutes after finishing bolus lipofundin injection: https://youtu.be/EN4mKejZItM

7 minutes after finishing bolus lipofundin injection: https://youtu.be/21rZ82wcYk4

9 minutes after finishing bolus lipofundin injection: https://youtu.be/cpTLMObFRn8

13 minutes after finishing bolus lipofundin injection: https://youtu.be/CBx-O5WQY4Y

30 minutes after finishing bolus lipofundin injection: https://youtu.be/bfi5AGjVcLc

32 minutes after finishing bolus lipofundin injection: https://youtu.be/vqjvxAO6bjA

35 minutes after finishing bolus lipofundin injection: https://youtu.be/NRxzoOr15Z0

48 minutes after finishing bolus lipofundin injection: https://youtu.be/q7NtxyBIqk

69 minutes after finishing bolus lipofundin injection: https://youtu.be/ThRqH00mwmo

74 minutes after finishing bolus lipofundin injection: https://youtu.be/PuUdmVpmkKo

### Case No. 3

A 41 years old Female had a Cesarean section on Mar. 15, 2018 at the Military Hospital 103, Vietnam Military Medical University. The patient was anesthetized by spinal anesthesia with 8 mg of bupivacaine plus 20 mcg fentanyl in the left lateral position at L2-L3 level. Point time of spinal block: 11 h AM.

Onset time: 3 mins.

Anesthesia effect was very good for surgery.

Case Reports T3 at 10 mins after the spinal block.

Duration of surgery: 50 mins.

Hemodynamic was stable during the surgery.

Surgery was completed at 11 h 55, meaning at 60 mins after the spinal block.

Lipid emulsion (Lipofundin 20%) bolus injection was started at 12 h 05 mins over 3 mins with the dose of 1.5 mg/kg (patient's weight 60 kg), then continuous infusion with 150 mi LE over 10 mins (0.25 mg/kg/min). Total volume of Lipid emulsion was 250 ml. Patient was monitored closely in the operating theatre during the LE treatment.

TABLE 6

| | From 12 h 05 | | | | | | | | |
|---|---|---|---|---|---|---|---|---|---|
| | (Lipid bolus | | 12 h | 12 h | 12 h | | | | |
| | injection) To | 12 h 08 | 10 | 15 | 18 | 12 h | 12 h | 13 h | 13 h |
| | 12 h 08 | Continuous infusion | | | | 26 | 53 | 07 | 10 |
| Bromage in | 3 | Little slight | 2 | 2 | 1 | 1 | 0 | 0 | 0 |
| the Right leg | | movement | | | | | | | |
| Bromage in | 3 | 3 | 3 | 3 | 3 | 1 | 1 | 0 | 0 |
| Left leg | | | | | | | | | |
| Sensory level | T4 | T4 | T5 | T6 | T8 | T8 | T12 | T12 | T12 |
| Mins after | | 3 | 5 | 10 | 13 | 18 | 35 | 48 | 62 |
| finishing | | | | | | | | | |
| bolus | | | | | | | | | |
| injection | | | | | | | | | |
| Mins after | 65 | 68 | 70 | 75 | 78 | 84 | 113 | 127 | 120 |
| spinal block | | | | | | | | | |

Only 1-2 minutes after starting the bolus injection, the patient could move slightly the right foot. The left foot could be moved slightly after finishing the bolus of the Lipofundin 20% injection at 8 mins. The patient got a full recovery movement function at 48 minutes after finishing the bolus Lipofundin injection without any pain (VAS=0) although sensory block was reduced to T12 (Table 7).

TABLE 7

| | From 12 h 05 | | | | | | | | |
|---|---|---|---|---|---|---|---|---|---|
| | (Lipid bolus | | 12 h | 12 h | 12 h | | | | |
| | injection) To | 12 h 08 | 10 | 15 | 18 | 12 h | 12 h | 13 h | 13 h |
| | 12 h 08 | Continuous infusion | | | | 26 | 53 | 07 | 10 |
| Bromage in | 3 | Little slight | 2 | 2 | 1 | 1 | 0 | 0 | 0 |
| the Right leg | | movement | | | | | | | |
| Bromage in | 3 | 3 | 3 | 3 | 3 | 1 | 1 | 0 | 0 |
| Left leg | | | | | | | | | |
| Sensory level | T4 | T4 | T5 | T6 | T8 | T8 | T12 | T12 | T12 |
| Mins after | | 3 | 5 | 10 | 13 | 18 | 35 | 48 | 62 |
| finishing | | | | | | | | | |
| bolus | | | | | | | | | |
| injection | | | | | | | | | |
| Mins after | 65 | 68 | 70 | 75 | 78 | 84 | 113 | 127 | 120 |
| spinal block | | | | | | | | | |

### Videos:

Starting bolus lipofundin injection: https://youtu.be/szUw4o3R3Oo

1 minute after starting bolus injection: https://youtu.be/LSSI5v9Tu-w

Finish bolus lipofundin injection: https://youtu.be/PIhxJ3-zfA

1 minute after finishing bolus injection: https://youtu.be/FNKKJUOVWOo

2 minutes after finishing bolus injection: https://youtu.be/xjS_PjtFpzY

4 minutes after finishing bolus injection: https://youtu.be/PDGEQYj11xY

5 minutes after finishing bolus injection: https://youtu.be/9TZGxqC-GOA

7 minutes after finishing bolus injection: https://youtu.be/mzR96YM637g

8 minutes after finishing bolus injection: https://youtu.be/pfVsS2ChjAI

10 minutes after finishing bolus injection: https://youtu.be/roGW5YOAbK8

11 minutes after finishing bolus injection: https://youtu.be/QvZrAqBcBnY

12 minutes after finishing bolus injection: https://youtu.be/ejFnL4rPamQ

15 minutes after finishing bolus injection: https://youtu.be/vOk8LoBDHvo

18 minutes after finishing bolus injection: https://youtu.be/ucmm8nfFqeo

21 minutes after finishing bolus injection: https://youtu.be/msGDpCzQrIs

25 minutes after finishing bolus injection: https://youtu.be/2L335DHPd-k

28 minutes after finishing bolus injection: https://youtu.be/Vo4vNlcgfV8

31 minutes after finishing bolus injection: https://youtu.be/sW0TI8PJSMk

43 minutes after finishing bolus injection: https://youtu.be/YjNbSJuwd4Q

45 minutes after finishing bolus injection: https://youtu.be/auPwEHaMMO8

48 minutes after finishing bolus injection: https://youtu.be/ZRFH2B7oDxk

59 minutes after finishing bolus injection: https://youtu.be/EQs5ggSEEls

62 minute2 after finishing bolus injection: https://youtu.be/L4U-WrQ6qnQ

### Discussion

Corning, a neurologist, was the first to attempt spinal anesthesia, although not in any way as we understand it today [1]. He was under the false impression that injecting cocaine between the spinous processes would result in rapid transportation of the drug to the spinal cord, thus producing anesthesia of the cord. Corning's experiments were carried out in a man and a dog. The man, receiving approximately 120 mg of cocaine which is about four times the lethal dose, was certainly lucky to survive the experiment and what was achieved was probably epidural anesthesia [2]; the dog, receiving approximately 13 mg, presumably had spinal anesthesia [2].

Both general and spinal anaesthesia with short-acting local anaesthetics are suitable and reliable for knee arthroscopy as an ambulatory procedure. Chloroprocaine (CP) 1% seems to be the ideal spinal local anaesthetic for this indication.

The aim of this study was to compare spinal anaesthesia using CP 1% with general for outpatient knee arthroscopy with regard to procedure times, occurrence of pain, patient satisfaction and recovery, and also costs [3].

A randomised controlled single-centre trial. University Medical Centre Mannheim, Department of Anaesthesiology and Surgical Intensive Care Medicine, Mannheim, Germany. April 2014 to August 2015.

A total of 50 patients (women/men, 18 to 80 years old, ASA I to III) undergoing outpatient knee arthroscopy were included. A contra-indication to an allocated anaesthetic technique or an allergy to medication required in the protocol led to exclusion.

Either general anaesthesia with sufentanil, propofol and a laryngeal mask for airway-management or spinal with 40-mg CP 1% were used. We noted procedure times, patient satisfaction/recovery and conducted a 7-day follow-up[3].

Primary outcome was duration of stay in the day-surgery centre. Secondary outcomes were first occurrence of pain, patient satisfaction, quality of recovery and adverse effects. In addition, we analysed treatment costs.

Spinal had faster recovery than general anaesthesia with patients reaching discharge criteria significantly earlier [117 min (66 to 167) versus 142 min (82 to 228), P=0.0047]. Pain occurred significantly earlier in the general anaesthesia group (P=0.0072). Costs were less with spinal anaesthesia (cost ratio spinal:general 0.57). Patients felt significantly more uncomfortable after general anaesthesia (P=0.0096). Spinal anaesthesia with 40 mg CP 1% leads to a significantly earlier discharge and is cheaper compared with general [3].

Bupivacaine is an amide local anesthetic with a slow onset (5-10 minutes, longer with isobaric forms). It is a long acting spinal anesthetic appropriate for procedures that last 2-2.5 hours. It is comparable to tetracaine; however, tetracaine exhibits a more profound motor block and increased duration when vasoconstrictors are added. Available hyperbaric forms include concentrations of 0.5% and 0.75%, with dextrose 8.25%. Isobaric formulations are available in concentrations of 0.5% and 0.75%. When using isobaric solutions, the total mg dose is more important than the total volume of medication administered.

Spinal anesthesia (SPA) has not been popular for daycase surgery because of prolonged neurologic blockade with long-acting local anesthetics such as bupivacaine, thereby delaying discharge. Although the intermediate duration of action of lidocaine and mepivacaine appears to be more suitable for day-case surgery, their use is not deemed appropriate by many because of a high incidence of transient neurologic symptoms (TNSs).

Prilocaine has a similar intrathecal pharmacokinetic profile as lidocaine but with a significantly lower risk of TNSs. Onset of spinal after 2-chloroprocaine is comparable with lidocaine or prilocaine, but with a considerably shorter duration of action. Also, TNS is clearly less frequent compared with lidocaine. Although its intrathecal use has recently been approved in Europe, this is still considered to be off-label in the USA. Articaine provides an extraordinary fast onset and a short duration of spinal block, the latter being approximately intermediate between chloroprocaine and prilocaine. However, articaine is associated with a high risk for intraoperative hypotension and a small risk for TNS, albeit but less frequent than after lidocaine. Concerns regarding possible neurotoxicity of articaine remain to be resolved.

SPA for day cases might become a most valuable method for ambulatory surgery when using short acting local anesthetics. This, however, not only depends on drugs being used but also on infrastructure (post anaesthesia care unit) and organizational issues [4].

An increasing number of day-case surgical patients is challenging the presently used methods of anaesthesia: reliable surgical anaesthesia should be fast, with rapid recovery and minimal side effects. To compete with modern ambulatory general anaesthesia a knowledge of special spinal anaesthesia techniques is essential. For surgical procedures in one lower limb, a low dose of hyperbaric bupivacaine with standardized spinal anaesthesia technique produces a reliable block, with low incidence of side effects and home-readiness equal to spinal anaesthesia with lidocaine (50 mg) or general anaesthesia (desflurane), whereas ropivacaine has not shown benefits over spinal anaesthesia with bupivacaine. ‘Walk-in, walk-out’ spinals with an extremely low dose of lidocaine and opioids for gynaecological laparoscopy created the concept of selective spinal anaesthesia. Reintroduction of chloroprocaine may provide a solution for bilateral, short-acting spinal anaesthesia in the future.

To produce reliable spinal anaesthesia with a reasonable recovery time it is essential to understand the factors affecting the spread of spinal block and to choose the optimal drug and adequate dose for specific surgical procedures [5].

The local anaesthetics used in day-case spinal anaesthesia should provide short recovery times. We aimed to compare hyperbaric prilocaine and bupivacaine in terms of sensory block resolution and time to home readiness in day-case spinal anaesthesia[6]. Fifty patients undergoing perianal surgery were randomized into two groups. The bupivacainefentanyl group (Group B) received 7.5 mg, 0.5% hyperbaric bupivacaine+20 μg fentanyl in total 1.9 mL. The prilocainefentanyl group (Group P) received 30 mg, 0.5% hyperbaric prilocaine+20 μg fentanyl in the same volume.

Time to L1 block and maximum block was shorter in Group P than in Group B (Group P 4.6±1.3 min versus Group B 5.9±01.9 min, P=0.017, and Group P 13.2±7.5 min versus Group B 15.3±6.6 min, P=0.04). The time to L1 regression and S3 regression of the sensorial block was significantly shorter in Group P than in Group B (45.7±21.9 min versus 59.7±20.9 min, P=0.024, and 133.8±41.4 min versus 200.4±64.8 min, P<0.001). The mean time to home readiness was shorter for Group P than for Group B (155±100.2 min versus 207.2±62.7 min (P<0.001)).

Day-case spinal anaesthesia with hyperbaric prilocaine+fentanyl is superior to hyperbaric bupivacaine in terms of earlier sensory block resolution and home readiness and the surgical conditions are comparable for perianal surgery [6].

The incidence of perianal surgery varies among institutions, accounting for up to 10% of general surgical procedures [7]. The procedure is suitable to perform on a day-case basis with spinal anaesthesia [8,9]. However, prolonged sensory and motor block and urinary retention can cause a delay in discharge [10,11]. Day-case spinal anaesthesia with short-acting local anaesthetics such as lidocaine and chloroprocaine can provide short times to discharge [12,13]. However, the association of lidocaine with transient neurologic symptoms (TNS) and chloroprocaine with neurologic injury has limited the use of these agents in spinal anaesthesia [14,15]. Bupivacaine is safe with a very low incidence of associated TNS, but the prolonged sensory and motor block are a disadvantage for day-case spinal anaesthesia[16]. The use of small doses of bupivacaine with the addition of opioids is proposed to enhance the recovery of the spinal block [17].

Spinal anaesthesia when compared to general anaesthesia has been shown to decrease postoperative morbidity in orthopaedic surgery. The aim of the present study was to assess the differences in thirty-day morbidity and mortality for patients undergoing hip fracture surgery with spinal versus general anaesthesia [18].

The American College of Surgeons National Surgical Quality and Improvement Program (NSQIP) database was used to identify patients who underwent hip fracture surgery with general or spinal anaesthesia between 2010 and 2012 using CPT codes 27245 and 27244. Patient characteristics, complications, and mortality rates were compared. Univariate analysis and multivariate logistic regression were used to identify predictors of thirty-day complications. Stratified propensity scores were employed to adjust for potential selection bias between cohorts.

6133 patients underwent hip fracture surgery with spinal or general anaesthesia; 4318 (72.6%) patients underwent fracture repair with general anaesthesia and 1815 (27.4%) underwent fracture repair with spinal anaesthesia. The spinal anaesthesia group had a lower unadjusted frequency of blood transfusions (39.34% versus 45.49%; p<0.0001), deep vein thrombosis (0.72% versus 1.64%; p=0.004), urinary tract infection (8.87% versus 5.76%; p<0.0001), and overall complications (45.75% versus 48.97%; p=0.001). The length of surgery was shorter in the spinal anaesthesia group (55.81 versus 65.36 min; p<0.0001). After multivariate logistic regression was used to adjust for confounders, general anaesthesia (odds ratio, 1.29; 95% confidence interval, 1.14-1.47; p=0.0002) was significantly associated with increased risk for complication after hip fracture surgery. Age, female sex, body mass index, hypertension, transfusion, emergency procedure, operation time, and ASA score were risk factors for complications after hip fracture repair (all p<0.05).

Patients who underwent hip fracture surgery with general anaesthesia had a higher risk of thirty-day complications as compared to patients who underwent hip fracture repair with spinal anaesthesia. Surgeons should consider using spinal anaesthesia for hip fracture surgery [18].

Spinal anaesthesia is an easy and reliable technique. Factors limiting its use in the ambulatory setting include delayed ambulation, risk of urinary retention and pain after block regression. On the contrary, general anaesthesia with fast-acting drugs provides a fast recovery that facilitates an early discharge. Although recovery after spinal anaesthesia has been improved by reducing the dose of the commonly used long acting local anaesthetics, discharge times are still prolonged compared with general anaesthesia. 2-Chloroprocaine is an amino-ester local anaesthetic with a very short half-life and a favourable evolution of spinal block for ultra-short outpatient procedures. Moreover, the preservative free 2-chloroprocaine solution showed a very low risk of urinary retention and transient neurological symptoms when compared with bupivacaine and lidocaine[19].

We compared the costs related to the clinical effectiveness of general anesthesia versus spinal anesthesia in inguinal hernioplasty ambulatory surgery[20]. An observational, retrospective cohort study measurement and analysis of cost-effectiveness, in the ambulatory surgery unit of a general hospital. All patients over 18 years of age diagnosed with primary inguinal hernia and scheduled for unilateral hernioplasty between January 2010 and December 2011 were included. Duration of anesthetic induction, length of stay in both the operating room, and in the post-anesthesia care unit, the anesthetic effectiveness (the incidence of adverse effects and the patient's comfort level), and variable economic costs associated with the use of drugs, as well as the use of human resources, were compared.

The final analysis included 218 patients, 87.2% male, with a mean age of 53 years (range: 18-85 years). Of these, 139 (63.76%) received subarachnoid anesthesia and 79(36.2%) general anesthesia. The length of time a patient remained in the post-anesthesia care unit was 337.6±160.2 min in the subarachnoid anesthesia group, and 210.0±97.5 min for the general anesthesia group (P<0.001). Costs of drugs for general anesthesia were higher than that for subarachnoid anesthesia (86.2±8.3 vs. 18.7±7.2). The total cost difference between the 2 techniques was €115.8 more for subarachnoid anesthesia (P<0.001).

Both techniques showed similar effectiveness. The overall costs for subarachnoid anesthesia were greater than for the general. The cost-effectiveness of general anesthesia is better for outpatient inguinal hernia repair surgery [20].

Length of stay after total hip arthroplasty (THA) has decreased over the last two decades. However, published studies that have examined same-day and early discharge protocols after THA have been done in highly selected patient groups operated on by senior surgeons in a nonrandomized fashion without control subjects.

The purpose of this study was to evaluate and compare patients undergoing THA who are discharged on the same day as the surgery (“outpatient,” less than 12-hour stay) with those who are discharged after an overnight hospital stay (“inpatient”) with regard to the following outcomes: (1) postoperative pain; (2) perioperative complications and healthcare provider visits (readmission, emergency department or physician office); and (3) relative work effort for the surgeon's office staff [21].

A prospective, randomized study was conducted at two high-volume adult reconstruction centers between July 2014 and September 2015. Patients who were younger than 75 years of age at surgery, who could ambulate without a walker, who were not on chronic opioids, and whose body mass index was less than 40 kg/m2 were invited to participate. All patients had a primary THA performed by the direct anterior approach with spinal anesthesia at a hospital facility. Study data were evaluated using an intentionto-treat analysis. A total of 220 patients participated, of whom 112 were randomized to the outpatient group and 108 were randomized to the inpatient group. Of the 112 patients randomized to outpatient surgery, 85 (76%) were discharged as planned. Of the remaining 27 patients, 26 were discharged after one night in the hospital and one was discharged after two nights. Of the 108 patients randomized to inpatient surgery with an overnight hospital stay, 81 (75%) were discharged as planned. Of the remaining 27 patients, 18 met the discharge criteria on the day of their surgery and elected to leave the same day, whereas nine patients stayed two or more nights.

On the day of surgery, there was no difference in visual analog scale (VAS) pain among patients who were randomized to discharge on the same day and those who were randomized to remain in the hospital overnight (outpatient 2.8±2.5, inpatient 3.3±2.3, mean difference −0.5, 95% confidence interval [CI], −1.1 to 0.1, p=0.12). On the first day after surgery, outpatients had higher VAS pain (at home) than inpatients (3.7±2.3 versus 2.8±2.1, mean difference 0.9, 95% CI, 0.3-1.5, p=0.005). With the numbers available, there was no difference in the number of reoperations, hospital readmissions without reoperation, emergency department visits without hospital readmission, or acute office visits. At 4-week follow up, there was no difference in the number of phone calls and emails with the surgeon's office (outpatient: 2.4±1.9, inpatient: 2.4±2.2, mean difference 0, 95% CI, −0.5 to 0.6, p=0.94).

Outpatient THA can be implemented in a defined patient population without requiring additional work for the surgeon's office. Because 24% (27 of 112) of patients planning to have outpatient surgery were not able to be discharged the same day, facilities to accommodate an overnight stay should be available [21].

### Conclusion

A 46 years old Male was diagnosed with penis fracture due to sexual intercourse. A 72 years old Male was diagnosed as having a tumor on the upper pole of the femur. A 41 years old Female had a Cesarean section. All of them underwent spinal anesthesia for their operations. All of them underwent at the end of their operations Local anesthesia reversal (LAR) of Spinal anesthesia by Lipid Emulsion (Lipofundin 20%) using Bolus and Infusion. The local anesthesia reversal was started in approximately 3 minutes after starting the bolus injection and completed in approximately 30 minutes afterwards.

This new modality of LAR can make a great change in the use of spinal anesthesia in day case surgery facilities.

It is the first time in the medical literature that spinal anesthesia motor block is successfully reversed by Lipid Emulsion (Lipofundin 20%) bolus and infusion.

What is claimed is:

1. A multidisciplinary and multipotent intralipid-iodine for use in imaging.

2. The intralipid-iodine according to claim 1, wherein said imaging is hysterosalpingography in infertile women.

3. The intralipid-iodine according to claim 1, wherein said imaging is X-ray imaging or cardiac- and limbs-catheterization.

4. The intralipid-iodine according to claim 1, wherein said intralipid-iodine is formulated for injection into the blood stream of a patient.

5. The intralipid-iodine according to claim 1, which is any lipid emulsion.

6. The intralipid-iodine according to claim 1, wherein said intralipid-iodine absorbs/attaches obstructive or potentially-obstructive particles located within blood vessels and/or other organs in the body, and removes same to thereby open clogged blood vessels.

7. A multidisciplinary and multipotent composition comprising intralipid-iodine for use in imaging.

8. The composition according to claim 7, further comprising a pharmaceutically acceptable carrier adapted for injection.

9. The composition according to claim 7, which is formulated for administration into the blood stream.

10. The composition according to claim 7, wherein said intralipid-iodine is any lipid emulsion.

11. The composition according to claim 7, wherein said intralipid-iodine absorbs/attaches obstructive or potentially-obstructive particles located within blood vessels and/or other organs in the body, and removes same to thereby open clogged blood vessels.

12. A method for imaging comprising administering to an individual in need thereof an effective amount of the intralipid-iodine of claim 1.

13. The method according to claim 12, comprising injecting said intralipid-iodine or said composition comprising same into the blood stream of said individual.

14. The method according to claim 12, wherein said intralipid-iodine is any lipid emulsion.

15. The method according to claim 12, wherein said intralipid-iodine absorbs/attaches obstructive or potentially-obstructive particles located within blood vessels and/or other organs in the body, and removes same to thereby open clogged blood vessels.

16. An intravenous composition comprising the intralipid-iodine of claim 1 for use in imaging.

17. The composition according to claim 16, further comprising a pharmaceutically acceptable carrier adapted for injection.

18. The composition according to claim 16, wherein said intralipid-iodine absorbs/attaches obstructive or potentially-obstructive particles located within blood vessels and/or other organs in the body, and removes same to thereby open clogged blood vessels.
